SEQUENCE LISTING (79 sequences)

SEQ ID NO: 1 - DNA, 9189 bp, Caprine arthritis-encephalitis virus
gagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctggcgcccaacgtggggcccgaggagaagaaaagaaagcggccctgagaactcggcttctgaaaaagaggaagaggacaagttgctatagcaacaagagagaagaagtagagcaaaggtccagtggctcggaaaaagaggaactgaaacttcggggacgcctgaaggagtaaggtaagtgactctgctgtacgcggggcgaggcagaggtttccttctaaattgaaagagaagtgttgctgcgagaggtcttggtggtcgagaatcctgtacaaaaaaaaggagggatctcggtcaggaccaggacccctgggagtaatacaacagcaacaccgtaagaaaatccgccatggtgagtctagatagagacatggcgaggcaagtctccggggggaaaagagattatcctgagctcgaaaaatgtatcaagcatgcatgcaagataaaagttcgactcagaggggagcacttgacagaaggaaattgtttatggtgccttaaaacattagattacatgtttgaggaccataaagaggaaccttggacaaaagtaaaatttaggacaatatggcagaaggtgaagaatctaactcctgaggagagtaacaaaaaagactttatgtctttgcaggccacattagcgggtctaatgtgttgccaaatggggatgagacctgagacattgcaagatgcaatggctacagtaatcatgaaagatgggttactggaacaagaggaaaagaaggaagacaaaagagaaaaggaagagagtgtcttcccaatagtagtgcaagcagcaggagggagaagctggaaagcagtagattctgtaatgttccagcaactgcaaacagtagcaatgcagcatggcctcgtgtctgaggactttgaaaggcagttggcatattatgctactacctggacaagtaaagacatactagaagtattggccatgatgcctggaaatagagctcaaaaggagttaattcaagggaaattaaatgaagaagcagaaaggtggagaaggaataatccaccacctccagcaggaggaggattaacagtggatcaaattatgggggtaggacaaacaaatcaagcagcagcacaagctaacatggatcaggcaaggcaaatatgcctgcaatgggtaataaatgcattaagagcagtaagacatatggcgcacaggccagggaatccaatgctagtaaagcaaaaaacgaatgagccatatgaagattttgcagcaagactgctagaagcaatagatgcagagccagttacacagcctataaaagattatctaaagctaacactatcttatacaaatgcatcagcagattgtcagaagcaaatggatagaacactaggacaaagagtacaacaagctagtgtagaagaaaaaatgcaagcatgtagagatgtgggatcagaagggttcaaaatgcaattgttagcacaagcattaaggccaggaaaaggaaaagggaatggacagccacaaaggtgttacaactgtggaaaaccgggacatcaagcaaggcaatgtagacaaggaatcatatgtcacaactgtggaaagagaggacatatgcaaaaagaatgcagaggaaagagagacataaggggaaaacagcagggaaacgggaggagggggatacgtgtggtgccgtccgctcctcctatggaataacttcagcaccacctatggttcaggtccgcataggttcccagcagaggaacttgttatttgataccggggcggaccgaactatagttagatggcatgagggctcgggaaacccagccggaaggataaaactgcaaggaataggaggaatagtagaaggagaaaaatggaataatgtagaattagaatataaaggagaaacaagaaagggaacaatagtagtgttaccacaaagtccagtagaagtattaggacgagataacatggcccgatttggaataaagataataatggcaaatttagaggaaaaaagaatcccaattacaaaagtaaaattgaaagagggatgtacgggtccacatgtcccacaatggccattaacagaagagaaattaaaaggtctaacagaaatcatagataaattagtggaagaaggaaaactaggaaaggcacccccacattggacatgtaatactccaatcttttgcataaaaaagaaatcagggaagtggagaatgttaatagatttcagagaattgaacaaacagacagaagatttaacagaagcgcagttaggactcccgcatccgggaggactacaaaagaaaaaacatgttacaatattggacataggagatgcatattttactatacccctatatgaaccatatcgagagtacacatgttttactctattaagtcctaataatctaggaccatgtaaaagatactattggaaagtgctgccacaaggttggaaattgagtccatctgtatatcaatttactatgcaggagatcttagaggattggatacagcagcatccagaaattcaatttggcatatatatggatgatatttacataggaagtgatttagaaattaaaaagcatagagaaatagtgaaagatttagccaattatattgcccaatatggattcactctgccagaagagaagagacaaaagggatatccagcaaaatggctaggatttgaactacacccgcagacctggaaatttcagaagcatacattacctgaattaacaaagggaacaataacattaaataaattacagaaattagtaggagaattagtatggagacaatccataattgggaaaagcattcctaacattctgaaattaatggaaggagatagagaattacaaagtgaaagaaaaattgaagaagtacatgtgaaagaatgggaagcatgtaggaaaaaattagaagaaatggaaggaaattattataataaagacaaagatgtctatggacaattggcttggggagacaaagctatagaatatatagtgtatcaggagaaagggaaaccattatgggtaaatgtggttcacaatataaagaacctaagcatcccgcaacaggttattaaagcagcgcaaaaattaacccaagaagtcatcattaggacaggaaaaataccatggatattgttgccagggaaagaagaagattggagactagaattgcaattagggaacatcacatggatgccaaaattttggtcctgttatcgaggacatacaagatggagaaaaagaaatataatagaagaagtagtagaagggcctacatattatacagatggaggaaaaaagaataaagtaggaagtctagggttcatagtatcaacaggggaaaaatttagaaagcatgaagagggcacaaaccagcaactagaattaagagccatagaggaagctctaaaacaagggcctcaaacaatgaatttagtaacagatagtagatatgcatttgaatttttattaagaaattgggatgaagaagtaataaagaatccaattcaagcaagaattatggaaattgcccacaagaaagataggataggagtgcattgggtgccaggacataaagggattccccaaaatgaagaaatagacaaatatatttcggaaatatttcttgcaaaagaaggagaaggaattctcccaaaaagagaagaggatgcagggtatgatttaatatgcccagaagaggttaccatagagccaggacaagtgaaatgcatccccatagagctaagattaaatttaaagaaatcacaatgggctatgattgctacaaaaagcagcatggctgccaaaggagtgttcacacaaggaggaatcatagactcaggatatcagggacaaatacaggtaataatgtataatagcaataaaatagcagtagtcataccccaagggagaaaatttgcacaattaatattaatggataaaaagcatggaaaattggaaccctggggggaaagcagaaaaacagaaaggggagaaaaaggatttgggtctacaggaatgtattggatagaaaatattcctctggcagaggaagaccacacaaaatggcatcaagatgcccgatcattgcatctagaatttgaaattccaagaacagcagcagaagacatagtaaatcaatgtgaaatatgcaaagaagcgaggacacctgcagtaattagaggcggaaacaaaaggggggtaaatcattggcaagtggattatacccattatgaaaatatcatactattagtatgggtagaaacaaattcaggactaatatatgcagaaaaagtaaaaggagaatcagggcaagaattcagaataaaagtgatgcattggtatgcattatttggtccagagtcattgcagtcagacaatggacctgcatttgcagcagagcccacacagctgttaatgcaatacctaggagtaaaacacacaacaggcataccttggaatccacagtctcaggctatagtagaaagggcacatcaactattgaaaagcactttaaagaagttccagccacaatttgtcgctgtagaatcagccatagcagcagccctagtcgccataaatataaaaagaaagggtgggctggggacaagccctatggatatttttatatataataaagaacagaaaagaataaataataaatataataaaaattctcaaaaaattcaattctgttattacagaataaggaaaagaggacatcaggagagtggaaaggaccaacccaggtactgtggaaaggggaaggagccaattgtggtaaaggatatagaaagtgaaaagtatttagtaataccttacaaagatgcaaaattcatcccgccaccaacaaaagaaaaggaataaaaaacctggaccagaattacccttagcactatggatacatatagcagaaagcattaatggggatagctcatggtacataacaatgagactgcaacagatgatgtggggaaaaagaggaaataagttacaatataagaatgaagacagggaatatgaaaattgggaaattacatcatggggatggaaaatgcacctaaggagagtgaaacaatggatacaagacaacaggagaggaagcccatggcagtacaaagtaggaggaacatggaaaagtataggagtgtggttcctgcaagcaggagattacagaaaggtagacaggcacttctggtgggcatggaggatactgatatgttcctgcaggaaagaaaagtttgatataagagaatttatgagaggaagacatagatgggatttgtgcaaatcctgtgctcaaggagaagtagtaaagcatactagaacaaaaagtctggaaagactagtactgctacagatggtagaacagcatgtgtttcaagtattgccattgtggagagccaggagaagtagtacaacagatttcccatggtgcagggacacaacgggatacacgcatgcgtggtctgtccaggagtgctggttgatggaatatctcttagaggatgagtgaagaactgcctcaaagaagggagacacatccagaagaacttgtaaggaacgtacgggaaagagaaagggatacatggcaatggacaagcatcagagtacctgcggaaatactgcaaagatggcttgctatgcttaggtcaggcagaaatagaaagaaagtgtatagagaaatgcaaaaatggatgtggatacatcccaaggcgcctgtgattagggcctgtggatgcagactatgtaacccggggtggggaacataatcaagggaataataaatgcaaataaatgtaactaacaagtagcaaaagtgtctgtgttagatggatgctggggccagatacatgcgcttaactgggaaggaaaactgggttgaagtaaccatggacggagagaaggaaaggaaaagagaaggtttcactgcgggacagcaaggtaagtatcaaccccaggtaagtaagcaaatagggaacagaaatactaacccatgctttgcctataaagggatattcctatggaggatatcactaacaatgtggatattgctagggataaatatgtgtgtcagtgcagaggattacataacactaatatcagatccctatgggttctcacccataaaaaatgtgtctggggtaccagtgacttgtgtaacaaaagaattcgcaaaatggggatgtcaaccactaggagcgtaccctgatccagaaatagaatacagaaatgtgagtcaggaagtagtgaaagaagtatatcaagagaattggccatggaatacatatcattggcctctctggcaaatggagaatgttaggtactggttaaaagaaaatatgcaagaaaatcaacagagaaaaaataatacaaaagagggtatagaggaattattagcaggaactataaggggaagattctgtgtaccatacccatttgccttgttaaaatgcacaaagtggtgctggtatacagcggccataaacaacgagtcaggaaaagcaggaaaaataaaaataaattgcacagaagcaagagcagtctcctgtacagaggacatgccattagcctcaatacaaagagcatattgggatgagaaagacagagagagcatggcctttatgaatatcaaagcatgtgatagcaacctaaggtgtcagaaaagacctggagggtgtatggaaggataccctatcccagtaggagcagaaataatccctgaaagtatgaaatacctaaggggagcaaagagtcagtatgggggaataaaagataagaatggagaattaaaattaccattaacattaagagtgtgggtaaaattagcaaatgtgtcagaatgggtaaatgggacacccccggattggcaagacagaattaacggatccaaaggaataaatgggacgctctggggagagcttaacagtatgcatcacctaggatttgcccttagccagaacggcaaatggtgtaactacaccggggaaataaaattagggcaagaaacattccaatatcattacaagccaaactggaactgtaccgggaattggacgcaatatccggtgtggcaagtgattagaaacctggatatggtggaacatatgacaggagaatgtgtgcagagaccacaaaggcacaatataacagtaggaaatggaaccataacagggaattgcagtacaacaaactgggatggatgtaattgctcacgatcaggaaactacctatataacagctctgagggaggattgttattaattctgtgcagacaaaacagcaccctaacaaggatcctgggaacaaatacaaattggacaactatgtggggaatatacaaaaattgttcaggatgcgagaatgcaacattagacaacacaggagaaggaaccttaggaggtgtagctaataagaactgtagcttgcctcataaaaatgagagcaacaagtggacttgtgccccaagacaaagagatggaaaaacagattcgctatacatagcaggaggaaaaaagttttggacacgaattaaggcccaattcagctgtgaaagtaacataggacaattagatggaatgttgcatcagcaaatactattgcaaaaatatcaagtaattaaggtaagagcttatacatatggggtgatagaaatgccagaaaactatgcaaaaacaagaatcataaacaggaaaaaaagagaactcagccacaagaggaagaagagaggcgttggcttggtcattatgctagttatcatggcaatagtagctgccgcaggggcttctctgggagtcgcaaacgcgattcagcagtcttacactaaggcagctgtccagacccttgctaatgcaactgctgcacagcaggatgtgttagaggcaacctatgccatggtacagcatgtggctaaaggcgtacgaatcttggaagctcgagtggctcgagtggaagctatcacagatagaataatgctataccaagaattggattgttggcactatcatcaatactgtataacctctacaaaaacagaagtagcaaaatatatcaattggacgaggtttaaggataattgcacatggcagcagtgggagagaggattacaggggtatgatacaaacttaacaatactgttaaaggaatcagcagcaatgacacaactagcagaagagcaagcaaggaggataccagaagtatgggaaagtttaaaagacgtctttgattggtcaggatggttctcatggctaaagtatattcctattatagtagtaggattattaggatgcattctgataagagctgtgatatgtgtatgtcaacctcttgtgcagatatacagaactctaagtaccccgacataccaacgggtcacagtcatcatggaaacaagagcagacgtcgcaggagaaaatcaggattttggcgatggcttagaggaatcagacaacagcgaaacaagcgaaagagtgacagtacagaaagcttggagccgtgcctgggagctttggcagaactcaccctggaaggagccatggaaaaggggcctgctgaggctgctcgtccttccgctgacgatgggaatctggataaatggatggcttggagaacaccacaaaaataaaaaaagaaagggtgactgtgagacatgggctaaagaggactaataacaagctaggccaaattcctgtaaatcacttggggggttataagaaaagcaagttcactatgacaaagcaaaatgtaaaggccaaattcctgtaaatcacttggggggttataagaaaagcaagttcactatgacaaagcaaaatgtaaccgcaagtgctgacagatgtaacagctgacatatcagctgatgcttgctcatgctgacactgtagctctgagctgtatataaggagaagcttgctgcttgcacttcagagttctaggagagtccctcctagtctctcctctccgaggaggtaccgagacctcaaaataaaggagtgattgccttactgccga

SEQ ID NO: 2 - DNA, 8919 bp, Caprine arthritis-encephalitis virus
gtgagtgctctgaggagctcgaaggaaagagtcctcagcctctcctctccgaggagcttcggctcataataaaggagtgcttgcttcaacagaactgagctggtcgtggttattatcggggaccgaagtcccgtgcaacaccggggcggttctcgcagctggcgcccaacgtggggctcgagtagcttgagaagctcgactgagatctgaatccaagagcgacatcagacagcaagaaatgagagtaatgagaccgcgagctctgctgctgtaaaaaagaggaagtagcgggttgccgaggcaactgctcagaagaaccaggggaaagggcttccagcaacctcaaaagaggaaccgagacttcggggacgcctgaagtaaggtaagtgactctgctgtacgcggggcgaggcataggagatccttctattctaggaagagaagcgctgttctgggaggtcttggcgaccgagaatcttgttaaataagccaggatctcgatcaggaccaagacccctcaggagagggtatagacagcgtggtaagaaatccgccgtggtgagtctagatagagacatggtgaggcaggcctccggaaggggaaaggagtaccccgagctaaaagaatgtctgaaaaaggcatgcaaaataaaagtaagggctgggggggagcgcctgacagaaggaaattgtctctggtgtataaaaacactagagtgtatgtatgaggattgtagggaggaaccttggaccccagaaaaatgtaaacaattatggaaaaagttgaagcaggtagagcctgaggagagtagcaaagcagactataactcgttaaaagcaaccttggcggggatagtctgtgtgcaaatgggaatgcagcccgagacactgcaggatgcgatagcaaccttaaacatgagagatgaagtaaaaggaaaggaaaagccatcagaagaaaagaagggaatatatcccatattagtgcaggcaggaggaggaagagcatggagagcggtagagcctgctacctttcagcagctccaaacagtggcaatgcagcatggactagtatcagaagaatttgaaaggcagctagcatactatgccaccacatggacaagcaaggatatcttagaagtattagccatgatgccaggaaatagagcgcaaaaagaactaatacaaggaaagttaaatgaggaagcagagagatggagaaggcagaatccacaacctgcgggcgggttaaccgtggatcagataatgggggtaggacaaacgaatcaggcagcggcacaggctaatatggatcaagcaagacaaatatgcctacaatgggttataacagcaataagaggagttaggcatatggcccatagaccaggaaatcccatgctggtaagacaaaaaccaaatgagaactatgaagagtttgccgcaaggttgttagaagcagtggatgcagaacccgttacccaacctataaaagaatatttaaaggtaactctgtcttacacaaatgcaaattcggaatgtcaaaaacatatggacagagtgttggggcaaagagtacagcaggcctcaatagaagaaaaaatgcaggcatgcagggacatcgggggaacagcatatcagatgcagttgcttgcacaagccctccgtggcggaaaagaagatgggaaaaaatctgtagggaagtgttataactgtggaaggcccggacacagagcaaaagaatgcagacaaggcattatatgtcacaactgtggaaaaagagggcatatacagaaaaactgcaaacagaaaagaagaaaggagcagggaaacatgaggagggggctacgtgtggtgccgtccgcaccccctatggagtaacgcaagcaccactaatagttagggtacaaatagggaatcaggagaaacaattattatttgacacaggggcagataaaacgatagtaagaatgcatgatggaacagggattccaaacggaagaataaaattacaagggataggaggaatagtagaaggagaaaaatggaataaagtacccatgacatataagggagaaacatcctgcccaagcttggttgtgctaagagatagcccagtagaagtattgggaagagataacatggaagcattcggcgtaaccctaataatggcaaatttagaagataagaaaattcccacaataccagtagaattgaaagaaggatgtaaagggccacatgtgccccagtggccattaacagcagagaaattacaaggactaacaggaatagtagaaaaattactacaggaaggaaaattggcagaggccccagagggatggacgtggaacacgcccatcttctgcataaaaaagaagtcaggaaaatggagaatgttaatagattttagggaattaaataagcaaacagcagatttagcagaagcgcagctaggactgccacacccaggagggttgcaaaggaaaaagaatgtaacaattctggacataggagatgcatatttcacaattcccttatacgagccctatcagaaatatacatgcttcacactcctaagtcctaacaatttgggaccatgtaaaaggtattattggaaagtattaccccagggatggaaattgagcccagctgtatatcaattcaccatgcaaaggttgttaaaaggatggatacaacagcataaaaacatacaatttggaatatatatggatgatatctatattggaagtgatctaacgatagcccaacataggaagataatagaagaattagcctcatttatagaacaatttgggtttacattaccagaagataagagacaagagggctatccagcaaaatggctaggattcgagctacatccagaaaaatggaaatatcaaaagcataaattgccggaattacaagagggggtaataaccctgaacaaattacagaagatagtaggggaattagtgtggagacaatccttgataggaaagagcatccccaatatcataaaattaatggaaggagatcgcgcattacaaagtgaaaggaaaatagaaagaatacatgtacaagaatgggaagcatgtcaaaagaaattagatgaaatggtaggaaattattacagagaagaagaagatatctatggacaaataacttggggggataaggcaataaaatacatagtattccaaaggaaaggggaacccctatgggtaaatgtagtacatgacataaaaaatttgagtctcccacagcaagtgataaaagcagcacagaaattaacccaggaagtaatcataagaacaggaaaaatcccatggctgctactaccaggaagagaagaagactggagattagaactgcaggtagggaacatcacgtggatgccatcattttggtcatgttatcgaggagcacccaagtggaaaagaaggaacatagtggcagcagtggtagatggaccgacatattatacagatgggggaaagaaaaacgcacagggaagctttggcttcatctccccaacaggagaaaagttcagaaggcatgaagatggaactaatcaggtattagaattaagggcaatagaagatccatgtaaacaaggacctgaaagcatgaacattgtaactgacagcaggtatgcttatgaattcatgctccgaaactgggatgaacaggtcataagaaaccccattcaggcaagaatcatggcagaagtgcacaagaaaaagcaggtaggaatacactgggtgccagggcataaaggaatacctcagaatgaagagatagaccagtacatatcagaagtattcttagcacgagaaggaacagggatatgtgaaaaaaggaaggaagatgctggatatgatttattatgcccgcatgaggtaatacttaaaccccaagaagtaaaacggatcccaatagacctaaaattaaaattgaaagaaaagcaatgggccatgataagtgggaaaagtagcgttgcagcaaaaggaatatttgtacaaggaggcataatagattcagggtatcagggacaagtacaagtcatcctatataatagtaataagatagaggtcaaaataccacaaggcaggaaatttgcccaattaatattaatgaacttacaacatgaagaattagaagaatggggaaaggaaagaaaaacagaaagaggaacaaaaggatttgggtctacaggagcattttggatagagaatattccccaagcagaggaagaacattacaaatggcatcaagatgctagatctctgcagctagaattcaagatacctagagcagcagcagaagacattatacagcactgtgaggtatgtcaagaaggcaaacccgcagcgatcacgagagggggaaataaaagaggaatagatcattggcaggtagactatacacattacaaagaacacataatattagtatgggtagagactaattcaggattaatatttgcagagaaagtaaaaggagaatcaggacaagaatttaggatgcagacattgaaatggtatgctttgtttcaaccaaaatcagtgcaatcagataatgggacagccttcacagctgaggctacgcagcatctaatgaagtatttagggattcagcacactacgggtattccgtggaacccccagtcacaaagtttagtagaaagagctcatcaaacattaaaacacatgttagaaaaattagaaccacaatttgtggccctacagtctgccatcgcagccactctagttgcgctcaatataaaaagaaagggtggactaggggcaagccctatggatatttacatatataataaggagcaacaaagacaacaagataatagtaataaattaattcagaaaaaattttgttattacaggatcagaaaaagaggccatccaggagagtggaacggcccaactgaggtactgtgggaaggggaaggagccatagtagttaaagacaaagaaagtgatagatatctagtcatcccatataaagatgcaaaatttattccgccaccgtcggaacagaagggatagaagaataggtccagaattgcctttatctttatggacttatacagcatacagcataaataaagatcccgcatggtatacaaccctaagactgcagcaaatgatgtggcataggaggggaaataaattgacatatgtcagggaaaatgcacagtacgaggagtgggaaatgacctcgtatgagtggaggataagaatgagaagggacaaaacaaaaagtcatccaagagggcatacttcgccatggcaatatcggagacaggatggatggaaggatgtgggaacgtggttcctacagccaggggactatagaaaggcggatcagcagttctggttcgcttggagaatagtgtcgtgttcatgtaaaaaggaaggatttaacataagagaatttatgctaggtacccatagatgggatttgtgtaagtcgtgttgccagggtgaagtagtaaagagaacacaaccctacaccttgcaaaggctcacgtggcttaaattaacagaagaccatgtatttcaagtaatgcccttgtggagagctcgcaaagggattaccatagactttccctggtgcagggacacaaaaggattcctggagccgtggacaacgcaagagtgttggcaaatagagtatcccttggaggatgagtgaggaaaccccagcaggaagagaaccgactgcagaggaaatatttgagcaagaagcagaaagttggaagagaacaagcgtgcgagtcccaaatgacatattacaaagatggctagcaatgcttaggcaaagaggaaatagaaagaaagtgcttagggaaatgcaaaaatgggcatggaggaatcccacggcgcgggtgattcggccgtgtggatgtcggctatgtaaccccggctgggggagtaattaatcataataaagcaaattgtaacatgctgtgtcaggtgtcttgcaggaatggcggagataagaaaagaagcaaaggagccactaatccagggtaagtataaaaaacaggtaagtagaataactatagttatattactaacagtaagagcagcactaggagcagaatacataaccataatatcagacccatatgggttctctcccgtgagaaatgtgtcaggagtacctgtaacttgtgtgacaaaagaatttagtaagtggggatgtcagccaataggagcctacccagacccagacttagaatacagaaatataagtaaagaaatattagaggaagtatatcaacaagactggccgtggaatacttatcattggccattatggcaaatggataatgtagtacaatgggcaaggcaaaatttacaggataaccgcaaggaaaaaagggacctggcagacctattagcaggaaaaataaggggaagattctgtgtaccctacccatttgcgctcctggagtgcatggaatggtgctggtgggttaagaacactaatgcaggggggtatggagaagcagacataagaataaattgctcaagggcaagagcagtgagctgcacaagtgaaatgcccttagcatccctacagagggtatattgggaaaaggaggaacgaaaaaacatggagaaaatgaccatcaaaccttgcaataaaaatttggaatgcaagaacagaaggggatgcgcagaagggtatccagtacctcccaaggcagagttattccctccagcgtttcaggatttacagccaaaagggtacgcatatggggcacttagagggaacagcaaatttccacaaagagtgtcgctaagaacatgggtgaaaatagctaacctgacaggatgggaaaaaggaaagccagcagaatggtggaataccagccaacaggttcattggtttgataccacgccacaatatcatttaggatatgtattatcccgagcgcctgagaacaggagttgtaatttcacaggggaaatacgaatagggcaacatcagtttgagtataattacaccctgacaaagaattgcacaaaggagaagtggaaagagtaccccatgtggcatgtctggaggcatttagatcaaaatgagcacttatctagcatatgtttcaaaagaccgagaagaaatgcaacacaaatagggaacagtacactgcaagggcaatgtaatagaagtaattggacaggatgccactgcaatgagacagggataaacacaacatggagaataaatggcacaaagggagcttatctcttaaatagcactaatggaaacatcatggtcttgttatgctggaacacaacagtggcaggggtatatgagagtcagctaaagtggaatgagagtcttaaagacggagactatgggctctgttttaattcaacaaacaggaattgtactagaaatggagctcggcactatgtaaacaagagagtgataaaaaacgacacagcagatcataattgtgatagcagcatatcagcaatagatggaatggtacatcaacaaatattactgcaaaggtatcaagtaattagagtaagagcttacacatacggagtgattgatatgccagacaattatgagaccctaccaggaaggagaaggagagatctcgcaaaggccaggaaaaagaggggcgtgggcctggtcatcatgttagctatcatggccatagtggctgctgcaggagcatctctgggagtcgcgaacgcgattcagcagtcctacaccagggacgctgtccagactcttgctaacgcgactgctgtgcaacagcaggtgttagaggcgtcctatgccatgatacagcatgtggctaagggaatacgcatccttgaagcacgcgtggcgagaatggaagttatgatggatagaatgatgttatatcaggaagtagactgctggcattatcaccaatattgtgtaacctctacaagagcagacatagtgaattacattaattggacaaggtttaaagataattgcacatggcaagagtgggaaagggagataagtgcgcatgaaggaaacatcactatattactcaaagaatcagcaaggataacacaattagcacaacaaaaggtacaaagaataccagatgtgtggacagcactaagggagtcactaggatggacacaatggctggcttggataaaataccttcccataatagtagtagggatattaggatgcataatcataagaataatgttgtgtgtagtacaaccagttcttcagatttacagaaccttgactcagaccaggtatcaacaagtcaacttggtgatggagacccgggtgcaactagaagaagaagaagaagaagacggaagggatggtggagatggctcagagagatgcagcgatcccgacaacaaaggaattatgaacgcctggaggagagcttgggtgacttggagaaactcaccttggcagaacacatggaagaatgtggtggtggcgccgttggtgattccgctgacaatcagaatttggctccttggagagaatggagagaacccctaaaagaaaaataaaaagggtggactgtgaggactgtgaggcctaggagcgagatagaaacttataggcctctcttcccggaaagctaactcactgtgagaggaatagcaagtcacagtgacactgctaattgtacccgcaaccctgagatcatgcaaaccacaatcctgagattatgctgacatgtgtaacagctgatgcctcagctgatgcttgctcatgctgacaatgtaactaggagctctatataaacagagccctagagcttgctacttcagagtgctctgaggagctcgaaggaaagagtcctcagcctctcctctccgaggagcttcggctcataataaaggagtgcttgcttcaacagaa

SEQ ID NO: 3 - DNA, 720 bp, Caprine arthritis-encephalitis virus
atggtgagtctagatagagacatggcgaggcaagtctccggggggaaaagagattatcctgagctcgaaaaatgtatcaagcatgcatgcaagataaaagttcgactcagaggggagcacttgacagaaggaaattgtttatggtgccttaaaacattagattacatgtttgaggaccataaagaggaaccttggacaaaagtaaaatttaggacaatatggcagaaggtgaagaatctaactcctgaggagagtaacaaaaaagactttatgtctttgcaggccacattagcgggtctaatgtgttgccaaatggggatgagacctgagacattgcaagatgcaatggctacagtaatcatgaaagatgggttactggaacaagaggaaaagaaggaagacaaaagagaaaaggaagagagtgtcttcccaatagtagtgcaagcagcaggagggagaagctggaaagcagtagattctgtaatgttccagcaactgcaaacagtagcaatgcagcatggcctcgtgtctgaggactttgaaaggcagttggcatattatgctactacctggacaagtaaagacatactagaagtattggccatgatgcctggaaatagagctcaaaaggagttaattcaagggaaattaaatgaagaagcagaaaggtggagaaggaataatccaccacctccagcaggaggaggattaacagtggat

SEQ ID NO: 4 - DNA, 720 bp, Caprine arthritis-encephalitis virus
atggtgaggcaggcctccggaaggggaaaggagtaccccgagctaaaagaatgtctgaaaaaggcatgcaaaataaaagtaagggctgggggggagcgcctgacagaaggaaattgtctctggtgtataaaaacactagagtgtatgtatgaggattgtagggaggaaccttggaccccagaaaaatgtaaacaattatggaaaaagttgaagcaggtagagcctgaggagagtagcaaagcagactataactcgttaaaagcaaccttggcggggatagtctgtgtgcaaatgggaatgcagcccgagacactgcaggatgcgatagcaaccttaaacatgagagatgaagtaaaaggaaaggaaaagccatcagaagaaaagaagggaatatatcccatattagtgcaggcaggaggaggaagagcatggagagcggtagagcctgctacctttcagcagctccaaacagtggcaatgcagcatggactagtatcagaagaatttgaaaggcagctagcatactatgccaccacatggacaagcaaggatatcttagaagtattagccatgatgccaggaaatagagcgcaaaaagaactaatacaaggaaagttaaatgaggaagcagagagatggagaaggcagaatccacaacctgcgggcgggttaaccgtggatcagataatgggggtaggacaaacgaatcaggcagcggca

SEQ ID NO: 5 - DNA, 1347 bp, Caprine arthritis-encephalitis virus
atggtgagtctagatagagacatggcgaggcaagtctccggggggaaaagagattatcctgagctcgaaaaatgtatcaagcatgcatgcaagataaaagttcgactcagaggggagcacttgacagaaggaaattgtttatggtgccttaaaacattagattacatgtttgaggaccataaagaggaaccttggacaaaagtaaaatttaggacaatatggcagaaggtgaagaatctaactcctgaggagagtaacaaaaaagactttatgtctttgcaggccacattagcgggtctaatgtgttgccaaatggggatgagacctgagacattgcaagatgcaatggctacagtaatcatgaaagatgggttactggaacaagaggaaaagaaggaagacaaaagagaaaaggaagagagtgtcttcccaatagtagtgcaagcagcaggagggagaagctggaaagcagtagattctgtaatgttccagcaactgcaaacagtagcaatgcagcatggcctcgtgtctgaggactttgaaaggcagttggcatattatgctactacctggacaagtaaagacatactagaagtattggccatgatgcctggaaatagagctcaaaaggagttaattcaagggaaattaaatgaagaagcagaaaggtggagaaggaataatccaccacctccagcaggaggaggattaacagtggatcaaattatgggggtaggacaaacaaatcaagcagcagcacaagctaacatggatcaggcaaggcaaatatgcctgcaatgggtaataaatgcattaagagcagtaagacatatggcgcacaggccagggaatccaatgctagtaaagcaaaaaacgaatgagccatatgaagattttgcagcaagactgctagaagcaatagatgcagagccagttacacagcctataaaagattatctaaagctaacactatcttatacaaatgcatcagcagattgtcagaagcaaatggatagaacactaggacaaagagtacaacaagctagtgtagaagaaaaaatgcaagcatgtagagatgtgggatcagaagggttcaaaatgcaattgttagcacaagcattaaggccaggaaaaggaaaagggaatggacagccacaaaggtgttacaactgtggaaaaccgggacatcaagcaaggcaatgtagacaaggaatcatatgtcacaactgtggaaagagaggacatatgcaaaaagaatgcagaggaaagagagacataaggggaaaacagcagggaaacgggaggagggggatacgtgtggtgccgtccgctcctcctatggaataa

SEQ ID NO: 6 - DNA, 1299 bp, Caprine arthritis-encephalitis virus
atggtgaggcaggcctccggaaggggaaaggagtaccccgagctaaaagaatgtctgaaaaaggcatgcaaaataaaagtaagggctgggggggagcgcctgacagaaggaaattgtctctggtgtataaaaacactagagtgtatgtatgaggattgtagggaggaaccttggaccccagaaaaatgtaaacaattatggaaaaagttgaagcaggtagagcctgaggagagtagcaaagcagactataactcgttaaaagcaaccttggcggggatagtctgtgtgcaaatgggaatgcagcccgagacactgcaggatgcgatagcaaccttaaacatgagagatgaagtaaaaggaaaggaaaagccatcagaagaaaagaagggaatatatcccatattagtgcaggcaggaggaggaagagcatggagagcggtagagcctgctacctttcagcagctccaaacagtggcaatgcagcatggactagtatcagaagaatttgaaaggcagctagcatactatgccaccacatggacaagcaaggatatcttagaagtattagccatgatgccaggaaatagagcgcaaaaagaactaatacaaggaaagttaaatgaggaagcagagagatggagaaggcagaatccacaacctgcgggcgggttaaccgtggatcagataatgggggtaggacaaacgaatcaggcagcggcacaggctaatatggatcaagcaagacaaatatgcctacaatgggttataacagcaataagaggagttaggcatatggcccatagaccaggaaatcccatgctggtaagacaaaaaccaaatgagaactatgaagagtttgccgcaaggttgttagaagcagtggatgcagaacccgttacccaacctataaaagaatatttaaaggtaactctgtcttacacaaatgcaaattcggaatgtcaaaaacatatggacagagtgttggggcaaagagtacagcaggcctcaatagaagaaaaaatgcaggcatgcagggacatcgggggaacagcatatcagatgcagttgcttgcacaagccctccgtggcggaaaagaagatgggaaaaaatctgtagggaagtgttataactgtggaaggcccggacacagagcaaaagaatgcagacaaggcattatatgtcacaactgtggaaaaagagggcatatacagaaaaactgcaaacagaaaagaagaaaggagcagggaaacatgaggagggggctacgtgtggtgccgtccgcaccccctatggagtaa

SEQ ID NO: 7 - DNA, 511 bp, Caprine arthritis-encephalitis virus
gagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctggcgcccaacgtggggcccgaggagaagaaaagaaagcggccctgagaactcggcttctgaaaaagaggaagaggacaagttgctatagcaacaagagagaagaagtagagcaaaggtccagtggctcggaaaaagaggaactgaaacttcggggacgcctgaaggagtaaggtaagtgactctgctgtacgcggggcgaggcagaggtttccttctaaattgaaagagaagtgttgctgcgagaggtcttggtggtcgagaatcctgtacaaaaaaaaggagggatctcggtcaggaccaggacccctgggagtaatacaacagcaacaccgtaagaaaatccgcc

SEQ ID NO: 8 - DNA, 576 bp, Caprine arthritis-encephalitis virus
gtgagtgctctgaggagctcgaaggaaagagtcctcagcctctcctctccgaggagcttcggctcataataaaggagtgcttgcttcaacagaactgagctggtcgtggttattatcggggaccgaagtcccgtgcaacaccggggcggttctcgcagctggcgcccaacgtggggctcgagtagcttgagaagctcgactgagatctgaatccaagagcgacatcagacagcaagaaatgagagtaatgagaccgcgagctctgctgctgtaaaaaagaggaagtagcgggttgccgaggcaactgctcagaagaaccaggggaaagggcttccagcaacctcaaaagaggaaccgagacttcggggacgcctgaagtaaggtaagtgactctgctgtacgcggggcgaggcataggagatccttctattctaggaagagaagcgctgttctgggaggtcttggcgaccgagaatcttgttaaataagccaggatctcgatcaggaccaagacccctcaggagagggtatagacagcgtggtaagaaatccgccgtggtgagtctagatagagac

SEQ ID NO: 9 - DNA, 3318 bp, Caprine arthritis-encephalitis virus
atgtcacaactgtggaaagagaggacatatgcaaaaagaatgcagaggaaagagagacataaggggaaaacagcagggaaacgggaggagggggatacgtgtggtgccgtccgctcctcctatggaataacttcagcaccacctatggttcaggtccgcataggttcccagcagaggaacttgttatttgataccggggcggaccgaactatagttagatggcatgagggctcgggaaacccagccggaaggataaaactgcaaggaataggaggaatagtagaaggagaaaaatggaataatgtagaattagaatataaaggagaaacaagaaagggaacaatagtagtgttaccacaaagtccagtagaagtattaggacgagataacatggcccgatttggaataaagataataatggcaaatttagaggaaaaaagaatcccaattacaaaagtaaaattgaaagagggatgtacgggtccacatgtcccacaatggccattaacagaagagaaattaaaaggtctaacagaaatcatagataaattagtggaagaaggaaaactaggaaaggcacccccacattggacatgtaatactccaatcttttgcataaaaaagaaatcagggaagtggagaatgttaatagatttcagagaattgaacaaacagacagaagatttaacagaagcgcagttaggactcccgcatccgggaggactacaaaagaaaaaacatgttacaatattggacataggagatgcatattttactatacccctatatgaaccatatcgagagtacacatgttttactctattaagtcctaataatctaggaccatgtaaaagatactattggaaagtgctgccacaaggttggaaattgagtccatctgtatatcaatttactatgcaggagatcttagaggattggatacagcagcatccagaaattcaatttggcatatatatggatgatatttacataggaagtgatttagaaattaaaaagcatagagaaatagtgaaagatttagccaattatattgcccaatatggattcactctgccagaagagaagagacaaaagggatatccagcaaaatggctaggatttgaactacacccgcagacctggaaatttcagaagcatacattacctgaattaacaaagggaacaataacattaaataaattacagaaattagtaggagaattagtatggagacaatccataattgggaaaagcattcctaacattctgaaattaatggaaggagatagagaattacaaagtgaaagaaaaattgaagaagtacatgtgaaagaatgggaagcatgtaggaaaaaattagaagaaatggaaggaaattattataataaagacaaagatgtctatggacaattggcttggggagacaaagctatagaatatatagtgtatcaggagaaagggaaaccattatgggtaaatgtggttcacaatataaagaacctaagcatcccgcaacaggttattaaagcagcgcaaaaattaacccaagaagtcatcattaggacaggaaaaataccatggatattgttgccagggaaagaagaagattggagactagaattgcaattagggaacatcacatggatgccaaaattttggtcctgttatcgaggacatacaagatggagaaaaagaaatataatagaagaagtagtagaagggcctacatattatacagatggaggaaaaaagaataaagtaggaagtctagggttcatagtatcaacaggggaaaaatttagaaagcatgaagagggcacaaaccagcaactagaattaagagccatagaggaagctctaaaacaagggcctcaaacaatgaatttagtaacagatagtagatatgcatttgaatttttattaagaaattgggatgaagaagtaataaagaatccaattcaagcaagaattatggaaattgcccacaagaaagataggataggagtgcattgggtgccaggacataaagggattccccaaaatgaagaaatagacaaatatatttcggaaatatttcttgcaaaagaaggagaaggaattctcccaaaaagagaagaggatgcagggtatgatttaatatgcccagaagaggttaccatagagccaggacaagtgaaatgcatccccatagagctaagattaaatttaaagaaatcacaatgggctatgattgctacaaaaagcagcatggctgccaaaggagtgttcacacaaggaggaatcatagactcaggatatcagggacaaatacaggtaataatgtataatagcaataaaatagcagtagtcataccccaagggagaaaatttgcacaattaatattaatggataaaaagcatggaaaattggaaccctggggggaaagcagaaaaacagaaaggggagaaaaaggatttgggtctacaggaatgtattggatagaaaatattcctctggcagaggaagaccacacaaaatggcatcaagatgcccgatcattgcatctagaatttgaaattccaagaacagcagcagaagacatagtaaatcaatgtgaaatatgcaaagaagcgaggacacctgcagtaattagaggcggaaacaaaaggggggtaaatcattggcaagtggattatacccattatgaaaatatcatactattagtatgggtagaaacaaattcaggactaatatatgcagaaaaagtaaaaggagaatcagggcaagaattcagaataaaagtgatgcattggtatgcattatttggtccagagtcattgcagtcagacaatggacctgcatttgcagcagagcccacacagctgttaatgcaatacctaggagtaaaacacacaacaggcataccttggaatccacagtctcaggctatagtagaaagggcacatcaactattgaaaagcactttaaagaagttccagccacaatttgtcgctgtagaatcagccatagcagcagccctagtcgccataaatataaaaagaaagggtgggctggggacaagccctatggatatttttatatataataaagaacagaaaagaataaataataaatataataaaaattctcaaaaaattcaattctgttattacagaataaggaaaagaggacatcaggagagtggaaaggaccaacccaggtactgtggaaaggggaaggagccaattgtggtaaaggatatagaaagtgaaaagtatttagtaataccttacaaagatgcaaaattcatcccgccaccaacaaaagaaaaggaataa

SEQ ID NO: 10 - DNA, 3324 bp, Caprine arthritis-encephalitis virus
atgcagacaaggcattatatgtcacaactgtggaaaaagagggcatatacagaaaaactgcaaacagaaaagaagaaaggagcagggaaacatgaggagggggctacgtgtggtgccgtccgcaccccctatggagtaacgcaagcaccactaatagttagggtacaaatagggaatcaggagaaacaattattatttgacacaggggcagataaaacgatagtaagaatgcatgatggaacagggattccaaacggaagaataaaattacaagggataggaggaatagtagaaggagaaaaatggaataaagtacccatgacatataagggagaaacatcctgcccaagcttggttgtgctaagagatagcccagtagaagtattgggaagagataacatggaagcattcggcgtaaccctaataatggcaaatttagaagataagaaaattcccacaataccagtagaattgaaagaaggatgtaaagggccacatgtgccccagtggccattaacagcagagaaattacaaggactaacaggaatagtagaaaaattactacaggaaggaaaattggcagaggccccagagggatggacgtggaacacgcccatcttctgcataaaaaagaagtcaggaaaatggagaatgttaatagattttagggaattaaataagcaaacagcagatttagcagaagcgcagctaggactgccacacccaggagggttgcaaaggaaaaagaatgtaacaattctggacataggagatgcatatttcacaattcccttatacgagccctatcagaaatatacatgcttcacactcctaagtcctaacaatttgggaccatgtaaaaggtattattggaaagtattaccccagggatggaaattgagcccagctgtatatcaattcaccatgcaaaggttgttaaaaggatggatacaacagcataaaaacatacaatttggaatatatatggatgatatctatattggaagtgatctaacgatagcccaacataggaagataatagaagaattagcctcatttatagaacaatttgggtttacattaccagaagataagagacaagagggctatccagcaaaatggctaggattcgagctacatccagaaaaatggaaatatcaaaagcataaattgccggaattacaagagggggtaataaccctgaacaaattacagaagatagtaggggaattagtgtggagacaatccttgataggaaagagcatccccaatatcataaaattaatggaaggagatcgcgcattacaaagtgaaaggaaaatagaaagaatacatgtacaagaatgggaagcatgtcaaaagaaattagatgaaatggtaggaaattattacagagaagaagaagatatctatggacaaataacttggggggataaggcaataaaatacatagtattccaaaggaaaggggaacccctatgggtaaatgtagtacatgacataaaaaatttgagtctcccacagcaagtgataaaagcagcacagaaattaacccaggaagtaatcataagaacaggaaaaatcccatggctgctactaccaggaagagaagaagactggagattagaactgcaggtagggaacatcacgtggatgccatcattttggtcatgttatcgaggagcacccaagtggaaaagaaggaacatagtggcagcagtggtagatggaccgacatattatacagatgggggaaagaaaaacgcacagggaagctttggcttcatctccccaacaggagaaaagttcagaaggcatgaagatggaactaatcaggtattagaattaagggcaatagaagatccatgtaaacaaggacctgaaagcatgaacattgtaactgacagcaggtatgcttatgaattcatgctccgaaactgggatgaacaggtcataagaaaccccattcaggcaagaatcatggcagaagtgcacaagaaaaagcaggtaggaatacactgggtgccagggcataaaggaatacctcagaatgaagagatagaccagtacatatcagaagtattcttagcacgagaaggaacagggatatgtgaaaaaaggaaggaagatgctggatatgatttattatgcccgcatgaggtaatacttaaaccccaagaagtaaaacggatcccaatagacctaaaattaaaattgaaagaaaagcaatgggccatgataagtgggaaaagtagcgttgcagcaaaaggaatatttgtacaaggaggcataatagattcagggtatcagggacaagtacaagtcatcctatataatagtaataagatagaggtcaaaataccacaaggcaggaaatttgcccaattaatattaatgaacttacaacatgaagaattagaagaatggggaaaggaaagaaaaacagaaagaggaacaaaaggatttgggtctacaggagcattttggatagagaatattccccaagcagaggaagaacattacaaatggcatcaagatgctagatctctgcagctagaattcaagatacctagagcagcagcagaagacattatacagcactgtgaggtatgtcaagaaggcaaacccgcagcgatcacgagagggggaaataaaagaggaatagatcattggcaggtagactatacacattacaaagaacacataatattagtatgggtagagactaattcaggattaatatttgcagagaaagtaaaaggagaatcaggacaagaatttaggatgcagacattgaaatggtatgctttgtttcaaccaaaatcagtgcaatcagataatgggacagccttcacagctgaggctacgcagcatctaatgaagtatttagggattcagcacactacgggtattccgtggaacccccagtcacaaagtttagtagaaagagctcatcaaacattaaaacacatgttagaaaaattagaaccacaatttgtggccctacagtctgccatcgcagccactctagttgcgctcaatataaaaagaaagggtggactaggggcaagccctatggatatttacatatataataaggagcaacaaagacaacaagataatagtaataaattaattcagaaaaaattttgttattacaggatcagaaaaagaggccatccaggagagtggaacggcccaactgaggtactgtgggaaggggaaggagccatagtagttaaagacaaagaaagtgatagatatctagtcatcccatataaagatgcaaaatttattccgccaccgtcggaacagaagggatag

SEQ ID NO: 11 - DNA, 402 bp, Caprine arthritis-encephalitis virus
atggatgctggggccagatacatgcgcttaactgggaaggaaaactgggttgaagtaaccatggacggagagaaggaaaggaaaagagaaggtttcactgcgggacagcaagatatacagaactctaagtaccccgacataccaacgggtcacagtcatcatggaaacaagagcagacgtcgcaggagaaaatcaggattttggcgatggcttagaggaatcagacaacagcgaaacaagcgaaagagtgacagtacagaaagcttggagccgtgcctgggagctttggcagaactcaccctggaaggagccatggaaaaggggcctgctgaggctgctcgtccttccgctgacgatgggaatctggataaatggatggcttggagaacaccacaaaaataa

SEQ ID NO: 12 - DNA, 321 bp, Caprine arthritis-encephalitis virus
atggcggagataagaaaagaagcaaaggagccactaatccaggaccaggtatcaacaagtcaacttggtgatggagacccgggtgcaactagaagaagaagaagaagaagacggaagggatggtggagatggctcagagagatgcagcgatcccgacaacaaaggaattatgaacgcctggaggagagcttgggtgacttggagaaactcaccttggcagaacacatggaagaatgtggtggtggcgccgttggtgattccgctgacaatcagaatttggctccttggagagaatggagagaacccctaaaagaaaaataa

SEQ ID NO: 13 - DNA, 690 bp, Caprine arthritis-encephalitis virus
atgcaaaattcatcccgccaccaacaaaagaaaaggaataaaaaacctggaccagaattacccttagcactatggatacatatagcagaaagcattaatggggatagctcatggtacataacaatgagactgcaacagatgatgtggggaaaaagaggaaataagttacaatataagaatgaagacagggaatatgaaaattgggaaattacatcatggggatggaaaatgcacctaaggagagtgaaacaatggatacaagacaacaggagaggaagcccatggcagtacaaagtaggaggaacatggaaaagtataggagtgtggttcctgcaagcaggagattacagaaaggtagacaggcacttctggtgggcatggaggatactgatatgttcctgcaggaaagaaaagtttgatataagagaatttatgagaggaagacatagatgggatttgtgcaaatcctgtgctcaaggagaagtagtaaagcatactagaacaaaaagtctggaaagactagtactgctacagatggtagaacagcatgtgtttcaagtattgccattgtggagagccaggagaagtagtacaacagatttcccatggtgcagggacacaacgggatacacgcatgcgtggtctgtccaggagtgctggttgatggaatatctcttagaggatgagtga

SEQ ID NO: 14 - DNA, 490 bp, Caprine arthritis-encephalitis virus
atgcaaaatttattccgccaccgtcggaacagaagggatagaagaataggcaaaaagtcatccaagagggcatacttcgccatggcaatatcggagacaggatggatggaaggatgtgggaacgtggttcctacagccaggggactatagaaaggcggatcagcagttctggttcgcttggagaatagtgtcgtgttcatgtaaaaaggaaggatttaacataagagaatttatgctaggtacccatagatgggatttgtgtaagtcgtgttgccagggtgaagtagtaaagagaacacaaccctacaccttgcaaaggctcacgtggcttaaattaacagaagaccatgtatttcaagtaatgcccttgtggagagctcgcaaagggattaccatagactttccctggtgcagggacacaaaaggattcctggagccgtggacaacgcaagagtgttggcaaatagagtatcccttggaggatgagtga

SEQ ID NO: 15 - DNA, 720 bp, Caprine arthritis-encephalitis virus; identical to SEQ ID NO: 3

SEQ ID NO: 16 - DNA, 591 bp, Caprine arthritis-encephalitis virus
gctgtagactctgtaatgttccaacaaatgcaaacagtagcaatgcagcatggcctcgtgtccgaggattttgaaagacagttagcatattatgctactacctggacaagtaaagacatactagaagtattggccatgatgcctgggaatagggctcagaaagaacttattcaagggaaattgaatgaagaagcagacaggtggagaaggaacaatccaccaggaggattaacagtggatcaaattatgggggtaggacaaacaaatcaagcagcagcacaagctaacatggatcaggcaagacaaatatgcctacaatgggtaataaacgccttaagagcagtaaggcatatggctcataggccagggaatccaatgctagtaaagcaaaaaacaaatgagccatatgaagaatttgcagcaagactgctagaagcaatagatgcagaagcggttacacagcccataaaagagtatctaaagctaacattatcctatacaaatgcagcctcagattgtcaaaagcaaatggagagagtgctaggacaaagagtacaacaggctagtgtagaaaaaaaaatgcaagcatgt

SEQ ID NO: 17 - DNA, 1347 bp, Caprine arthritis-encephalitis virus; identical to SEQ ID NO: 5

SEQ ID NO: 18 - DNA, 591 bp, Caprine arthritis-encephalitis virus; identical to SEQ ID NO: 16

SEQ ID NO: 19 - DNA, 720 bp, Caprine arthritis-encephalitis virus; identical to SEQ ID NO: 3

SEQ ID NO: 20 - DNA, 662 bp, Caprine arthritis-encephalitis virus
tcaagcagcaggagggagaagctggaaagcagtagactcagtgatgttccagcaactgcaaaatgtagcaatgcagcatggcctcgtgtccgaggattttgaaaggcagttagtatattatgctactacctggacaagtaaagatatattagaagtattggccatgatgcctggaaatagagctcaaaaagagttaattcaagggaaattgaatgaggaagcagaaaggtggagaagaaataatccaccacctcaagcaggcggaggattaacagtggatcaaattatgggggtaggacaaacaaatcaagcagcggcacaggctaacatggatcaggcaagacaaatatgtctgcaatgggtaataacagcactaagagcagtgagacatatggctcacaaaccagggaatccaatgctagtaaagcaaaaaacaaatgagtcatatgaagattttgccgcaagactgctagaagcaatagatgcagaaccagttacacagcaaataaaagaatatttaaagttaacattatcttacacaaatgcatcctcagactgtcagaaacagatggatagagtactaggacagagagtgcaacaagctagtgtggaagaaaaaatgcaagcatgcagagatgtgggatcagaaggattcagaatgc

SEQ ID NO: 21 - DNA, 662 bp, Caprine arthritis-encephalitis virus
gcaagcagcaggagggagaagctggaaagcagtagactcagtgatgttccagcaactgcaaaatgtagcaatgcagcatggcctcgtgtccgaggattttgaaaggcagttagcatattatgctactacctggacaagtaaagatatattagaagtattggccatgatgcctggaaatagagctcaaaaagagttaattcaagggaaattgaatgaggaagcagaaaggtggagaaggaataatccaccacctcaagcaggcggaggattaacagtggatcaaattatgggggtaggacaaacaaatcaagcagcggcacaggctaacatggatcaggcaagacaaatatgcctgcaatgggtaataacagcactaagagcagtgagacatatggctcacaaaccagggaatccaatgctagtaaagcaaaagacaaatgagtcatatgaagattttgccgcaagactgctagaagcaatagatgcagaaccagttacacagcaaataaaagaatatttaaagttaacattatcttacacaaatgcatcctcagactgtcaaaaacaaatggatagaatactaggacagagagtgcaacaagctagtgtggaagaaaaaatgcaagcatgcagagatgtgggatcagaagggttcagaatgc

SEQ ID NO: 22 - DNA, 651 bp, Caprine arthritis-encephalitis virus
gcaagcagcaggagggagaagctggaaagcagtagactcagtgatgttccagcaactgcaaaatgtagcaatgcagcatggcctcgtgtccgaggattttgaaaggcagttggcatattatgctactacctggacaagtaaagatatattagaagtattggccatgatgcctggaaacagagctcaaaaagagttaattcaggggaaattgaataaggaagcagaaaggtggagaaggaataatccaccacctcaagcacaaggaggattaacagtggatcaaattatgggggtaggacaaacaaatcaggcagcggcacaggctaacatggatcaggcaagacaaatatgcctgcaatgggtaataacagcactaagagcagtgagacatatggctcacaaaccagggaatccaatgctagtaaagcaaaagacaaatgagtcatatgaagattttgccgcaagactgctagaagcaatagatgcagaaccagttacacagcaaataaaagaatatttaaagttaacattatcttacacaaatgcatcctcagactgtcagaaacaaatggatagagtactaggacagagagtgcaacaagctagtgtggaagaaaaaatgcaagcatgcagagatgtgggatcagaagg

SEQ ID NO: 23 - DNA, 520 bp, Caprine arthritis-encephalitis virus
gcaagcagcaggagggagaagctggaaagcagtagactcagtgatgttccagcaactgcaaaatgtagcaatgcagcatggcctcgtgtccgaggattttgaaaggcagttagcatattatgctactacctggacaagtaaagatatattagaagtattggccatgatgcctggaaatagagctcaaaaagagttaattcaagggaaattgaatgaggaagcagaaaggtggagaaggaataatccaccacctcaagcaggcggaggattaacagtggatcaaattatgggggtaggacaaacaaatcaagcagcggcacaggctaacatggatcaggcaagacaaatatgcctgcaatgggtaataacagcactaagagcagtgagacatatggctcacaaaccagggaatccaatgctagtaaagcaaaagacaaatgagtcatatgaagattttgccgcaagactgctagaagcaatagatgcagaaccagttacacagcaaataaagaatatttaa

SEQ ID NO: 24 - DNA, 663 bp, Caprine arthritis-encephalitis virus
gcaagcagcaggagggagaagctggaaagcagtagactcagtgatgttccagcaactgcaaaatgtagcaatgcagcatggcctcgtgtccgaggattttgaaaggcagttagcatattatgctactacctggacaagtaaagatatattagaagtattggccatgatgcctggaaatagagctcaaaaagagttaattcaagggaaattgaatgaggaagcagaaaggtggagaaggaataatccaccacctcaagcaggcggaggattaacagtggatcaaattatgggggtaggacaaacaaatcaagcagcggcacaggctaacatggatcaggcaagacaaatatgcctgcaatgggtaataacagcgactaagagcagtgagacatatggctcacaaaccagggaatccaatgctagtaaagcaaaagacaaatgagtcatatgaaaaattttcagcaagactcctagaagcaatagatgcagaaccagttacacagcctataaaagaatatttaaagttaacattatcttacacaaatgcatcctcagactgtcaaaaacaaatggatagagtactaggacagagagtgcaacaagctagtgtggaagaaaaaatgcaagcatgcagagatgtgggatcagaaggattcagaatgc

SEQ ID NO: 25 - DNA, 1347 bp, Caprine arthritis-encephalitis virus; identical to SEQ ID NO: 5

SEQ ID NO: 26 - DNA, 662 bp, Caprine arthritis-encephalitis virus; identical to SEQ ID NO: 20

SEQ ID NO: 27 - DNA, 662 bp, Caprine arthritis-encephalitis virus; identical to SEQ ID NO: 21

SEQ ID NO: 28 - DNA, 651 bp, Caprine arthritis-encephalitis virus; identical to SEQ ID NO: 22

SEQ ID NO: 29 - DNA, 520 bp, Caprine arthritis-encephalitis virus; identical to SEQ ID NO: 23

SEQ ID NO: 30 - DNA, 662 bp, Caprine arthritis-encephalitis virus
gcaagcagcaggagggagaagctggaaagcagtagactcagtgatgttccagcaactgcaaaatgtagcaatgcagcatggcctcgtgtccgaggattttgaaaggcagttagcatattatgctactacctggacaagtaaagatatattagaagtattggccatgatgcctggaaatagagctcaaaaagagttaattcaagggaaattgaatgaggaagcagaaaggtggagaaggaataatccaccacctcaagcaggcggaggattaacagtggatcaaattatgggggtaggacaaacaaatcaagcagcggcacaggctaacatggatcaggcaagacaaatatgcctgcaatgggtaataacagcactaagagcagtgagacatatggctcacaaaccagggaatccaatgctagtaaagcaaaagacaaatgagtcatatgaaaaattttcagcaagactcctagaagcaatagatgcagaaccagttacacagcctataaaagaatatttaaagttaacattatcttacacaaatgcatcctcagactgtcaaaaacaaatggatagagtactaggacagagagtgcaacaagctagtgtggaagaaaaaatgcaagcatgcagagatgtgggatcagaaggattcagaatgc

SEQ ID NO: 31 - DNA, 720 bp, Caprine arthritis-encephalitis virus; identical to SEQ ID NO: 3

SEQ ID NO: 32 - DNA, 597 bp, Caprine arthritis-encephalitis virus
gcagtcgatgctgtaatgttccagcaaatgcaaacagtagccatgcagcatggtcttgtgtctgaggactttgaaaggcagttagcatattgtgctactacctggacaagtaaagatatattagaagtattggccatgatgcctggaaatagagctcaaaaagagttaattcaaggaaaattaaacgaggaagcagaaaggtggagaaggaataatccaccgcctccacaaggagggggattaacagtggatcaaattatggggataggacaaacaaatcaagcagcagcacaagctaacatggatcaggcaagacacatatgcctgcaatgggtaataacagcattaagagcagtaagacatatggctcacagaccagggaatccaatgctcgtaaaacaaaaaacaaatgagccatatgaagagtttgcagcaaaactattagaagcaatagatgcagaaccagtaacacagcccataaaagactatctaaagttaacattatcttatacaaatgcgtcctcagactgtcaaaagcaaatggatagagtgctgggacaaagagtgcaacaagctagtgtagacgagaaaatgcaa

SEQ ID NO: 33 - DNA, 597 bp, Caprine arthritis-encephalitis virus
gcagtagactcagtaatgttccagcaactgcaaacagtagcaatgcagcatggcctcgtgtccgaggattttgaaaggcagttggcatattatgctactacctggacgagtaaagacatactagaagtattggccatgatgcctggaaacagagctcaaaaggagttaattcaagggaaattaaatgaagaggcagaaaggtggagaagacataatccaccccctccggcgggaggaggattaacagtggatcaaattatgggggtaggacaaacaaatcaagcagcagcacaagctaacatggatcaggcaagacaaatatgcctgcaatgggtaataacagcattaagagcagtgaggtatatgactcacaaaccagggaatccaatgctagtaaaacaaaaaacaaatgaagcatatgaagagtttacagcgagactgctagaagcaatagatgcagagccagtaacacagcccacaaaagaatatctaaaactaacattatcttatacaaatgcatcctcagactgtcaaaagcaaatggatagagtactaggacaaagagtgcaacaagctagtgtagaagaaaaaatgcaa

SEQ ID NO: 34 - DNA, 597 bp, Caprine arthritis-encephalitis virus
gcagtcgatgctgtaatgttccagcaaatgcaaacagtagccatgcagcatggtcttgtgtctgaggactttgaaaggcagttagcatattatgctactacctggacaagtaaagatatattagaagtattggccatgatgcctggaaatagagctcaaaaagagttaattcaaggaaaattaaatgaggaagcagaaaggtggagaaggaataatccaccgcctccacagggagggggattaacagtggatcaaattatggggataggacaaacaaatcaagcagcagcacaagctaacatggatcaggcaagacacatatgcctgcaatgggtaataacagcattaagagcagtaagacatatggctcacagaccagggaatccaatgctcgtaaaacaaaaaacaaatgagccatatgaagagtttgcagcaaaactattagaagcaatagatgcagaaccagtaacacagctcataaaagactatctaaagttaacattatcttatacaaatgcgtcctcagactgtcaaaagcaaatggatagagtgctgggacaaagagtgcaacaagctagtgtagacgagaagatgcaa

SEQ ID NO: 35 - DNA, 1347 bp, Caprine arthritis-encephalitis virus; identical to SEQ ID NO: 5

SEQ ID NO: 36 - DNA, 597 bp, Caprine arthritis-encephalitis virus; identical to SEQ ID NO: 32

SEQ ID NO: 37 - DNA, 597 bp, Caprine arthritis-encephalitis virus; identical to SEQ ID NO: 33

SEQ ID NO: 38 - DNA, 597 bp, Caprine arthritis-encephalitis virus; identical to SEQ ID NO: 34

SEQ ID NO: 39 - DNA, 720 bp, Caprine arthritis-encephalitis virus; identical to SEQ ID NO: 3

SEQ ID NO: 40 - DNA, 524 bp, Caprine arthritis-encephalitis virus
taaagatatattagaagtattggccatgatgcctggaaatagagctcaaaaagagttaattcaagggaaattgaatgaagaagcagaaaggtggagaaggaataatccaccacctcaagcaggcggaggattaacagtggatcaaattatgggggtaggacaaacaaatcaagcagcggcacaggctaacatggatcaggcaagacaaatatgcctgcaatgggtaataacagcactaagagcagtgagacatatggctcacaaaccagggaatccgatgctagtaaagcaaaaaacaaatgagtcatatgaagattttgccgcaagactgctagaagcaatagatgcagaaccagttacaaagcaaataaaagaatatttaaagttaacattatcttacacaaatgcatcctcagactgtaagaaacagatggatagagtactaggacagagagtgcaacaagctagtgtggaagaaaaaatgcaagcatgcagagatgtgggatcagaaggattcagaatgc

SEQ ID NO: 41 - DNA, 1347 bp, Caprine arthritis-encephalitis virus; identical to SEQ ID NO: 5

SEQ ID NO: 42 - DNA, 524 bp, Caprine arthritis-encephalitis virus; identical to SEQ ID NO: 40

SEQ ID NO: 43 - DNA, 720 bp, Caprine arthritis-encephalitis virus; identical to SEQ ID NO: 3

SEQ ID NO: 44 - DNA, 593 bp, Caprine arthritis-encephalitis virus
tgccgtagactctgtgatgttccaccagctgcatacagtagcaatgccgcatggcctcgtgtctgaggactttgaaaggcagttggcatattatgctactacctggacaagtaaagatatactggaagtattggccatgatgcctgggaatagagctcaaaaagaattaattcaaggaaaattaaatgaagaagcagaaaggtggagaaggaataatccaccacctcaagcaggcggaggattaacagtggatcaaattatgggggtaggacaaacaaatcaagcagctgcacaagctaacatggatcaggcaagacaaatatgcctgcaatgggtaatatcagccttaagagcagtgagacatatgtctcataaaccagggaatccgctgctagtaaagcaaaaaacaaatgagtcatatgaagattttgcagctagactgctagaagcaatagatccagccccagtagcacatcctataaaagattatttaaagttaacactatcttatacgaatgcatcatcagattgtcaaaagcaaatgggtagaatgctaggatcgagagtccatcaagccagtgtgggccaaaaaa

SEQ ID NO: 45 - DNA, 1347 bp, Caprine arthritis-encephalitis virus; identical to SEQ ID NO: 5

SEQ ID NO: 46 - DNA, 593 bp, Caprine arthritis-encephalitis virus; identical to SEQ ID NO: 44

SEQ ID NO: 47 - DNA, 720 bp, Caprine arthritis-encephalitis virus; identical to SEQ ID NO: 3

SEQ ID NO: 48 - DNA, 593 bp, Caprine arthritis-encephalitis virus
agcagtagattctgtaatgttccagcaactgcaaacagtagcaatgcagcatggactcgtgtatgaagactttgaaaggctgtcggcatattatgctactacctggacaagtaaagatatactggaagtattggccatgatgcctgggaatagagctcaaaaagaattaattcaaggaaaattaaatgaagaagcagaaaggtggagaaggaataatccaccacctcaagcaggcggaggattaacagtggatcaaattatgggggtaggacaaacaaatcaagcagctgcacaagctaacatggatcaggcaagacaaatatgcctgcaatgggtaatatcagccttaagagcagtgagacatatgtctcataaaccagggaatccgctgctagtaaagcaaaaaacaaatgagtcatatgaagattttgcagcaagactgctagaagcaatagatgcagagccagtagcacatcctataaaagaatacttaaagttaacactatcttatacgaatgcatcatcagattgtcaaaagcaaatggatagaatgctggaatcaagagtacaacaagctagtgtagaacaaaaaa

SEQ ID NO: 49 - DNA, 593 bp, Caprine arthritis-encephalitis virus
agccgtagattctgtaatgttccagcagctgcaaacagtagcaatgcagcatggcctcgtgtcagaggactttgaaaggcttccagcatatcatgctactacctgggcaagtaaagatatcttagaagtactggccatgatgcctggaaatagagctcaaaaagagttaattcaagggaaattaaatgaagaagcagagaggtggagaaggaataatccaccacctccagcaggaggagggttaacagtggatcaaattatgggagtaggacaaacaaatcaggcagcggcacaagcaaacatggatcaggcaagacaaatatgcctacaatgggtgatatcagcactaagagcagtaaggcatatggctcacaagccagggaatccaatgttagtaaagcaaaaagcaaatgagccatatgaagaatttgcagcaaggctgctggaagcaatagatgccgagccagttaatcagcccataaaagaatatctaaaactaacgttgtcttatacgaatgcatcctcagattgtcagaagcaaatggatagaacactaggacaaagagtcaaacaagctagtgtagaacaaaaaa

SEQ ID NO: 50 - DNA, 1347 bp, Caprine arthritis-encephalitis virus; identical to SEQ ID NO: 5

SEQ ID NO: 51 - DNA, 593 bp, Caprine arthritis-encephalitis virus
agcagtagattctgtaatgttccagcaactgcaaacagtagcaatgcagcatggactcgtgtatgaagactttgaaaggctgtcggcatattatgctactacctggacaagtaaagatatactggaagtattggccatgatgcctgggaatagagctcaaaaagaattaattcaaggaaaattaaatgaagaagcagaaaggtggagaaggaataatccaccacctcaagcaggcggaggattaacagtggatcaaattatgggggtaggacaaacaaatcaagcagctgcacaagctaacatggatcaggcaagacaaatatgcctgcaatgggtaatatcagccttaagagcagtgagacatatgtctcataaaccagggaatccgctgctagtaaagcaaaaaacaaatgagtcatatgaagattttgcagcaagactgctagaagcaatagatgcagagccagtagcacatcctataaaagaatatctaaaactaacgttgtcttatacgaatgcatcctcagattgtcagaagcaaatggatagaacactaggacaaagagtcaaacaagctagtgtagaacaaaaaa

SEQ ID NO: 52 - DNA, 593 bp, Caprine arthritis-encephalitis virus; identical to SEQ ID NO: 49

SEQ ID NO: 53 - DNA, 720 bp, Caprine arthritis-encephalitis virus
atggtgagtctagatagagacatggcgaggcaagtctccggggggaaaagagattatcctgagctcgaaaaatgtatcaagcatgcatgcaagataaaagttcgactcagaggggagcacttgacagaaggaaattgtttatggtgccttattacatgtttgaggaccataaagaggaaccttggacaaaaaaacattagagtaaaatttaggacaatatggcagaaggtgaagaatctaactcctgaggagagtaacaaaaaagactttatgtctttgcaggccacattagcgggtctaatgtgttgccaaatggggatgagacctgagacattgcaagatgcaatggctacagtaatcatgaaagatgggttactggaacaagaggaaaagaaggaagacaaaagagaaaaggaagagagtgtcttcccaatagtagtgcaagcagcaggagggagaagctggaaagcagtagattctgtaatgttccagcaactgcaaacagtagcaatgcagcatggcctcgtgtctgaggactttgaaaggcagttggcatattatgctactacctggacaagtaaagacatactagaagtattggccatgatgcctggaaatagagctcaaaaggagttaattcaagggaaattaaatgaagaagcagaaaggtggagaaggaataatccaccacctccagcaggaggaggattaacagtggat

SEQ ID NO: 54 - DNA, 129 bp, Caprine arthritis-encephalitis virus
cagcatggcctcgtgtccgaggactttgaaaggcagttggcatattatgctactacctggacaagtaaggacatattagaagtattggccatgatgccaggaaatagagctcaaaaggagctaattcaa

SEQ ID NO: 55 - DNA, 129 bp, Caprine arthritis-encephalitis virus
cagcatggaatagtatcagaagagtttgagaggcaactgtcttattatgctaccacttggacaagcaaggatatcttagaggtactagccatgatgcctggcaatagagcattaaaagagctaatacaa

SEQ ID NO: 56 - DNA, 129 bp, Caprine arthritis-encephalitis virus
caacatgggatagtatcagaggaatttgagagacaaatgtcttattatgctaccacatggacaagtaaggatattttagaagtactagcaatgatgcccgggaacagagcattaaaggagctgatacaa

SEQ ID NO: 57 - DNA, 129 bp, Caprine arthritis-encephalitis virus
cagcatggactagtatcagaagaatttgaaaggcagctagcatactatgccacaacgtggacaagcaaagacatactagaggtgttagccatgatgccagggaatagagcacaaaaagaactaatacaa

SEQ ID NO: 58 - DNA, 129 bp, Caprine arthritis-encephalitis virus
cagcatggacttgtgtccgaagattttgagaggcaattggcatattatgctacaacctggactagtgaagatatattagaagtattggctatgatgcctgggaatagagcacagaaagaattaatacaa

SEQ ID NO: 59 - DNA, 1347 bp, Caprine arthritis-encephalitis virus; identical to SEQ ID NO: 5

SEQ ID NO: 60 - DNA, 129 bp, Caprine arthritis-encephalitis virus; identical to SEQ ID NO: 54

SEQ ID NO: 61 - DNA, 129 bp, Caprine arthritis-encephalitis virus; identical to SEQ ID NO: 55

SEQ ID NO: 62 - DNA, 129 bp, Caprine arthritis-encephalitis virus; identical to SEQ ID NO: 56

SEQ ID NO: 63 - DNA, 129 bp, Caprine arthritis-encephalitis virus; identical to SEQ ID NO: 57

SEQ ID NO: 64 - DNA, 129 bp, Caprine arthritis-encephalitis virus; identical to SEQ ID NO: 58

SEQ ID NO: 65 - DNA, 20 bp, Artificial Sequence
  Primer used to construct dig-labeled probe
ctggcgtaatagcgaagagg

SEQ ID NO: 66 - DNA, 20 bp, Artificial Sequence
  Primer used to construct dig-labeled probe
aactcgccgcacatctgaac

SEQ ID NO: 67 - DNA, 3911 bp, Artificial
  pCAH/SINd0
atccagcacagtggcggccgctagcacaaaaataaaaaaagaaagggtggactgtgagacatgggctaaagaggagcggccgctcgagtctagaactagtggatcagctttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagccgacctcgagggggggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgatcaggactcttccttttcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattcgcgcaattaaccctcactaaagggaacaaaagctgggtaccgggcccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctggcgcccaacgtggggcccgaggagaagaaaagaaagcggccctgagaactcggcttctgaaaaagaggaagaggacaagttgctatagcaacaagagagaagaagtagagcaaaggtccagtggctcggaaaaagaggaactgaaacttcggggacgcctgaaggagtaaggtaagtgactctgctgtacgcggggcgaggcagaggtttccttctaaattgaaagagaagtgttgctgcgagaggtcttggtggtcgagaatcctgtacaaaaaaaaggagggatctcggtcaggaccaggacccctgggagtaatacaacagcaacaccgtaagaaaatccgcctagggaattcgattctagaggtgatagaaatgccagaaaactatgcaaaaacaagaatcataaacaggaaaaaaagagaactcagccacaagaggaagaagagaggcgttggcttggtcattatgctagttatcatggcaatagtagctgccgcaggggcttctctgggagtcgcaaacgcgattcagcagtcttacactaaggcagctgtccagacccttgctaatgcaactgctgcacagcaggatgtgttagaggcaacctatgccatggtacagcatgtggctaaaggcgtacgaatcttggaagctcgagtggctcgagtggaagctatcacagatagaataatgctataccaagaattggattgttggcactaggatccatcgccacc

SEQ ID NO: 68 - DNA, 4238 bp, Artificial
  pCAH/SINd1
atccagcacagtggcggccgctagcacaaaaataaaaaaagaaagggtggactgtgagacatgggctaaagaggagcggccgctcgagtctagaactagtggatcagctttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagccgacctcgagggggggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgatcaggactcttccttttcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattcgcgcaattaaccctcactaaagggaacaaaagctgggtaccgggcccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctggcgcccaacgtggggcccgaggagaagaaaagaaagcggccctgagaactcggcttctgaaaaagaggaagaggacaagttgctatagcaacaagagagaagaagtagagcaaaggtccagtggctcggaaaaagaggaactgaaacttcggggacgcctgaaggagtaaggtaagtgactctgctgtacgcggggcgaggcagaggtttccttctaaattgaaagagaagtgttgctgcgagaggtcttggtggtcgagaatcctgtacaaaaaaaaggagggatctcggtcaggaccaggacccctgggagtaatacaacagcaacaccgtaagaaaatccgcctaggtgagtctagatagagactaggcgaggcaagtctccggggggaaaagagattatcctgagctcgaaaaatgtatcaagcatgcatgcaagataaaagttcgactcagaggggagcacttgacagaaggaaattgtttatggtgccttaaaacattagattacatgtttgaggaccataaagaggaaccttggacaaaagtaaaatttaggacaatatggcagaaggtgaagaatctaactcctgaggagagtaacaaaaaagactttatgtctttgcaggccacattagcgggtctaatgtgttgccaaatggggatgagacctgcaggaattcgattctagaggtgatagaaatgccagaaaactatgcaaaaacaagaatcataaacaggaaaaaaagagaactcagccacaagaggaagaagagaggcgttggcttggtcattatgctagttatcatggcaatagtagctgccgcaggggcttctctgggagtcgcaaacgcgattcagcagtcttacactaaggcagctgtccagacccttgctaatgcaactgctgcacagcaggatgtgttagaggcaacctatgccatggtacagcatgtggctaaaggcgtacgaatcttggaagctcgagtggctcgagtggaagctatcacagatagaataatgctataccaagaattggattgttggcactaggatccatcgccacc

SEQ ID NO: 69 - DNA, 4523 bp, Artificial
  pCAH/SINd2
atccagcacagtggcggccgctagcacaaaaataaaaaaagaaagggtggactgtgagacatgggctaaagaggagcggccgctcgagtctagaactagtggatcagctttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagccgacctcgagggggggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgatcaggactcttccttttcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattcgcgcaattaaccctcactaaagggaacaaaagctgggtaccgggcccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctggcgcccaacgtggggcccgaggagaagaaaagaaagcggccctgagaactcggcttctgaaaaagaggaagaggacaagttgctatagcaacaagagagaagaagtagagcaaaggtccagtggctcggaaaaagaggaactgaaacttcggggacgcctgaaggagtaaggtaagtgactctgctgtacgcggggcgaggcagaggtttccttctaaattgaaagagaagtgttgctgcgagaggtcttggtggtcgagaatcctgtacaaaaaaaaggagggatctcggtcaggaccaggacccctgggagtaatacaacagcaacaccgtaagaaaatccgcctaggtgagtctagatagagactaggcgaggcaagtctccggggggaaaagagattatcctgagctcgaaaaatgtatcaagcatgcatgcaagataaaagttcgactcagaggggagcacttgacagaaggaaattgtttatggtgccttaaaacattagattacatgtttgaggaccataaagaggaaccttggacaaaagtaaaatttaggacaatatggcagaaggtgaagaatctaactcctgaggagagtaacaaaaaagactttatgtctttgcaggccacattagcgggtctaatgtgttgccaaatggggatgagacctgagacattgcaagatgcaatggctacagtaatcatgaaagatgggttactggaacaagaggaaaagaaggaagacaaaagagaaaaggaagagagtgtcttcccaatagtagtgcaagcagcaggagggagaagctggaaagcagtagattctgtaatgttccagcaactgcaaacagtagcaatgcagcatggcctcgtgtctgaggactttgaaaggcagttggcatattatgctactacctggacaagtaaagacatactagaagtattggccatgatgcctgcaggaattcgattctagaggtgatagaaatgccagaaaactatgcaaaaacaagaatcataaacaggaaaaaaagagaactcagccacaagaggaagaagagaggcgttggcttggtcattatgctagttatcatggcaatagtagctgccgcaggggcttctctgggagtcgcaaacgcgattcagcagtcttacactaaggcagctgtccagacccttgctaatgcaactgctgcacagcaggatgtgttagaggcaacctatgccatggtacagcatgtggctaaaggcgtacgaatcttggaagctcgagtggctcgagtggaagctatcacagatagaataatgctataccaagaattggattgttggcactaggatccatcgccacc

SEQ ID NO: 70 - DNA, 4819 bp, Artificial
  pCAH/SINd3
atccagcacagtggcggccgctagcacaaaaataaaaaaagaaagggtggactgtgagacatgggctaaagaggagcggccgctcgagtctagaactagtggatcagctttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagccgacctcgagggggggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgatcaggactcttccttttcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattcgcgcaattaaccctcactaaagggaacaaaagctgggtaccgggcccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctggcgcccaacgtggggcccgaggagaagaaaagaaagcggccctgagaactcggcttctgaaaaagaggaagaggacaagttgctatagcaacaagagagaagaagtagagcaaaggtccagtggctcggaaaaagaggaactgaaacttcggggacgcctgaaggagtaaggtaagtgactctgctgtacgcggggcgaggcagaggtttccttctaaattgaaagagaagtgttgctgcgagaggtcttggtggtcgagaatcctgtacaaaaaaaaggagggatctcggtcaggaccaggacccctgggagtaatacaacagcaacaccgtaagaaaatccgcctaggtgagtctagatagagactaggcgaggcaagtctccggggggaaaagagattatcctgagctcgaaaaatgtatcaagcatgcatgcaagataaaagttcgactcagaggggagcacttgacagaaggaaattgtttatggtgccttaaaacattagattacatgtttgaggaccataaagaggaaccttggacaaaagtaaaatttaggacaatatggcagaaggtgaagaatctaactcctgaggagagtaacaaaaaagactttatgtctttgcaggccacattagcgggtctaatgtgttgccaaatggggatgagacctgagacattgcaagatgcaatggctacagtaatcatgaaagatgggttactggaacaagaggaaaagaaggaagacaaaagagaaaaggaagagagtgtcttcccaatagtagtgcaagcagcaggagggagaagctggaaagcagtagattctgtaatgttccagcaactgcaaacagtagcaatgcagcatggcctcgtgtctgaggactttgaaaggcagttggcatattatgctactacctggacaagtaaagacatactagaagtattggccatgatgcctggaaatagagctcaaaaggagttaattcaagggaaattaaatgaagaagcagaaaggtggagaaggaataatccaccacctccagcaggaggaggattaacagtggatcaaattatgggggtaggacaaacaaatcaagcagcagcacaagctaacatggatcaggcaaggcaaatatgcctgcaatgggtaataaatgcattaagagcagtaagacatatggcgcacaggccagggaatccaatgctagtaaagcaaaaaacgaatgagccatatgaagattttgcagcaagactgcaggaattcgattctagaggtgatagaaatgccagaaaactatgcaaaaacaagaatcataaacaggaaaaaaagagaactcagccacaagaggaagaagagaggcgttggcttggtcattatgctagttatcatggcaatagtagctgccgcaggggcttctctgggagtcgcaaacgcgattcagcagtcttacactaaggcagctgtccagacccttgctaatgcaactgctgcacagcaggatgtgttagaggcaacctatgccatggtacagcatgtggctaaaggcgtacgaatcttggaagctcgagtggctcgagtggaagctatcacagatagaataatgctataccaagaattggattgttggcactaggatccatcgccacc

SEQ ID NO: 71 - DNA, 5112 bp, Artificial
  pCAH/SINd4
atccagcacagtggcggccgctagcacaaaaataaaaaaagaaagggtggactgtgagacatgggctaaagaggagcggccgctcgagtctagaactagtggatcagctttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagccgacctcgagggggggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgatcaggactcttccttttcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattcgcgcaattaaccctcactaaagggaacaaaagctgggtaccgggcccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctggcgcccaacgtggggcccgaggagaagaaaagaaagcggccctgagaactcggcttctgaaaaagaggaagaggacaagttgctatagcaacaagagagaagaagtagagcaaaggtccagtggctcggaaaaagaggaactgaaacttcggggacgcctgaaggagtaaggtaagtgactctgctgtacgcggggcgaggcagaggtttccttctaaattgaaagagaagtgttgctgcgagaggtcttggtggtcgagaatcctgtacaaaaaaaaggagggatctcggtcaggaccaggacccctgggagtaatacaacagcaacaccgtaagaaaatccgcctaggtgagtctagatagagactaggcgaggcaagtctccggggggaaaagagattatcctgagctcgaaaaatgtatcaagcatgcatgcaagataaaagttcgactcagaggggagcacttgacagaaggaaattgtttatggtgccttaaaacattagattacatgtttgaggaccataaagaggaaccttggacaaaagtaaaatttaggacaatatggcagaaggtgaagaatctaactcctgaggagagtaacaaaaaagactttatgtctttgcaggccacattagcgggtctaatgtgttgccaaatggggatgagacctgagacattgcaagatgcaatggctacagtaatcatgaaagatgggttactggaacaagaggaaaagaaggaagacaaaagagaaaaggaagagagtgtcttcccaatagtagtgcaagcagcaggagggagaagctggaaagcagtagattctgtaatgttccagcaactgcaaacagtagcaatgcagcatggcctcgtgtctgaggactttgaaaggcagttggcatattatgctactacctggacaagtaaagacatactagaagtattggccatgatgcctggaaatagagctcaaaaggagttaattcaagggaaattaaatgaagaagcagaaaggtggagaaggaataatccaccacctccagcaggaggaggattaacagtggatcaaattatgggggtaggacaaacaaatcaagcagcagcacaagctaacatggatcaggcaaggcaaatatgcctgcaatgggtaataaatgcattaagagcagtaagacatatggcgcacaggccagggaatccaatgctagtaaagcaaaaaacgaatgagccatatgaagattttgcagcaagactgctagaagcaatagatgcagagccagttacacagcctataaaagattatctaaagctaacactatcttatacaaatgcatcagcagattgtcagaagcaaatggatagaacactaggacaaagagtacaacaagctagtgtagaagaaaaaatgcaagcatgtagagatgtgggatcagaagggttcaaaatgcaattgttagcacaagcattaaggccaggaaaaggaaaagggaatggacagccacaaaggtgttacaactgtggaaaaccgggacatcaagcaaggcactgcaggaattcgattctagaggtgatagaaatgccagaaaactatgcaaaaacaagaatcataaacaggaaaaaaagagaactcagccacaagaggaagaagagaggcgttggcttggtcattatgctagttatcatggcaatagtagctgccgcaggggcttctctgggagtcgcaaacgcgattcagcagtcttacactaaggcagctgtccagacccttgctaatgcaactgctgcacagcaggatgtgttagaggcaacctatgccatggtacagcatgtggctaaaggcgtacgaatcttggaagctcgagtggctcgagtggaagctatcacagatagaataatgctataccaagaattggattgttggcactaggatccatcgccacc

SEQ ID NO: 72 - DNA, 7579 bp, Artificial
  pMYKEF1/env
aacaggaaagttccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttccctggaaattccatattggcacgcattctattggctgagctgcgttcacgtgggtataagaggcgcgaccagcgtcggtaccgtcgcagtcttcggtctgaccaccgtagaacgcagagctcctcgctgcaggcatgcaagcttggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcaaggccttgcgcttaaggagccccttcgccttttgcttgagttgaggcctggcctgggcgctggggccgccgcgtgcaaatctggtggcaccttcgcgcctgtctcgctgctttcgataagtctctagccatttaaaatttttgatgacctgctgcgacgctttttttctggcaagatantcttgtaaatgcgggccaagatctgcacactggtatttcggtttttggggccgcgggcggctacggggcccgtgcgtcccagcgcacatgttcggcgaggaggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcagggatccactagtaacggccgccagtgtgctggaattcgatcatacctggtgttgctgactaccccgaccgcggtaaaagtcgatggtattgctgcctgggtccatgcttctcacctcaaacctgcaccaccttcggcaccagatgagtcctgggagctggaaaagactgatcatcctcttaagctgcgtattcggcggcggcgggacgagtctgcaaaataagaacccccaccagcccatgaccctcacttggcaggtactgtcccaaactggagacgttgtctgggatacaaaggcagtccagcccccttggacttggtggcccacacttaaacctgatgtatgtgccttggcggctagtcttgagtcctgggatatcccgggaaccgatgtctcgtcctctaaacgagtcagacctccggactcagactatactgccgcttataagcaaatcacctggggagccatagggtgcagctaccctcgggctaggactagaatggcaagctctaccttctacgtatgtccccgggatggccggaccctttcagaagctagaaggtgcggggggctagaatccctatactgtaaagaatgggattgtgagaccacggggaccggttattggctatctaaatcctcaaaagacctcataactgtaaaatgggaccaaaatagcgaatggactcaaaaatttcaacagtgtcaccagaccggctggtgtaacccccttaaaatagatttcacagacaaaggaaaattatccaaggactggataacgggaaaaacctggggattaagattctatgtgtctggacatccaggcgtacagttcaccattcgcttaaaaatcaccaacatgccagctgtggcagtaggtcctgacctcgtccttgtggaacaaggacctcctagaacgtccctcgctctcccacctcctcttcccccaagggaagcgccaccgccatctctccccgactctaactccacagccctggcgactagtgcacaaactcccacggtgagaaaaacaattgttaccctaaacactccgcctcccaccacaggcgacagactttttgatcttgtgcagggggccttcctaaccttaaatgctaccaacccaggggccactgagtcttgctggctttgtttggccatgggccccccttattatgaagcaatagcctcatcaggagaggtcgcctactccaccgaccttgaccggtgccgctgggggacccaaggaaagctcaccctcactgaggtctcaggacacgggttgtgcataggaaaggtgccctttacccatcagcatctctgcaatcagaccctatccatcaattcctccggagaccatcagtatctgctcccctccaaccatagctggtgggcttgcagcactggcctcaccccttgcctctccacctcagtttttaatcagactagagatttctgtatccaggtccagctgattcctcgcatctattactatcctgaagaagttttgttacaggcctatgacaattctcaccccaggactaaaagagaggctgtctcacttaccctagctgttttactggggttgggaatcacggcgggaataggtactggttcaactgccttaattaaaggacctatagacctccagcaaggcctgacaagcctccagatcgccatagatgctgacctccgggccctccaagactcagtcagcaagttagaggactcactgacttccctgtccgaggtagtgctccaaaataggagaggccttgacttgctgtttctaaaagaaggtggcctctgtgcggccctaaaggaagagtgctgtttttacatagaccactcaggtgcagtacgggactccatgaaaaaactcaaagaaaaactggataaaagacagttagagcgccagaaaagccaaaactggtatgaaggatggttcaataactccccttggttcactaccctgctatcaaccatcgctgggcccctattactcctccttctgttgctcatcctcgggccatgcatcatcaataagttagttcaattcatcaatgataggataagtgcagttaaaattctggtccttagacaaaaatatcaggccctagagaacgaaggtaacctttaattttgctctaagattagagctattcacaagagaaatggggatcactagtgaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttgatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaataaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaat

SEQ ID NO: 73 - DNA, 3566 bp, Artificial
  pCAH/SINd
atccagcacagtggcggccgctagcacaaaaataaaaaaagaaagggtggactgtgagacatgggctaaagaggagcggccgctcgagtctagaactagtggatcagctttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagccgacctcgagggggggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgatcaggactcttccttttcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattcgcgcaattaaccctcactaaagggaacaaaagctgggtaccgggcccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaagctttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctggcgggaattcgattctagaggtgatagaaatgccagaaaactatgcaaaaacaagaatcataaacaggaaaaaaagagaactcagccacaagaggaagaagagaggcgttggcttggtcattatgctagttatcatggcaatagtagctgccgcaggggcttctctgggagtcgcaaacgcgattcagcagtcttacactaaggcagctgtccagacccttgctaatgcaactgctgcacagcaggatgtgttagaggcaacctatgccatggtacagcatgtggctaaaggcgtacgaatcttggaagctcgagtggctcgagtggaagctatcacagatagaataatgctataccaagaattggattgttggcactaggatccatcgccacc

SEQ ID NO: 74 - DNA, 7623 bp, Artificial
  pHGVSV-G
gcgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccactagtaacggccgccagtgtgctggaattcgatgatcctgagaacttcagggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattaggcccttttgctaatcatgttcatacctcttatcttcctcccacagctcctgggcaacgtgctggtctgtgtgctggcccatcactttggcaaagaatcactagtgaattctgcagatatccatcacactggcggccgctcgaggaattctgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaaaaaaaaaaaacggaattcctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttgatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaataaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttac

SEQ ID NO: 75 - DNA, 5419 bp, Artificial
  pHYK/rev
gcgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttagatggatgctggggccagatacatgcgcttaactgggaaggaaaactgggttgaagtaaccatggacggagagaaggaaaggaaaagagaaggtttcactgcgggacagcaagatatacagaactctaagtaccccgacataccaacgggtcacagtcatcatggaaacaagagcagacgtcgcaggagaaaatcaggattttggcgatggcttagaggaatcagacaacagcgaaacaagcgaaagagtgacagtacagaaagcttggagccgtgcctgggagctttggcagaactcaccctggaaggagccatggaaaaggggcctgctgaggctgctcgtccttccgctgacgatgggaatctggataaatggatggcttggagaacaccacaaaaataagaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttgatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaataaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttac

SEQ ID NO: 76 - DNA, 5729 bp, Artificial
  pHYK/vif
gcgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccgcgatgcaaaattcatcccgccaccaacaaaagaaaaggaataaaaaacctggaccagaattacccttagcactatggatccatatagcagaaagcattaatggggatagctcatggtacataacaatgagactgcaacagatgatgtggggaaaaagaggaaataagttacaatataagaatgaagacagggaatatgaaaattgggaaattacatcatggggatggaaaatgcacctaaggagagtgaaacaatggatacaagacaacaggagaggaagcccatggcagtacaaagtaggaggaacatggaaaagtataggagtgtggttcctgcaagcaggagattacagaaaggtagacaggcacttctggtgggcatggaggatactgatatgttcctgcaggaaagaaaagtttgatataagagaatttatgagaggaagacatagatgggatttgtgcaaatcctgtgctcaaggagaagtagtaaagcatactagaacaaaaagtctggaaagactagtactgctacagatggtagaacagcatgtgtttcaagtattgccattgtggagagccaggagaagtagtacaacagatttcccatggtgcagggacacaacgggatacacgcatgcgtggtctgtccaggagtgctggttgatggaatatctcttagaggatgagtgaccggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttgatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaataaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttac

SEQ ID NO: 77 - DNA, 9446 bp, Artificial
  pMGP/RRE
aacaggaaagttccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttccctggaaattccatattggcacgcattctattggctgagctgcgttcacgtgggtataagaggcgcgaccagcgtcggtaccgtcgcagtcttcggtctgaccaccgtagaacgcagagctcctcgctgcaggcaagcttggtaccgagctcggatcccggggaggtaccaaaatccgccatggtgagtctagatagagacatggcgaggcaagtctccggggggaaaagagattatcctgagctcgaaaaatgtatcaagcatgcatgcaagataaaagttcgactcagaggggagcacttgacagaaggaaattgtttatggtgccttaaaacattagattacatgtttgaggaccataaagaggaaccttggacaaaagtaaaatttaggacaatatggcagaaggtgaagaatctaactcctgaggagagtaacaaaaaagactttatgtctttgcaggccacattagcgggtctaatgtgttgccaaatggggatgagacctgagacattgcaagatgcaatggctacagtaatcatgaaagatgggttactggaacaagaggaaaagaaggaagacaaaagagaaaaggaagagagtgtcttcccaatagtagtgcaagcagcaggagggagaagctggaaagcagtagattctgtaatgttccagcaactgcaaacagtagcaatgcagcatggcctcgtgtctgaggactttgaaaggcagttggcatattatgctactacctggacaagtaaagacatactagaagtattggccatgatgcctggaaatagagctcaaaaggagttaattcaagggaaattaaatgaagaagcagaaaggtggagaaggaataatccaccacctccagcaggaggaggattaacagtggatcaaattatgggggtaggacaaacaaatcaagcagcagcacaagctaacatggatcaggcaaggcaaatatgcctgcaatgggtaataaatgcattaagagcagtaagacatatggcgcacaggccagggaatccaatgctagtaaagcaaaaaacgaatgagccatatgaagattttgcagcaagactgctagaagcaatagatgcagagccagttacacagcctataaaagattatctaaagctaacactatcttatacaaatgcatcagcagattgtcagaagcaaatggatagaacactaggacaaagagtacaacaagctagtgtagaagaaaaaatgcaagcatgtagagatgtgggatcagaagggttcaaaatgcaattgttagcacaagcattaaggccaggaaaaggaaaagggaatggacagccacaaaggtgttacaactgtggaaaaccgggacatcaagcaaggcaatgtagacaaggaatcatatgtcacaactgtggaaagagaggacatatgcaaaaagaatgcagaggaaagagagacataaggggaaaacagcagggaaacgggaggagggggatacgtgtggtgccgtccgctcctcctatggaataacttcagcaccacctatggttcaggtccgcataggttcccagcagaggaacttgttatttgataccggggcggaccgaactatagttagatggcatgagggctcgggaaacccagccggaaggataaaactgcaaggaataggaggaatagtagaaggagaaaaatggaataatgtagaattagaatataaaggagaaacaagaaagggaacaatagtagtgttaccacaaagtccagtagaagtattaggacgagataacatggcccgatttggaataaagataataatggcaaatttagaggaaaaaagaatcccaattacaaaagtaaaattgaaagagggatgtacgggtccacatgtcccacaatggccattaacagaagagaaattaaaaggtctaacagaaatcatagataaattagtggaagaaggaaaactaggaaaggcacccccacattggacatgtaatactccaatcttttgcataaaaaagaaatcagggaagtggagaatgttaatagatttcagagaattgaacaaacagacagaagatttaacagaagcgcagttaggactcccgcatccgggaggactacaaaagaaaaaacatgttacaatattggacataggagatgcatattttactatacccctatatgaaccatatcgagagtacacatgttttactctattaagtcctaataatctaggaccatgtaaaagatactattggaaagtgctgccacaaggttggaaattgagtccatctgtatatcaatttactatgcaggagatcttagaggattggatacagcagcatccagaaattcaatttggcatatatatggatgatatttacataggaagtgatttagaaattaaaaagcatagagaaatagtgaaagatttagccaattatattgcccaatatggattcactctgccagaagagaagagacaaaagggatatccagcaaaatggctaggatttgaactacacccgcagacctggaaatttcagaagcatacattacctgaattaacaaagggaacaataacattaaataaattacagaaattagtaggagaattagtatggagacaatccataattgggaaaagcattcctaacattctgaaattaatggaaggagatagagaattacaaagtgaaagaaaaattgaagaagtacatgtgaaagaatgggaagcatgtaggaaaaaattagaagaaatggaaggaaattattataataaagacaaagatgtctatggacaattggcttggggagacaaagctatagaatatatagtgtatcaggagaaagggaaaccattatgggtaaatgtggttcacaatataaagaacctaagcatcccgcaacaggttattaaagcagcgcaaaaattaacccaagaagtcatcattaggacaggaaaaataccatggatattgttgccagggaaagaagaagattggagactagaattgcaattagggaacatcacatggatgccaaaattttggtcctgttatcgaggacatacaagatggagaaaaagaaatataatagaagaagtagtagaagggcctacatattatacagatggaggaaaaaagaataaagtaggaagtctagggttcatagtatcaacaggggaaaaatttagaaagcatgaagagggcacaaaccagcaactagaattaagagccatagaggaagctctaaaacaagggcctcaaacaatgaatttagtaacagatagtagatatgcatttgaatttttattaagaaattgggatgaagaagtaataaagaatccaattcaagcaagaattatggaaattgcccacaagaaagataggataggagtgcattgggtgccaggacataaagggattccccaaaatgaagaaatagacaaatatatttcggaaatatttcttgcaaaagaaggagaaggaattctcccaaaaagagaagaggatgcagggtatgatttaatatgcccagaagaggttaccatagagccaggacaagtgaaatgcatccccatagagctaagattaaatttaaagaaatcacaatgggctatgattgctacaaaaagcagcatggctgccaaaggagtgttcacacaaggaggaatcatagactcaggatatcagggacaaatacaggtaataatgtataatagcaataaaatagcagtagtcataccccaagggagaaaatttgcacaattaatattaatggataaaaagcatgaaaaattggaaccctggggggaaagcagaaaaacagaaaggggagaaaaaggatttgggtctacaggaatgtattggatagaaaatattcctctggcagaggaagaccacacaaaatggcatcaagatgcccgatcattgcatctagaatttgaaattccaagaacagcagcagaagacatagtaaatcaatgtgaaatatgcaaagaaggcaggacacctgcagtaattagaggcggaaacaaaaggggggtagatcattggcaagtggattatacccattatgaaaatatcatactattagtatgggtagaaacaaattcaggactaatatatgcagaaaaagtaaaaggagaatcagggcaagaattcagaataaaagtgatgcaatggtatgcattatttggtccagagtcattgcagtcagacaatggacctgcatttgcagcagagcccacacagctgttaatgcaatacctaggagtaaaacacacaacaggcataccttggaatccacagtctcaggctatagtagaaagggcacatcaactattgaaaagcactttaaagaagttccagccacaatttgtcgctgtagaatcagccatagcagcagccctagtcgccataaatataaaaagaaagggtgggctggggacaagccctatggatatttttatatataataaagaacagaaaagaataaataataaatataataaaaattctcaaaaaattcaattctgttattacagaataaggaaaagaggacatccaggagagtggaaaggaccaacccaggtactgtggaaaggggaaggagcaattgtggtaaaggatatagaaagtgaaaagtatttagtaataccttacaaagatgcaaaattcatcccgccaccaacaaaagaaaaggaataaaaaacctggaccagaattacccttagcactatggatccactagtaacggccgccagtgtgctggaattctgcagatatccatcacactggcggccgggctgcaggaattcgatagaaaagatatcaaaaacaagaatcataaacaggaaaaaaagagaactcagccacaagaggaagaagagaggcgttggcttggtcattatgctagttatcatggcaatagtagctgccgcaggggcttctctgggagtcgcaaacgcgattcagcagtcttacactaaggcagctgtccagacccttgctaatgcaactgctgcacagcaggatgtgttagaggcaacctatgccatggtacagcatgtggctaaaggcgtacgaatcttggaagctcgagtggctcgagtggaagctatcacagatagaatagcggccgcccatcaagcttatcgataccgtcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttgatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaataaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaat

SEQ ID NO: 78 - DNA, 7856 bp, Artificial
  pCAH/SINd60/hlacZ
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctggcgcccaacgtggggcccgaggagaagaaaagaaagcggccctgagaactcggcttctgaaaaagaggaagaggacaagttgctatagcaacaagagagaagaagtagagcaaaggtccagtggctcggaaaaagaggaactgaaacttcggggacgcctgaaggagtaaggtaagtgactctgctgtacgcggggcgaggcagaggtttccttctaaattgaaagagaagtgttgctgcgagaggtcttggtggtcgagaatcctgtacaaaaaaaaggagggatctcggtcaggaccaggacccctgggagtaatacaacagcaacaccgtaagaaaatccgcctaggtgagtctagatagagactaggcgaggcaagtctccggggggaaaagagattatcctgcaggaattcgattctagaggtgatagaaatgccagaaaactatgcaaaaacaagaatcataaacaggaaaaaaagagaactcagccacaagaggaagaagagaggcgttggcttggtcattatgctagttatcatggcaatagtagctgccgcaggggcttctctgggagtcgcaaacgcgattcagcagtcttacactaaggcagctgtccagacccttgctaatgcaactgctgcacagcaggatgtgttagaggcaacctatgccatggtacagcatgtggctaaaggcgtacgaatcttggaagctcgagtggctcgagtggaagctatcacagatagaataatgctataccaagaattggattgttggcactaggatccatcagccaccattaacgcttacaatttacgcgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgcttaccatggggggttctcatcatcatcatcatcatggtatggctagcatgactggtggacagcaaatgggtcgggatctgtacgacgatgacgataaggtacctaaggatcagcttggagttgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataataaagccgaattctgcagatatccagcacagtggcggccgctagcacaaaaataaaaaaagaaagggtgactgtgagacatgggctaaagaggagcggccgctcgagtctagaactagtggatcagctttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagccgacctcgagggggggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgatcaggactcttccttttcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattcgcgcaattaaccctcactaaagggaacaaaagctgggtaccgggccc

SEQ ID NO: 79 - DNA, 8127 bp, Artificial
  pCAH/SINd1/hlacZ
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaagcttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagctggcgcccaacgtggggcccgaggagaagaaaagaaagcggccctgagaactcggcttctgaaaaagaggaagaggacaagttgctatagcaacaagagagaagaagtagagcaaaggtccagtggctcggaaaaagaggaactgaaacttcggggacgcctgaaggagtaaggtaagtgactctgctgtacgcggggcgaggcagaggtttccttctaaattgaaagagaagtgttgctgcgagaggtcttggtggtcgagaatcctgtacaaaaaaaaggagggatctcggtcaggaccaggacccctgggagtaatacaacagcaacaccgtaagaaaatccgcctaggtgagtctagatagagactaggcgaggcaagtctccggggggaaaagagattatcctgagctcgaaaaatgtatcaagcatgcatgcaagataaaagttcgactcagaggggagcacttgacagaaggaaattgtttatggtgccttaaaacattagattacatgtttgaggaccataaagaggaaccttggacaaaagtaaaatttaggacaatatggcagaaggtgaagaatctaactcctgaggagagtaacaaaaaagactttatgtctttgcaggccacattagcgggtctaatgtgttgccaaatggggatgagaccgggctgcaggaattcgattctagaggtgatagaaatgccagaaaactatgcaaaaacaagaatcataaacaggaaaaaaagagaactcagccacaagaggaagaagagaggcgttggcttggtcattatgctagttatcatggcaatagtagctgccgcaggggcttctctgggagtcgcaaacgcgattcagcagtcttacactaaggcagctgtccagacccttgctaatgcaactgctgcacagcaggatgtgttagaggcaacctatgccatggtacagcatgtggctaaaggcgtacgaatcttggaagctcgagtggctcgagtggaagctatcacagatagaataatgctataccaagaattggattgttggcactaggatccatcagccaccattaacgcttacaatttacgcgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctttaagcttaccatggggggttctcatcatcatcatcatcatggtatggcatgactggtggacagcaaatgggtcgggatctgtacgacgatgacgataaggtacctaaggatcagcttggagttgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataataaagccgaattctgcagatatccagcacagtggcggccgctagcacaaaaataaaaaaagaaagggtgactgtgagacatgggctaaagaggagcggccgctcgagtctagaactagtggatcagctttgctgcttgcacttcagagttctaggagagtccctcctagtctctcctctccgaggaggtaccgagacctcaaaataaaggagtgattgccttactgccgagtggagagtgattactgagcggccggtgtatcgggagtcgtcccttaatctgtgcaataccagagcggctctcgcagccgacctcgagggggggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgatcaggactcttccttttcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattcgcgcaattaaccctcactaaagggaacaaaagctgggtaccgggccc

(19) United States
(12) Patent Application Publication
(10) Pub. No.: US 2006/0199266 A1
(43) Pub. Date: Sep. 7, 2006

(54) CAEV-based vector systems

(75) Inventors: Yeon-Soo Kim, Seoul (KR); Jong-pil Kim, Daejeon (KR); Sukyung Lee, Seoul (KR)

Correspondence Address: SUGHRUE MION, PLLC, 2100 PENNSYLVANIA AVENUE, N.W., SUITE 800, WASHINGTON, DC, 20037, US

(73) Assignee: VECTORCORE A CO., LTD.

(21) Appl. No.: 11/351,102

(22) Filed: Feb. 10, 2006

Related U.S. Application Data
(63) Continuation-in-part of application No. PCT/KR04/02030, filed on Aug. 12, 2004.

(51) Int. Cl. C12N 15/86

(52) U.S. Cl. 435/456

(57) ABSTRACT

This invention relates to caprine arthritis encephalitis virus-based vectors and vector systems that are useful in the delivery of nucleic acids to both non-dividing and dividing cells. Methods for delivering nucleic acids to both non-dividing and dividing cells using the vector systems are also disclosed.

### BACKGROUND OF THE INVENTION

1. Field of the Invention

This invention relates to lentiviral vectors useful in polynucleotide delivery, and more specifically to caprine arthritis encephalitis virus-based vectors useful in polynucleotide delivery to non-dividing and dividing cells.

2. Related Art

Lentiviruses are a subgroup of retroviruses that are capable of infecting non-dividing, as well as dividing cells. Vectors derived from lentiviruses are ideal tools for delivering exogenous genes to target cells because of their ability to stably integrate into the genome of dividing and non-dividing cells and to mediate long-term gene expression (Gilbert and Wong-Staal, 2001; Mitrophanous et al., 1999; Naldini et al., 1996; Sauter and Gasmi, 2001).

Lentiviruses have been isolated from many vertebrate species including primates, e.g., human and simian immunodeficiency viruses (HIV-1, HIV-2, SIV), as well as non-primates, e.g., feline immunodeficiency virus (FIV), bovine immunodeficiency virus (BIV), equine infectious virus (EIAV), caprine arthritis encephalitis virus (CAEV) and the visna virus. Of these, HIV and SIV are presently best understood. However, use of such systems in humans raises serious safety concerns, due to the possibility of recombination by the vector into a virulent and disease-causing form. Accordingly, non-primate lentiviruses are preferred for use in gene therapy.

Among non-primate lentiviral vectors, vectors derived from FIV (Curran and Nolan, 2002) and EIAV [US 2001/0044149] are best characterized, and little progress has been made for other non-primate lentiviral vectors.

CAEV, like all lentiviruses, can infect and replicate in dividing cells as well as in terminally differentiated and non-dividing cells. Several features of CAEV biology make this virus an attractive candidate to develop into a gene transfer/therapy vector. First, the normal host of CAEV is goats, and there are no reported cases of human infection by CAEV. Second, the CAEV genome is phylogenetically most distant from HIV-1 among lentiviruses. Third, the genome organization of the CAEV is relatively simple compared with other lentiviruses. The CAEV genome contains three structural genes (gag, pol, env) and three regulatory/accessory genes (vif, tat and rev).

Despite these advantages, however, efforts to develop CAEV-based delivery systems have not been successful, resulting only in unsafe and inefficient recombinant viral vector production systems, rendering the use of CAEV-based gene delivery systems impractical.

In 1998, L. Mselli-Lakhal et al. reported on the first generation CAEV-based vector system, but the viral titers of the system (i.e., 10-187 TU/ml) were below useful levels. The authors attributed the inefficiency to a lack of accumulation of genomic RNA into the cytoplasm, and the low packaging efficiency of the vector RNA. Another shortcoming of the study was the use of an infectious wild-type virus (“helper virus”) as its packaging system, which is of little practical value in human applications.

Accordingly, a need remains for a safe and efficient CAEV-based lentiviral vector system capable of mediating gene transfer into a broad range of dividing and non-dividing cells.

### SUMMARY OF THE INVENTION

The present invention is broadly directed to the production of CAEV-based lentiviral vector particles useful for delivering exogenous polynucleotides into target cells. These vector particles find use in anti-viral, anti-tumor and/or gene therapies.

The present invention provides in one aspect a transfer vector for use in a CAEV-based vector production system described herein, the transfer vector comprises (a) a CAEV packaging sequence consisting essentially of (i) the untranslated region between the CAEV 5′ LTR and the CAEV gag-encoding sequence, and (ii) nucleotides 1 to X of the CAEV gag-encoding sequence linked to the 3′ end of said untranslated region, wherein X is less than 613, and (b) cis-acting elements required for polyadenylation, RNA transport, reverse transcription, and integration, in operable association with said packaging sequence.

In one embodiment of the invention, X is selected from the group consisting of: 60, 100, 125, 150, 175, 200, 225, 250, 275, 300, 325, 350, 375, 400, 425, 450, 475, 500, 525, 550, 575 and 600.

In another embodiment of the invention, X is selected from the group consisting of:
X is greater than 25 and less than 600,
X is greater than 25 and less than 500,
X is greater than 25 and less than 400,
X is greater than 25 and less than 300,
X is greater than 25 and less than 200,
X is greater than 50 and less than 600,
X is greater than 50 and less than 500,
X is greater than 50 and less than 400,
X is greater than 50 and less than 300,
X is greater than 50 and less than 200,
X is greater than 75 and less than 600,
X is greater than 75 and less than 500,
X is greater than 75 and less than 400,
X is greater than 75 and less than 300,
X is greater than 75 and less than 200,
X is greater than 100 and less than 600,
X is greater than 100 and less than 500,
X is greater than 100 and less than 400,
X is greater than 100 and less than 300,
X is greater than 100 and less than 200,
X is greater than 125 and less than 600,
X is greater than 125 and less than 500,
X is greater than 125 and less than 400,
X is greater than 125 and less than 300,
X is greater than 125 and less than 200,
X is greater than 150 and less than 600,
X is greater than 150 and less than 500,
X is greater than 150 and less than 400,
X is greater than 150 and less than 300,
X is greater than 150 and less than 200,
X is greater than 200 and less than 600,
X is greater than 200 and less than 500,
X is greater than 200 and less than 400,
X is greater than 200 and less than 300,
X is greater than 200 and less than 200,
X is greater than 250 and less than 600,
X is greater than 250 and less than 500,
X is greater than 250 and less than 400, and
X is greater than 250 and less than 300.

In another embodiment, X is greater than 40 and less than 613.

In another embodiment, X is greater than 57 and less than 613.

In yet another embodiment, X is about 327.

In one embodiment of the invention, the start codon of the gag-encoding sequence is mutated to prevent translation of gag protein. In a further embodiment, the start codon is mutated to TAG.

In another embodiment of the transfer vector of the invention, the ATG codon of the gag-encoding sequence is located X base pairs downstream of the start codon ATG, wherein start codon is mutated to prevent translation of gag protein, and wherein X is less than 30. In a further embodiment X is about 21.

The transfer vector of the invention may further comprise an RRE region.

In another embodiment of the invention, the transfer vector comprises the CAEV 3′ LTR wherein the U3 region is deleted.

The transfer vector of the present invention may further comprise a heterologous promoter. In one embodiment of the invention, the heterologous promoter is the human cytomegalovirus major immediate early promoter (HCMV MIEP). In a further embodiment, the transfer vector is pCAH/SINd1 (SEQ ID NO: 68).

The transfer vector of the present invention may further comprise a transcription cassette comprising a heterologous polynucleotide of interest operably linked to a heterologous promoter (e.g., human cytomegalovirus major immediate-early promoter HCMV MIEP, or murine cytomegalovirus major immediate-early promoter MCMV MIEP). Such a transfer vector permits the incorporation of the polynucleotide of interest into virus particles, thereby providing a means for amplifying the number of infected host cells containing the polynucleotide therein.

The present invention also provides a CAEV-based lentiviral vector system for producing CAEV-based, replication-defective vector particles useful in delivering exogenous polynucleotides into mammalian cells. The vector particles are capable of infecting and transducing mammalian cells. The vector system comprises the transfer vector described above, and a packaging vector system, wherein said packaging vector system comprises: a first polynucleotide comprising a CAEV gag-pol-encoding sequence and an RRE, and a second polynucleotide comprising a viral envelope encoding sequence.

In one embodiment, the second polynucleotide comprises a non-CAEV env-encoding sequence. In one embodiment the second polynucleotide comprises a VSV-G- or GaLV-encoding sequence.

In another embodiment, the CAEV vector system comprises a third polynucleotide sequence comprising a rev-encoding sequence.

In another embodiment, the CAEV vector system comprises a fourth polynucleotide sequence comprising a vif-encoding sequence.

In a further embodiment, the first polynucleotide of each of the CAEV vector systems described above further comprises a heterologous regulatory sequence operably linked to the CAEV gag-pol-encoding sequence.

In a further embodiment, the second polynucleotide of the above-described CAEV vector systems further comprises a heterologous regulatory sequence operable linked to said viral envelope-encoding sequence.

In a further embodiment, the third polynucleotide further comprises a heterologous regulatory sequence operably linked to the rev-encoding sequence.

In a further embodiment, the fourth polynucleotide further comprises a heterologous regulatory sequence operably linked to the vif-encoding sequence.

In one embodiment of the invention, the CAEV vector system comprises a packaging vector system which is devoid of a competent CAEV packaging sequence. In a further embodiment, the packaging vector system is devoid of the 5′ end of the CAEV genome between the splice donor site and the gag start codon.

In one embodiment, the CAEV vector system comprises a first vector comprising the first polynucleotide and a second vector comprising the second polynucleotide. In another embodiment, the vector system comprises a first vector comprising the first polynucleotide, a second vector comprising the second polynucleotide, and a third vector comprising the third polynucleotide. In another embodiment, the vector system comprises a first vector comprising the first polynucleotide, a second vector comprising the second polynucleotide, a third vector comprising the third polynucleotide, and a fourth vector comprising the fourth polynucleotide. The third vector may be pHYK/rev (SEQ ID NO: 75), and the fourth vector may be pHYK/vif (SEQ ID NO: 76).

In yet another embodiment, the vector system comprises a first vector comprising the first polynucleotide, the third polynucleotide and the fourth polynucleotide, and a second vector comprising the second polynucleotide.

In one embodiment, the first vector of the CAEV vector system comprises a CAEV gag-encoding sequence and an RRE operable linked to a heterologous promoter. The promoter may be an MCMV MIEP. In a further embodiment, the CAEV vector system comprises the first vector pMGP/RRE (SEQ ID NO: 77).

In one embodiment, the second vector of the CAEV vector system is a VSV-G-encoding sequence operably linked to a heterologous promoter. The promoter may be an HCMV MIEP. The second vector may further comprise a beta globin intron. In a further embodiment, the CAEV vector system comprises the second vector pHGVSV-G (SEQ ID NO: 74).

In one embodiment, the second vector of the CAEV vector system is a GaLV env-encoding sequence operably linked to a heterologous promoter. The promoter may be an MCMV MIEP. The second vector may further comprise a eukaryotic elongation factor-1 alpha intron. In a further embodiment, the CAEV vector system comprises the second vector pMYKEF-1/env (SEQ ID NO: 72).

Another aspect of the invention is a method of producing a CAEV-based lentiviral vector particle useful for infecting mammalian cells. The method comprises (a) transfecting a cell with the vector system described supra, under conditions suitable for production of CAEV-based particles, where the vector particle is infection- and transduction-competent, and replication-defective, and (b) recovering the vector particle.

The present invention also provides a composition comprising a CAEV-based lentiviral vector particle and optionally a carrier, where the vector particle is produced by the methods described supra.

The present invention also provides a kit comprising the transfer vector or the CAEV-based lentiviral vector system described supra.

The present invention also provides a packaging cell comprising a CAEV gag-pol-encoding sequence and RRE, and optionally a viral env-encoding sequence. The packaging cell may further comprise a rev-encoding and/or a vif-encoding sequence. The cell is useful for packaging the RNA form of the transfer vector into an infection- and transduction-competent vector particle, which is replication-defective.

In one embodiment, the vector system comprises a cell comprising the first polynucleotide described supra. The vector system may further comprise the third and/or the fourth polynucleotide described supra.

In another embodiment, the vector system comprises a cell comprising the first polynucleotide and second polynucleotides described supra. The vector system may further comprise the third and/or the fourth polynucleotide described supra.

In another embodiment, the vector system comprises a cell comprising a first vector that comprises a CAEV gag-pol-encoding sequence and an RRE. The first vector may further comprise a rev-encoding and/or a vif-encoding sequence. Alternatively, the cell may comprise a first vector comprising a CAEV gag-pol-encoding sequence and an RRE, a second vector comprising a rev-encoding sequence and/or a third vector comprising a vif-encoding sequence.

In some embodiments, the vector system comprises a cell comprising a first vector that comprises a CAEV gag-pol-encoding sequence and an RRE, and a second vector that comprises a viral env-encoding sequence. The first vector may further comprise a rev-encoding and/or a vif-encoding sequence. Alternatively, the cell may comprise a first vector comprising the CAEV gag-pol-encoding sequence and an RRE, a second vector comprising a viral env-encoding sequence, and optionally a third vector comprising a rev-encoding sequence and/or a fourth vector comprising a vif-encoding sequence.

Another aspect of the present invention is a method of delivering a polynucleotide or polypeptide into a mammalian cell or replicating a polynucleotide molecule encoding said polypeptide, comprising contacting a mammalian cell with the vector particle described supra under conditions which may allow for integration of said polynucleotide into the genome of said cell and optionally under conditions allowing expansion of said polypeptide encoded by said polynucleotide. The mammalian cell may be a dividing cell, a non-dividing cell or a CD34+ stem cell. The method of delivering a polynucleotide or a polypeptide into a mammalian cell or replicating a polynucleotide molecule encoding said polypeptide, may further comprise isolating the cell from a mammal prior to contacting the cell with the vector particle. The method may further comprise expanding said cell in culture after contacting it with the vector particle. The method may further comprise reintroducing the cell into a mammal before or after expanding the contacted cell.

The present invention further provides a method for delivering a polypeptide into a vertebrate, comprising administering to the vertebrate a CAEV-based lentiviral vector particle comprising a heterologous polynucleotide of interest, where the vector particle is produced by the method described supra, such that the polypeptide encoded by the delivered polynucleotide is expressed in the vertebrate, in an amount sufficient to be detectable or to elicit a biological response in the vertebrate.

The present invention further provides a vector comprising a CAEV packaging sequence consisting essentially of (a) the untranslated region between the CAEV 5′ LTR and the CAEV gag-encoding sequence, and (b) nucleotides 1 to X of the CAEV gag-encoding sequence linked to the 3′ end of the untranslated region, wherein X is less than 613.

The inventors have discovered that the production of the CAEV-based lentiviral vector particles, as described herein, results in enhanced efficiency and safety in the lentiviral vector design over the existing CAEV-based vector particles. The enhanced efficiency is achieved through the discovery of the optimal length of the untranslated region between the 5′LTR and the gag start codon and the gag-encoding region, which serves as an efficient packaging sequence by allowing efficient encapsidation, which then results in increased viral titers. Viral titer is also improved by using a strong heterologous promoter in the design of the packaging plasmids. The enhanced safety is achieved through the construction of a tat-independent transfer vector and a plasmid-based packaging system.

### BRIEF DESCRIPTION OF THE DRAWINGS/FIGURES

FIG. 1 is a schematic illustration of the CAEV proviral genomic organization.

FIG. 2A is a schematic illustration of the plasmid pMGP/RRE (SEQ ID NO: 77). pMGP/RRE (SEQ ID NO: 77) is a 9,446 bp plasmid which contains an MCMV MIEP region (located at bp 1-660) located upstream of the CAEV gag-pol coding region (bp 709-5,243), the RRE region (5,426-5,627 or bp 5,368-5,669), and the bovine growth hormone (BGH) polyadenylation signal (bp 5,751-5,984). The vector also contains a neomycin resistance gene coding region (bp 8,151-7,155), a SV40 origin of replication (bp 8,509-8,152), a Col E1 origin of replication (bp 6,115-6,698), and an ampicillin resistance gene region (bp 9,362-8,528).

FIG. 2B is a schematic illustration of the plasmid pMGP/REV/RRE. pMGP/REV/RRE is a 9,924 bp plasmid which contains an MCMV MIEP region (located at bp 1-660) and the major splicing donor of CAEV (bp 688-704) located upstream of the CAEV gag-pol coding region (bp 726-5,258), the first exon rev coding region (bp 5,383-5,494), the RRE region (bp 5,540-5,841), the second exon rev coding region (bp 5,888-6,177), and the bovine growth hormone (BGH) polyadenylation signal (bp 6,229-6,462). The vector also contains a neomycin resistance gene coding region (bp 7,633-8,629), a SV40 origin of replication (bp 8,987-8,630), a Col E1 origin of replication (bp 6,593-7,176), and an ampicillin resistance gene region (bp 9,840-9,006).

FIG. 3A is a schematic illustration of the plasmid pCAH/SINd (SEQ ID NO: 73). pCAH/SINd (SEQ ID NO: 73) is a 3,566 bp plasmid which contains the HCMV MIEP (bp 1-588), the R-U5 sequence regions in the CAEV 5′LTR (bp 611-772), the RRE region (bp 796-1,154), and the U3-deleted CAEV 3′LTR region (bp 1,275-1,458). The vector also contains a Col E1 origin of replication (bp 1,863-2,466), and a kanamycin resistance gene coding region (bp 2,698-3,510).

FIG. 3B is a schematic illustration of the plasmid pCAH/SINd0 (SEQ ID NO: 67). pCAH/SINd0 (SEQ ID NO: 67) is a 3,911 bp plasmid which contains the HCMV MIEP (bp 1-588), the R-U5 sequence regions in CAEV 5′LTR (bp 611-772), the residual untranslated sequences containing the primer binding site (PBS) (bp 773-789), the RRE region (bp 1,141-1,499), and the U3-deleted CAEV 3′LTR region (bp 1,620-1,803). The vector also contains a Col E1 origin of replication (bp 2,208-2,791) and a kanamycin resistance gene coding region (bp 3,043-3,855).

FIG. 3C is a schematic illustration of the plasmid pCAH/SINd1 (SEQ ID NO: 68). pCAH/SINd1 (SEQ ID NO: 68) is a 4,238 bp plasmid which contains the HCMV MIEP (bp 1-588) promoter, the R-U5 sequence regions in the CAEV 5′LTR (bp 610-772), the residual untranslated sequences containing the PBS site (bp 773-789), the 327 bp fragment of the gag gene (bp 1,121-1,448) with ATG to TAG point mutations at the start ATG codon (bp1121-1123) and the ATG codon (bp1142-1144) located downstream of the start ATG codon, the RRE region (bp 1,468-1,826) and the U3-deleted CAEV 3′LTR region (bp 1,947-2,130). The vector also contains a Col E1 origin of replication (bp 2,535-3,118) and a kanamycin resistance gene region (bp 3,370-4,182).

FIG. 3D is a schematic illustration of the plasmid pCAH/SINd2 (SEQ ID NO: 69). Plasmid pCAH/SINd2 (SEQ ID NO: 69) is a 4,523 bp plasmid which contains the HCMV MIEP (bp 1-588), the R-U5 sequence regions in the CAEV 5′LTR (bp 610-772), the residual untranslated sequences containing the PBS site (bp 773-789), the 612 bp fragment of the gag gene (bp 1,121-1,733), with point mutations at the start ATG codon (bp 1121-1123) and the ATG codon (bp 1142-1144) located downstream of the start ATG codon, the RRE region (bp 1,753-2,111) and the U3-deleted CAEV 3′LTR region (bp 2,232-2,415). The vector also contains a Col E1 origin of replication (bp 2,820-3,403) and a kanamycin resistance gene coding region (bp 3,655-4,467).

FIG. 3E is a schematic illustration of the plasmid pCAH/SINd3 (SEQ ID NO: 70). pCAH/SINd3 (SEQ ID NO: 70) is a 4,819 bp plasmid which contains the HCMV MIEP (bp 1-588), the R-U5 sequence regions in CAEV 5′LTR (bp 610-772), the residual untranslated sequences containing PBS site (bp 773-789), the 908 bp fragment of the gag gene (bp 1,121-2,029) with point mutations at the start ATG codon (bp 1121-1123) and the ATG codon (bp 1142-1144) located downstream of the start ATG codon, the RRE region (bp 2,049-2,407) and the U3-deleted CAEV 3′LTR region (bp 2,549-2,711). The vector also contains a Col E1 origin of replication (bp 3,116-3,699) and a kanamycin resistance gene coding region (bp 3,951-4,763).

FIG. 3F is a schematic illustration of the plasmid pCAH/SINd4 (SEQ ID NO: 71). pCAH/SINd4 (SEQ ID NO: 71) is a 5,112 bp plasmid which contains the HCMV MIEP (bp 1-588), the R-U5 sequence regions in the CAEV 5′LTR (bp 610-772), the residual untranslated sequences containing the PBS site (bp 773-1,120), the 1198 bp fragment of the gag gene (bp 1,121-2,319) with point mutations at the start ATG codon (bp 1121-1123) and the ATG codon (bp 1142-1144) located downstream of the start ATG codon, the RRE region (bp 2,342-2,700) and the U3-deleted CAEV 3′LTR region (bp 2,842-3,004). The vector also contains a Col E1 origin of replication (bp 3,409-3,992), and a kanamycin resistance gene coding region (bp 4,244-5,056).

FIG. 3G is a schematic illustration of the plasmid pCAH/SINd1/hlacZ (SEQ ID NO: 79). pCAH/SINd1/hlacZ (SEQ ID NO: 79) is an 8,127 bp plasmid derived from the pCAH/SINd1 (SEQ ID NO: 68) that expresses the lacZ reporter gene. The vector contains two HCMV MIEP promoter regions (located at bp 1-588 and bp 1,866-2,460, respectively), the R-U5 sequence regions in the CAEV 5′LTR (bp 610-772), the residual untranslated sequences containing the PBS site (bp 773-789), the 325 bp fragment of gag gene (bp 1,121-1,446) with point mutations at the start ATG codon (bp 1121-1123) and the ATG codon (bp 1142-1144) located downstream of the start ATG codon, the RRE region (bp 1,466-1,836), the lacZ gene coding sequence (bp 2,541-5,711), and the U3-deleted CAEV 3′LTR region (bp 5,782-6,019). The vector also contains a Col E1 origin of replication (bp 6,424-7,007), and a kanamycin resistance gene coding region (bp 7,259-8,071).

FIG. 3H is a schematic illustration of the plasmid pCAH/SINd60/hlacZ (SEQ ID NO: 78). Plasmid pCAH/SINd60/hlacZ (SEQ ID NO: 78) is a 7,856 bp which contains two promoter regions, HCMV MIEP (located at bp 1-588 and bp 1,595-2,189, respectively), the R-U5 sequence regions in the CAEV 5′LTR (bp 610-772), the residual untranslated sequences containing the PBS site (bp 773-789 bp), the 60 bp fragment of gag gene (bp 1,121-1,181) with point mutations at the start ATG codon (bp 1121-1123) and the ATG codon (bp 1142-1144) located downstream of the start ATG codon, the RRE region (bp 1,195-1,565), the lacZ gene coding sequence (bp 2,270-5,440), and the U3-deleted CAEV 3′LTR region (bp 5,511-5,748). The vector also contains a Col E1 origin of replication (bp 6,153-6,736), and a kanamycin resistance gene coding region (bp 6,988-7,800).

FIG. 4 is a schematic illustration of the plasmid pHYK/vif (SEQ ID NO: 76). pHYK/vif (SEQ ID NO: 76) is a 5,729 bp plasmid which contains the HCMV MIEP (bp 1-596), the vif gene coding region (bp 691-1,380), the BGH polyadenylation signal (bp 1,467-1,695), a Col E1 origin of replication (bp 1,826-2,409), a neomycin resistance gene coding region (bp 3,862-2,866), and an ampicillin resistance gene coding region (bp 5,270-4,239).

FIG. 5 is a schematic illustration of the plasmid pHYK/rev (SEQ ID NO: 75). pHYK/rev (SEQ ID NO: 75) is a 5,419 bp plasmid which contains the HCMV MIEP (bp 1-596), the rev gene coding region (bp 672-1,073), the BGH polyadenylation signal (bp 1,157-1,385), a Col E1 origin of replication (bp 1,516-2,099), a neomycin resistance gene coding region (bp 3,552-2,556), and an ampicillin resistance gene coding region (bp 4,960-3,929).

FIG. 6A is a schematic illustration of the plasmid pHGVSV-G (SEQ ID NO: 74). pHGVSV-G (SEQ ID NO: 74) is a 7,623 bp plasmid which contains the HCMV MIEP (bp 1-596), the β-globin intron region (bp 714-1,599), the VSV-G coding region (bp 1,632-3,312), the BGH polyadenylation signal (bp 3,361-3,589), a Col E1 origin of replication (bp 3,720-4,303), a neomycin resistance gene coding region (bp 5,756-4,760), an ampicillin resistance gene coding region (bp 7,164-6,133), and a F1 origin of replication (bp 7,165-7,621).

FIG. 6B is a schematic illustration of the plasmid pMYKEF1/env (SEQ ID NO: 72). pMYKEF1/env (SEQ ID NO: 72) is a 7,579 bp plasmid which contains the MCMV MIEP (bp 1-665), a human EF1-α intron region (bp 668-1,618), the GaLV env coding region (bp 1,699-3701), the BGH polyadenylation signal (bp 3,885-4,118), a Col E1 origin of replication (bp 4,349-4,832), a neomycin resistance gene coding region (bp 6,290-5,284), and an ampicillin resistance gene coding region (bp 7,496-6,666).

FIG. 7 shows a photograph illustrating the relative amount of transfer vector RNA transcribed from gene transfer vectors transfected into human 293T target cells.

FIG. 8 shows two photographs illustrating gene transfer into human 293T target cells by CAEV (A) and MuLV (B) vectors.

FIG. 9 shows a photographic illustration of the relative amount of transfer vector RNA expressed in the transfected 293T cells (lanes 1, 2 and 3), and encapsidated in and released from the 293T packaging cells (lanes 4, 5 and 6).

FIG. 10 shows a photograph illustrating the relative amount of transfer vector RNA encapsidated in and released from human 293T packaging cells. FIG. 11 shows a photograph illustrating the relative amount of integrated retroviral cDNA after infection and reverse transcription of lentiviral vectors pseudotyped by VSV-G or GaLV envelope protein.

FIG. 12 shows a photograph illustrating the relative amount of viral vector cDNA integrated into the infected host cell chromosome.

FIG. 13 shows two graphs illustrating the FACS analysis of (A) the control cells, and (B) the G1-arrested cells.

FIG. 14 shows two graphs illustrating (A) the number of transduced cells and (B) the relative transduction efficiencies of HIV-1-, CAEV-, and MuLV-derived viral vectors on dividing and non-dividing cells.

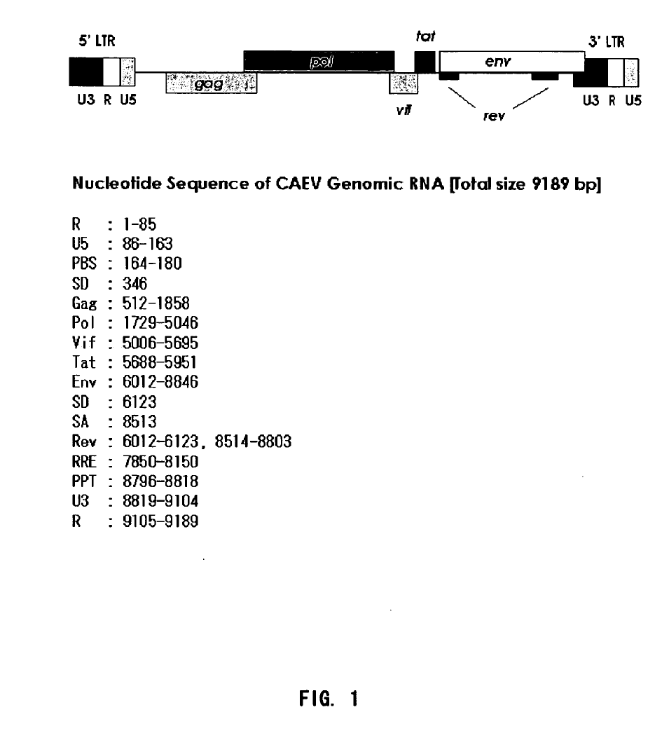

### DETAILED DESCRIPTION OF THE INVENTION

The invention relates to, inter alia, CAEV-based lentiviral vector systems and methods employing said vectors to deliver polypeptides of interest into dividing and non-dividing cells.

The CAEV Genome

The wild-type CAEV virus has a dimeric RNA genome (single-stranded, positive polarity) that is replicated through a double-stranded DNA intermediates and is packaged into a spherical enveloped virion containing a nucleoprotein core. The genome contains three genes that encode the structural and enzymatic proteins Gag, Pol, and Env, and long terminal repeats (LTR) at each end of the integrated viral genome. In addition, the genome encodes three regulatory proteins, vif, tat, and rev.

The gag gene encodes the internal structural proteins, the pol gene encodes viral replication enzymes, and the env gene encodes an envelope glycoprotein that mediates attachment of virus to the cell surface. The Vif protein is associated with viral infectivity, and the Tat protein with transactivation of the 5′ LTR. The Rev protein and its target sequence RRE (Rev responsive element) are associated with the stability of viral RNA, regulation of viral RNA splicing, and transport of large RNA (unspliced and singly-spliced) from the nucleus to the cytoplasm. The proviral LTR sequences contain the U3 (unique sequence element located downstream from the structural proteins), R (short repeat at each end of the genome), and U5 (unique sequence element immediately after the R sequence) regions. The U3 region of 5′LTR contains the viral promoter and enhancers. The 3′ end of the genome contains polyadenylation signal in the 3′LTR.

The wild-type genome of CAEV also contains several cis-acting elements, including atts (attachment site) at the end of LTRs for provirus integration); promoter elements that control transcriptional initiation of the integrated provirus at the 5′LTR; a PBS (primer binding site) located downstream of the 5′LTR; a 5′-splice donor site; a packaging sequence (herein referred to interchangeably as a packaging site or a packaging signal); a ppt (polypurine tract) site located near the 3′LTR; and polyadenylation signals at the 3′LTR.

As used herein, the term “cis” is used in reference to the presence of genes on the same chromosome or linear portion of a nucleic acid. Therefore, the term “cis-defect” refers to a defect found on a linear sequence of a nucleic acid. The term “cis-acting” is used in reference to the controlling effect of a regulatory gene on a gene present on the same chromosome or linear portion of a nucleic acid. For example, promoters, which affect the synthesis of downstream mRNA are cis-acting control elements.

The complete genomic sequence for two isolates of CAEV are known and the sequences are deposited in the National Center for Biotechnology Information (NCBI) database as NC_{—}001463 (SEQ ID NO: 1) and AF322109 (SEQ ID NO: 2) (Saltarelli et al., 1990, and Gjerset, B. J. et al. unpublished, respectively). The nucleic acids of the claimed invention are not limited to a particular isolate of CAEV, but rather to a sequence that retains the known function of that genomic sequence. For example, it is known in the art that natural variations in a gene sequence may occur during viral replication, resulting in a similar nucleic acid sequence that encodes proteins having a similar function.

A sequence alignment of the NC_{—}001463 (SEQ ID NO: 1) and AF322109 (SEQ ID NO: 2) genomic sequences is shown in TABLE 1. As is visible in TABLE 1, there is considerable nucleic acid identity between the sequences, however differences at the nucleic acid level are apparent. Of particular importance is the variability of the CAEV gag region denoted in TABLE 2 (SEQ ID NOs: 3-6). Sequence alignments of NC_{—}001463 5′LTR, pol, rev, and vif genes and the corresponding genes from AF322109 can be found in TABLES 3-6 (SEQ ID NOs: 7-14), respectively. Many partial sequences of the CAEV genome are also known and have been deposited. For example, accession numbers AY081139, AY101347, AY101348, AY047362, AF402668, AF402667, AF402666, AF402665, AF402664, AJ305042, AJ305041, and AJ305040 all provide for sequences of the gag gene from Brazilian isolates of CAEV. Accession numbers AF015181, L78453, L78451, L78450, L78447, and L78446 also contain the sequences of gag genes from a variety of CAEV isolates. Accession numbers X64828 and M63106 contain the sequences of rev genes from a variety of CAEV isolates. Accession numbers AF015182, AJ305053, K03327, L78448, L78452 and U35814 contain pol genes from a variety of CAEV isolates. A sequence alignment between the NC_{—}001463 gag gene (SEQ ID NOs: 15, 17) and the AF015181 gag gene (SEQ ID NOs: 16, 18) is found in TABLE 7. A sequence alignment between the NC_{—}001463 gag gene (SEQ ID NOs: 19, 25) and the gag genes from AF402664 (SEQ ID NOs: 20, 26), AF402665 (SEQ ID NOs: 21, 27), AF402666 (SEQ ID NOs: 22, 28), AF402667 (SEQ ID NOs: 23, 29), AF402668 (SEQ ID NOs: 24, 30) is found in TABLE 8. A sequence alignment between the NC_{—}001463 gag gene (SEQ ID NOs: 31, 35) and the gag genes from AJ305040 (SEQ ID NOs: 32, 36), AJ305041 (SEQ ID NOs: 33, 37), AJ305042 (SEQ ID NOs: 34, 38) is found in TABLE 9. A sequence alignment between the NC_{—}001463 gag gene (SEQ ID NOs: 39, 41) and the gag gene from AY047362 (SEQ ID NOs: 40, 42) is found in TABLE 10. A sequence alignment between the NC_{—}001463 (SEQ ID NOs: 43, 45) gag gene and the gag gene from AY081139 (SEQ ID NOs: 44, 46) is found in TABLE 11. A sequence alignment between the NC_{—}001463 (SEQ ID NOs: 47, 50) gag gene and the gag genes from AY101347 (SEQ ID NOs: 48, 51) and AY101348 (SEQ ID NOs: 49, 52) is found in TABLE 12. A sequence alignment between the NC_{—}001463 gag gene (SEQ ID NOs: 53, 59) and the gag genes from L78446 (SEQ ID NOs: 54, 60), L78447 (SEQ ID NOs: 55, 61), L78450 (SEQ ID NOs: 56, 62), L78451 (SEQ ID NOs: 57, 63), and L78453 (SEQ ID NOs: 58, 64) is found in TABLE 13.

The alignments were performed using VectorNTI (Informax, USA) using the following parameters:

For pairwise alignment: gap opening penalty: 15
Gap extension penalty: 6.6

For multiple alignment: gap opening penalty: 15
Gap extension penalty : 6.6
Gap separation penalty range : 8

TABLE 14 is a summary of the percent identity values for the sequence alignments of gag gene sequences listed above. TABLE 15 is a summary of the percent identity of the full genomic alignment, and alignments of the gag, 5′ LTR, pol, rev, and vif regions of NC_{—}001463 (SEQ ID NO: 1) and AF322109 (SEQ ID NO: 2). Given that the genomic sequence of two CAEV isolates, in addition to a large number of partial sequences from a variety of CAEV isolates are known and consensus sequences can be easily discerned, it would not require undue experimentation to practice the claimed invention using a variety of CAEV sequences.

CAEV Vectors of the Invention

The vectors of the present invention provide a means for replicating and expressing polynucleotides or genes independent of the host cell nucleus in a broad phylogenetic range of host cells. This vector-mediated incorporation of heterologous nucleic acid into a host cell is referred to as transfection or infection of the host cell, wherein infection means the use of virus particles, and transfection means the use of naked molecules of nucleic acid.

The term “gene” refers to a DNA sequence that comprises control and coding sequences necessary for the production of a polypeptide or precursor. The term “polynucleotide” or “nucleic acid molecule”, as used interchangeably herein, refers to nucleotide polymers of any length, such as two or more, and includes both DNA and RNA. The nucleotides can be deoxyribonucleotides, ribonucleotides, nucleotide analogs (including modified phosphate moieties, bases, or sugars), or any substrate that can be incorporated into a polymer by a suitable enzyme, such as a DNA polymerase or an RNA polymerase. The polypeptide can be encoded by a full-length coding sequence or by any portion of the coding sequence so long as the desired activity of the polypeptide is retained.

The term “wild-type” refers to a gene or gene product which has the characteristics of that gene or gene product when isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product which displays modifications in sequence and or functional properties (i.e., altered characteristics) when compared to the wild-type gene or gene product. Naturally-occurring mutants can be isolated, and are identified by the fact that they have altered characteristics when compared to the wild-type gene or gene product.

It must be noted that as used in this specification and the appended claims, the singular forms “a”, “an”, “the”, and the like, include plural references unless the context clearly dictates otherwise. Thus, for example, reference to “a polynucleotide” includes polynucleotides and “a stem cell” includes a plurality of cells.

As used herein, the term “retrovirus” is used in reference to RNA viruses that utilize reverse transcriptase during their replication cycle. The retroviral genomic RNA is converted into double-stranded DNA by reverse transcriptase. This double-stranded DNA form of the virus is capable of being integrated into the chromosome of the infected cell; once integrated, it is referred to as a “provirus.” The provirus serves as a template for RNA polymerase II and directs the expression of RNA molecules which encode the structural proteins and enzymes needed to produce new viral particles.

As used herein, the term “lentivirus” refers to a group (or genus) of retroviruses that give rise to slowly developing disease. Viruses included within this group include human immunodeficiency virus (HIV); visna-maedi, which causes encephalitis (visna) or pneumonia (maedi) in sheep, caprine arthritis encephalitis virus (CAEV); equine infectious anemia virus (EIAV); feline immunodeficiency virus (FIV); bovine immune deficiency virus (BIV); and simian immunodeficiency virus (SIV). Diseases caused by these viruses are characterized by a long incubation period and protracted course. Usually, the viruses latently infect monocytes and macrophages, from which they spread to other cells.

As used herein, the term “vector” is used in reference to nucleic acid molecules that transfer polynucleotide (e.g. DNA) segments from one cell to another. The term “vehicle” is sometimes used interchangeably with “vector.” It is intended that any form of vehicle or vector be encompassed within this definition. For example, vectors include, but are not limited to viral particles, plasmids, transposons, etc.

Standard techniques for the construction of the vectors of the present invention are well-known to those of ordinary skill in the art and can be found in such references as Sambrook et al., Molecular Cloning: A Laboratory Manual 2nd Ed. (Cold Spring Harbor, N.Y., 1989). A variety of strategies are available for ligating fragments of DNA, the choice of which depends on the nature of the termini of the DNA fragments and which choices can be readily made by the skilled artisan.

Suitable polyadenylation sequences of the present invention include, but are not limited to the bovine growth hormone (BGH) polyadenylation signal (Pfarr et al., 1986), the SV40 early region polyadenylation site (Hall et al., 1983) and the SV40 late region polyadenylation site (Carswell and Alwine, 1989), β-globin polyA, and herpes simplex virus thymidine kinase polyA.

A promoter of the present invention may comprise a promoter of mammalian or viral origin, and will be sufficient to direct the transcription of a distally located sequence (i.e. a sequence linked to the 5′ end of the promoter sequence) in a cell. The promoter region may also include control elements for the enhancement or repression of transcription. Suitable promoters include, but are not limited to, the human or murine cytomegalovirus immediate-early promoter (HCMV MIEP or MCMV MIEP), elongation factor 1 alpha (ef-1α), and Rous Sarcoma virus long terminal repeat promoter (pRSV). Intron sequences may also be combined with a promoter. Intron sequences include, but are not limited to ef-1α intron and β-globin intron. Inducible expression systems may also be used. Examples of inducible systems include, but are not limited to ecdysone-inducible mammalian expression system (Invitrogen, CA, USA) and Tet-On and Tet-Off gene expression systems (Clontech, CA, USA). Cell or tissue specific promoters can be utilized to target expression of gene sequences in specific cell populations.

Enhancer sequences upstream from the promoter or terminator sequences and downstream of the coding region may be optionally included in the vectors of the present invention to facilitate expression. Vectors of the present invention may also contain additional nucleic acid sequences, such as an intron sequence, a localization sequence, or a signal sequence, sufficient to permit a cell to efficiently and effectively process the protein expressed by the nucleic acid of the vector. Examples of intron sequences include the β-globin intron (Kim et al., 2002) and the human EF-1α intron (Kim et al., 2002). Such additional sequences are inserted into the vector such that they are operably linked with the promoter sequence, if transcription is desired, or additionally with the initiation and processing sequence if translation and processing are desired. Alternatively, the inserted sequences may be placed at any position in the vector.

The term “operably linked” is used to describe a linkage between a gene sequence and a promoter or other regulatory or processing sequence such that the transcription of the gene sequence is directed by an operably linked promoter sequence, the translation of the gene sequence is directed by an operably linked translational regulatory sequence, and the post-translational processing of the gene sequence is directed by an operably linked processing sequence.

The term “SIN vector” refers to the self-inactivating vector that has a truncated U3 region in the 3′ LTR. During reverse transcription, a truncated U3 is duplicated in the 5′LTR, resulting in the loss of the transcription capacity and the interference effect on an internal promoter.

The packaging sequence of the transfer vector consists essentially of (i) the untranslated region between the CAEV 5′ LTR and the CAEV gag-encoding sequence, and (ii) nucleotides 1 to X of the CAEV gag-encoding sequence linked to the 3′ end of said untranslated region, wherein X is less than 613. In one embodiment of the invention, X is selected from the group consisting of: 60, 100, 125, 150, 175, 200, 225, 250, 275, 300, 325, 350, 375, 400, 425, 450, 475, 500, 525, 550, 575 and 600.

In another embodiment of the invention, X is selected from the group consisting of:

X is greater than 25 and less than 600,

X is greater than 25 and less than 500,

X is greater than 25 and less than 400,

X is greater than 25 and less than 300,

X is greater than 25 and less than 200,

X is greater than 50 and less than 600,

X is greater than 50 and less than 500,

X is greater than 50 and less than 400,

X is greater than 50 and less than 300,

X is greater than 50 and less than 200,

X is greater than 75 and less than 600,

X is greater than 75 and less than 500,

X is greater than 75 and less than 400,

X is greater than 75 and less than 300,

X is greater than 75 and less than 200,

X is greater than 100 and less than 600,

X is greater than 100 and less than 500,

X is greater than 100 and less than 400,

X is greater than 100 and less than 300,

X is greater than 100 and less than 200,

X is greater than 125 and less than 600,

X is greater than 125 and less than 500,

X is greater than 125 and less than 400,

X is greater than 125 and less than 300,

X is greater than 125 and less than 200,

X is greater than 150 and less than 600,

X is greater than 150 and less than 500,

X is greater than 150 and less than 400,

X is greater than 150 and less than 300,

X is greater than 150 and less than 200,

X is greater than 200 and less than 600,

X is greater than 200 and less than 500,

X is greater than 200 and less than 400,

X is greater than 200 and less than 300,

X is greater than 200 and less than 200,

X is greater than 250 and less than 600,

X is greater than 250 and less than 500,

X is greater than 250 and less than 400, and

X is greater than 250 and less than 300.

In another embodiment, X is greater than 40 and less than 613. In yet another embodiment, X is about 327. In one embodiment of the transfer vector, the codon which initiates gag translation has been mutated (e.g. ATG changed to TAG, TTG, CTG, or ATT) or deleted. The term “codon” refers to a sequence of three nucleotides in a DNA or messenger RNA molecule that represents the instruction for incorporation of a specific amino acid into a growing polypeptide chain. The transfer vector further comprises a heterologous promoter and one or more cis-acting sequences.

As used herein, the term “packaging signal” or “packaging sequence” refers to sequences located adjacent to the 5′ LTR of the CAEV genome which are required for encapsidation of the viral RNA into the viral capsid or particle. Several retroviral vectors use the minimal packaging signal (also referred to as the psi [ψ] sequence) needed for encapsidation of the viral genome. Thus, as used herein, the terms “packaging sequence”, “packaging signal”, “psi”, and the symbol “ψ” are used in reference to the non-coding sequence required for encapsidation of CAEV RNA strands during viral particle formation.

In another embodiment of the invention, the transfer vector further comprises a transcription cassette. The term “transcription cassette” as used herein refers to a fragment or segment of nucleic acid containing a particular grouping of genetic elements, generally a polynucleotide which expresses a polypeptide of interest, operably linked to a heterologous promoter. The cassette can be removed and inserted into a vector or plasmid as a single unit.

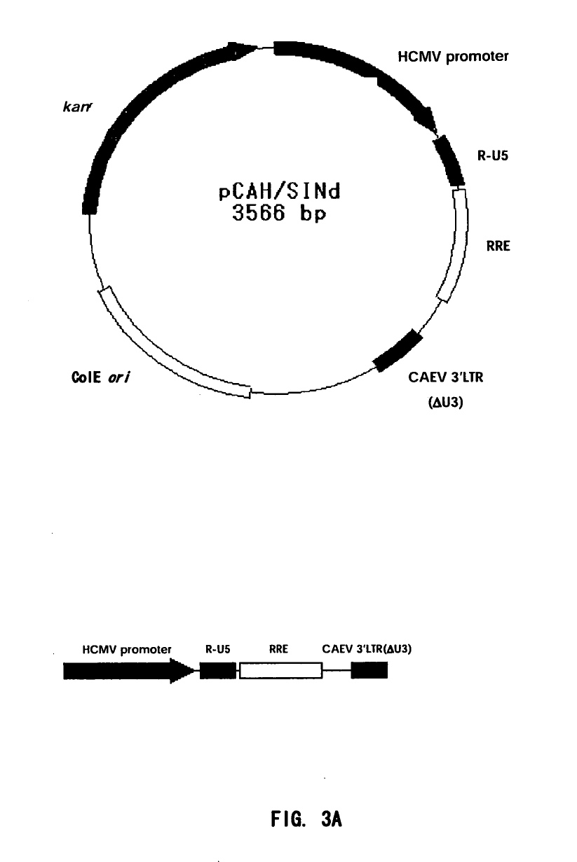

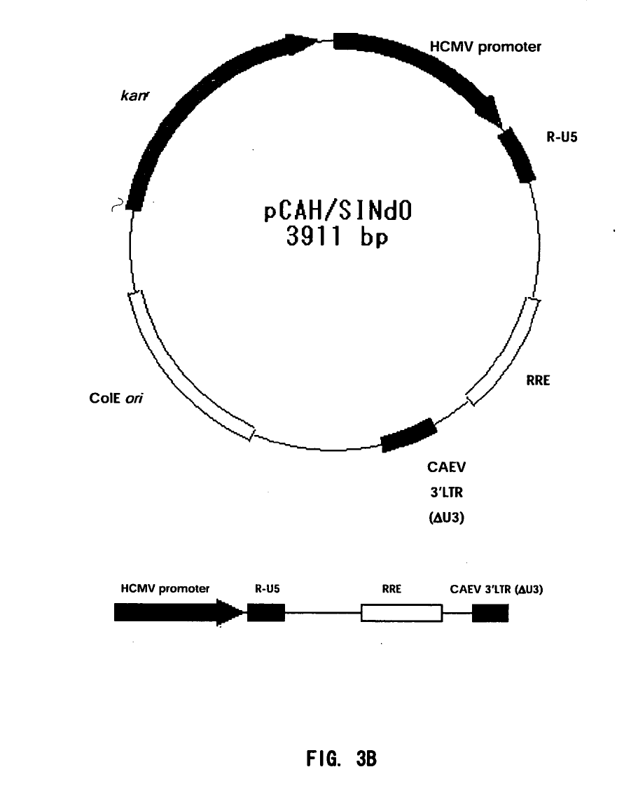

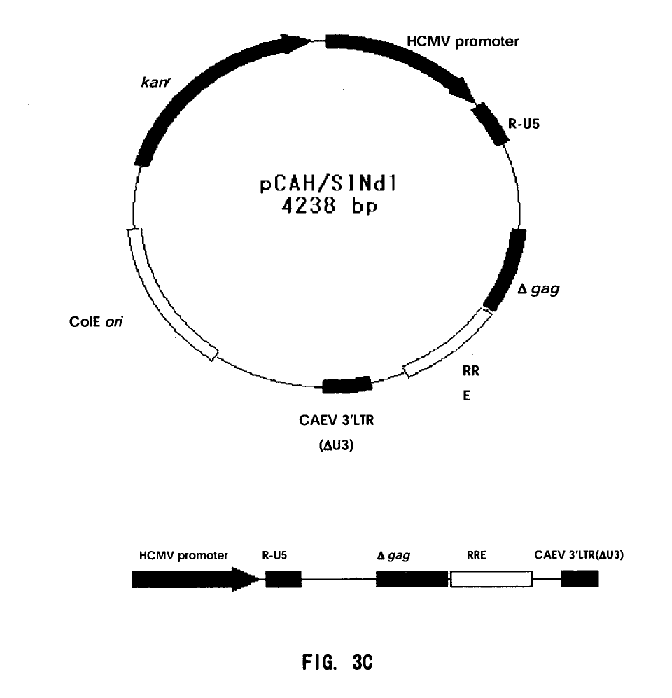

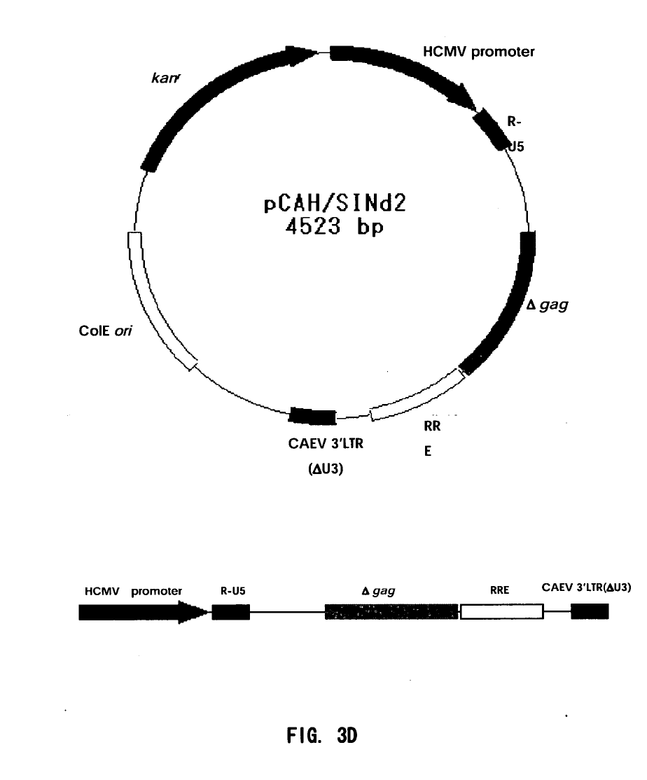

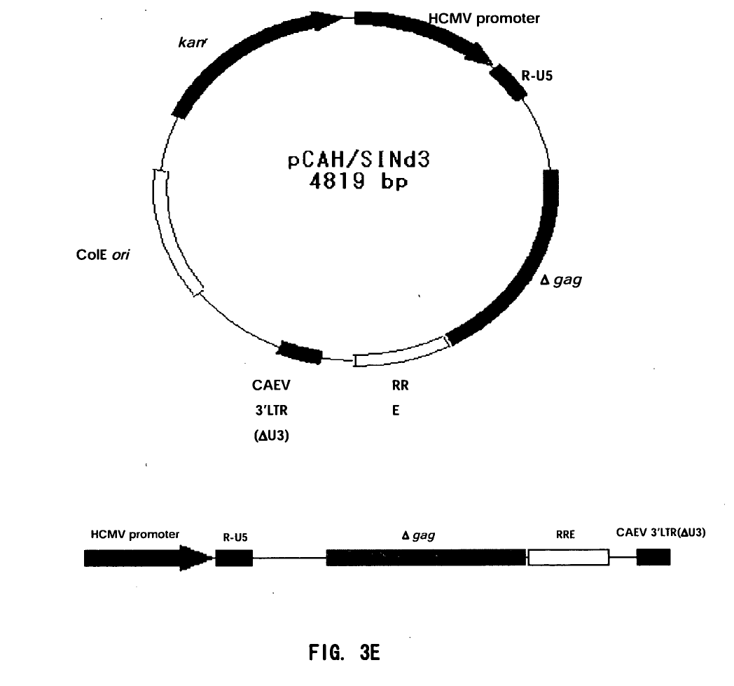

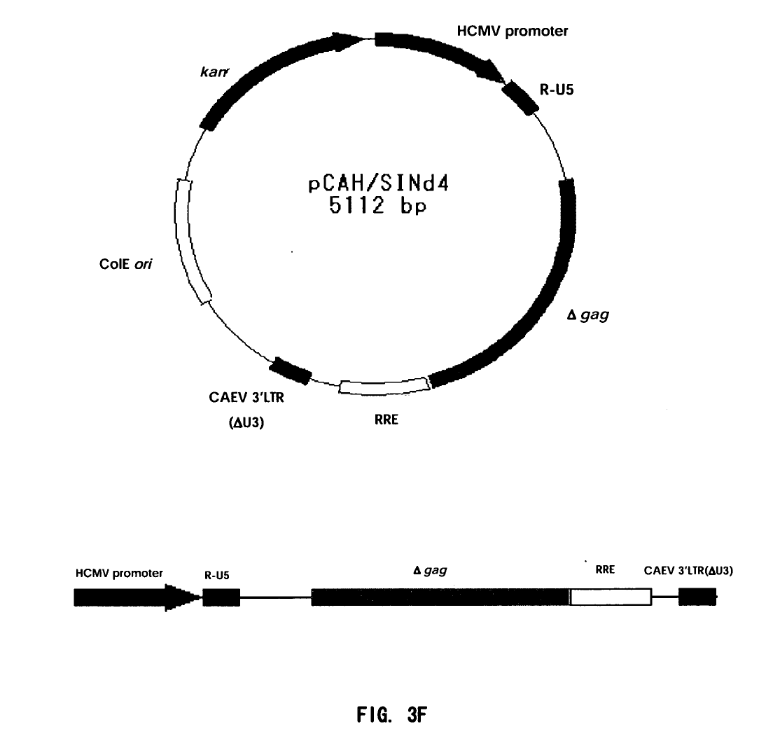

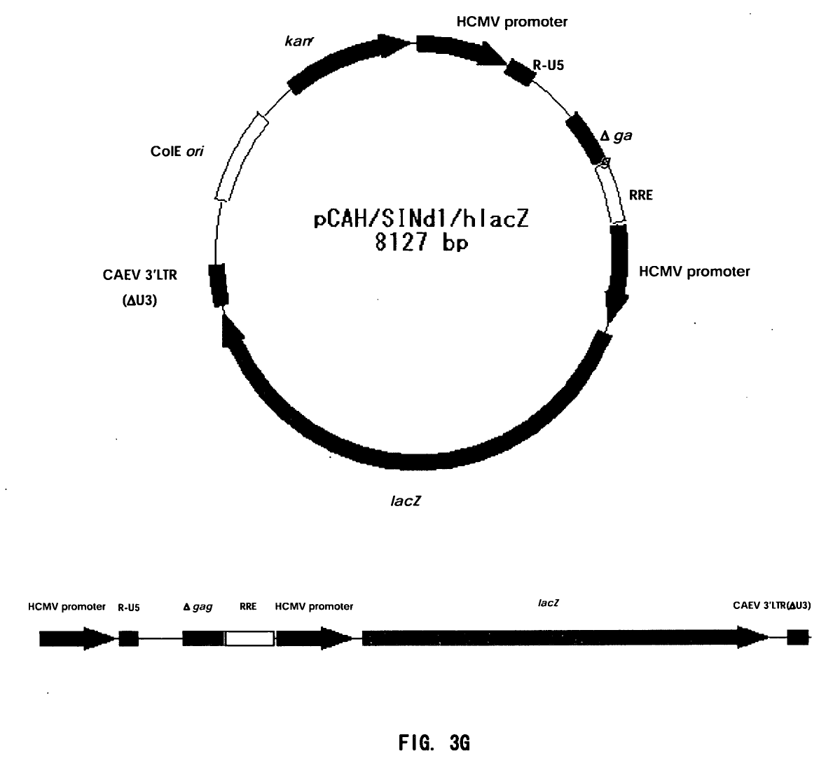

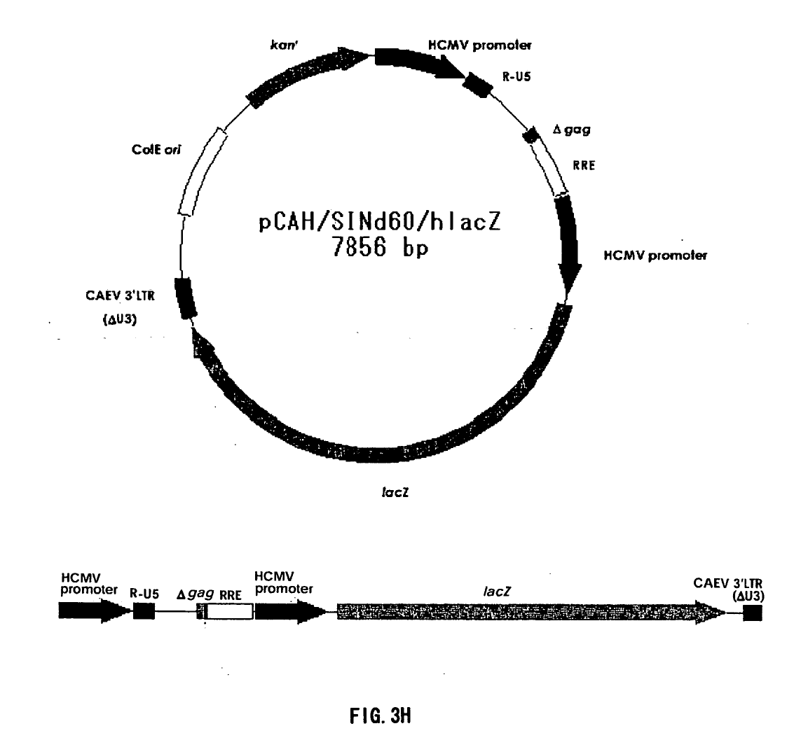

An illustrative example of a transfer vector of the present invention is shown in FIG. 3C. FIG. 3C illustrates the plasmid pCAH/SINd1 (SEQ ID NO: 68). PCAH/SINd1 (SEQ ID NO: 68) is a 4,238 bp plasmid that contains the HCMV MIEP promoter, the R-U5 sequence regions in the CAEV 5′LTR, the residual untranslated sequences containing a PBS site, the 327 bp fragment of the gag gene with the ATG→TAG double point mutations, the RRE region and the U3-deleted CAEV 3′LTR region. The vector also contains a Col E1 origin of replication (bp 2535-3118) and a kanamycin resistance gene region (bp 3370-4182). The other illustrative examples of transfer vectors are shown in FIG. 3A-3H.

The invention provides a CAEV vector system comprising the above described transfer vector and a packaging vector system. The packaging vector system comprises a first and second polynucleotide vector sequence. The first polynucleotide sequence comprises CAEV gag-pol and RRE-encoding sequence and the second polynucleotide comprises a viral envelope encoding sequence. In one embodiment, the second polynucleotide encodes a non-CAEV envelope.

The phrases “structural gene” as used herein refer to the polynucleotide sequence encode proteins which are required for encapsidation (e.g., packaging) of the viral genome, and include gag, pol and env.

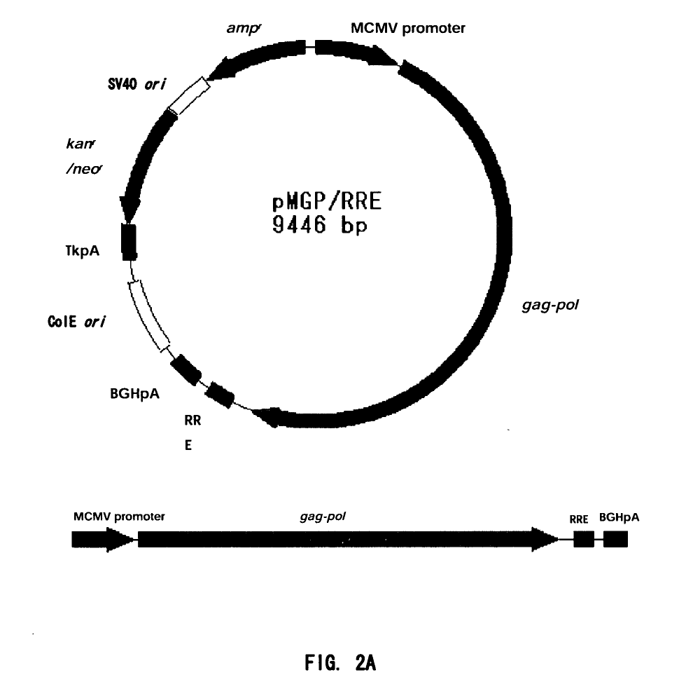

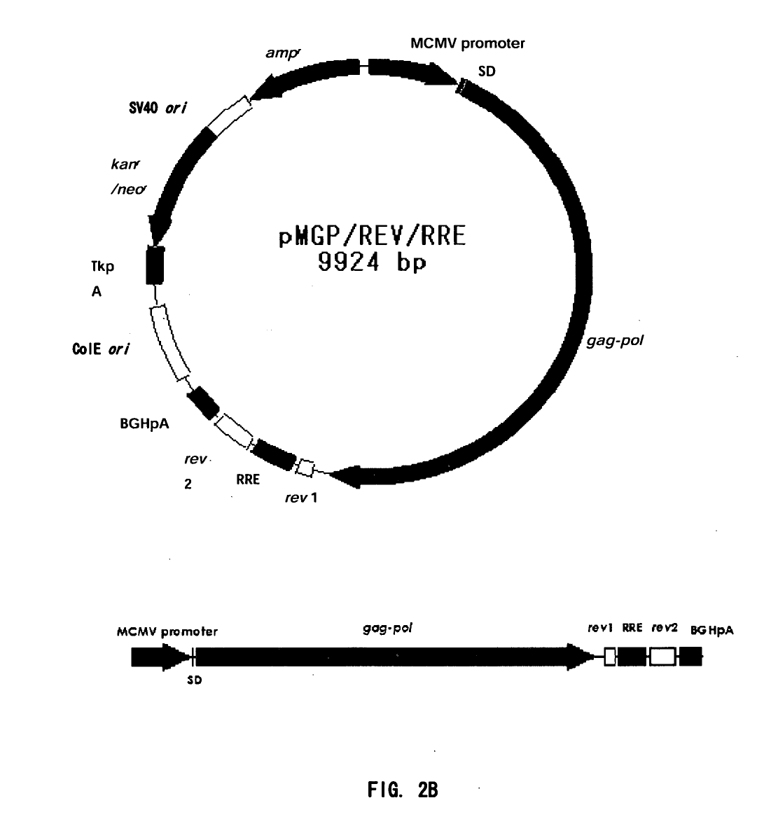

An illustrative example of a first packaging vector of the present invention is shown in FIG. 2A. FIG. 2A illustrates the plasmid pMGP/RRE (SEQ ID NO: 77). The plasmid contains 9,446 base pairs and includes a MCMV MIEP region, the CAEV gag-pol coding region, the RRE region, and the bovine growth hormone (BGH) polyadenylation signal. The vector also contains a neomycin resistant gene coding region, a SV40 origin of replication, a Col E1 origin of replication, and an ampicillin resistance gene region.

It is possible to alter the host range of cells that the viral vectors of the present invention can infect by utilizing an envelope gene from another closely related virus. In other words, it is possible to expand the host range of the CAEV vectors of the present invention by taking advantage of the capacity of the envelope proteins of certain viruses to participate in the encapsidation of other viruses. Examples of retroviral-derived env gene include, but are not limited to: the G-protein of vesicular-stomatitis virus (VSV-G), gibbon ape leukemia virus (GaLV), rous sarcoma virus (RSV), moloney murine leukemia virus (MoMuLV), mouse mammary tumor virus (MMTV), and human immunodeficiency virus (HIV). All of these viral envelope proteins efficiently form pseudotyped virions with genome and matrix components of other viruses. As used herein, the term “pseudotype” refers to a viral particle that contains nucleic acid of one virus but the envelope protein of another virus. In general, either VSV-G or GaLV pseudotyped vectors have a very broad host range, and may be pelleted to titers of high concentration by ultracentrifugation (Burns et al., 1993), while still retaining high levels of infectivity.

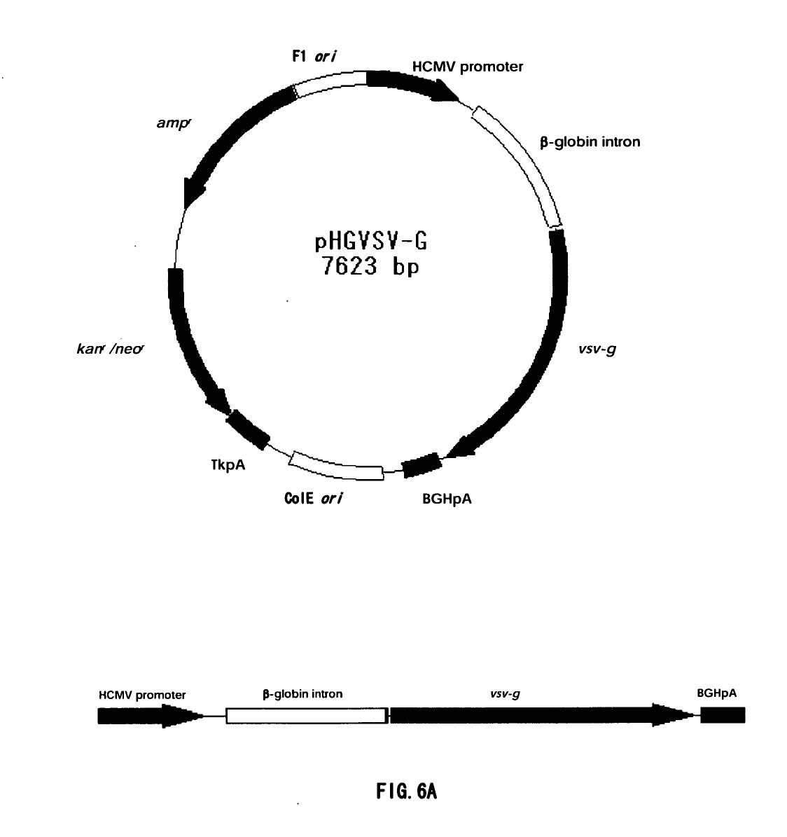

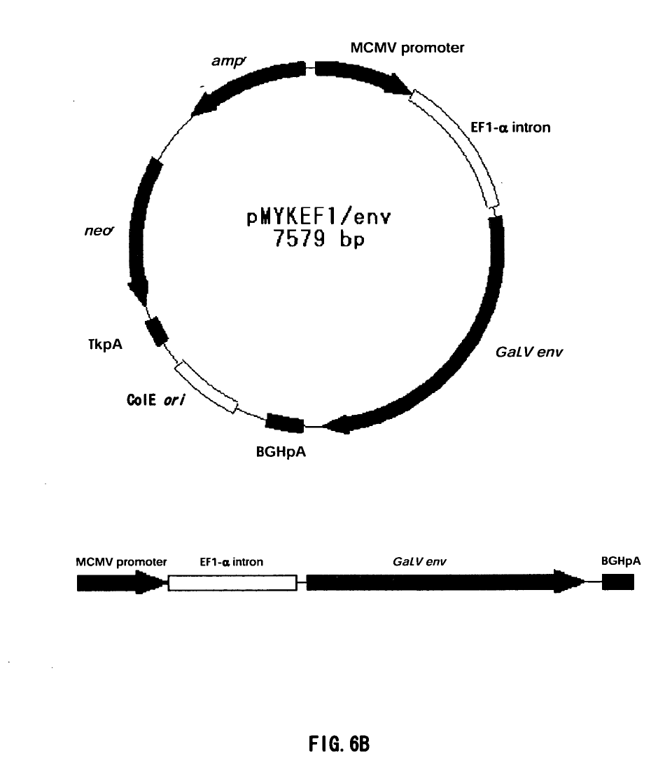

Other illustrative examples of second packaging vectors of the present invention are shown in FIGS. 6A and 6B. FIG. 6A illustrates the plasmid pHGVSV-G (SEQ ID NO: 74). pHGVSV-G (SEQ ID NO: 74) is a 7,623 bp plasmid which contains the HCMV MIEP, the β-globin intron region, the VSV-G coding region, the BGH polyadenylation signal, a Col E1 origin of replication, a neomycin resistance gene coding region, an ampicillin resistance gene coding region, and an F1 origin of replication. FIG. 6B illustrates the plasmid pMYKEF1/env (SEQ ID NO: 72). This plasmid contains 7,579 bp which includes the MCMV MIEP, a human EF1-α intron region, the GaLV env coding region, the BGH polyadenylation signal, a Col E1 origin of replication, a neomycin resistance gene coding region, and an ampicillin resistance gene coding region.

In another embodiment of the invention, the packaging vector comprises a third polynucleotide which encodes Rev. In infected cells, Rev binds to the Rev-responsive element (RRE) in viral transcripts and causes the transcription of both singly-spliced and unspliced transcripts characteristic of the viral structural proteins in the late stage of replication. Accordingly, Rev mediates temporal regulation of viral gene expression. Because mammalian cell splicing mechanisms are coupled to transport of mRNA from the site of synthesis in the nucleus to the cytoplasm, Rev also influences transport of viral transcripts containing RRE.

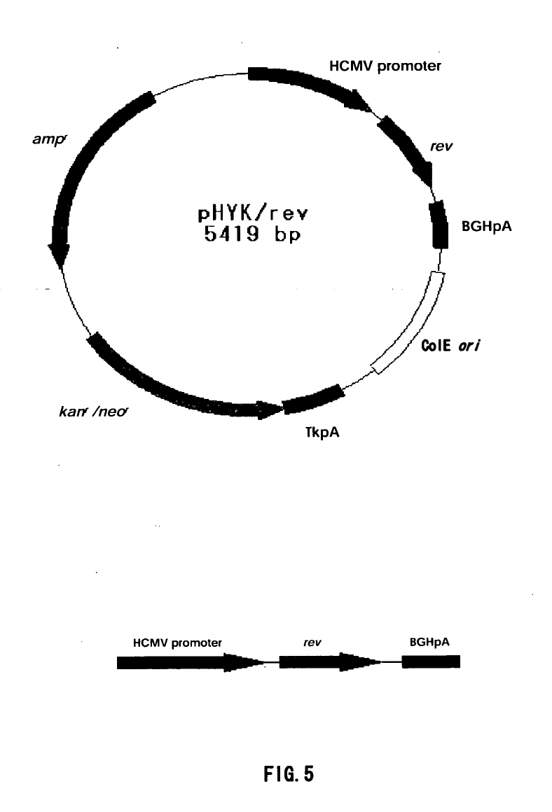

An illustrative example of a third packaging vector of the present invention is shown in FIG. 5. FIG. 5 illustrates the plasmid pHYK/rev (SEQ ID NO: 75). pHYK/rev (SEQ ID NO: 75) is a 5,419 bp plasmid which contains HCMV MIEP, the rev gene coding region, BGH polyadenylation signal, a Col E1 origin of replication, a neomycin resistant gene coding region, and an ampicillin resistant gene coding region.

In yet another embodiment of the invention, the packaging vector comprises a fourth polynucleotide encoding Vif. Incorporation of Vif may be necessary for infection and packaging of virions, depending on the packaging cell line chosen.

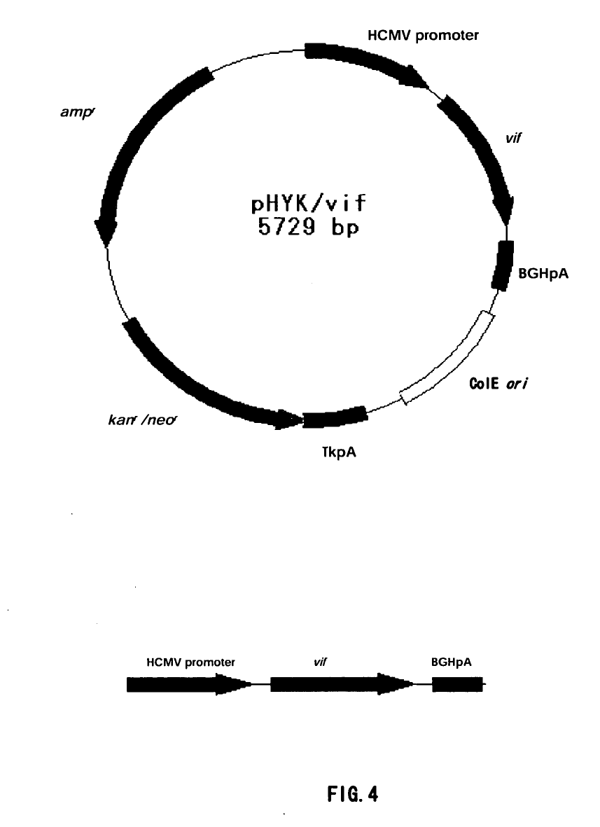

An illustrative example of a fourth packaging vector of the present invention is shown in FIG. 4. pHYK/vif (SEQ ID NO: 76) is a 5,729 bp plasmid which contains the HCMV MIEP, the vif gene coding region, the BGH polyadenylation signal, a Col E1 origin of replication, a neomycin resistance gene coding region, and an ampicillin resistance gene coding region.

When retroviral vector DNA is transfected into the cells, it may or may not become integrated into the chromosomal DNA and becomes transcribed, thereby producing full-length retroviral vector RNA that contains a ψ sequence. Under these conditions, only the vector RNA is packaged into the viral capsid structures. These complete, yet replication-defective, virus particles can then be used to deliver the retroviral vector to target cells with relatively high efficiency.

As used herein, the term “replication-defective” refers to a virus that is not capable of complete, effective replication such that infective virions are not produced (e.g. replication-defective lentiviral progeny). The term “replication-competent” refers to wild-type virus or mutant virus that is capable of replication, such that viral replication of the virus is capable of producing infective virions (e.g., replication-competent lentiviral progeny).

It is also contemplated that packaging may be inducible, as well as non-inducible. In inducible packaging cells and packaging cell lines, CAEV particles are produced in response to at least one inducer. In preferred embodiments with inducible cell lines, the inducer is Tat. In non-inducible packaging cell lines and packaging cells, no inducer is required in order for lentiviral particle production to occur.

CAEV Vector Sequences

Functionally equivalent sequences of the present invention also encompass various fragments of a CAEV genome that retain substantially the same function as the respective native sequence. Such fragments will comprise at least about 10, 15 contiguous nucleotides, at least about 20 contiguous nucleotides, at least about 24, 50, 60, 80, 100, 120, 140, 160, 180, 200, 220, 240, 260, 280, 300, 340, 360, 380, or up to the entire contiguous nucleotides of the specific genetic element of interest. Such fragments may be obtained by use of restriction enzymes to cleave the native viral genome; by synthesizing a nucleotide sequence from the native nucleotide sequence of the virus genome; or may be obtained through the use of PCR technology. See particularly (Mullis and Faloona, 1987) and (Erlich, 1989). Again, variants of the various vector components, such as those resulting from site-directed mutagenesis, are encompassed by the methods of the present invention. As described in more detail below, methods are available in the art for determining functional equivalence.

By “variant” it is intended to include substantially similar sequences. Thus, for nucleotide sequences or amino acid sequences, variants include sequences that are functionally equivalent to the various components of the viral vector system. Variant nucleotide sequences also include synthetically derived nucleotide sequences that have been generated, for example, by site directed mutagenesis, but which still retain the function of the native sequence. Generally, nucleotide sequence variants or amino acid sequence variants of the invention will have at least 70%, generally 80%, 85%, 90%, 91%, 92%, 93%, 94%, 95%, 96%, 97%, 98%, or 99% sequence identity to its respective native nucleotide sequence.

Variants of the invention include polynucleotides (e.g., vectors) comprising, consisting essentially of, or consisting of, sequences at least 70%, 80%, 85%, 90%, 91%, 92%, 93%, 94%, 95%, 96%, 97%, 98%, or 99% identical to the sequences of the vectors disclosed herein (SEQ ID NOs: 67-79).

One of skill will appreciate that many conservative variations of the nucleic acid constructs disclosed yield a functionally identical construct. Conservative variations of a particular nucleic acid sequence refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given polypeptide. For example, due to the degeneracy of the genetic code, “silent substitutions” (i.e., substitutions of a nucleic acid sequence which do not result in an alteration in an encoded polypeptide) are an implied feature of every nucleic acid sequence which encodes an amino acid. Similarly, “conservative amino acid substitutions,” in one or a few amino acids in an amino acid sequence of a packaging or packageable construct are substituted with different amino acids with highly similar properties, are also readily identified as being highly similar to a disclosed construct. For instance, the codons CGU, CGC, CGA, COG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every nucleic acid sequence herein which encodes a polypeptide also describes every possible silent variation. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in any described sequence. Furthermore, one of skill will recognize that individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton (1984) Proteins W. H. Freeman and Company. Finally, the addition of sequences which do not alter the activity of a nucleic acid molecule, such as a non-functional sequence is a conservative modification of the basic nucleic acid. Such conservatively substituted variations of each disclosed sequence are a feature of the present invention.

With respect to the amino acid sequences for the various full-length or mature polypeptides used in the vector system of the present invention, variants include those polypeptides that are derived from the native polypeptides by deletion (so-called truncation) or addition of one or more amino acids to the N-terminal and/or C-terminal end of the native polypeptide; deletion or addition of one or more amino acids at one or more sites in the native polypeptide; or substitution of one or more amino acids at one or more sites in the native polypeptide. Such variants may result from, for example, genetic polymorphism or from human manipulation. Methods for such manipulations are generally known in the art.

One of skill will recognize many ways of generating alterations in a given nucleic acid construct. Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques. See, (Gillam and Smith, 1979), (Roberts, Cheetham, and Rees, 1987), and Sambrook, Innis, Ausbel, Berger, Needham VanDevanter and Mullis (all supra).

A variant of a native nucleotide sequence or native polypeptide has substantial identity to the native sequence or native polypeptide. A variant may differ by as few as 1 to 10 amino acid residues, such as 6-10, as few as 5, as few as 4, 3, 2, or even 1 amino acid residue. A variant of a nucleotide sequence may differ by as low as 1 to 30 nucleotides, such as 6 to 20, as low as 5, as few as 4, 3, 2, or even 1 nucleotide residue.

It is intended by “sequence identity” that the same nucleotides or amino acid residues are found within the variant sequence and a reference sequence when a specified, contiguous segment of the nucleotide sequence or amino acid sequence of the variant is aligned and compared to the nucleotide sequence or amino acid sequence of the reference sequence. Methods for sequence alignment and for determining identity between sequences are well known in the art. With respect to optimal alignment of two nucleotide sequences, the contiguous segment of the variant nucleotide sequence may have additional nucleotides or deleted nucleotides with respect to the reference nucleotide sequence. Likewise, for purposes of optimal alignment of two amino acid sequences, the contiguous segment of the variant amino acid sequence may have additional amino acid residues or deleted amino acid residues with respect to the reference amino acid sequence. The contiguous segment used for comparison to the reference nucleotide sequence or reference amino acid sequence will comprise at least 20 contiguous nucleotides, or amino acid residues, and may be 30, 40, 50, 100, or more nucleotides or amino acid residues. Corrections for increased sequence identity associated with inclusion of gaps in the variant's nucleotide sequence or amino acid sequence can be made by assigning gap penalties.

The determination of percent identity between two sequences can be accomplished using a mathematical algorithm. For example, percent identity of an amino acid sequence can be determined using the Smith-Waterman homology search algorithm using an affine 6 gap search with a gap open penalty of 12 and a gap extension penalty of 2, BLOSUM matrix 62. Alternatively, percent identity of a nucleotide sequence is determined using the Smith-Waterman homology search algorithm using a gap open penalty of 25 and a gap extension penalty of 5. Such a determination of sequence identity can be performed using, for example, the DeCypher Hardware Accelerator from TimeLogic Version G. The Smith-Waterman homology search algorithm is taught in Smith and Waterman, herein incorporated by reference. Alternatively, the alignment program GCG Gap (Wisconsin Genetic Computing Group, Suite Version 10.1) using the default parameters may be used. The GCG Gap program applies the Needleman and Wunch algorithm and for the alignment of nucleotide sequences with an open gap penalty of 3 and an extend gap penalty of 1 may be used. Another preferred, non-limiting example of a mathematical algorithm utilized for the comparison of two sequences is the algorithm of Karlin and Altschul (Karlin and Altschul, 1990), modified as in Karlin and Altschul (Karlin and Altschul, 1993). Such an algorithm is incorporated into the NBLAST and XBLAST programs of Altschul et al. (Altschul et al., 1990). BLAST nucleotide searches can be performed with the NBLAST program, score=100, wordlength=12, to obtain nucleotide sequences having sufficient sequence identity. BLAST protein searches can be performed with the XBLAST program, score=50, wordlength=3, to obtain amino acid sequences having sufficient sequence identity. To obtain gapped alignments for comparison purposes, Gapped BLAST can be utilized as described in Altschul et al. (Altschul et al., 1997). Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See http://www.ncbi.nlm.nih.gov. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used. Percent identity of an amino acid sequence can also be determined using the VectorNTI (Informax, USA).

One of skill can select a desired nucleic acid of the invention based upon the sequences provided and upon knowledge in the art regarding CAEV generally. The life-cycle, genomic organization, developmental regulation and associated molecular biology of lentiviruses have been the focus of over a decade of intense research. The specific effects of many mutations in many lentiviral genomes are known. In addition, the nucleic acid sequence variations of some CAEV strains are known. Moreover, general knowledge regarding the nature of proteins and nucleic acids allows one of skill to select appropriate sequences with activity similar or equivalent to the nucleic acids and polypeptides disclosed in the sequence listings herein.

Finally, most modifications to nucleic acids are evaluated by routine screening techniques in suitable assays for the desired characteristic. For instance, changes in the immunological character of encoded polypeptides can be detected by an appropriate immunological assay. Modifications of other properties such as nucleic acid hybridization to a complementary nucleic acid, redox or thermal stability of encoded proteins, hydrophobicity, susceptibility to proteolysis, or the tendency to aggregate are all assayed according to standard techniques.

Polynucleotides of Interest

As will be appreciated by one skilled in the art, the nucleotide sequence of the inserted polynucleotide of interest may be of any nucleotide sequence. For example, the polynucleotide sequence may be a reporter gene sequence or a selectable marker gene sequence. A reporter gene sequence, as used herein, is any gene sequence which, when expressed, results in the production of a protein whose presence or activity can be monitored. Examples of suitable reporter genes include the gene for galactokinase, galactosidase, chloramphenicol acetyltransferase, β-lactamase, green fluorescent protein, enhanced green fluorescent protein, etc. Alternatively, the reporter gene sequence may be any gene sequence whose expression produces a gene product that affects cell physiology. Polynucleotide sequences of the present invention may comprise one or more gene sequences that already possess on or more promoters, initiation sequences, or processing sequences.

A selectable marker gene sequence is any gene sequence capable of expressing a protein whose presence permits one to selectively propagate a cell which contains it. Examples of selectable marker genes include gene sequences capable of conferring host resistance to antibiotics (e.g., puromycin, hygromycin, neomycin, zeocin and the like), or of conferring host resistance to amino acid analogues, or of permitting the growth of bacteria on additional carbon sources or under otherwise impermissible culture conditions.

Reporter or selectable marker gene sequences are sufficient to permit the recognition or selection of the vector in normal cells. In one embodiment of the invention, the reporter gene sequence may encode an enzyme or other protein which is normally absent from mammalian cells, and whose presence can, therefore, definitively establish the presence of the vector in such a cell.

The transfer vectors of the present invention additionally permit the incorporation of heterologous nucleic acid, or polynucleotides, into virus particles, thereby providing a means for amplifying the number of infected host cells containing heterologous nucleic acid therein. The incorporation of the heterologous polynucleotide facilitates the replication of the heterologous nucleic acid within the viral particle, and the subsequent production of a heterologous protein therein. A heterologous protein is herein defined as a protein or fragment thereof wherein all or a portion of the protein is not expressed by the host cell. A nucleic acid or gene sequence is said to be heterologous if it is not naturally present in the wild-type of the viral vector used to deliver the gene into a cell. The term heterologous nucleic acid sequence or polynucleotide sequence, as used herein, is intended to refer to a nucleic acid molecule (preferably DNA). The polynucleotide sequence or heterologous polynucleotide sequence may also comprise the coding sequence of a desired product such as a suitable biologically active protein or polypeptide, immunogenic or antigenic protein or polypeptide, or a therapeutically active protein or polypeptide. The polypeptide may supplement deficient or nonexistent expression of an endogenous protein in a host cell. Such gene sequences may be derived from a variety of sources including DNA, cDNA, synthetic DNA, RNA or combinations thereof. Such gene sequences may comprise genomic DNA which may or may not include naturally occurring introns. Moreover, such genomic DNA may be obtained in association with promoter sequences or polyadenylation sequences. The gene sequences of the present invention are preferably cDNA. Genomic or cDNA may be obtained in any number of ways. Genomic DNA can be extracted and purified from suitable cells by means well-known in the art. Alternatively, mRNA can be isolated from a cell and used to prepare cDNA by reverse transcription, or other means. Alternatively, the polynucleotide sequence may comprise a sequence complementary to an RNA sequence, such as an antisense RNA sequence, which antisense sequence can be administered to an individual to inhibit expression of a complementary polynucleotide in the cells of the individual.

Expression of the heterologous gene may provide an immunogenic or antigenic protein or polypeptide to achieve an antibody response. The antibodies thus raised may be collected from an animal in a body fluid such as blood, serum or ascites.

The heterologous gene can also be any nucleic acid of interest that can be transcribed. Generally the foreign gene encodes a polypeptide. Preferably the polypeptide has some therapeutic benefit. The polypeptide may supplement deficient or nonexistent expression of an endogenous protein in a host cell. The polypeptide can confer new properties on the host cell, such as a chimeric signaling receptor, see U.S. Pat. No. 5,359,046. One of ordinary skill can determine the appropriateness of a foreign gene practicing techniques taught herein and known in the art. For example, the artisan would know whether a foreign gene is of a suitable size for encapsidation and whether the foreign gene product is expressed properly.

The particular heterologous protein that can be employed in the present invention is not critical thereto.

Specific examples of such heterologous proteins which can be employed in the present invention include dystrophin (Hoffman, Brown, and Kunkel, 1987), coagulation factor VIII (Wion et al., 1985), Cystic Fibrosis Transmembrane Regulator Protein (CFTR) (Anderson et al., 1991; Crawford, 1991), Ornithine Transcarbamylase (OTC) (Murakami et al., 1988), and α1-antitrypsin (Fagerhol and Cox, 1981).

The genes encoding many heterologous proteins are well-known in the art, and can be cloned from genomic or cDNA libraries [Sambrook et al, supra]. Examples of such genes include the dystrophin gene (Lee et al., 1991), the Factor VIII gene (Toole et al., 1984), the CFTR gene (Rommens et al., 1989; Riordan, 1989), the OTC gene (Horwich et al., 1984), and the α1-antitrypsin gene (Lemarchand et al., 1992).

In addition, genes encoding heterologous proteins such as Rb, for the treatment of vascular proliferative disorders like atherosclerosis (Chang et al., 1995), and p53 for the treatment of cancer (Wills et al., 1994; Clayman, 1995), and HIV disease (Bridges and Sarver, 1995), can be employed in the present invention.

The vector does not always need to code for a functional, heterologous gene product, i.e., it may also code for a partial gene product which acts as an inhibitor of a eukaryotic enzyme (Warne, Viciana, and Downward, 1993; Wang, 1991).

It may also be desirable to modulate the expression of a gene regulating molecule in a cell by the introduction of a molecule by the method of the invention. The term “modulate” envisions the suppression of expression of a gene when it is over-expressed or augmentation of expression when it is under-expressed. Where a cell proliferative disorder is associated with the expression of a gene, nucleic acid sequences that interfere with the expression of a gene at the translational level can be used. The approach can utilize, for example, antisense nucleic acid, ribozymes or triplex agents to block transcription or translation of a specific mRNA, either by masking that mRNA with an antisense nucleic acid or triplex agent, or by cleaving same with a ribozyme.

Antisense nucleic acids are DNA or RNA molecules which are complementary to at least a portion of a specific mRNA molecule . In the cell, the antisense nucleic acids hybridize to the corresponding mRNA forming a double-stranded molecule. The antisense nucleic acids interfere with the translation of the mRNA since the cell will not translate an mRNA that is double-stranded. Antisense oligomers of about 15 nucleotides or more are preferred since such are synthesized easily and are less likely to cause problems than larger molecules when introduced into the target cell. The use of antisense methods to inhibit the in vitro translation of genes is well known in the art (Marcus-Sekura, 1988).

The antisense nucleic acid can be used to block expression of a mutant protein or a dominantly active gene product, such as amyloid precursor protein that accumulates in Alzheimer's disease. Such methods are also useful for the treatment of Huntington's disease, hereditary Parkinsonism and other diseases. Antisense nucleic acids are also useful for the inhibition of expression of proteins associated with toxicity.

Use of an oligonucleotide to stall transcription can be by the mechanism known as the triplex strategy since the oligomer winds around double-helical DNA, forming a three-strand helix. Therefore, the triplex compounds can be designed to recognize a unique site on a chosen gene (Maher, Wold, and Dervan, 1991; Helene, 1991).

Ribozymes are RNA molecules possessing the ability to specifically cleave other single-stranded RNA in a manner analogous to DNA restriction endonucleases. Through the modification of nucleotide sequences which encode those RNA's, it is possible to engineer molecules that recognize and cleave specific nucleotide sequences in an RNA molecule (Cech, 1988). A major advantage of that approach is only mRNA's with particular sequences are inactivated.

It may be desirable to transfer a nucleic acid encoding a biological response modifier. Included in that category are immunopotentiating agents including nucleic acids encoding a number of the cytokines classified as “interleukins”, for example, interleukins 1 through 12. Also included in that category, although not necessarily working according to the same mechanism, are interferons, and in particular gamma interferon (γ-IFN), tumor necrosis factor (TNF) and granulocyte-macrophage colony stimulating factor (GM-CSF). It may be desirable to deliver such nucleic acids to bone marrow cells or macrophages to treat inborn enzymatic deficiencies or immune defects. Nucleic acids encoding growth factors, toxic peptides, ligands, receptors or other physiologically important proteins also can be introduced into specific non-dividing cells.

Thus, the recombinant CAEV vector system of the invention can be used to treat an HIV-infected cell (e.g., T-cell or macrophage) with an anti-HIV molecule. In addition, respiratory epithelium, for example, can be infected with a recombinant lentivirus of the invention having a gene for cystic fibrosis transmembrane conductance regulator (CFTR) for treatment of cystic fibrosis.

Thus, the recombinant CAEV vector system of the invention can be used to treat many human diseases. Specific examples of possible application of the CAEV vector system in human diseases include, but are limited to: Alzheimer's diseases, Parkinson's diseases, amyotrophic lateral sclerosis disease, Huntington's disease, beta-thalassemia, retinitis pigmentosa, mucopolysaccharide disease, leukodystrophy diseases, X-linked SCID, phenylketonuria, tryosinemia, hemophilia A and B, Wilson's diseases, LDL receptor deficiency, Human Immunodeficiency, and Duchenne's dystrophy.

CAEV Vector Particles

In a method of the invention, infectious and replication-defective CAEV vector particles may be prepared according to the methods disclosed herein in combination with techniques known to those skilled in the art. The method includes transfecting a lentivirus-permissive cell with the vector expression system of the present invention; producing the CAEV-derived particles in the transfected cell; and collecting the virus particles from the cell.

The term “transfection” as used herein refers to the introduction of foreign DNA into eukaryotic cells. Transfection may be accomplished by a variety of means known to the art including but not limited to calcium phosphate-DNA co-precipitation, DEAE-dextran-mediated transfection, polybrene-mediated transfection, electroporation, microinjection, liposome fusion, lipofection and protoplast fusion. These techniques are well known in the art.

As used herein, the term “transduction” refers to the delivery of a gene using a viral or retroviral vector particles by means of infection rather than by transfection. In some embodiments, retroviral vectors are transduced. Thus, a “transduced gene” is a gene that has been introduced into the cell via lentiviral or vector infection and provirus integration. In certain embodiments, the CAEV viral vector particles transduce genes into “target cells” or host cells.

The step of facilitating the production of the infectious viral particles in the cells may also be carried out using conventional techniques, such as by standard cell culture growth techniques.

The step of collecting the infectious virus particles may also be carried out using conventional techniques. For example, the infectious particles may be collected by collection of the supernatant of the cell culture, as is known in the art. Optionally, the collected virus particles may be purified if desired. Suitable purification techniques are well known to those skilled in the art.

If desired by the skilled artisan, CAEV stock solutions may be prepared using the vectors and methods of the present invention. Methods of preparing viral stock solutions are known in the art and are illustrated by, e.g., (Soneoka et al., 1995) and (Landau and Littman, 1992). In a method of producing a stock solution in the present invention, lentiviral-permissive cells are transfected with the vector system of the present invention. The cells are then grown under suitable cell culture conditions, and the CAEV particles are collected from the cell culture media as described above. Suitable permissive cell lines include, but are not limited to, the human cell lines 293, 293T, and HeLa the monkey cell line Vero, and the goat cell lines GSM and Ch1Es.

The vectors of the present invention are also useful in preparing stable packaging cells (i.e. cells that stably express CAEV structural proteins, which cells, by themselves, cannot generate infectious virus particles) and virus producer cells (VPC). Methods for preparing packaging cells that express retrovirus proteins are known in the art and are exemplified by the methods set forth in, for example, U.S. Pat. No. 4,650,764 to Temin et al., which disclosure is incorporated herein in its entirety. Within the scope of the present invention, a packaging cell will comprise a lentivirus-permissive host cell comprising a CAEV nucleic acid sequence from at least one CAEV packaging vector described in this invention, which nucleic acid sequence is packaging-signal defective, thus rendering the cell itself capable of producing at least one CAEV structural protein, but not capable of producing replication-competent infectious virus. A packaging cell may be made by transfecting a CAEV-permissive host cell (e.g., a human embryonic kidney 293 or 293T cells) with a suitable CAEV nucleic acid sequence as provided above according to known procedures. The resulting packaging cell is thus able to express and produce at least one CAEV structural protein. However, the packaging cell is still not able to produce recombinant CAEV virus. The packaging cell may then be transfected with other nucleic acid sequences, i.e., a transfer vector, which may contain heterologous genes of interest and an appropriate packaging signal. Once transfected with the additional sequence or sequences, the packaging cell may thus be used to provide stocks of CAEV viruses that contain heterologous genes, but which viruses are themselves replication-incompetent. The resulting virus producing cell (VPC) is thus able to produce infectious virus particles containing heterologous gene of interest.

Gene Transfer and Therapy

A number of human genetic diseases that result from an alteration in a single gene are prime candidates for gene therapy. As used herein, the terms “gene therapy” or “gene transfer” are defined as the insertion of genes into cells for the purpose of medicinal therapy. There are many applications of gene therapy, particularly via stem cell genetic insertion, and thus are well known and have been extensively reviewed. The term “target” is used to indicate that the CAEV vector is intended to transduce the cells. Target cells for therapeutic gene transfer, either ex vivo or in vivo, include, but are not limited to hematopoietic stem cell, lymphocyte, vascular endothelial cell, respiratory epithelial cell, keratinocyte, skeletal and muscle cells, liver cell, neuron cell, and cancer cell .

The gene transfer technology of the present invention may also be used in elucidating the processing of peptides and identification of the functional domains of various proteins. Cloned cDNA or genomic sequences for proteins can be introduced into different target cells ex vivo, or in vivo, in order to study cell-specific differences in processing and cellular fate. By placing the coding sequences under the control of a strong promoter, a substantial amount of the desired protein can be made. Furthermore, the specific residues involved in protein processing, intracellular sorting, or biological activity can be determined by mutational change in discrete residues of the coding sequences.

Gene transfer technology of the present invention can also be applied to provide a means to control expression of a protein and to assess its capacity to modulate cellular events. Some functions of proteins, such as their role in differentiation, may be studied in tissue culture, whereas others will require reintroduction into in vivo systems at different times in development in order to monitor changes in relevant properties.

Gene transfer provides a means to study the nucleic acid sequences and cellular factors that regulate expression of specific genes. One approach to such a study would be to fuse the regulatory elements to be studied to reported genes and subsequently assaying the expression of the reporter gene.

Gene transfer also possesses substantial potential use in understanding and providing therapy for disease states. There are a number of inherited diseases in which defective genes are known and have been cloned. In some cases, the function of these cloned genes is known. In general, the above disease states fall into two classes: deficiency states, usually of enzymes, which are generally inherited in a recessive manner, and unbalanced states, at least sometimes involving regulatory or structural proteins, which are inherited in a dominant manner. For deficiency state diseases, gene transfer could be used to bring a normal gene into affected tissues for replacement therapy, as well as to create animal models for the disease using antisense mutations. For unbalanced disease states, gene transfer could be used to create a disease state in a model system, which could then be used in efforts to counteract the disease state. Thus the methods of the present invention permit the treatment of genetic diseases. As used herein, a disease state is treated by partially or wholly remedying the deficiency or imbalance which causes the disease or makes it more severe. The use of site-specific integration of nucleic sequences to cause mutations or to correct defects is also possible.

The method of the invention may also be useful for neuronal, glial, fibroblast or mesenchymal cell transplantation, or “grafting”, which involves transplantation of cells infected with the recombinant lentivirus of the invention ex vivo, or infection in vivo into the central nervous system or into the ventricular cavities or subdurally onto the surface of a host brain. Such methods for grafting will be known to those skilled in the art and are described in Neural Grafting in the Mammalian CNS, Bjorklund & Stenevi, eds. (1985).

For diseases due to deficiency of a protein product, gene transfer could introduce a normal gene into the affected tissues for replacement therapy, as well as to create animal models for the disease using antisense mutations. For example, it may be desirable to insert a Factor VIII or IX encoding nucleic acid into a CAEV particle for infection of a muscle, spleen or liver cell.

There are many applications of gene therapy, particularly via stem cell genetic insertion, and thus are well known and have been extensively reviewed. As used herein, the term “stem cells” includes but is not limited to hematopoietic stem cells, neuronal stem cells, mesenchymal (particularly muscular) stem cells, and liver stem cells. Stem cells are capable of repopulating tissues in vivo. Hematopoietic stem cells are progenitor cells derived from primitive human hematopoietic cells.

Gene therapy using hematopoietic stem cells is also useful to treat a genetic abnormality in lymphoid and myeloid cells that results generally in the production of a defective protein or abnormal levels of expression of the gene.

For a number of these diseases, the introduction of a normal copy or functional homolog of the defective gene and the production of even small amounts of the missing gene product would have a beneficial effect. At the same time, overexpression of the gene product would not be expected to have deleterious effects. The following provides a non-exhaustive list of diseases for which gene transfer into hematopoietic stem cells is potentially useful. These diseases generally include bone marrow disorders, erythroid cell defects, metabolic disorders and the like. Hematopoietic stem cell gene therapy is beneficial for the treatment of genetic disorders of blood cells such as α- and β-thalassemia, sickle cell anemia and hemophilia A and B in which the globin gene or clotting factor genes (e.g., Factor IX and Factor X genes) are defective. Another good example is the treatment of severe combined immunodeficiency disease (SCIDS), in which patients lack the adenosine deaminase (ADA) enzyme which helps eliminate certain byproducts that are toxic to T and B lymphocytes and render the patients defenseless against infection. Such patients are ideal candidates to receive gene therapy by introducing the ADA gene into their hematopoietic stem cells instead of the patient's lymphocytes as done in the past. Other diseases include chronic granulomatosis where the neutrophils express a defective cytochrome b and Gaucher disease resulting from an abnormal glucocerebrosidase gene product in macrophages.

Additionally, neurological degenerative disorder, e.g., Parkinson's disease, is an attractive target for gene therapy by introducing the GDNF (Glial cell line-derived neurotrophic factor) gene into the striatum and the substantia (Kordower et al., 2000).

Strategies to treat various forms of cancer are also included in gene therapy. The CAEV vector can carry a gene that encodes, for example, a toxin or an apoptosis inducer effective to specifically kill the cancerous cells. Specific killing of tumor cells can also be accomplished by introducing a suicide gene to cancerous hematopoietic cells under conditions that only the tumor cells express the suicide gene. The suicide gene product confers lethal sensitivity to the cells by converting a normally nontoxic drug to a toxic derivative. For example, the enzyme cytosine deaminase converts the nontoxic substance 5′-fluorocytosine to a toxic derivative, 5-fluorouracil (Mullen, Kilstrup, and Blaese, 1992). Tumor-specific lymphocytes can be genetically modified for example, to locally deliver gene products with anti-tumor activity to sites of the tumor to circumvent the toxicity associated with the systemic delivery of these gene products. A gene therapy approach can also be applied to render bone marrow cells resistant to the toxic effects of chemotherapy.

Gene therapy can also be used to prevent or combat viral infections such as HIV and HTLV-1 infection. For example, hematopoietic stem cells can be genetically modified to render them resistant to infection by HIV. One approach is to inhibit viral gene expression specifically by using antisense RNA or by subverting existing viral regulatory pathways. Antisense RNAs complementary to retroviral RNAs have been shown to inhibit the replication of a number of retroviruses (To, Booth, and Neiman, 1986) including HIV (Rhodes and James, 1991) and HTLV-1 (von Ruden and Gilboa, 1989).

Another area where gene therapy in hematopoietic stem cells may find use is in alleviating autoimmune disease. The therapeutic gene can encode, e.g., a B or T cell signaling molecule capable of reconstituting the normal apoptotic signal that results in the death and elimination of autoreactive cells.

Ex vivo cell transformation for diagnostics, research, or for gene therapy (e.g., via re-infusion of the transformed cells into the host organism) is well known to those of skill in the art. In one embodiment of the invention, cells are isolated from the subject organism, transfected with a vector of the invention comprising a polypeptide of interest, and re-infused back into the subject organism (e.g., patient).

Various cell types suitable for ex vivo transformation are well known to those of skill in the art. Particular preferred cells are stem cells described supra (see, e.g., Freshney (1994) Culture of Animal Cells, a Manual of Basic Technique, third edition Wiley-Liss, New York, and the references cited therein for a discussion of how to isolate and culture cells from patients). Transformed cells are cultured by means well known in the art. See, also Kuchler (1977) Biochemical Methods in Cell Culture and Virology, Kuchler, R. J., Dowden, Hutchinson and Ross, Inc., and Atlas (1993) CRC Handbook of Microbiological Media (Parks ed) CRC press, Boca Raton, Fla. Mammalian cell systems often will be in the form of monolayers of cells, although mammalian cell suspensions are also used. Alternatively, cells can be derived from those stored in a cell bank (e.g., a blood bank). Illustrative examples of mammalian cell lines include the HEC-1-B cell line, VERO and Hela cells, Chinese hamster ovary (CHO) cell lines, W138, BHK, Cos-7 or MDCK cell lines (see, e.g., Freshney, supra).

T cells or B cells are also used in some ex vivo gene transfer procedures. Several techniques are known for isolating T and B cells. The expression of surface markers facilitates identification and purification of such cells.

In summary, the viral vectors of the present invention can be used to stably transduce either dividing or non-dividing cells, and stably express a heterologous gene. Using this vector system, it is now possible to introduce into dividing or non-dividing cells, genes that encode proteins that can affect the physiology of the cells. The vectors of the present invention can thus be useful in gene therapy for disease states, or for experimental modification of cell physiology.

Kits

It is a further object of this invention to provide a kit or drug delivery system comprising the vectors for use in the methods described herein. All the essential materials and reagents required for administration of the targeted retroviral particle may be assembled in a kit (e.g., packaging cell construct or cell line). The components of the kit may be provided in a variety of formulations. The one or more CAEV particles may be formulated with one or more agents (e.g., a chemotherapeutic agent) into a single pharmaceutically acceptable composition or separate pharmaceutically acceptable compositions.

The components of these kits or drug delivery systems may also be provided in dried or lyophilized forms. When reagents or components are provided as a dried form, reconstitution generally is by the addition of a suitable solvent, which may also be provided in another container means. The kits of the invention may also comprise instructions regarding the dosage and or administration information for the targeted CAEV particle. The kits or drug delivery systems of the present invention also will typically include a means for containing the vials in close confinement for commercial sale such as, e.g., injection or blow-molded plastic containers into which the desired vials are retained. Irrespective of the number or type of containers, the kits may also comprise, or be packaged with, an instrument for assisting with the injection/administration or placement of the ultimate complex composition within the body of a subject. Such an instrument may be an applicator, inhalant, syringe, pipette, forceps, measured spoon, eye-dropper or any such medically approved delivery vehicle.

The following examples illustrate various aspects of the invention, but in no way are intended to limit the scope thereof.

### EXAMPLES

The following examples serve to illustrate certain embodiments and aspects of the present invention and are not to be construed as limiting the scope thereof.

The following examples demonstrate the finding that the recombinant CAEV-based lentiviral vector system of the present invention is as effective in expression as the well-known HIV-1 based lentiviral system. The examples show that the level of genomic RNA transcription, encapsidation, transduction, reverse transcription, and integration of the CAEV-based vector particle production system of the present invention is comparable to that of HIV-1-based lentiviral vector system, which has long been accepted as a highly efficient gene transfer system (Naldini et al., 1996).

This is the first report on the construction of a high titer CAEV-based vector system, which is based on a minimum of three-plasmid co-transfection method, requiring the expression of just the gag-pol and env genes, and optionally a rev gene.

Materials and Methods

Plasmid Construction

The Parent Plasmids.

The parent plasmids from which the CAEV vectors of the present invention were derived are the plasmid pWTE-BM and the plasmid pCAEV-LTR, kindly provided by Dr. Marie Suzan (Institut National de la Sante et de la Recherche Medicale “INSERM”, France) The pWTE-BM plasmid contains a full-length genomic CAEV cDNA except for the 0.4 kb Hind III fragment which contains parts of env, rev, and U3 regions and a 1337 base pair stuffer fragment. The plasmid pCAEV-LTR contains the 0.4 kb Hind III fragment lacking in the pWTE-BM (Saltarelli et al., 1990; Saltarelli, 1993). Neither of the vectors can generate a wild-type virus.

CAEV Gag-Pol Expression Vector (pMGP/RRE) (SEQ ID NO: 77).

The pMGP/RRE (SEQ ID NO: 77) plasmid is a PWTE-BM derived gag-pol expression plasmid (shown in FIG. 2A). The pMGP/RRE (SEQ ID NO: 77) plasmid contains a strong and heterologous MCMV major immediate-early promoter (MCMV MIEP), the gag-pol gene, and the rev responsive element (RRE). The pMGP/RRE (SEQ ID NO: 77) plasmid also encodes the neomycin resistant gene as an antibiotic selection marker. For the construction of the plasmid, the gag-pol gene fragment (nucleotide 512 through nucleotide 5046 of the CAEV genome) from pWTE-BM was subcloned into the pGL2-Basic (Promega, WI, USA) cloning vector by using standard protocols for several PCR and subcloning steps. The MCMV MIEP fragment was excised from the plasmid pMYK (Kim et al., 2002) was then inserted upstream of the gag gene, and the RRE region (from nucleotide 7824 to nucleotide 8183 or nucleotide 7849 to nucleotide 8150 of the CAEV genome) was inserted downstream of the pol gene. The pMGP/REV/RRE is another gag-pol expressing plasmid (shown in FIG. 2B) containing the CAEV rev gene. In addition, the major splicing donor site of the CAEV (from nucleotide 330 to nucleotide 346 of the CAEV genome) was inserted downstream of the MCMV promoter.

Transfer Vectors (pCAH/SINd Series).

The plasmids in the pCAH/SINd series (shown in FIGS. 3A-3H) (SEQ ID NOs: 67-71, 73, 78, and 79) were constructed to identify an optimal packaging sequence for the design of the transfer vectors of the present invention. Each of the plasmids in the series were designed to contain different lengths of the 5′untranslated region and the beginning of the gag-encoding region to allow for the side by side comparison of the effects of the various lengths in this region. To address certain safety concerns, these plasmids were designed as SIN (self-inactivation) vectors having the U3 region of the 3′LTR deleted. To allow high level expression of vector RNA from the transfer vectors in the absence of a trans-acting factor, tat, the U3 region of the 5′LTR was replaced with an HCMV MIEP. In addition, all known cis-acting sequence elements required for polyadenylation, RNA transportation, reverse transcription, and integration were included in the transfer vector series.

The plasmids of the pCAH/SINd series (SEQ ID NOs: 67-71, 73, 78, 79) were constructed as follows. pCAH/SINd (PBS-deficient negative control vector) (SEQ ID NO: 73) (FIG. 3A) was designed to contain only the 5′ untranslated sequences (R and U5 region) in the 5′ LTR (from nucleotide 1 to nucleotide 163 of the CAEV genome). pCAH/SINd0 (SEQ ID NO: 67) (FIG. 3B) was designed to contain the entire 5′ untranslated region (from nucleotide 1 to nucleotide 511 of the CAEV genome). pCAH/SINd1 (SEQ ID NO: 68) (FIG. 3C) was designed to contain the entire 5′ untranslated region and the 327 bp fragment of the gag gene (from nucleotide 1 to nucleotide 839 of the CAEV genome) with point mutations. pCAH/SINd2 (SEQ ID NO: 69) (FIG. 3D) was designed to contain the entire 5′ untranslated region and the 612 bp fragment of the gag gene (from nucleotide 1 to nucleotide 1124 of the CAEV genome) with point mutations. Plasmid pCAH/SINd3 (SEQ ID NO: 70) (FIG. 3E) was designed to contain the entire 5′ untranslated region and the 908 bp fragment of the gag gene (from nucleotide 1 to nucleotide 1420 of the CAEV genome) with point mutations. Plasmid pCAH/SINd4 (SEQ ID NO: 71) (FIG. 3F) was designed to contain the entire 5′ untranslated region and the 1,198 bp fragment of the gag gene (from nucleotide 1 to nucleotide 1710 of the CAEV genome) with point mutations. pCAH/SINd1/hlacZ (SEQ ID NO: 78) (FIG. 3G) was constructed by inserting the expression cassette consisting of the HCMV MIEP and the lacZ gene into the pCAH/SINd1 (SEQ ID NO: 68). The plasmid pCAH/SINd60/hlacZ (SEQ ID NO: 78) (FIG. 3H) has the same design as the pCAH/SINd1 (SEQ ID NO: 68) except for the length of the gag gene, where it contains the first 60 bp fragment of the gag gene with point mutations (from nucleotide 1 to nucleotide 569 of the CAEV genome).

CAEV Vif Expression Vector (pHYK/vif) (SEQ ID NO. 76).

The vif gene (from nucleotide 5006 to nucleotide 5695 of the CAEV genome), which is known to be required for rapid and efficient virus replication, was cloned into a eukaryotic expression vector pHYK (Kim et al., 2002) (FIG. 4).

CAEV Rev Expression Vector (pHYK/rev) (SEQ ID NO. 75).

The rev gene, which regulates viral gene expression at the post-transcriptional level by interacting with the RRE, consists of two exons (the first exon is positioned from nucleotide 6,012 to 6,123, and the second exon is from nucleotide 8514 to 8803 of the CAEV genome). The Rev/RRE system promotes the nuclear export of unspliced RNA and is known to be essential for lentiviral replication. The full-length cDNA of rev gene was synthesized by RT-PCR and subcloned into the pHYK vector (FIG. 5).

Viral Envelope Gene Expression Vector.

The envelope gene expression vector systems used herein are the plasmid pHGVSV-G (SEQ ID NO: 74) and the plasmid pMYKEF1/env (SEQ ID NO: 72) (FIGS. 6A and 6B). The plasmid pHGVSV-G (SEQ ID NO: 74) was designed to express the vesicular stomatitis virus G (VSV-G) glycoprotein and contains the HCMV MIEP with β-globin intron as a promoter. The pMYKEF-1/env (SEQ ID NO: 72) was designed to express the gibbon ape leukemia virus (GaLV) envelope protein and contains the MCMV MIEP with eukaryotic elongation factor-1α intron as a promoter.

MuLV- and HIV-1-Based Plasmids.

As control vector systems, pMFG/lac/Zpuro and pHR/lacZ vectors were used in the present invention, that were lacZ-containing retrovirus vectors derived from the murine leukemia virus (MuLV) (Kim et al., 1997) and the human immunodeficiency virus type 1 (HIV-1) (Naldini et al., 1996), respectively. For the packaging plasmids of MuLV and HIV-1 vector systems, pEQPAM3 (Persons et al., 1998) and pCMVΔR8-2 were used, respectively. The HIV-1 packaging plasmid pCMVΔR8-2 is identical with pCMVΔR9 (Naldini et al., 1996) except for encoding a functional HIV-1 vpu gene and deletion of the 1.3-kb BglII fragment in env gene.

Vector Particle Production

Pseudotyped CAEV-based lentiviral vector particles were produced by liposome mediated transient transfection of three or more plasmids into 293T cells plated one day prior to transfection at a density of 5×10⁵ cells per 6-well culture dish. Three plasmid cotransfections were performed at a 1:1:1 molar ratio of a gag-pol expressing plasmid, a transfer vector plasmid, and an env-encoding plasmid. Four plasmid cotransfections were performed at a 3:3:3:1 molar ratio of a gag-pol expressing plasmid, a transfer vector plasmid, an env-encoding plasmid, and a rev-expressing plasmid. Five plasmid cotransfections were performed at a 3:3:3:1:1 molar ratio of a gag-pol expressing plasmid, a transfer vector plasmid, and an env-encoding plasmid, a rev-expressing plasmid and a vif-expressing plasmid. The culture supernatant containing viral vector particles was harvested 48 hours later, clarified with a 0.45 μM membrane filter (Nalgene, NY, USA), and either used immediately or stored at −70° C. deep-freezer.

In Vitro Transduction

Transduction was carried out by adding the viral vector particles onto 293T cells for 4 hours, in the presence of 8 μg/ml polybrene followed by the addition of fresh media. After 48 hours Beta-Gal expression was assayed after the cells were fixed in a solution consisting of 1% formaldehyde and 0.2% glutaraldehyde and stained for 12 hours at 37° C. in a solution containing 300 μg of 5-bromo-4-chloro-3-indolyly b-D-galactoside (X-Gal, Promega, WI, USA), 4 mM potassium ferrocyanide, 4 mM potassium ferricyanide, and 2 mM Mgcl₂. Titers can be determined by counting the number of blue foci as LacZ-forming units per ml (LFU/ml).

RT-PCR Assay

Total RNA was extracted from cultured cells or culture supernatant by the method using TRIzol LS Reagent (GIBCO BRL, CA, USA). The total RNA was treated with RNase free-DNase I (1 unit/μg of DNA for 20 minutes at 37° C.) (Promega, WI, USA) to eliminate DNA contamination. The DNase I reaction was stopped by adding RQ1 DNase stop solution provided with DNase I, and the RNA was cleaned up by the method using RNasy mini kit (Qiagen, Germany). The purified RNA was reverse transcribed into cDNA by reverse transcription (RT) reaction (90 min at 37° C.). In particular, the RT reaction was carried out in the presence of MuLV reverse transcriptase, oligo-dT primer or C-terminal specific primer, and dNTPs mix. PCR amplification was carried out for semi-quantitative analysis of template DNA with specific primers. In particular, PCR product DNA was synthesized from the cDNA or chromosomal DNA in the presence of heat stable Ex Taq polymerase, sequence specific DNA primers, and dNTPs mix.

Southern Blot Analysis

Genomic DNA was prepared from cells transduced with either pseudotyped HIV-1 or CAEV vector particles, and mock-transduced control cells using the DNeasy Tissue Kit (Qiagen, Germany). Ten μg of genomic DNA from the HIV-1 vector transduced cells were digested with BamH I and Kpn I. Ten μg each of the genomic DNA from the CAEV vector transduced cells and the negative control cells were double digested with EcoR I and Ssp I. The digested genomic DNAs were separated by electrophoresis on 0.7% agarose gel and transferred onto positive charged nylon membrane (Roche, Germany). Dig-labeled probes were prepared by PCR with primers specific for lacZ gene (Forward primer: CTGGCGTAATAGCGAAGAGG (SEQ ID NO: 65), Reverse primer: AACTCGCCGCACATCTGAAC (SEQ ID NO: 66)), and southern hybridization was carried out according to Dig application manual (Roche, Germany).

Growth Arrest of Cells and FACS Analysis of the Growth-Arrested Cells

293T cells were growth-arrested with aphidicolin (Sigma, USA) treatment(25 μg/ml), then transduced with CAEV viral vector particles. As a positive or negative control, cells were transduced side-by-side with either an HIV-1 vector or MuLV retrovirus vector. Two days after transduction, cells were stained with X-gal for beta-gal activity. In the aphidicolin treated culture, aphidicolin was present before and after infection.

The growth arrest of cells was confirmed by FACS analysis. The aphidicolin treated or untreated control cells were washed in PBS, fixed overnight in 70% ethanol at −20° C., and were followed by treatment of propidium iodide (100 μg/ml) (Sigma, USA) and RNAse A (100 μg/ml) (Qiagen, Germany) at RT for 1 hour. The cells were evaluated by FACS analysis, and the percent of total viable cells in G1, S and G2/M phase of the cell cycle was calculated (Becton Dickinson, Sanjose, Calif.).

### EXAMPLE 1

### Production of CAEV-Based Lentiviral Vector Particles

Replication defective lentiviral vector particles were generated by transient co-transfection of human 293T cells with a minimum of three-plasmid system of a CAEV gag-pol expressing plasmid, a CAEV env-expressing plasmid and a transfer vector plasmid. In a four-plasmid system, a CAEV rev expressing plasmid is added, and in a five-plasmid system, a CAEV vif expressing plasmid is added. For efficient packaging, transfer vectors were designed to contain the beginning of the gag-encoding sequence, where mutations were introduced into the start ATG codon and an ATG codon located downstream (ATG to TAG) to prevent the expression of gag proteins. RRE was included to boost packaging efficiency and the rev in the four- and five-plasmid systems was expressed from the vector to support the CAEV mRNA export. The internal HCMV-MIEP promoter-driven β-galactosidase gene in the transfer vector plasmid was inserted to serve as a reporter gene. The U3 region of the 5′LTR was replaced with the strong viral promoter, HCMV-MIEP, allowing the vector genome to be tat independent.

Transfer Vector RNA Transcription Level.

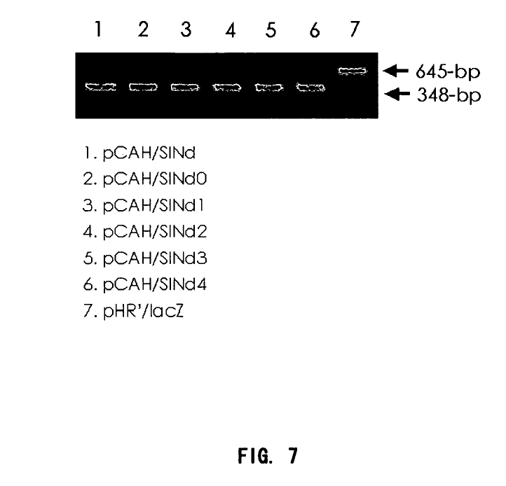

Transcription level of genomic RNA from a transfer vector is one of the critical factors mediating high titer production of recombinant viral vectors from packaging cells. In the present invention, HCMV enhancer/promoter element was used to construct the HCMV/CAEV hybrid LTR promoter system for safe and efficient transcription of the transfer vector RNA. To examine the transcription level of the transfer vector plasmids of the pCAH/SINd (SEQ ID NOs: 67-71, 73, 78, and 79) series containing the hybrid LTR promoter, each of the transfer vector plasmids was introduced into human T cells, together with the packaging plasmids (pMGP/RRE (SEQ ID NO: 77), pHYK/rev (SEQ ID NO: 75), pHYK/vif (SEQ ID NO: 76), pHGVSV-G (SEQ ID NO: 74) or pMYKEF1/env (SEQ ID NO: 72)), by liposome-mediated transfection. After 48 hours of incubation, total RNA was purified from the transfected cells and was subjected to Reverse Transcriptase Polymerase Chain Reaction (RT-PCR) analysis for the vector RNA transcript measurement. The PCR primer set (RRE primer set) for the CAEV transfer vectors was designed for synthesizing 348-bp PCR product coding for a part of RRE region. Another PCR primer set (lacZ primer set) for the HIV-1 transfer vector, pHRlacZ (Naldini et al., 1996), was designed for synthesizing the 645 bp PCR product coding for a part of the lacZ gene. As shown in FIG. 7, the CAEV transfer vectors of the present invention produced RNA transcript at a level comparable to that of the HIV- 1-based lentiviral transfer vector.

Formation and Release of the Vector Particles.

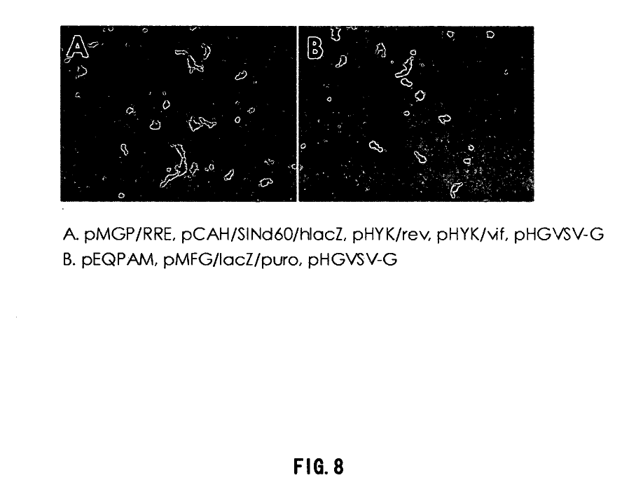

To examine the formation and release of mature and infectious virus vector particles, CAEV vector particles were produced following liposome-mediated co-transfection of the pMGP/RRE (SEQ ID NO: 77) gag-pol expression plasmid, the pHGVSV-G (SEQ ID NO: 74) env expression plasmid, the pHYK/rev (SEQ ID NO: 75) rev expression plasmid, pHYK/vif (SEQ ID NO: 76) vif expression plasmid, and the pCAH/SINd60/hlacZ (SEQ ID NO: 78) transfer vector plasmid into human 293T cells (DuBridge et al., 1987). Forty eights hours after transfection, the culture supernatant was harvested from the transfected cells and applied to fresh human 293T cells in the presence of 8 μg/ml polybrene for infection. The results indicated that the five plasmids system of the present invention was capable of producing comparable viral vector particle titers to that of the MuLV-based retroviral vector system (pEQPAM3, pMFG/lacZ/puro, pHGVSV-G (SEQ ID NO: 74)) (Ory, Neugeboren, and Mulligan, 1996; Persons et al., 1998) (shown in FIG. 8).

### EXAMPLE 2

### Effect of Rev and Vif Expression on Vector Particle Production

To determine the effect of CAEV rev and vif regulatory gene expression on vector particle production, the vector particle production system of (1) the three-plasmid system (pCAH/SIN, pMGP/RRE (SEQ ID NO: 77), pHGVSV-G (SEQ ID NO: 74) or pMYKEF1/env (SEQ ID NO: 72)), which is devoid of rev- and vif-encoding sequences, (2) the four-plasmids system (pCAH/SIN, pMGP/RRE (SEQ ID NO: 77), pHGVSV-G (SEQ ID NO: 74) or pMYKEF1/env (SEQ ID NO: 72), pHYK/rev (SEQ ID NO: 75)), which is devoid of vif-encoding sequence and (3) the five-plasmid system (pCAH/SIN, pMGP/RRE (SEQ ID NO: 77), pHGVSV-G (SEQ ID NO: 74) or pMYKEF1/env (SEQ ID NO: 72), pHYK/rev (SEQ ID NO: 75), pHYK/vif (SEQ ID NO: 76)), which contains both rev- and vif-encoding sequences were tested side by side for their efficiency in vector particle production. The plasmids of each system were transfected into 293T cells. At day 2 post-transfection, the transfer vector RNA and the virion RNA were extracted from the transfected cells and the culture medium of the transfected cells, respectively, and used as RT-PCR templates with the lacZ primer set to detect the transfer vector RNA genome.

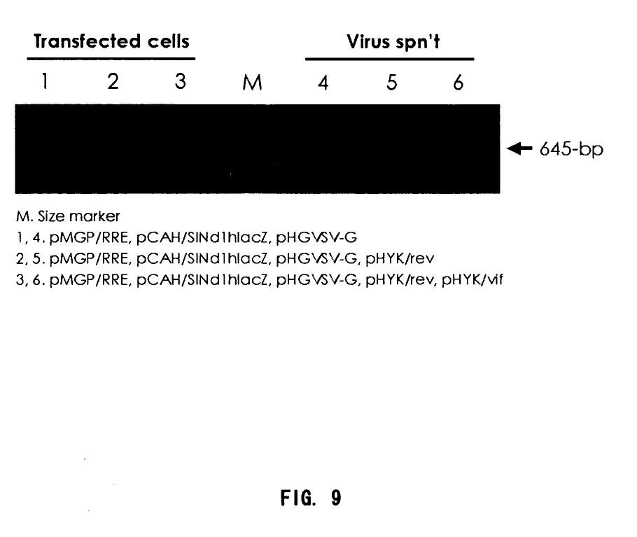

As shown in FIG. 9, although the expression level of the transfer vector RNA in the packaging cells was independent of the expression of the rev or the vif genes (Lane 1, 2 and 3 in FIG. 9), the amount of the encapsidated transfer vector RNA in the absence of rev (Lane 4, FIG. 9) was much lower than that in the presence of rev (Lane 5 in FIG. 9). Surprisingly, however, the titer of the vector particles measured by RT-PCR with the encapsidated RNA in the presence of vif (Lane 6 in FIG. 9) was lower than the vector particles measured by the RT-PCR with the encapsidated RNA in the absence of CAEV vif (Lane 5 in FIG. 9). These data indicate that CAEV rev and vif are not required for vector particle production, but rev is preferred for efficient vector particle production.

Of note is that the results of the present invention regarding the vif expression is inconsistent with the observations reported by Harmache et al. (Harmache et al., 1995; Harmache et al., 1996), where the vif gene was reported to be essential for efficient replication of CAEV in the goat synovial membrane cells and to be affecting the late steps of the virus replication cycle (e.g., RNA encapsidation, release of virus particles from host cells). One plausible explanation for the inconsistency may be in the use of the human 293T cells instead of the goat cells in the production of the recombinant CAEV vector particles. This interpretation supports the hypothesis proposed by Seroude et al. that the species-specific restrictions between vif and the virus-producing cells may modulate the vif function on viral infectivity (Seroude et al., 2002).

### EXAMPLE 3

### Identification of the Optimal Packaging Signal Sequence

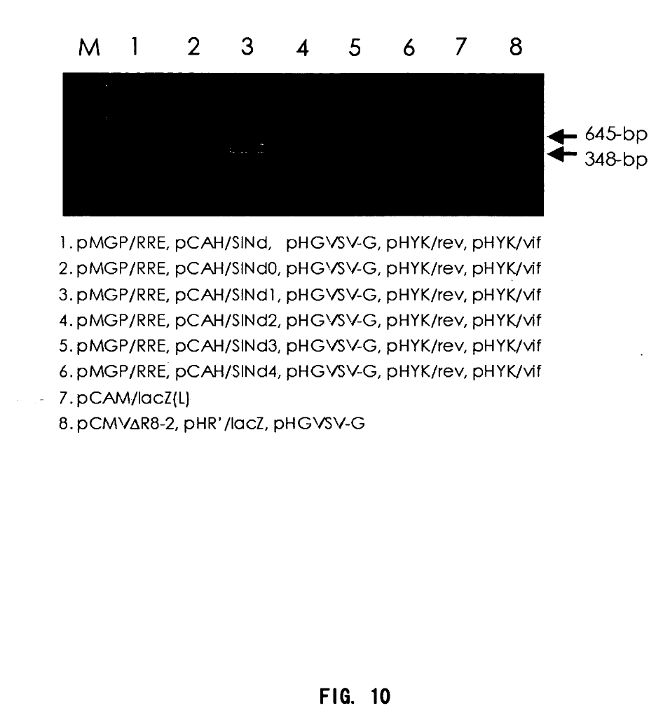

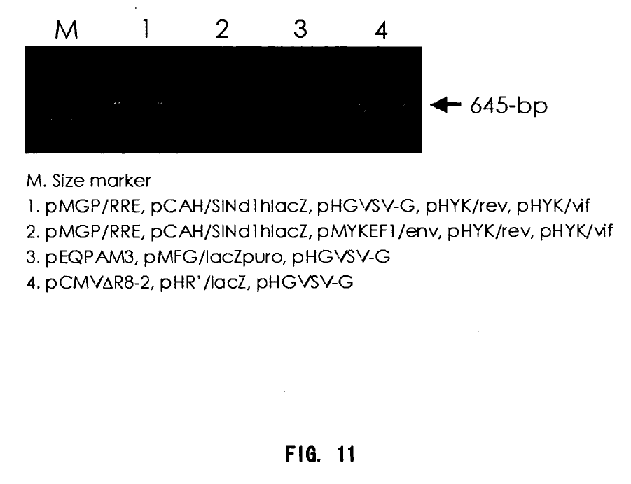

To identify the optimal packaging signal sequence for the encapsidation of CAEV transfer vector RNA, a series of plasmids containing different portions of the CAEV gag-coding region and the untranslated region between the 5′LTR and the gag start codon were compared for their vector particle production efficiency as follows. Human 293T cells were co-transfected with the pMGP/RRE (SEQ ID NO: 77) gag-pol expression plasmid, the pHGVSV-G (SEQ ID NO: 74) env expression plasmid, the pHYK/rev (SEQ ID NO: 75) rev expression plasmid, the pHYK/vif (SEQ ID NO: 76) vif expression plasmid, and the pCAH/SINd (SEQ ID NOs: 67-71, 73, 78, and 79) transfer vector series plasmid. As a negative control, a CAEV transfer vector pCAM/lacZ(L) was transfected in the absence of packaging plasmids. On day 2 post-transfection, virion RNA was extracted from the culture medium of the transfected cells and used as an RT-PCR template with the RRE primer set to detect the CAEV transfer vector series RNA genome, or with the lacZ primer set to detect the HIV-1 transfer vector RNA genome. As shown in FIG. 10, a strong PCR product signal, indicating efficient release of the virus particles containing the viral RNA, was obtained from the culture medium harvested from the virus producing 293T cells transfected with pCAH/SINd1 (SEQ ID NO: 68), which contained the complete 5′LTR as well as the first 327 bp of the gag region (lane 3 in FIG. 10). This signal was comparable to that obtained with the positive control, the HIV-1 vector, indicating that the amount of the encapsidated CAEV transfer vector RNA of the present invention is comparable to that of the HIV-1-based transfer vectors (lane 8 in FIG. 10). The packaging efficiency of the CAEV transfer vectors with gag-coding region of the first 612 bp or longer was significantly reduced (lanes 4, 5, and 6). The PCR product signals were not detectable when the transfer vectors used were devoid of the gag-coding sequences (lane 1 and 2 in FIG. 10). Negative control was transfected with a transfer vector only, and the positive control, HIV-1 vector, was transfected with pCMVΔR8-2, pHR′/lacZ and pHGVSV-G (SEQ ID NO: 74) (lanes 7 and 8 in FIG. 10).

In conclusion, the transfer vector RNAs were encapsidated efficiently in the packaging cells only when the transfer vectors included less than about 600 bp of the N-terminal gag-coding sequences as well as the entire untranslated region between the 5′LTR and the gag start codon. These results indicate that the role of the secondary structure of the RNA within the packaging signal is more important than the primary structure in RNA encapsidation.

### EXAMPLE 4

### Pseudotyping of the CAEV Vector Virion

To determine whether the recombinant CAEV vector virion can be pseudotyped with the GaLV glycoprotein as well as the VSV-G glycoprotein, either the GaLV expression vector, pMYKEF1/env (SEQ ID NO: 72), or the VSV-G expression vector, pHGVSV-G (SEQ ID NO: 74), was cotransfected with a transfer vector plasmid and the packaging plasmids into human 293T cells. Forty eights hours after transfection, culture supernatant containing pseudotyped virion particles released from the transfected cells was harvested, clarified with a 0.45 μm membrane filter, and used for infecting 293T human target cells. One day after infection, genomic DNA was purified by using a Genomic DNA Isolation kit (Qiagen, HL, Germany) and subjected to PCR experimentation to detect the integrated proviral cDNA. As expected, CAEV vector (Lane 1 in FIG. 11) was pseudotyped efficiently with the VSV-G protein, comparable to the MuLV- (Lane 3 in FIG. 11) and the HIV-1-based vector (Lane 4 in FIG. 11). In addition, inconsistent to the HIV-1 lentiviral vector system, the CAEV vector of the present invention was pseudotyped successfully with the GaLV envelope (Lane 2 in FIG. 11). This pseudotyping ability of the CAEV vectors with the GaLV envelope can afford a great advantage in the development of a clinical grade lentiviral vector system. MuLV (transfected with pEQPAM3, pMFG/lacZ/puro and pHGVSV-G (SEQ ID NO: 74)) and HIV-1 (transfected with pCMVΔR8-2, pHR′/lacZ and pHGVSV-G (SEQ ID NO: 74)) vector controls in lanes 3 and 4, respectively.

### EXAMPLE 5

### Generation of a CAEV Packaging Cell Line

Both the pMGP/RRE (SEQ ID NO: 77) and the pHYK/rev (SEQ ID NO: 75) vectors encode a neo^{r} gene for selection in eukaryotic cells. For efficient selection after cotransfection with a gag-pol and a rev expression vectors, another CAEV gag-pol expression vector may be constructed by replacing the neo^{r} gene with the other antibiotic resistance genes such as bacterial gpt gene, or one packaging plasmid system encoding the gag, pol and rev genes can be used. To determine if stable 293T cells expressing CAEV packaging proteins could be generated, antibiotic resistant colonies are selected under selective medium. Production of recombinant CAEV vector from the stable 293T cells suggests the feasibility of generating stable packaging cell lines for CAEV vector production.

### EXAMPLE 6

### Integration of the CAEV-based Vector cDNA into the Host Chromosome

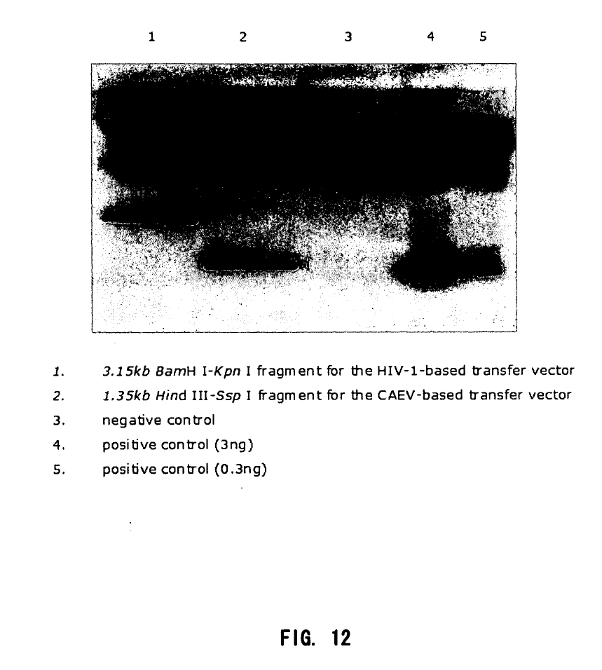

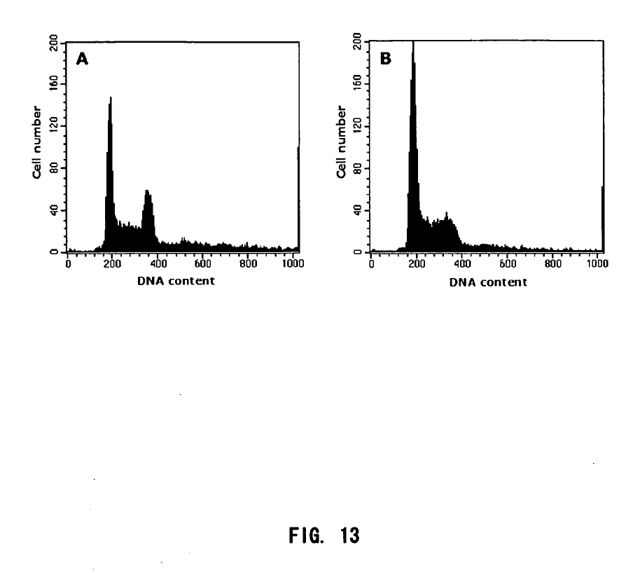

To examine the integration of the CAEV vector cDNA after transduction, the CAEV vector particles were produced by liposome-mediated co-transfection of the pMGP/REV/RRE gag-pol expression plasmid, the pHGVSV-G (SEQ ID NO: 74) env expression plasmid, and the pCAH/SINd1/hlacZ (SEQ ID NO: 79) transfer vector plasmid into human 293T cells. As a positive control, the pCMVΔR8.2 gag-pol expression plasmid, the pHGVSV-G (SEQ ID NO: 74) env expression plasmid, and the pHR/lacZ transfer vector were co-transfected into the 293T cells to produce the HIV-1 vector particles. As a negative control, only the pCAH/SINd1/hlacZ (SEQ ID NO: 79) transfer vector plasmid was transfected. Forty eight hours after transfection, the culture supernatants were harvested from each of the transfected cells and applied to fresh 293T cells in the presence of 8 μg/ml polybrene for infection. After 48 hours, genomic DNA was prepared from each of the transduced cells, followed by southern blot assay after restriction enzyme digestion. The Dig-labeled lacZ probes detected 3.15 kb BamH I-Kpn I fragment for the HIV-1-based transfer vector, and 1.35 kb Hind III-Ssp I fragment for the CAEV-based transfer vector and the negative control. For the positive controls, the 0.3 ng and 3 ng of Hind III-Ssp I DNA fragment of the pCAH/SINd1/hlacZ (SEQ ID NO: 79) transfer vector plasmid were used. As shown in FIG. 12, the CAEV-based transfer vector of the present invention was integrated at a level comparable to that of the HIV-1-based lentiviral transfer vector.

### EXAMPLE 7

### Gene Transfer to Non-dividing Cells

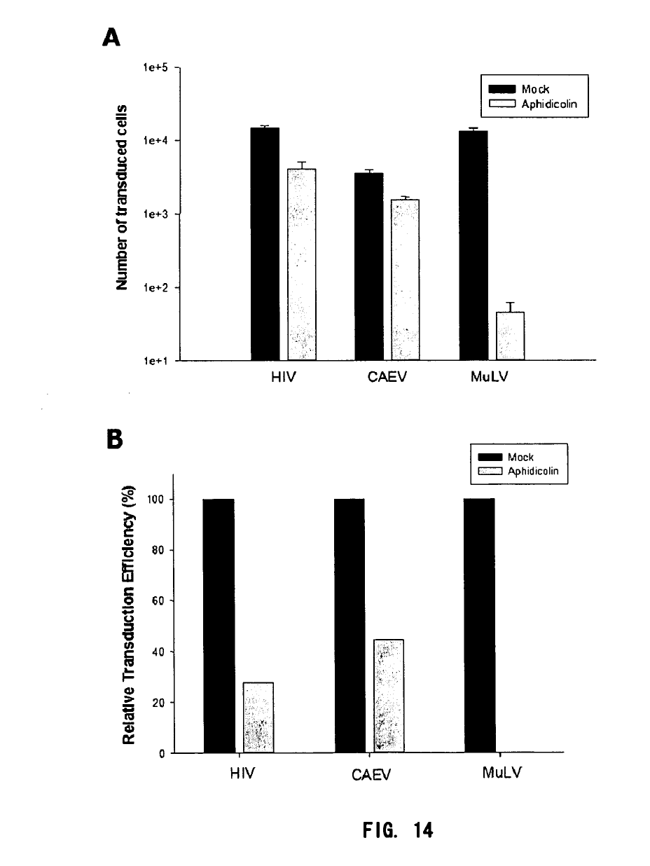

293T cells were treated with the DNA synthesis inhibitor, aphidicolin, plated on a 6-well Culture plate, and then transduced with the CAEV vector particles encoding a lacZ marker gene. As controls, cells were infected side-by-side with a lacZ expressing MuLV retroviral vector and HIV-1 lentiviral vector. At 48 hours after infection, in order to examine the trasduction efficiency, expression of the transduced lacZ gene was counted by X-gal staining. As shown in FIG. 14, the MuLV-derived vector efficiently infected cells not treated with the DNA synthesis inhibitor. However, when cells were arrested in the cell cycle by the DNA synthesis inhibitor treatment, the transduction efficiency was dropped markedly. In contrast, the CAEV-based vector was capable of efficiently transducing non-dividing human cells as well as dividing cells at a level comparable to that of the HIV-1-based vector.

### EXAMPLE 8

### In Vivo Transduction of Muscle Cells

In this example, the CAH/SINd1/hlacZ (SEQ ID NO: 79) CAEV vector is used to transduce muscle cells in vivo. The hind-legs of mice (Beige strain) are intramuscularly injected with 100 μl of the CAEV vectors in the presence of 4 μg/ml of polybrene. The mice are sacrificed two days later and the injected tissue is prepared for frozen section and for β-galactosidase analysis. The expected result is that CAH/SINd1lacZ (SEQ ID NO: 79) CAEV vector transduces muscle cells efficiently in vivo.

The foregoing specification, including the specific embodiments and examples, is intended to be illustrative of the invention and is not to be taken as limiting. Numerous other variations and modifications can be effected without departing from the true spirit and scope of the invention. All publications, including sequences deposited in the NCBI database, patents and patent applications cited herein are incorporated by reference in their entirety into the disclosure.

### REFERENCES

Altschul, S. F., Gish, W., Miller, W., Myers, E. W., and Lipman, D. J. (1990). Basic local alignment search tool. J Mol Biol 215(3), 403-10.

Altschul, S. F., Madden, T. L., Schaffer, A. A., Zhang, J., Zhang, Z., Miller, W., and Lipman, D. J. (1997). Gapped BLAST and PSI-BLAST: a new generation of protein database search programs. Nucleic Acids Res 25(17), 3389-402.

Anderson, M. P., Rich, D. P., Gregory, R. J., Smith, A. E., and Welsh, M. J. (1991). Generation of cAMP-activated chloride currents by expression of CFTR. Science 251(4994), 679-82.

Bridges, S. H., and Sarver, N. (1995). Gene therapy and immune restoration for HIV disease. Lancet 345(8947), 427-32.

Bums, J. C., Friedmann, T., Driever, W., Burrascano, M., and Yee, J. K. (1993). Vesicular stomatitis virus G glycoprotein pseudotyped retroviral vectors: concentration to very high titer and efficient gene transfer into mammalian and nonmammalian cells. Proc Natl Acad Sci USA 90(17), 8033-7.

Carswell, S., and Alwine, J. C. (1989). Efficiency of utilization of the simian virus 40 late polyadenylation site: effects of upstream sequences. Mol Cell Biol 9(10), 4248-58.

Cech, T. R. (1988). Ribozymes and their medical implications. Jama 260(20), 3030-4.

Chang, M. W., Barr, E., Seltzer, J., Jiang, Y. Q., Nabel, G. J., Nabel, E. G., Parmacek, M. S., and Leiden, J. M. (1995). Cytostatic gene therapy for vascular proliferative disorders with a constitutively active form of the retinoblastoma gene product. Science 267(5197), 518-22.

Curran, M. A., and Nolan, G. P. (2002). Nonprimate lentiviral vectors. Curr Top Microbiol Immunol 261, 75-105.

Crawford, I., Maloney, P. C., Zeitlin, P. L., Guggino, W. B., Hyde, S. C., Turley, H., Gatter, K. C., Harris, A., and Higgins, C. F. (1991). Immunocytochemical localization of the cystic fibrosis gene product CFTR. Proc Natl Acad Sci USA 88(20), 9262-6.

DuBridge, R. B., Tang, P., Hsia, H. C., Leong, P. M., Miller, J. H., and Calos, M. P. (1987). Analysis of mutation in human cells by using an Epstein-Barr virus shuttle system. Mol Cell Biol 7(1), 379-87.

Erlich, H. A. (1989). Polymerase chain reaction. J Clin Immunol 9(6), 437-47.

Fagerhol, M. K., and Cox, D. W. (1981). The Pi polymorphism: genetic, biochemical, and clinical aspects of human alpha 1-antitrypsin. Adv Hum Genet 11, 1-62, 371-2.

Gilbert, J. R., and Wong-Staal, F. (2001). HIV-2 and SIV vector systems. Somat Cell Mol Genet 26(1-6), 83-98.

Gillam, S., and Smith, M. (1979). Site-specific mutagenesis using synthetic oligodeoxyribonucleotide primers: I. Optimum conditions and minimum ologodeoxyribonucleotide length. Gene 8(1), 81-97.

Hall, C. V., Jacob, P. E., Ringold, G. M., and Lee, F. (1983). Expression and regulation of Escherichia coli lacZ gene fusions in mammalian cells. J Mol Appl Genet 2(1), 101-9.

Harmache, A., Bouyac, M., Audoly, G., Hieblot, C., Peveri, P., Vigne, R., and Suzan, M. (1995). The vif gene is essential for efficient replication of caprine arthritis encephalitis virus in goat synovial membrane cells and affects the late steps of the virus replication cycle. J Virol 69(6), 3247-57.

Harmache, A., Russo, P., Guiguen, F., Vitu, C., Vignoni, M., Bouyac, M., Hieblot, C., Pepin, M., Vigne, R., and Suzan, M. (1996). Requirement of caprine arthritis encephalitis virus vif gene for in vivo replication. Virology 224(1), 246-55.

Helene, C. (1991). The anti-gene strategy: control of gene expression by triplex-forming-oligonucleotides. Anticancer Drug Des 6(6), 569-84.

Hoffman, E. P., Brown, R. H., Jr., and Kunkel, L. M. (1987). Dystrophin: the protein product of the Duchenne muscular dystrophy locus. Cell 51(6), 919-28.

Horwich, A. L., Fenton, W. A., Williams, K. R., Kalousek, F., Kraus, J. P., Doolittle, R. F., Konigsberg, W., and Rosenberg, L. E. (1984). Structure and expression of a complementary DNA for the nuclear coded precursor of human mitochondrial omithine transcarbamylase. Science 224(4653), 1068-74.

Karlin, S., and Altschul, S. F. (1990). Methods for assessing the statistical significance of molecular sequence features by using general scoring schemes. Proc Natl Acad Sci USA 87(6), 2264-8.

Karlin, S., and Altschul, S. F. (1993). Applications and statistics for multiple high-scoring segments in molecular sequences. Proc Natl Acad Sci USA 90(12), 5873-7.

Kim, S. J., Sadelain, M., Choi, K. H., Kim, H. K., Lee, J. S., and Chung, H. Y. (1997). Tetracycline-mediated suppression of gene expression with a new dicistronic retroviral vector. Mol Cells 7(4), 514-20.

Kim, S. Y., Lee, J. H., Shin, H. S., Kang, H. J., and Kim, Y. S. (2002). The human elongation factor 1 alpha (EF-1 alpha) first intron highly enhances expression of foreign genes from the murine cytomegalovirus promoter. J Biotechnol 93(2), 183-7.

Kordower, J. H., Emborg, M. E., Bloch, J., Ma, S. Y., Chu, Y., Leventhal, L., McBride, J., Chen, E. Y., Palfi, S., Roitberg, B. Z., Brown, W. D., Holden, J. E., Pyzalski, R., Taylor, M. D., Carvey, P., Ling, Z., Trono, D., Hantraye, P., Deglon, N., and Aebischer, P. (2000). Neurodegeneration prevented by lentiviral vector delivery of GDNF in primate models of Parkinson's disease. Science 290(5492), 767-73.

Landau, N. R., and Littman, D. R. (1992). Packaging system for rapid production of murine leukemia virus vectors with variable tropism. J Virol 66(8), 5110-3.

Lee, C. C., Pearlman, J. A., Chamberlain, J. S., and Caskey, C. T. (1991). Expression of recombinant dystrophin and its localization to the cell membrane. Nature 349(6307), 334-6.

Lemarchand, P., Jaffe, H. A., Danel, C., Cid, M. C., Kleinman, H. K., Stratford-Perricaudet, L. D., Perricaudet, M., Pavirani, A., Lecocq, J. P., and Crystal, R. G. (1992). Adenovirus-mediated transfer of a recombinant human alpha 1-antitrypsin cDNA to human endothelial cells. Proc Natl Acad Sci USA 89(14), 6482-6.

Maher, L. J., 3rd, Wold, B., and Dervan, P. B. (1991). Oligonucleotide-directed DNA triple-helix formation: an approach to artificial repressors? Antisense Res Dev 1(3), 277-81.

Marcus-Sekura, C. J. (1988). Techniques for using antisense oligodeoxyribonucleotides to study gene expression. Anal Biochem 172(2), 289-95.

Miller, A. D. (1992). Human gene therapy comes of age. Nature 357(6378), 455-60.

Mitrophanous, K., Yoon, S., Rohll, J., Patil, D., Wilkes, F., Kim, V., Kingsman, S., Kingsman, A., and Mazarakis, N. (1999). Stable gene transfer to the nervous system using a non-primate lentiviral vector. Gene Ther 6(11), 1808-18.

Mselli-Lakhal, L., Favier, C., Da Silva Teixeira, M. F., Chettab, K., Legras, C., Ronfort, C., Verdier, G., Momex, J. F., and Chebloune, Y. (1998). Defective RNA packaging is responsible for low transduction efficiency of CAEV-based vectors. Arch Virol 143(4), 681-95.

Mullen, C. A., Kilstrup, M., and Blaese, R. M. (1992). Transfer of the bacterial gene for cytosine deaminase to mammalian cells confers lethal sensitivity to 5-fluorocytosine: a negative selection system. Proc Natl Acad Sci USA 89(1), 33-7.

Mulligan, R. C. (1993). The basic science of gene therapy. Science 260(5110), 926-32.

Mullis, K. B., and Faloona, F. A. (1987). Specific synthesis of DNA in vitro via a polymerase-catalyzed chain reaction. Methods Enzymol 155, 335-50.

Murakami, K., Amaya, Y., Takiguchi, M., Ebina, Y., and Mori, M. (1988). Reconstitution of mitochondrial protein transport with purified omithine carbamoyltransferase precursor expressed in Escherichia coli. J Biol Chem 263(34), 18437-42.

Naldini, L., Blomer, U., Gallay, P., Ory, D., Mulligan, R., Gage, F. H., Verma, I. M., and Trono, D. (1996). In vivo gene delivery and stable transduction of nondividing cells by a lentiviral vector. Science 272(5259), 263-7.

Ory, D. S., Neugeboren, B. A., and Mulligan, R. C. (1996). A stable human-derived packaging cell line for production of high titer retrovirus/vesicular stomatitis virus G pseudotypes. Proc Natl Acad Sci USA 93(21), 11400-6.

Persons, D. A., Mehaffey, M. G., Kaleko, M., Nienhuis, A. W., and Vanin, E. F. (1998). An improved method for generating retroviral producer clones for vectors lacking a selectable marker gene. Blood Cells Mol Dis 24(2), 167-82.

Pfarr, D. S., Rieser, L. A., Woychik, R. P., Rottman, F. M., Rosenberg, M., and Reff, M. E. (1986). Differential effects of polyadenylation regions on gene expression in mammalian cells. DNA 5(2), 115-22.

Rhodes, A., and James, W. (1991). Inhibition of heterologous strains of HIV by antisense RNA. Aids 5(2), 145-51.

Riordan, J. R., Rommens, J. M., Kerem, B., Alon, N., Rozmahel, R., Grzelczak, Z., Zielenski, J., Lok, S., Plavsic, N., Chou, J. L., and et al. (1989). Identification of the cystic fibrosis gene: cloning and characterization of complementary DNA. Science 245(4922), 1066-73.

Roberts, S., Cheetham, J. C., and Rees, A. R. (1987). Generation of an antibody with enhanced affinity and specificity for its antigen by protein engineering. Nature 328(6132), 731-4.

Rommens, J. M., Iannuzzi, M. C., Kerem, B., Drumm, M. L., Melmer, G., Dean, M., Rozmahel, R., Cole, J. L., Kennedy, D., Hidaka, N., and et al. (1989). Identification of the cystic fibrosis gene: chromosome walking and jumping. Science 245(4922), 1059-65.

Saltarelli, M., Querat, G., Konings, D. A., Vigne, R., and Clements, J. E. (1990). Nucleotide sequence and transcriptional analysis of molecular clones of CAEV which generate infectious virus. Virology 179(1), 347-64.

Saltarelli, M. J., Schoborg, R., Gdovin, S. L., and Clements, J. E. (1993). The CAEV tat gene trans-activates the viral LTR and is necessary for efficient viral replication. Virology 197(1), 35-44.

Saltarelli, M. J., Schoborg, R., Pavlakis, G. N., and Clements, J. E. (1994). Identification of the caprine arthritis encephalitis virus Rev protein and its cis-acting Rev-responsive element. Virology 199(1), 47-55.

Sauter, S. L., and Gasmi, M. (2001). FIV vector systems. Somat Cell Mol Genet 26(1-6); 99-129.

Seroude, V., Audoly, G., Gluschankof, P., and Suzan, M. (2002). Viral and cellular specificities of caprine arthritis encephalitis virus Vif protein. Virology 292(1), 156-61.

Smith, T. F., Waterman, M. S., and Fitch, W. M. (1981). Comparative biosequence metrics. J Mol Evol 18(1), 38-46.

Soneoka, Y., Cannon, P. M., Ramsdale, E. E., Griffiths, J. C., Romano, G., Kingsman, S. M., and Kingsman, A. J. (1995). A transient three-plasmid expression system for the production of high titer retroviral vectors. Nucleic Acids Res 23(4), 628-33.

To, R. Y., Booth, S. C., and Neiman, P. E. (1986). Inhibition of retroviral replication by anti-sense RNA. Mol Cell Biol 6(12), 4758-62.

Toole, J. J., Knopf, J. L., Wozney, J. M., Sultzman, L. A., Buecker, J. L., Pittman, D. D., Kaufman, R. J., Brown, E., Shoemaker, C., Orr, E. C., and et al. (1984). Molecular cloning of a cDNA encoding human antihaemophilic factor. Nature 312(5992), 342-7.

von Ruden, T., and Gilboa, E. (1989). Inhibition of human T-cell leukemia virus type I replication in primary human T cells that express antisense RNA. J Virol 63(2), 677-82.

Wang, C. C. (1991). A novel suicide inhibitor strategy for antiparasitic drug development. J Cell Biochem 45(1), 49-53.

Wame, P. H., Viciana, P. R., and Downward, J. (1993). Direct interaction of Ras and the amino-terminal region of Raf-1 in vitro. Nature 364(6435), 352-5.

Weintraub, H. M. (1990). Antisense RNA and DNA. Sci Am 262(1), 40-6.

Wills, K. N., Maneval, D. C., Menzel, P., Harris, M. P., Sutjipto, S., Vaillancourt, M. T., Huang, W. M., Johnson, D. E., Anderson, S. C., Wen, S. F., and et al. (1994). Development and characterization of recombinant adenoviruses encoding human p53 for gene therapy of cancer. Hum Gene Ther 5(9), 1079-88.

Wion, K. L., Kelly, D., Summerfield, J. A., Tuddenham, E. G., and Lawn, R. M. (1985). Distribution of factor VIII mRNA and antigen in human liver and other tissues. Nature 317(6039), 726-9.

TABLE 1

Pileup
MSF: 9300 Type: N Check: 9398 . . .
Name: NC_001463 (SEQ ID NO: 1) Len: 9300 Check: 3957 Weight: 0
Name: AF322109 (SEQ ID NO: 2) Len: 9300 Check: 5441 Weight: 0
//

| | |
|---|---|
| | 1 50 |
| NC_001463 | .......... ....GAGTTC TAGG...AGA GTCCCTCCTA GTCTCTCCTC |
| AF322109 | GTGAGTGCTC TGAGGAGCTC GAAGGAAAGA GTCC.TC..A GCCTCTCCTC |
| | 51 100 |
| NC_001463 | TCCGAGGAGG TACCGAGACC TCAAAATAAA GGAGTGATTG CCTTACTGCC |
| AF322109 | TCCGAGGAGC TTCGG....C TCATAATAAA GGAGTGCTTG CTTCA..ACA |
| | 101 150 |
| NC_001463 | GAGTGGAGAG TGATTACTGA GCGGCCGGTG TATCGGGAGT CGTCCCTTAA |
| AF322109 | GAACTGAG.. ......CTGG TCGTGGTTAT TATCGGG... .GACCGAAGT |
| | 151 200 |
| NC_001463 | TCTGTGCAAT ACCAGAGCGG CTCTCGCAGC TGGCGCCCAA CGTGGGGCCC |
| AF322109 | CCCGTGCAAC ACCGGGGCGG TTCTCGCAGC TGGCGCCCAA CGTGGGGCTC |
| | 201 250 |
| NC_001463 | GAGGAG.... .......... .......... .......... .......... |
| AF322109 | GAGTAGCTTG AGAAGCTCGA CTGAGATCTG AATCCAAGAG CGACATCAGA |
| | 251 300 |
| NC_001463 | ....AAGAAA AGAAAGC... GGCCCTGAGA ACTCGGCTTC TG..AAAAAG |
| AF322109 | CAGCAAGAAA TGAGAGTAAT GAGACCGCGA GCTCTGCTGC TGTAAAAAAG |
| | 301 350 |
| NC_001463 | AGGAAGAGGA CAAGTTGCTA TAGCAACAAG AGAGAAGAAG TAGAGCAAAG |
| AF322109 | AGGAAGTAG. CGGGTTGCCG AGGCAACTGC TCAGAAGAAC CAGGGGAAAG |
| | 351 400 |
| NC_001463 | GTCCAGTGGC T.CGGAAAAA GAGGAACTGA AACTTCGGGG ACGCCTGAAG |
| AF322109 | GGCTTCCAGC AACCTCAAAA GAGGAACCGA GACTTCGGGG ACGCCTGAA. |
| | 401 450 |
| NC_001463 | GAGTAAGGTA AGTGACTCTG CTGTACGCGG GGCGAGGCAG AGGTT.TCCT |
| AF322109 | ..GTAAGGTA AGTGACTCTG CTGTACGCGG GGCGAGGCAT AGGAGATCCT |
| | 451 500 |
| NC_001463 | TCTAAATT.G AAAGAGAAGT GTTGCTGCGA GAGGTCTTGG TGGTCGAGAA |
| AF322109 | TCTATTCTAG GAAGAGAAGC GCTGTTCTGG GAGGTCTTGG CGACCGAGAA |
| | 501 550 |
| NC_001463 | TCCTGTACAA AAAAAAGGAG GGATCTCGGT CAGGACCAGG ACCCCTGGGA |
| AF322109 | TCTTGTT... AAATAAGCCA GGATCTCGAT CAGGACCAAG ACCCCTCAGG |
| | 551 600 |
| NC_001463 | GTAATACAAC AGCAACACCG TAAGAAAATC CGCCATGGTG AGTCTAGATA |
| AF322109 | AGAGGGTATA GACAGCGTGG TAAGAAA.TC CGCCGTGGTG AGTCTAGATA |
| | 601 650 |
| NC_001463 | GAGACATGGC GAGGCAAGTC TCCGGGGGGA AAAGAGATTA TCCTGAGCTC |
| AF322109 | GAGACATGGT GAGGCAGGCC TCCGGAAGGG GAAAGGAGTA CCCCGAGCTA |
| | 651 700 |
| NC_001463 | GAAAAATGTA TCAAGCATGC ATGCAAGATA AAAGTTCGAC TCAGAGGGGA |
| AF322109 | AAAGAATGTC TGAAAAAGGC ATGCAAAATA AAAGTAAGGG CTGGGGGGGA |
| | 701 750 |
| NC_001463 | GCACTTGACA GAAGGAAATT GTTTATGGTG CCTTAAAACA TTAGATTACA |
| AF322109 | GCGCCTGACA GAAGGAAATT GTCTCTGGTG TATAAAAACA CTAGAGTGTA |
| | 751 800 |
| NC_001463 | TGTTTGAGGA CCATAAAGAG GAACCTTGGA CAAAAGTAAA ATTTAGGACA |
| AF322109 | TGTATGAGGA TTGTAGGGAG GAACCTTGGA CCCCAGAAAA ATGTAAACAA |
| | 801 850 |
| NC_001463 | ATATGGCAGA AGGTGAAGAA TCTAACTCCT GAGGAGAGTA ACAAAAAAGA |
| AF322109 | TTATGGAAAA AGTTGAAGCA GGTAGAGCCT GAGGAGAGTA GCAAAGCAGA |
| | 851 900 |
| NC_001463 | CTTTATGTCT TTGCAGGCCA CATTAGCGGG TCTAATGTGT TGCCAAATGG |
| AF322109 | CTATAACTCG TTAAAAGCAA CCTTGGCGGG GATAGTCTGT GTGCAAATGG |
| | 901 950 |
| NC_001463 | GGATGAGACC TGAGACATTG CAAGATGCAA TGGCTACAGT AATCATGAAA |
| AF322109 | GAATGCAGCC CGAGACACTG CAGGATGCGA TAGCAACCTT AAACATGAGA |
| | 951 1000 |
| NC_001463 | GATGGGTTAC TGGAACAAGA GGAAAAGAAG GAAGACAAAA GAGAAAAGGA |
| AF322109 | GA........ TGAAGTAAAA GGAAAGGAA. .AAGCCATCA GAAGAAAAGA |
| | 1001 1050 |
| NC_001463 | AGAGAGTGTC TTCCCAATAG TAGTGCAAGC AGCAGGAGGG AGAAGCTGGA |
| AF322109 | AGGGAATATA TCCC..ATAT TAGTGCAGGC AGGAGGAGGA AGAGCATGGA |
| | 1051 1100 |
| NC_001463 | AAGCAGTAGA TTCTGTAATG TTCCAGCAAC TGCAAACAGT AGCAATGCAG |
| AF322109 | GAGCGGTAGA GCCTGCTACC TTTCAGCAGC TCCAAACAGT GGCAATGCAG |
| | 1101 1150 |
| NC_001463 | CATGGCCTCG TGTCTGAGGA CTTTGAAAGG CAGTTGGCAT ATTATGCTAC |
| AF322109 | CATGGACTAG TATCAGAAGA ATTTGAAAGG CAGCTAGCAT ACTATGCCAC |
| | 1151 1200 |
| NC_001463 | TACCTGGACA AGTAAAGACA TACTAGAAGT ATTGGCCATG ATGCCTGGAA |
| AF322109 | CACATGGACA AGCAAGGATA TCTTAGAAGT ATTAGCCATG ATGCCAGGAA |
| | 1201 1250 |
| NC_001463 | ATAGAGCTCA AAAGGAGTTA ATTCAAGGGA AATTAAATGA AGAAGCAGAA |
| AF322109 | ATAGAGCGCA AAAAGAACTA ATACAAGGAA AGTTAAATGA GGAAGCAGAG |
| | 1251 1300 |
| NC_001463 | AGGTGGAGAA GGAATAATCC ACCACCTCCA GCAGGAGGAG GATTAACAGT |
| AF322109 | AGATGGAGAA GGCAGAATCC ACAACCT... ...GCGGGCG GGTTAACCGT |
| | 1301 1350 |
| NC_001463 | GGATCAAATT ATGGGGGTAG GACAAACAAA TCAAGCAGCA GCACAAGCTA |
| AF322109 | GGATCAGATA ATGGGGGTAG GACAAACGAA TCAGGCAGCG GCACAGGCTA |
| | 1351 1400 |
| NC_001463 | ACATGGATCA GGCAAGGCAA ATATGCCTGC AATGGGTAAT AAATGCATTA |
| AF322109 | ATATGGATCA AGCAAGACAA ATATGCCTAC AATGGGTTAT AACAGCAATA |
| | 1401 1450 |
| NC_001463 | AGAGCAGTAA GACATATGGC GCACAGGCCA GGGAATCCAA TGCTAGTAAA |
| AF322109 | AGAGGAGTTA GGCATATGGC CCATAGACCA GGAAATCCCA TGCTGGTAAG |
| | 1451 1500 |
| NC_001463 | GCAAAAAACG AATGAGCCAT ATGAAGATTT TGCAGCAAGA CTGCTAGAAG |
| AF322109 | ACAAAAACCA AATGAGAACT ATGAAGAGTT TGCCGCAAGG TTGTTAGAAG |
| | 1501 1550 |
| NC_001463 | CAATAGATGC AGAGCCAGTT ACACAGCCTA TAAAAGATTA TCTAAAGCTA |
| AF322109 | CAGTGGATGC AGAACCCGTT ACCCAACCTA TAAAAGAATA TTTAAAGGTA |
| | 1551 1600 |
| NC_001463 | ACACTATCTT ATACAAATGC ATCAGCAGAT TGTCAGAAGC AAATGGATAG |
| AF322109 | ACTCTGTCTT ACACAAATGC AAATTCGGAA TGTCAAAAAC ATATGGACAG |
| | 1601 1650 |
| NC_001463 | AACACTAGGA CAAAGAGTAC AACAAGCTAG TGTAGAAGAA AAAATGCAAG |
| AF322109 | AGTGTTGGGG CAAAGAGTAC AGCAGGCCTC AATAGAAGAA AAAATGCAGG |
| | 1651 1700 |
| NC_001463 | CATGTAGAGA TGTGGGATCA GAAGGGTTCA AAATGCAATT GTTAGCACAA |
| AF322109 | CATGCAGGGA CATCGGGGGA ACAGCATATC AGATGCAGTT GCTTGCACAA |
| | 1701 1750 |
| NC_001463 | GCATTAAGGC CAGGAAAAGG AAAAGGGAAT GGACAGCCAC AAAGGTGTTA |
| AF322109 | GCCCTCCGTG GCGGAAAAGA AGATGGGAAA AAATCTGTAG GGAAGTGTTA |
| | 1751 1800 |
| NC_001463 | CAACTGTGGA AAACCGGGAC ATCAAGCAAG GCAATGTAGA CAAGGAATCA |
| AF322109 | TAACTGTGGA AGGCCCGGAC ACAGAGCAAA AGAATGCAGA CAAGGCATTA |
| | 1801 1850 |
| NC_001463 | TATGTCACAA CTGTGGAAAG AGAGGACATA TGCAAAAAGA ATGCAGAGGA |
| AF322109 | TATGTCACAA CTGTGGAAAA AGAGGGCATA TACAGAAAAA CTGCA....A |
| | 1851 1900 |
| NC_001463 | AAGAGAGACA TAAGGGGAAA ACAGCAGGGA AACGGGAGGA GGGGGATACG |
| AF322109 | AC.AGAA... .AAGAAGAAA GGAGCAGGGA AACATGAGGA GGGGGCTACG |
| | 1901 1950 |
| NC_001463 | TGTGGTGCCG TCCGCTCCTC CTATGGAATA ACTTCAGCAC CACCTATGGT |
| AF322109 | TGTGGTGCCG TCCGCACCCC CTATGGAGTA ACGCAAGCAC CACTAATAGT |
| | 1951 2000 |
| NC_001463 | TCAGGTCCGC ATAGGTTCCC AGCAGAGGAA CTTGTTATTT GATACCGGGG |
| AF322109 | TAGGGTACAA ATAGGGAATC AGGAGAAACA ATTATTATTT GACACAGGGG |
| | 2001 2050 |
| NC_001463 | CGGACCGAAC TATAGTTAGA TGGCATGAGG GCTCGGGAAA CCCAGCCGGA |
| AF322109 | CAGATAAAAC GATAGTAAGA ATGCATGATG GAACAGGGAT TCCAAACGGA |
| | 2051 2100 |
| NC_001463 | AGGATAAAAC TGCAAGGAAT AGGAGGAATA GTAGAAGGAG AAAAATGGAA |
| AF322109 | AGAATAAAAT TACAAGGGAT AGGAGGAATA GTAGAAGGAG AAAAATGGAA |
| | 2101 2150 |
| NC_001463 | TAATGTAGAA TTAGAATATA AAGGAGAAAC AAGAAAGGGA ACAATAGTAG |
| AF322109 | TAAAGTACCC ATGACATATA AGGGAGAAAC ATCCTGCCCA AGCTTGGTTG |
| | 2151 2200 |
| NC_001463 | TGTTACCACA AAGTCCAGTA GAAGTATTAG GACGAGATAA CATGGCCCGA |
| AF322109 | TGCTAAGAGA TAGCCCAGTA GAAGTATTGG GAAGAGATAA CATGGAAGCA |
| | 2201 2250 |
| NC_001463 | TTTGGAATAA AGATAATAAT GGCAAATTTA GAGGAAAAAA GAATCCCAAT |
| AF322109 | TTCGGCGTAA CCCTAATAAT GGCAAATTTA GAAGATAAGA AAATTCCCAC |
| | 2251 2300 |
| NC_001463 | TACAAAAGTA AAATTGAAAG AGGGATGTAC GGGTCCACAT GTCCCACAAT |
| AF322109 | AATACCAGTA GAATTGAAAG AAGGATGTAA AGGGCCACAT GTGCCCCAGT |
| | 2302 2350 |
| NC_001463 | GGCCATTAAC AGAAGAGAAA TTAAAAGGTC TAACAGAAAT CATAGATAAA |
| AF322109 | GGCCATTAAC AGCAGAGAAA TTACAAGGAC TAACAGGAAT AGTAGAAAAA |
| | 2352 2400 |
| NC_001463 | TTAGTGGAAG AAGGAAAACT AGGAAAGGCA CCCCCACATT GGACATGTAA |
| AF322109 | TTACTACAGG AAGGAAAATT GGCAGAGGCC CCAGAGGGAT GGACGTGGAA |
| | 2402 2450 |
| NC_001463 | TACTCCAATC TTTTGCATAA AAAAGAAATC AGGGAAGTGG AGAATGTTAA |
| AF322109 | CACGCCCATC TTCTGCATAA AAAAGAAGTC AGGAAAATGG AGAATGTTAA |
| | 2451 2500 |
| NC_001463 | TAGATTTCAG AGAATTGAAC AAACAGACAG AAGATTTAAC AGAAGCGCAG |
| AF322109 | TAGATTTTAG GGAATTAAAT AAGCAAACAG CAGATTTAGC AGAAGCGCAG |
| | 2502 2550 |
| NC_001463 | TTAGGACTCC CGCATCCGGG AGGACTACAA AAGAAAAAAC ATGTTACAAT |
| AF322109 | CTAGGACTGC CACACCCAGG AGGGTTGCAA AGGAAAAAGA ATGTAACAAT |
| | 2551 2600 |
| NC_001463 | ATTGGACATA GGAGATGCAT ATTTTACTAT ACCCCTATAT GAACCATATC |
| AF322109 | TCTGGACATA GGAGATGCAT ATTTCACAAT TCCCTTATAC GAGCCCTATC |
| | 2601 2650 |
| NC_001463 | GAGAGTACAC ATGTTTTACT CTATTAAGTC CTAATAATCT AGGACCATGT |
| AF322109 | AGAAATATAC ATGCTTCACA CTCCTAAGTC CTAACAATTT GGGACCATGT |
| | 2651 2700 |
| NC_001463 | AAAAGATACT ATTGGAAAGT GCTGCCACAA GGTTGGAAAT TGAGTCCATC |
| AF322109 | AAAAGGTATT ATTGGAAAGT ATTACCCCAG GGATGGAAAT TGAGCCCAGC |
| | 2701 2750 |
| NC_001463 | TGTATATCAA TTTACTATGC AGGAGATCTT AGAGGATTGG ATACAGCAGC |
| AF322109 | TGTATATCAA TTCACCATGC AAAGGTTGTT AAAAGGATGG ATACAACAGC |
| | 2751 2800 |
| NC_001463 | ATCCAGAAAT TCAATTTGGC ATATATATGG ATGATATTTA CATAGGAAGT |
| AF322109 | ATAAAAACAT ACAATTTGGA ATATATATGG ATGATATCTA TATTGGAAGT |
| | 2801 2850 |
| NC_001463 | GATTTAGAAA TTAAAAAGCA TAGAGAAATA GTGAAAGATT TAGCCAATTA |
| AF322109 | GATCTAACGA TAGCCCAACA TAGGAAGATA ATAGAAGAAT TAGCCTCATT |
| | 2851 2900 |
| NC_001463 | TATTGCCCAA TATGGATTCA CTCTGCCAGA AGAGAAGAGA CAAAAGGGAT |
| AF322109 | TATAGAACAA TTTGGGTTTA CATTACCAGA AGATAAGAGA CAAGAGGGCT |
| | 2901 2950 |
| NC_001463 | ATCCAGCAAA ATGGCTAGGA TTTGAACTAC ACCCGCAGAC CTGGAAATTT |
| AF322109 | ATCCAGCAAA ATGGCTAGGA TTCGAGCTAC ATCCAGAAAA ATGGAAATAT |
| | 2951 3000 |
| NC_001463 | CAGAAGCATA CATTACCTGA ATTAACAAAG GGAACAATAA CATTAAATAA |
| AF322109 | CAAAAGCATA AATTGCCGGA ATTACAAGAG GGGGTAATAA CCCTGAACAA |
| | 3001 3050 |
| NC_001463 | ATTACAGAAA TTAGTAGGAG AATTAGTATG GAGACAATCC ATAATTGGGA |
| AF322109 | ATTACAGAAG ATAGTAGGGG AATTAGTGTG GAGACAATCC TTGATAGGAA |
| | 3051 3100 |
| NC_001463 | AAAGCATTCC TAACATTCTG AAATTAATGG AAGGAGATAG AGAATTACAA |
| AF322109 | AGAGCATCCC CAATATCATA AAATTAATGG AAGGAGATCG CGCATTACAA |
| | 3101 3150 |
| NC_001463 | AGTGAAAGAA AAATTGAAGA AGTACATGTG AAAGAATGGG AAGCATGTAG |
| AF322109 | AGTGAAAGGA AAATAGAAAG AATACATGTA CAAGAATGGG AAGCATGTCA |
| | 3151 3200 |
| NC_001463 | GAAAAAATTA GAAGAAATGG AAGGAAATTA TTATAATAAA GACAAAGATG |
| AF322109 | AAAGAAATTA GATGAAATGG TAGGAAATTA TTACAGAGAA GAAGAAGATA |
| | 3201 3250 |
| NC_001463 | TCTATGGACA ATTGGCTTGG GGAGACAAAG CTATAGAATA TATAGTGTAT |
| AF322109 | TCTATGGACA AATAACTTGG GGGGATAAGG CAATAAAATA CATAGTATTC |
| | 3251 3300 |
| NC_001463 | CAGGAGAAAG GGAAACCATT ATGGGTAAAT GTGGTTCACA ATATAAAGAA |
| AF322109 | CAAAGGAAAG GGGAACCCCT ATGGGTAAAT GTAGTACATG ACATAAAAAA |
| | 3301 3350 |
| NC_001463 | CCTAAGCATC CCGCAACAGG TTATTAAAGC AGCGCAAAAA TTAACCCAAG |
| AF322109 | TTTGAGTCTC CCACAGCAAG TGATAAAAGC AGCACAGAAA TTAACCCAGG |
| | 3351 3400 |
| NC_001463 | AAGTCATCAT TAGGACAGGA AAAATACCAT GGATATTGTT GCCAGGGAAA |
| AF322109 | AAGTAATCAT AAGAACAGGA AAAATCCCAT GGCTGCTACT ACCAGGAAGA |
| | 3401 3450 |
| NC_001463 | GAAGAAGATT GGAGACTAGA ATTGCAATTA GGGAACATCA CATGGATGCC |
| AF322109 | GAAGAAGACT GGAGATTAGA ACTGCAGGTA GGGAACATCA CGTGGATGCC |
| | 3451 3500 |
| NC_001463 | AAAATTTTGG TCCTGTTATC GAGGA.CATA CAAGATGGAG AAAAAGAAAT |
| AF322109 | ATCATTTTGG TCATGTTATC GAGGAGCACC CAAG.TGGAA AAGAAGGAAC |
| | 3501 3550 |
| NC_001463 | ATAATAGAAG AAGTAGTAGA AGGGCCTACA TATTATACAG ATGGAGGAAA |
| AF322109 | ATAGTGGCAG CAGTGGTAGA TGGACCGACA TATTATACAG ATGGGGGAAA |
| | 3551 3600 |
| NC_001463 | AAAGAATAAA GTAGGAAGTC TAGGGTTCAT AGTATCAACA GGGGAAAAAT |
| AF322109 | GAAAAACGCA CAGGGAAGCT TTGGCTTCAT CTCCCCAACA GGAGAAAAGT |
| | 3601 3650 |
| NC_001463 | TTAGAAAGCA TGAAGAGGGC ACAAACCAGC AACTAGAATT AAGAGCCATA |
| AF322109 | TCAGAAGGCA TGAAGATGGA ACTAATCAGG TATTAGAATT AAGGGCAATA |
| | 3651 3700 |
| NC_001463 | GAGGAAGCTC TAAAACAAGG GCCTCAAACA ATGAATTTAG TAACAGATAG |
| AF322109 | GAAGATCCAT GTAAACAAGG ACCTGAAAGC ATGAACATTG TAACTGACAG |
| | 3701 3750 |
| NC_001463 | TAGATATGCA TTTGAATTTT TATTAAGAAA TTGGGATGAA GAAGTAATAA |
| AF322109 | CAGGTATGCT TATGAATTCA TGCTCCGAAA CTGGGATGAA CAGGTCATAA |
| | 3751 3800 |
| NC_001463 | AGAATCCAAT TCAAGCAAGA ATTATGGAAA TTGCCCACAA GAAAGATAGG |
| AF322109 | GAAACCCCAT TCAGGCAAGA ATCATGGCAG AAGTGCACAA GAAAAAGCAG |
| | 3801 3850 |
| NC_001463 | ATAGGAGTGC ATTGGGTGCC AGGACATAAA GGGATTCCCC AAAATGAAGA |
| AF322109 | GTAGGAATAC ACTGGGTGCC AGGGCATAAA GGAATACCTC AGAATGAAGA |
| | 3851 3900 |
| NC_001463 | AATAGACAAA TATATTTCGG AAATATTTCT TGCAAAAGAA GGAGAAGGAA |
| AF322109 | GATAGACCAG TACATATCAG AAGTATTCTT AGCACGAGAA GGAACAGGGA |
| | 3901 3950 |
| NC_001463 | TTCTCCCAAA AAGAGAAGAG GATGCAGGGT ATGATTTAAT ATGCCCAGAA |
| AF322109 | TATGTGAAAA AAGGAAGGAA GATGCTGGAT ATGATTTATT ATGCCCGCAT |
| | 3951 4000 |
| NC_001463 | GAGGTTACCA TAGAGCCAGG ACAAGTGAAA TGCATCCCCA TAGAGCTAAG |
| AF322109 | GAGGTAATAC TTAAACCCCA AGAAGTAAAA CGGATCCCAA TAGACCTAAA |
| | 4001 4050 |
| NC_001463 | ATTAAATTTA AAGAAATCAC AATGGGCTAT GATTGCTACA AAAAGCAGCA |
| AF322109 | ATTAAAATTG AAAGAAAAGC AATGGGCCAT GATAAGTGGG AAAAGTAGCG |
| | 4051 4100 |
| NC_001463 | TGGCTGCCAA AGGAGTGTTC ACACAAGGAG GAATCATAGA CTCAGGATAT |
| AF322109 | TTGCAGCAAA AGGAATATTT GTACAAGGAG GCATAATAGA TTCAGGGTAT |
| | 4101 4150 |
| NC_001463 | CAGGGACAAA TACAGGTAAT AATGTATAAT AGCAATAAAA TAGCAGTAGT |
| AF322109 | CAGGGACAAG TACAAGTCAT CCTATATAAT AGTAATAAGA TAGAGGTCAA |
| | 4151 4200 |
| NC_001463 | CATACCCCAA GGGAGAAAAT TTGCACAATT AATATTAATG GATAAAAAGC |
| AF322109 | AATACCACAA GGCAGGAAAT TTGCCCAATT AATATTAATG AACTTACAAC |
| | 4201 4250 |
| NC_001463 | ATGGAAAATT GGAACCCTGG GGGGAAAGCA GAAAAACAGA AAGGGGAGAA |
| AF322109 | ATGAAGAATT AGAAGAATGG GGAAAGGAAA GAAAAACAGA AAGAGGAACA |
| | 4251 4300 |
| NC_001463 | AAAGGATTTG GGTCTACAGG AATGTATTGG ATAGAAAATA TTCCTCTGGC |
| AF322109 | AAAGGATTTG GGTCTACAGG AGCATTTTGG ATAGAGAATA TTCCCCAAGC |
| | 4301 4350 |
| NC_001463 | AGAGGAAGAC CACACAAAAT GGCATCAAGA TGCCCGATCA TTGCATCTAG |
| AF322109 | AGAGGAAGAA CATTACAAAT GGCATCAAGA TGCTAGATCT CTGCAGCTAG |
| | 4351 4400 |
| NC_001463 | AATTTGAAAT TCCAAGAACA GCAGCAGAAG ACATAGTAAA TCAATGTGAA |
| AF322109 | AATTCAAGAT ACCTAGAGCA GCAGCAGAAG ACATTATACA GCACTGTGAG |
| | 4401 4450 |
| NC_001463 | ATATGCAAAG AAGCGAGGAC ACCTGCAGTA ATTAGAGGCG GAAACAAAAG |
| AF322109 | GTATGTCAAG AAGGCAAACC CGCAGCGATC ACGAGAGGGG GAAATAAAAG |
| | 4451 4500 |
| NC_001463 | GGGGGTAAAT CATTGGCAAG TGGATTATAC CCATTATGAA AATATCATAC |
| AF322109 | AGGAATAGAT CATTGGCAGG TAGACTATAC ACATTACAAA GAACACATAA |
| | 4501 4550 |
| NC_001463 | TATTAGTATG GGTAGAAACA AATTCAGGAC TAATATATGC AGAAAAAGTA |
| AF322109 | TATTAGTATG GGTAGAGACT AATTCAGGAT TAATATTTGC AGAGAAAGTA |
| | 4551 4600 |
| NC_001463 | AAAGGAGAAT CAGGGCAAGA ATTCAGAATA AAAGTGATGC ATTGGTATGC |
| AF322109 | AAAGGAGAAT CAGGACAAGA ATTTAGGATG CAGACATTGA AATGGTATGC |
| | 4601 4650 |
| NC_001463 | ATTATTTGGT CCAGAGTCAT TGCAGTCAGA CAATGGACCT GCATTTGCAG |
| AF322109 | TTTGTTTCAA CCAAAATCAG TGCAATCAGA TAATGGGACA GCCTTCACAG |
| | 4651 4700 |
| NC_001463 | CAGAGCCCAC ACAGCTGTTA ATGCAATACC TAGGAGTAAA ACACACAACA |
| AF322109 | CTGAGGCTAC GCAGCATCTA ATGAAGTATT TAGGGATTCA GCACACTACG |
| | 4701 4750 |
| NC_001463 | GGCATACCTT GGAATCCACA GTCTCAGGCT ATAGTAGAAA GGGCACATCA |
| AF322109 | GGTATTCCGT GGAACCCCCA GTCACAAAGT TTAGTAGAAA GAGCTCATCA |
| | 4751 4800 |
| NC_001463 | ACTATTGAAA AGCACTTTAA AGAAGTTCCA GCCACAATTT GTCGCTGTAG |
| AF322109 | AACATTAAAA CACATGTTAG AAAAATTAGA ACCACAATTT GTGGCCCTAC |
| | 4801 4850 |
| NC_001463 | AATCAGCCAT AGCAGCAGCC CTAGTCGCCA TAAATATAAA AAGAAAGGGT |
| AF322109 | AGTCTGCCAT CGCAGCCACT CTAGTTGCGC TCAATATAAA AAGAAAGGGT |
| | 4851 4900 |
| NC_001463 | GGGCTGGGGA CAAGCCCTAT GGATATTTTT ATATATAATA AAGAACAGAA |
| AF322109 | GGACTAGGGG CAAGCCCTAT GGATATTTAC ATATATAATA AGGAGCAACA |
| | 4901 4950 |
| NC_001463 | AAGAATAAAT AATAAATATA ATAAAAATTC TCAAAAAATT CAATTCTGTT |
| AF322109 | AAGACAACAA GATAATAGTA ATAAATTAAT TCAGAAAA.. .AATTTTGTT |
| | 4951 5000 |
| NC_001463 | ATTACAGAAT AAGGAAAAGA GGACATC.AG GAGAGTGGAA AGGACCAACC |
| AF322109 | ATTACAGGAT CAGAAAAAGA GGCCATCCAG GAGAGTGGAA CGGCCCAACT |
| | 5001 5050 |
| NC_001463 | CAGGTACTGT GGAAAGGGGA AGGAGCCAAT TGTGGTAAAG GATATAGAAA |
| AF322109 | GAGGTACTGT GGGAAGGGGA AGGAGCCA.T AGTAGTTAAA GACAAAGAAA |
| | 5051 5100 |
| NC_001463 | GTGAAAAGTA TTTAGTAATA CCTTACAAAG ATGCAAAATT CATCCCGCCA |
| AF322109 | GTGATAGATA TCTAGTCATC CCATATAAAG ATGCAAAATT TATTCCGCCA |
| | 5101 5150 |
| NC_001463 | CCAACAAAAG AAAAGGAATA AAAAACCTGG ACCAGAATTA CCCTTAGCAC |
| AF322109 | CCGTCGGAAC AGAAGGGATA GAAGAATAGG TCCAGAATTG CCTTTATCTT |
| | 5151 5200 |
| NC_001463 | TATGGATACA TATAGCAGAA AGCATTAATG GGGATAGCTC ATGGTACATA |
| AF322109 | TATGGACTTA TACAGCATAC AGCATAAATA AAGATCCCGC ATGGTATACA |
| | 5201 5250 |
| NC_001463 | ACAATGAGAC TGCAACAGAT GATGTGGGGA AAAAGAGGAA ATAAGTTACA |
| AF322109 | ACCCTAAGAC TGCAGCAAAT GATGTGGCAT AGGAGGGGAA ATAAATTGAC |
| | 5251 5300 |
| NC_001463 | ATATAAGAAT GAAGACAGGG AATATGAAAA TTGGGAAATT ACATCATGGG |
| AF322109 | ATATGTCAGG GAAAATGCAC AGTACGAGGA GTGGGAAATG ACCTCGTATG |
| | 5301 5350 |
| NC_001463 | GATGGAAAAT GCACCTAAGG AGAGTGAAAC AATGGATACA AGACAACAGG |
| AF322109 | AGTGGAGGAT AAGAATGAGA AGGGACAAAA CAAAAAGTCA TC.CAAGAGG |
| | 5351 5400 |
| NC_001463 | AGAGGAAGC. CCATGGCAGT ACAAAGTAGG AGGAACATGG AAAAGTATAG |
| AF322109 | GCATACTTCG CCATGGCAAT ATCGGAGACA GGATGGATGG AAGGATGTGG |
| | 5401 5450 |
| NC_001463 | GAGTGTGGTT CCTGCAAGCA GGAGATTACA GAAAGGTAGA CAGGCACTTC |
| AF322109 | GAACGTGGTT CCTACAGCCA GGGGACTATA GAAAGGCGGA TCAGCAGTTC |
| | 5451 5500 |
| NC_001463 | TGGTGGGCAT GGAGGATACT GATATGTTCC TGCAGGAAAG AAAAGTTTGA |
| AF322109 | TGGTTCGCTT GGAGAATAGT GTCGTGTTCA TGTAAAAAGG AAGGATTTAA |
| | 5501 5550 |
| NC_001463 | TATAAGAGAA TTTATGAGAG GAAGACATAG ATGGGATTTG TGCAAATCCT |
| AF322109 | CATAAGAGAA TTTATGCTAG GTACCCATAG ATGGGATTTG TGTAAGTCGT |
| | 5551 5600 |
| NC_001463 | GTGCTCAAGG AGAAGTAGTA AAGCATACTA GAACAAAAAG TCTGGAAAGA |
| AF322109 | GTTGCCAGGG TGAAGTAGTA AAGAGAACAC AACCCTACAC CTTGCAAAGG |
| | 5601 5650 |
| NC_001463 | CTAGTACTGC TACAGATGGT AGAACAGCAT GTGTTTCAAG TATTGCCATT |
| AF322109 | CTCACGTGGC TTAAATTAAC AGAAGACCAT GTATTTCAAG TAATGCCCTT |
| | 5651 5700 |
| NC_001463 | GTGGAGAGCC AGGAGAAGTA GTACAACAGA TTTCCCATGG TGCAGGGACA |
| AF322109 | GTGGAGAGCT CGCAAAGGGA TTACCATAGA CTTTCCCTGG TGCAGGGACA |
| | 5701 5750 |
| NC_001463 | CAACGGGATA CACGCATGCG TGGTCTGTCC AGGAGTGCTG GTTGATGGAA |
| AF322109 | CAAAAGGATT CCTGGAGCCG TGGACAACGC AAGAGTGTTG GCAAATAGAG |
| | 5751 5800 |
| NC_001463 | TATCTCTTAG AGGATGAGTG AAGAACTGCC TCAAAGAAGG GAGACACATC |
| AF322109 | TATCCCTTGG AGGATGAGTG AGGAAACCCC AGCAGGAAGA GAACCGACTG |
| | 5801 5850 |
| NC_001463 | CAGAAGAACT .TGTAAGGAA CGTACGGGAA AGAGAAAGGG ATACATGGCA |
| AF322109 | CAGAGGAAAT ATTTGAGCAA .......GAA GCAGAAAGT. .....TGGAA |
| | 5851 5900 |
| NC_001463 | ATGGACAAGC ATCAGAGTAC CTGCGGAAAT ACTGCAAAGA TGGCTTGCTA |
| AF322109 | GAGAACAAGC GTGCGAGTCC CAAATGACAT ATTACAAAGA TGGCTAGCAA |
| | 5901 5950 |
| NC_001463 | TGCTTAGGTC AGGCAGAAAT AGAAAGAAAG TGTATAGAGA AATGCAAAAA |
| AF322109 | TGCTTAGGCA AAGAGGAAAT AGAAAGAAAG TGCTTAGGGA AATGCAAAAA |
| | 5951 6000 |
| NC_001463 | TGGATGTGGA TACATCCCAA GGCGCCTGTG ATTAGGGCCT GTGGATGCAG |
| AF322109 | TGGGCATGGA GGAATCCCAC GGCGCGGGTG ATTCGGCCGT GTGGATGTCG |
| | 6001 6050 |
| NC_001463 | ACTATGTAAC CCGGGGTGGG GAACATAATC AAGGGAATAA TAAATGCAAA |
| AF322109 | GCTATGTAAC CCCGGCTGGG GGAG.TAATT AAT..CATAA TAAA.GCAAA |
| | 6051 6100 |
| NC_001463 | TAAATGTAAC TAACAAGTAG CAAAAGTGTC TGTGTTAGAT GGATGCTGGG |
| AF322109 | T...TGTAAC .......... .......... .......... ..ATGCTGTG |
| | 6101 6150 |
| NC_001463 | GCCAGATACA TGCGCTTAAC TGGGAAGGAA AACTGGGTTG AAGTAACCAT |
| AF322109 | TC.....A............. ..GG...... ...TGTCTTG CAGGAA...T |
| | 6151 6200 |
| NC_001463 | GGACGGAGAG AAGGAAAGGA AAAGAGAAGG TTTCACTGCG GGACAGCAAG |
| AF322109 | GG.CGGAGAT AAGAAAAG.. AA.GCAAAGG AGCCACT... ..AATCCAGG |
| | 6201 6250 |
| NC_001463 | GTAAGTATCA ACCCCAGGTA AGTAAGCAAA TAGGGAACAG AAATACTAAC |
| AF322109 | GTAAGTATAA AAAACAGGTA AGTA...... ...G...... AA....TAAC |
| | 6251 6300 |
| NC_001463 | CCATGCTTTG CCTATAAAGG GATATTCCTA TGGAGGATAT CACTAACAAT |
| AF322109 | .......... ....TATAGT TATATT.... ......A... ..CTAACAGT |
| | 6301 6350 |
| NC_001463 | GTGGATATTG CTAGGGATAA ATATGTGTGT CAGTGCAGAG GATTACATAA |
| AF322109 | AAGAGCAGCA CTAGG..... ..A....... ....GCAGAA ...TACATAA |
| | 6351 6400 |
| NC_001463 | CACTAATATC AGATCCCTAT GGGTTCTCAC CCATAAAAAA TGTGTCTGGG |
| AF322109 | CCATAATATC AGACCCATAT GGGTTCTCTC CCGTGAGAAA TGTGTCAGGA |
| | 6401 6450 |
| NC_001463 | GTACCAGTGA CTTGTGTAAC AAAAGAATTC GCAAAATGGG GATGTCAACC |
| AF322109 | GTACCTGTAA CTTGTGTGAC AAAAGAATTT AGTAAGTGGG GATGTCAGCC |
| | 6451 6500 |
| NC_001463 | ACTAGGAGCG TACCCTGATC CAGAAATAGA ATACAGAAAT GTGAGTCAGG |
| AF322109 | AATAGGAGCC TACCCAGACC CAGACTTAGA ATACAGAAAT ATAAGTAAAG |
| | 6501 6550 |
| NC_001463 | AAGTAGTGAA AGAAGTATAT CAAGAGAATT GGCCATGGAA TACATATCAT |
| AF322109 | AAATATTAGA GGAAGTATAT CAACAAGACT GGCCGTGGAA TACTTATCAT |
| | 6551 6600 |
| NC_001463 | TGGCCTCTCT GGCAAATGGA GAATGTTAGG TACTGGTTAA AAGAAAATAT |
| AF322109 | TGGCCATTAT GGCAAATGGA TAATGTAGTA CAATGGGCAA GGCAAAATTT |
| | 6601 6650 |
| NC_001463 | GCAAGAAAAT CAACAGAGAA AAAATAATAC AAAAGAGGGT ATAGAGGAAT |
| AF322109 | ACAGGATAAC CGCAAG.GAA AAAAG..... ......GGAC CTGGCAGACC |
| | 6651 6700 |
| NC_001463 | TATTAGCAGG AACTATAAGG GGAAGATTCT GTGTACCATA CCCATTTGCC |
| AF322109 | TATTAGCAGG AAAAATAAGG GGAAGATTCT GTGTACCCTA CCCATTTGCG |
| | 6701 6750 |
| NC_001463 | TTGTTAAAAT GCACAAAGTG GTGCTGGTAT ACAGCGGCCA TAAA..CAAC |
| AF322109 | CTCCTGGAGT GCATGGAATG GTGCTGGTGG GTTAAGAACA CTAATGCAGG |
| | 6751 6800 |
| NC_001463 | GAGTCA.GGA AAAGCAGGAA AAATAAAAAT AAATTGCACA GAAGCAAGAG |
| AF322109 | GGGGTATGGA GAAGCAG..A .CATAAGAAT AAATTGCTCA AGGGCAAGAG |
| | 6801 6850 |
| NC_001463 | CAGTCTCCTG TACAGAGGAC ATGCCATTAG CCTCAATACA AAGAGCATAT |
| AF322109 | CAGTGAGCTG CACAAGTGAA ATGCCCTTAG CATCCCTACA GAGGGTATAT |
| | 6851 6900 |
| NC_001463 | TGGGATGAGA AAGACAGAGA GAGCATGGCC TTTATGAATA TCAAAGCATG |
| AF322109 | TGGGAAAAGG AGGAACGAAA AAACATGGAG AAAATGACCA TCAAACCTTG |
| | 6901 6950 |
| NC_001463 | TGATAGCAAC CTAAGGTGTC AGAAAAGACC TGGAGGGTGT ATGGAAGGAT |
| AF322109 | CAATAAAAAT TTGGAATGCA AGAACAGAA. .G.GGGATGC GCAGAAGGGT |
| | 6951 7000 |
| NC_001463 | ACCCTATCCC AGTAGGAGCA GAAATAATCC CTGAAAGTAT GAAATACCTA |
| AF322109 | ATCCAGTACC TCCCAAGGCA GAGTTATTCC CTCCAGCGTT TCAGGATTTA |
| | 7001 7050 |
| NC_001463 | AGGGGAGCAA AGAGTCAG.. TATGGGGGAA TAAAAGATAA GAATGGAGAA |
| AF322109 | CAGCCA..AA AGGGTACGCA TATGGGGCAC TTAGAG...G GAACAGCAAA |
| | 7051 7100 |
| NC_001463 | TTAAAATTAC CATTAACATT AAGAGTGTGG GTAAAATTAG CAAATGTGTC |
| AF322109 | TTTCCACAAA GAGTGTCGCT AAGAACATGG GTGAAAATAG CTAACCTGAC |
| | 7101 7150 |
| NC_001463 | AGAATGGGTA AATGGGACAC CCCCGGATTG GCAAGACAGA ATTAACGGAT |
| AF322109 | AGGATGGGAA AAAGGAAAGC CAGCAGAATG GT......GG AATACCAG.. |
| | 7151 7200 |
| NC_001463 | CCAAAGGAAT AAATGGGACG CTCTGGGGAG AGCTTAACAG TATGCATCAC |
| AF322109 | CCAACAGGTT CATTGGTTTG ATACCACGCC ACAATATCAT TTAGGAT... |
| | 7201 7250 |
| NC_001463 | CTAGGATTTG CCCTTAGCCA GAACGGCAAA TGGTGTAACT ACACCGGGGA |
| AF322109 | .ATGTATTAT CCCGAGCGCC TGAGAACAGG AGTTGTAATT TCACAGGGGA |
| | 7251 7300 |
| NC_001463 | AATAAAATTA GGGCAAGAAA CATTCCAATA TCATTACAAG CCAAACTGGA |
| AF322109 | AATACGAATA GGGCAACATC AGTTTGAGTA TAATTACACC CTGACAAAGA |
| | 7301 7350 |
| NC_001463 | ACTGTACC.. .GGGAATTGG ACGCAATATC CGGTGTGGCA AGTGATTAGA |
| AF322109 | ATTGCACAAA GGAGAAGTGG AAAGAGTACC CCATGTGGCA TGTCTGGAGG |
| | 7351 7400 |
| NC_001463 | AACCTGGATA TGGTGGAACA TATGACAGGA GAATGTGTGC AGAGACCACA |
| AF322109 | CATTTAGATC AAAATGAGCA CTTATCTAGC ATATGTTTCA AAAGACCGAG |
| | 7401 7450 |
| NC_001463 | AAGGCACAAT ATAACAGTAG GAAATGGAAC CATAACAGGG AATTGCAGTA |
| AF322109 | AAGAAATGCA ACACAAATAG GGAACAGTAC ACTGCAAGGG CAATGTAATA |
| | 7451 7500 |
| NC_001463 | CAACAAACTG GGATGGATGT AATTGCTCAC GATCAGGAAA CTACCTATAT |
| AF322109 | GAAGTAATTG GACAGGATGC CACTGCAATG AGACAGGGAT AAAC..AC.. |
| | 7501 7550 |
| NC_001463 | AACAGCTCTG AGGGAGGATT GTTATTAATT CTGTGCAGAC AAAACAGCAC |
| AF322109 | AACA...... .......... .......... ...TGGAGAA TAAATGGCAC |
| | 7551 7600 |
| NC_001463 | CCTAACAAGG ATCCTGGGAA CAAATACAAA TTGGACAACT ATGTGGGGAA |
| AF322109 | ....AAAGGG AGC.TT..AT CTCTTA..AA TAGCACTAAT .....GGAAA |
| | 7601 7650 |
| NC_001463 | TATACAAAAA TTGTTCAGGA TGCGAGAATG CAACATTAGA CAACACAGGA |
| AF322109 | CATCATGGTC TTGTT....A TGCTGGAACA CAACAGTGG. .....CAGGG |
| | 7651 7700 |
| NC_001463 | GAAGGAACCT TAGGAGGTGT AGCTAATAAG AACTGTAGCT TGCCTCATAA |
| AF322109 | GTA....... TATGAGAGTC AGCTAA.... A.GTGGAATG AGAGTCTTAA |
| | 7701 7750 |
| NC_001463 | AAATGAGAGC AACAAGTGGA CTTGTGCCCC AAGACAAAGA GATGGAAAAA |
| AF322109 | AGACGGAGAC TATGGGCTCT GTTTTAATTC AACAAACAGG AATTGTACTA |
| | 7751 7800 |
| NC_001463 | CAGATTC.GC TATACATAGC AGGAGGAAAA AAGTTTTGGA CACGAATTAA |
| AF322109 | GAAATGGAGC TCGGCACTAT GTAAACAAGA GAGTGATAAA AAACGAC.AC |
| | 7801 7850 |
| NC_001463 | GGCCCAATTC AGCTGTGAAA GTAACATAGG ACAATTAGAT GGAATGTTGC |
| AF322109 | AGCAGATCAT AATTGTGATA GCAGCATATC AGCAATAGAT GGAATGGTAC |
| | 7851 7900 |
| NC_001463 | ATCAGCAAAT ACTATTGCAA AAATATCAAG TAATTAAGGT AAGAGCTTAT |
| AF322109 | ATCAACAAAT ATTACTGCAA AGGTATCAAG TAATTAGAGT AAGAGCTTAC |
| | 7901 7950 |
| NC_001463 | ACATATGGGG TGATAGAAAT GCCAGAAAAC TATGCAAAAA CAAGAATCAT |
| AF322109 | ACATACGGAG TGATTGATAT GCCAGACAAT TATG.AGACC CTACCAGGA. |
| | 7951 8000 |
| NC_001463 | AAACAGGAAA AAAAGAGAAC TCAGCCACAA GAGGAAGAAG AGAGGCGTTG |
| AF322109 | ....AGGAGA AGGAGAGATC TCGCAAAGGC CAGGAAAAAG AGGGGCGTGG |
| | 8001 8050 |
| NC_001463 | GCTTGGTCAT TATGCTAGTT ATCATGGCAA TAGTAGCTGC CGCAGGGGCT |
| AF322109 | GCCTGGTCAT CATGTTAGCT ATCATGGCCA TAGTGGCTGC TGCAGGAGCA |
| | 8051 8100 |
| NC_001463 | TCTCTGGGAG TCGCAAACGC GATTCAGCAG TCTTACACTA AGGCAGCTGT |
| AF322109 | TCTCTGGGAG TCGCGAACGC GATTCAGCAG TCCTACACCA GGGACGCTGT |
| | 8101 8150 |
| NC_001463 | CCAGACCCTT GCTAATGCAA CTGCTGCACA GCAGGATGTG TTAGAGGCAA |
| AF322109 | CCAGACTCTT GCTAACGCGA CTGCTGTGCA ACAGCAGGTG TTAGAGGCGT |
| | 8151 8200 |
| NC_001463 | CCTATGCCAT GGTACAGCAT GTGGCTAAAG GCGTACGAAT CTTGGAAGCT |
| AF322109 | CCTATGCCAT GATACAGCAT GTGGCTAAGG GAATACGCAT CCTTGAAGCA |
| | 8201 8250 |
| NC_001463 | CGAGTGGCTC GAGTGGAAGC TATCACAGAT AGAATAATGC TATACCAAGA |
| AF322109 | CGCGTGGCGA GAATGGAAGT TATGATGGAT AGAATGATGT TATATCAGGA |
| | 8251 8300 |
| NC_001463 | ATTGGATTGT TGGCACTATC ATCAATACTG TATAACCTCT ACAAAAACAG |
| AF322109 | AGTAGACTGC TGGCATTATC ACCAATATTG TGTAACCTCT ACAAGAGCAG |
| | 8301 8350 |
| NC_001463 | AAGTAGCAAA ATATATCAAT TGGACGAGGT TTAAGGATAA TTGCACATGG |
| AF322109 | ACATAGTGAA TTACATTAAT TGGACAAGGT TTAAAGATAA TTGCACATGG |
| | 8351 8400 |
| NC_001463 | CAGCAGTGGG AGAGAGGATT ACAGGGGTAT GATACAAACT TAACAATACT |
| AF322109 | CAAGAGTGGG AAAGGGAGAT AAGTGCGCAT GAAGGAAACA TCACTATATT |
| | 8401 8450 |
| NC_001463 | GTTAAAGGAA TCAGCAGCAA TGACACAACT AGCAGAAGAG CAAGCAAGGA |
| AF322109 | ACTCAAAGAA TCAGCAAGGA TAACACAATT AGCACAACAA AAGGTACAAA |
| | 8451 8500 |
| NC_001463 | GGATACCAGA AGTATGGGAA AGTTTAAAAG ACGTCTTTGA TTGGTCAGGA |
| AF322109 | GAATACCAGA TGTGTGGACA GCACTAAGGG AGTCACTAGG ATGGACACAA |
| | 8501 8550 |
| NC_001463 | TGGTTCTCAT GGCTAAAGTA TATTCCTATT ATAGTAGTAG GATTATTAGG |
| AF322109 | TGGCTGGCTT GGATAAAATA CCTTCCCATA ATAGTAGTAG GGATATTAGG |
| | 8551 8600 |
| NC_001463 | ATGCATTCTG ATAAGAGCTG TGATATGTGT ATGTCAACCT CTTGTGCAGA |
| AF322109 | ATGCATAATC ATAAGAATAA TGTTGTGTGT AGTACAACCA GTTCTTCAGA |
| | 8601 8650 |
| NC_001463 | TATACAGAAC TCTAAGTACC CCGACATACC AACGGGTCAC AGTCATCATG |
| AF322109 | TTTACAGAAC CTTGACTCAG ACCAGGTATC AACAAGTCAA CTTGGTGATG |
| | 8651 8700 |
| NC_001463 | GAAACAAGAG CAGACGTCGC AGGAGAAAAT CAGGATTTTG .....GATGG |
| AF322109 | GAGACCCGGG TGCAACTAGA AGAAGAAGAA GAAGAAGACG GAAGGGATGG |
| | 8701 8750 |
| NC_001463 | CTTAGAGGAA TCAGACAA.. .CAGCGAAAC AAGCGAAAGA GTGACAGTAC |
| AF322109 | TGGAGATGGC TCAGAGAGAT GCAGCGATCC CGACAACAAA .....AATTA |
| | 8751 8800 |
| NC_001463 | AGAAAGCTTG GAGCCGTGCC TGGGAGCTTT GGCAGAACTC ACCCTGGAAG |
| AF322109 | TGAACGCCTG GAGGAGAGCT TGGGTGACTT GGAGAAACTC ACCTTGGCAG |
| | 8801 8850 |
| NC_001463 | GAGCCATGGA AAAGGGGCCT GCTGAGGCTG CTCGTCCTTC CGCTGACGAT |
| AF322109 | AACACATGGA AGAATGTGGT GGTGGCGCCG TTGGTGATTC CGCTGACAAT |
| | 8851 8900 |
| NC_001463 | GGGAATCTGG ATAAATGGAT GGCTTGGAGA ACACCACAAA AATAAAAAAA |
| AF322109 | CAGAATTTGG CTCCTTGGAG AGAATGGAGA GAACCCCTAA AAGAAAAATA |
| | 8901 8950 |
| NC_001463 | GAAAGGGTG. ACTGTGAGAC ATGGGCTAAA GAGGACTAAT AACAAGCTAG |
| AF322109 | AAAAGGGTGG ACTGTGAGGA CTGTG..... .AGGCCTAGG AGCGAGATAG |
| | 8951 9000 |
| NC_001463 | GCCAAATTCC TGTAAATCAC TTGGGGGGTT ATAAGAAAAG CAAGTTCACT |
| AF322109 | ...AAACTTA TAGGCCTCTC TTCCCGG... ......AAAG CTAACTCACT |
| | 9001 9050 |
| NC_001463 | ATGACAAAGC AAAATGTAAA GGCCAAATTC CTGTAAATCA CTTGGGGGGT |
| AF322109 | GTG....... .AGAGGAATA G..CAAGTCA CAGTGA..CA CT.....GCT |
| | 9051 9100 |
| NC_001463 | TATAAGAAAA GCAAGTTCAC TATGACAAAG CAAAATGTAA CCGCAAG... |
| AF322109 | AATTGTACCC GCAA...CCC TGAGATCATG CAAACCACAA TCCTGAGATT |
| | 9101 9150 |
| NC_004463 | .TGCTGACAG ATGTAACAGC TGACATATCA GCTGATGCTT GCTCATGCTG |
| AF322109 | ATGCTGACAT GTGTAACAGC TGATGCCTCA GCTGATGCTT GCTCATGCTG |
| | 9151 9200 |
| NC_001463 | ACACTGTAGC TCTGAGCTGT ATATAAGGAG AAGCTTGCTG CTTGC.ACTT |
| AF322109 | ACAATGTAAC TAGGAGCTCT ATATAAACAG AGCCCTAGAG CTTGCTACTT |
| | 9201 9250 |
| NC_001463 | CAGAGTTCTA GGAGAGTCCC .......... .TCCT.AGTC TCTCCTCTCC |
| AF322109 | CAGAGTGCTC TGAGGAGCTC GAAGGAAAGA GTCCTCAGCC TCTCCTCTCC |
| | 9251 9300 |
| NC_001463 | GAGGAGGTAC CGAGACCTCA AAATAAAGGA GTGATTGCCT TACTGCCGA. |
| AF322109 | GAGGAGCTTC GG....CTCA TAATAAAGGA GTGCTTGCTT CA..ACAGAA |

TABLE 2

Pileup
MSF: 759 Type: N Check: 1376 . . .
Name: NC_001463 (gag720 bp) (SEQ ID NO: 3) Len: 759 Check: 9060 Weight: 0
Name: AF322109 (gag720 bp) (SEQ ID NO: 4) Len: 759 Check: 2316 Weight: 0
//

| | |
|---|---|
| | 1 50 |
| NC_001463 (gag720bp) | ATGGTGAGTC TAGATAGAGA CATGGCGAGG CAAGTCTCCG GGGGGAAAAG |
| AF322109 (gag720bp) | .......... .......... .ATGGTGAGG CAGGCCTCCG GAAGGGGAAA |
| | 51 100 |
| NC_001463 (gag720bp) | AGATTATCCT GAGCTCGAAA AATGTATCAA GCATGCATGC AAGATAAAAG |
| AF322109 (gag720bp) | GGAGTACCCC GAGCTAAAAG AATGTCTGAA AAAGGCATGC AAAATAAAAG |
| | 101 150 |
| NC_001463 (gag720bp) | TTCGACTCAG AGGGGAGCAC TTGACAGAAG GAAATTGTTT ATGGTGCCTT |
| AF322109 (gag720bp) | TAAGGGCTGG GGGGGAGCGC CTGACAGAAG GAAATTGTCT CTGGTGTATA |
| | 151 200 |
| NC_001463 (gag720bp) | AAAACATTAG ATTACATGTT TGAGGACCAT AAAGAGGAAC CTTGGACAAA |
| AF322109 (gag720bp) | AAAACACTAG AGTGTATGTA TGAGGATTGT AGGGAGGAAC CTTGGACCCC |
| | 201 250 |
| NC_001463 (gag720bp) | AGTAAAATTT AGGACAATAT GGCAGAAGGT GAAGAATCTA ACTCCTGAGG |
| AF322109 (gag720bp) | AGAAAAATGT AAACAATTAT GGAAAAAGTT GAAGCAGGTA GAGCCTGAGG |
| | 251 300 |
| NC_001463 (gag720bp) | AGAGTAACAA AAAAGACTTT ATGTCTTTGC AGGCCACATT AGCGGGTCTA |
| AF322109 (gag720bp) | AGAGTAGCAA AGCAGACTAT AACTCGTTAA AAGCAACCTT GGCGGGGATA |
| | 301 350 |
| NC_001463 (gag720bp) | ATGTGTTGCC AAATGGGGAT GAGACCTGAG ACATTGCAAG ATGCAATGGC |
| AF322109 (gag720bp) | GTCTGTGTGC AAATGGGAAT GCAGCCCGAG ACACTGCAGG ATGCGATAGC |
| | 351 400 |
| NC_001463 (gag720bp) | TACAGTAATC ATGAAAGATG GGTTACTGGA ACAAGAGGAA AAGAAGGAAG |
| AF322109 (gag720bp) | AACCTTAAAC ATGAGAGATG AAGT...... ..AAAAGGAA AGGAA..AAG |
| | 401 450 |
| NC_001463 (gag720bp) | ACAAAAGAGA AAAGGAAGAG AGTGTCTTCC CAATAGTAGT GCAAGCAGCA |
| AF322109 (gag720bp) | CCATCAGAAG AAAAGAAGGG AATATAT..C CCATATTAGT GCAGGCAGGA |
| | 451 500 |
| NC_001463 (gag720bp) | GGAGGGAGAA GCTGGAAAGC AGTAGATTCT GTAATGTTCC AGCAACTGCA |
| AF322109 (gag720bp) | GGAGGAAGAG CATGGAGAGC GGTAGAGCCT GCTACCTTTC AGCAGCTCCA |
| | 501 550 |
| NC_001463 (gag720bp) | AACAGTAGCA ATGCAGCATG GCCTCGTGTC TGAGGACTTT GAAAGGCAGT |
| AF322109 (gag720bp) | AACAGTGGCA ATGCAGCATG GACTAGTATC AGAAGAATTT GAAAGGCAGC |
| | 551 600 |
| NC_001463 (gag720bp) | TGGCATATTA TGCTACTACC TGGACAAGTA AAGACATACT AGAAGTATTG |
| AF322109 (gag720bp) | TAGCATACTA TGCCACCACA TGGACAAGCA AGGATATCTT AGAAGTATTA |
| | 601 650 |
| NC_001463 (gag720bp) | GCCATGATGC CTGGAAATAG AGCTCAAAAG GAGTTAATTC AAGGGAAATT |
| AF322109 (gag720bp) | GCCATGATGC CAGGAAATAG AGCGCAAAAA GAACTAATAC AAGGAAAGTT |
| | 651 700 |
| NC_001463 (gag720bp) | AAATGAAGAA GCAGAAAGGT GGAGAAGGAA TAATCCACCA CCTCCAGCAG |
| AF322109 (gag720bp) | AAATGAGGAA GCAGAGAGAT GGAGAAGGCA GAATCCACAA CCTGCGG... |
| | 701 750 |
| NC_001463 (gag720bp) | GAGGAGGATT AACAGTGGAT .......... .......... .......... |
| AF322109 (gag720bp) | ...GCGGGTT AACCGTGGAT CAGATAATGG GGGTAGGACA AACGAATCAG |
| | 751 |
| NC_001463 (gag720bp) | ......... |
| AF322109 (gag720bp) | GCAGCGGCA |

Pileup
MSF: 1347 Type: N Check: 2008 . . .
Name: NC_001463 (gag) (SEQ ID NO: 5) Len: 1347 Check: 6959 Weight: 0
Name: AF322109 (gag) (SEQ ID NO: 6) Len: 1347 Check: 5049 Weight: 0
//

| | |
|---|---|
| | 1 50 |
| NC_001463 (gag) | ATGGTGAGTC TAGATAGAGA CATGGCGAGG CAAGTCTCCG GGGGGAAAAG |
| AF322109 (gag) | .......... .......... .ATGGTGAGG CAGGCCTCCG GAAGGGGAAA |
| | 51 100 |
| NC_001463 (gag) | AGATTATCCT GAGCTCGAAA AATGTATCAA GCATGCATGC AAGATAAAAG |
| AF322109 (gag) | GGAGTACCCC GAGCTAAAAG AATGTCTGAA AAAGGCATGC AAAATAAAAG |
| | 101 150 |
| NC_001463 (gag) | TTCGACTCAG AGGGGAGCAC TTGACAGAAG GAAATTGTTT ATGGTGCCTT |
| AF322109 (gag) | TAAGGGCTGG GGGGGAGCGC CTGACAGAAG GAAATTGTCT CTGGTGTATA |
| | 151 200 |
| NC_001463 (gag) | AAAACATTAG ATTACATGTT TGAGGACCAT AAAGAGGAAC CTTGGACAAA |
| AF322109 (gag) | AAAACACTAG AGTGTATGTA TGAGGATTGT AGGGAGGAAC CTTGGACCCC |
| | 201 250 |
| NC_001463 (gag) | AGTAAAATTT AGGACAATAT GGCAGAAGGT GAAGAATCTA ACTCCTGAGG |
| AF322109 (gag) | AGAAAAATGT AAACAATTAT GGAAAAAGTT GAAGCAGGTA GAGCCTGAGG |
| | 251 300 |
| NC_001463 (gag) | AGAGTAACAA AAAAGACTTT ATGTCTTTGC AGGCCACATT AGCGGGTCTA |
| AF322109 (gag) | AGAGTAGCAA AGCAGACTAT AACTCGTTAA AAGCAACCTT GGCGGGGATA |
| | 301 350 |
| NC_001463 (gag) | ATGTGTTGCC AAATGGGGAT GAGACCTGAG ACATTGCAAG ATGCAATGGC |
| AF322109 (gag) | GTCTGTGTGC AAATGGGAAT GCAGCCCGAG ACACTGCAGG ATGCGATAGC |
| | 351 400 |
| NC_001463 (gag) | TACAGTAATC ATGAAAGATG GGTTACTGGA ACAAGAGGAA AAGAAGGAAG |
| AF322109 (gag) | AACCTTAAAC ATGAGAGATG ........AA GTAAAAGGAA AGGAA..AAG |
| | 401 450 |
| NC_001463 (gag) | ACAAAAGAGA AAAGGAAGAG AGTGTCTTCC CAATAGTAGT GCAAGCAGCA |
| AF322109 (gag) | CCATCAGAAG AAAAGAAGGG AATATATCCC ..ATATTAGT GCAGGCAGGA |
| | 451 500 |
| NC_001463 (gag) | GGAGGGAGAA GCTGGAAAGC AGTAGATTCT GTAATGTTCC AGCAACTGCA |
| AF322109 (gag) | GGAGGAAGAG CATGGAGAGC GGTAGAGCCT GCTACCTTTC AGCAGCTCCA |
| | 501 550 |
| NC_001463 (gag) | AACAGTAGCA ATGCAGCATG GCCTCGTGTC TGAGGACTTT GAAAGGCAGT |
| AF322109 (gag) | AACAGTGGCA ATGCAGCATG GACTAGTATC AGAAGAATTT GAAAGGCAGC |
| | 551 600 |
| NC_001463 (gag) | TGGCATATTA TGCTACTACC TGGACAAGTA AAGACATACT AGAAGTATTG |
| AF322109 (gag) | TAGCATACTA TGCCACCACA TGGACAAGCA AGGATATCTT AGAAGTATTA |
| | 601 650 |
| NC_001463 (gag) | GCCATGATGC CTGGAAATAG AGCTCAAAAG GAGTTAATTC AAGGGAAATT |
| AF322109 (gag) | GCCATGATGC CAGGAAATAG AGCGCAAAAA GAACTAATAC AAGGAAAGTT |
| | 651 700 |
| NC_001463 (gag) | AAATGAAGAA GCAGAAAGGT GGAGAAGGAA TAATCCACCA CCTCCAGCAG |
| AF322109 (gag) | AAATGAGGAA GCAGAGAGAT GGAGAAGGCA GAATCCACAA CCTGCGG... |
| | 701 750 |
| NC_001463 (gag) | GAGGAGGATT AACAGTGGAT CAAATTATGG GGGTAGGACA AACAAATCAA |
| AF322109 (gag) | ...GCGGGTT AACCGTGGAT CAGATAATGG GGGTAGGACA AACGAATCAG |
| | 751 800 |
| NC_001463 (gag) | GCAGCAGCAC AAGCTAACAT GGATCAGGCA AGGCAAATAT GCCTGCAATG |
| AF322109 (gag) | GCAGCGGCAC AGGCTAATAT GGATCAAGCA AGACAAATAT GCCTACAATG |
| | 801 850 |
| NC_001463 (gag) | GGTAATAAAT GCATTAAGAG CAGTAAGACA TATGGCGCAC AGGCCAGGGA |
| AF322109 (gag) | GGTTATAACA GCAATAAGAG GAGTTAGGCA TATGGCCCAT AGACCAGGAA |
| | 851 900 |
| NC_001463 (gag) | ATCCAATGCT AGTAAAGCAA AAAACGAATG AGCCATATGA AGATTTTGCA |
| AF322109 (gag) | ATCCCATGCT GGTAAGACAA AAACCAAATG AGAACTATGA AGAGTTTGCC |
| | 901 950 |
| NC_001463 (gag) | GCAAGACTGC TAGAAGCAAT AGATGCAGAG CCAGTTACAC AGCCTATAAA |
| AF322109 (gag) | GCAAGGTTGT TAGAAGCAGT GGATGCAGAA CCCGTTACCC AACCTATAAA |
| | 951 1000 |
| NC_001463 (gag) | AGATTATCTA AAGCTAACAC TATCTTATAC AAATGCATCA GCAGATTGTC |
| AF322109 (gag) | AGAATATTTA AAGGTAACTC TGTCTTACAC AAATGCAAAT TCGGAATGTC |
| | 1001 1050 |
| NC_001463 (gag) | AGAAGCAAAT GGATAGAACA CTAGGACAAA GAGTACAACA AGCTAGTGTA |
| AF322109 (gag) | AAAAACATAT GGACAGAGTG TTGGGGCAAA GAGTACAGCA GGCCTCAATA |
| | 1051 1100 |
| NC_001463 (gag) | GAAGAAAAAA TGCAAGCATG TAGAGATGTG GGATCAGAAG GGTTCAAAAT |
| AF322109 (gag) | GAAGAAAAAA TGCAGGCATG CAGGGACATC GGGGGAACAG CATATCAGAT |
| | 1101 1150 |
| NC_001463 (gag) | GCAATTGTTA GCACAAGCAT TAAGGCCAGG AAAAGGAAAA GGGAATGGAC |
| AF322109 (gag) | GCAGTTGCTT GCACAAGCCC TCCGTGGCGG AAAAGAAGAT GGGAAAAAAT |
| | 1151 1200 |
| NC_001463 (gag) | AGCCACAAAG GTGTTACAAC TGTGGAAAAC CGGGACATCA AGCAAGGCAA |
| AF322109 (gag) | CTGTAGGGAA GTGTTATAAC TGTGGAAGGC CCGGACACAG AGCAAAAGAA |
| | 1201 1250 |
| NC_001463 (gag) | TGTAGACAAG GAATCATATG TCACAACTGT GGAAAGAGAG GACATATGCA |
| AF322109 (gag) | TGCAGACAAG GCATTATATG TCACAACTGT GGAAAAAGAG GGCATATACA |
| | 1251 1300 |
| NC_001463 (gag) | AAAAGAATGC AGAGGAAAGA GAGACATAAG GGGAAAACAG CAGGGAAACG |
| AF322109 (gag) | GAAAAACTGC A.....AACA GAAA....AG AAGAAAGGAG CAGGGAAACA |
| | 1301 1347 |
| NC_001463 (gag) | GGAGGAGGGG GATACGTGTG GTGCCGTCCG CTCCTCCTAT GGAATAA |
| AF322109 (gag) | TGAGGAGGGG GCTACGTGTG GTGCCGTCCG CACCCCCTAT GGAGTAA |

TABLE 3

PileUp
MSF: 605 Type: N Check: 9138 . . .
Name: NC_001463 (5′) (SEQ ID NO: 7) Len: 605 Check: 5398 Weight: 0
Name: AF322109 (5′) (SEQ ID NO: 8) Len: 605 Check: 3740 Weight: 0
//

| | |
|---|---|
| | 1 50 |
| NC_001463 (5′) | .......... ....GAGTTC TAGG...ACA GTCCCTCCTA GTCTCTCCTC |
| AF322109 (5′) | GTGAGTGCTC TGAGGAGCTC GAAGGAAAGA GTCC.TC..A GCCTCTCCTC |
| | 51 100 |
| NC_001463 (5′) | TCCGAGGAGG TACCGAGACC TCAAAATAAA GGAGTGATTG CCTTACTGCC |
| AF322109 (5′) | TCCGAGGAGC TTCGG....C TCATAATAAA GGAGTGCTTG CTTCA..ACA |
| | 101 150 |
| NC_001463 (5′) | GAGTGGAGAG TGATTACTGA GCGGCCGGTG TATCGGGAGT CGTCCCTTAA |
| AF322109 (5′) | GAACTGAG.. ......CTGG TCGTGGTTAT TATCGGG... .GACCGAAGT |
| | 151 200 |
| NC_001463 (5′) | TCTGTGCAAT ACCAGAGCGG CTCTCGCAGC TGGCGCCCAA CGTGGGGCCC |
| AF322109 (5′) | CCCGTGCAAC ACCGGGGCGG TTCTCGCAGC TGGCGCCCAA CGTGGGGCTC |
| | 201 250 |
| NC_001463 (5′) | GAGGAG.... .......... .......... .......... .......... |
| AF322109 (5′) | GAGTAGCTTG AGAAGCTCGA CTGAGATCTG AATCCAAGAG CGACATCAGA |
| | 251 300 |
| NC_001463 (5′) | ....AAGAAA AGAAAGC... GGCCCTGAGA ACTCGGCTTC TG..AAAAAG |
| AF322109 (5′) | CAGCAAGAAA TGAGAGTAAT GAGACCGCGA GCTCTGCTGC TGTAAAAAAG |
| | 301 350 |
| NC_001463 (5′) | AGGAAGAGGA CAAGTTGCTA TAGCAACAAG AGAGAAGAAG TAGAGCAAAG |
| AF322109 (5′) | AGGAAGTAG. CGGGTTGCCG AGGCAACTGC TCAGAAGAAC CAGGGGAAAG |
| | 351 400 |
| NC_001463 (5′) | GTCCAGTGGC T.CGGAAAAA GAGGAACTGA AACTTCGGGG ACGCCTGAAG |
| AF322109 (5′) | GGCTTCCAGC AACCTCAAAA GAGGAACCGA GACTTCGGGG ACGCCTGAA. |
| | 401 450 |
| NC_001463 (5′) | GAGTAAGGTA AGTGACTCTG CTGTACGCGG GGCGAGGCAG AGGTT.TCCT |
| AF322109 (5′) | ..GTAAGGTA AGTGACTCTG CTGTACGCGG GGCGAGGCAT AGGAGATCCT |
| | 451 500 |
| NC_001463 (5′) | TCTAAATT.G AAAGAGAAGT GTTGCTGCGA GAGGTCTTGG TGGTCGAGAA |
| AF322109 (5′) | TCTATTCTAG GAAGAGAAGC GCTGTTCTGG GAGGTCTTGG CGACCGAGAA |
| | 501 550 |
| NC_001463 (5′) | TCCTGTACAA AAAAAAGGAG GGATCTCGGT CAGGACCAGG ACCCCTGGGA |
| AF322109 (5′) | TCTTGTT... AAATAAGCCA GGATCTCGAT CAGGACCAAG ACCCCTCAGG |
| | 551 600 |
| NC_001463 (5′) | GTAATACAAC AGCAACACCG TAAGAAAATC CGCC...... .......... |
| AF322109 (5′) | AGAGGGTATA GACAGCGTGG TAAGAAA.TC CGCCGTGGTG AGTCTAGATA |
| | 601 |
| NC_001463 (5′) | ..... |
| AF322109 (5′) | GAGAC |

TABLE 4

Pileup
MSF: 3338 Type: N Check: 5428 . . .
Name: NC_001463 (pol) (SEQ ID NO: 9) Len: 3338 Check: 8114 Weight: 0
Name: AF322109 (pol) (SEQ ID NO: 10) Len: 3338 Check: 7314 Weight: 0
//

| | |
|---|---|
| | 1 50 |
| NC_001463 (pol) | .......... ........AT GTCACAACTG TGGAAAGAGA GGACATATGC |
| AF322109 (pol) | ATGCAGACAA GGCATTATAT GTCACAACTG TGGAAAAAGA GGGCATATAC |
| | 51 100 |
| NC_001463 (pol) | AAAAAGAATG CAGAGGAAAG AGAGACATAA GGGGAAAACA GCAGGGAAAC |
| AF322109 (pol) | AGAAAAACTG CA.....AAC AGAAA....A GAAGAAAGGA GCAGGGAAAC |
| | 101 150 |
| NC_001463 (pol) | GGGAGGAGGG GGATACGTGT GGTGCCGTCC GCTCCTCCTA TGGAATAACT |
| AF322109 (pol) | ATGAGGAGGG GGCTACGTGT GGTGCCGTCC GCACCCCCTA TGGAGTAACG |
| | 151 200 |
| NC_001463 (pol) | TCAGCACCAC CTATGGTTCA GGTCCGCATA GGTTCCCAGC AGAGGAACTT |
| AF322109 (pol) | CAAGCACCAC TAATAGTTAG GGTACAAATA CGGAATCAGG AGAAACAATT |
| | 201 250 |
| NC_001463 (pol) | GTTATTTGAT ACCGGGGCGG ACCGAACTAT AGTTAGATGG CATGAGGGCT |
| AF322109 (pol) | ATTATTTGAC ACAGGGGCAG ATAAAACGAT AGTAAGAATG CATGATGGAA |
| | 251 300 |
| NC_001463 (pol) | CGGGAAACCC AGCCGGAAGG ATAAAACTGC AAGGAATAGG AGGAATAGTA |
| AF322109 (pol) | CAGGGATTCC AAACGGAAGA ATAAAATTAC AAGGGATAGG AGGAATAGTA |
| | 301 350 |
| NC_001463 (pol) | GAAGGAGAAA AATGGAATAA TGTAGAATTA GAATATAAAG GAGAAACAAG |
| AF322109 (pol) | GAAGGAGAAA AATGGAATAA AGTACCCATG ACATATAAGG GAGAAACATC |
| | 351 400 |
| NC_001463 (pol) | AAAGGGAACA ATAGTAGTGT TACCACAAAG TCCAGTAGAA GTATTAGGAC |
| AF322109 (pol) | CTGCCCAAGC TTGGTTGTGC TAAGAGATAG CCCAGTAGAA GTATTGGGAA |
| | 401 450 |
| NC_001463 (pol) | GAGATAACAT GGCCCGATTT GGAATAAAGA TAATAATGGC AAATTTAGAG |
| AF322109 (pol) | GAGATAACAT GGAAGCATTC GGCGTAACCC TAATAATGOC AAATTTAGAA |
| | 451 500 |
| NC_001463 (pol) | GAAAAAAGAA TCCCAATTAC AAAAGTAAAA TTGAAAGAGG GATGTACGGG |
| AF322109 (pol) | GATAAGAAAA TTCCCACAAT ACCAGTAGAA TTGAAAGAAG GATGTAAAGG |
| | 501 550 |
| NC_001463 (pol) | TCCACATGTC CCACAATGGC CATTAACAGA AGAGAAATTA AAAGGTCTAA |
| AF322109 (pol) | GCCACATGTG CCCCAGTGGC CATTAACAGC AGAGAAATTA CAAGGACTAA |
| | 551 600 |
| NC_001463 (pol) | CAGAAATCAT AGATAAATTA GTGGAAGAAG GAAAACTAGG AAAGGCACCC |
| AF322109 (pol) | CAGGAATAGT AGAAAAATTA CTACAGGAAG GAAAATTGGC AGAGGCCCCA |
| | 601 650 |
| NC_001463 (pol) | CCACATTGGA CATGTAATAC TCCAATCTTT TGCATAAAAA AGAAATCAGG |
| AF322109 (pol) | GAGGGATGGA CGTGGAACAC GCCCATCTTC TGCATAAAAA AGAAGTCAGG |
| | 651 700 |
| NC_001463 (pol) | GAAGTGGAGA ATGTTAATAG ATTTCAGAGA ATTGAACAAA CAGACAGAAG |
| AF322109 (pol) | AAAATGGAGA ATGTTAATAG ATTTTAGGGA ATTAAATAAG CAAACAGCAG |
| | 701 750 |
| NC_001463 (pol) | ATTTAACAGA AGCGCAGTTA GGACTCCCGC ATCCGGGAGG ACTACAAAAG |
| AF322109 (pol) | ATTTAGCAGA AGCGCAGCTA GGACTGCCAC ACCCAGGAGG GTTGCAAAGG |
| | 751 800 |
| NC_001463 (pol) | AAAAAACATG TTACAATATT GGACATAGGA GATGCATATT TTACTATACC |
| AF322109 (pol) | AAAAAGAATG TAACAATTCT GGACATAGGA GATGCATATT TCACAATTCC |
| | 801 850 |
| NC_001463 (pol) | CCTATATGAA CCATATCGAG AGTACACATG TTTTACTCTA TTAAGTCCTA |
| AF322109 (pol) | CTTATACGAG CCCTATCAGA AATATACATG CTTCACACTC CTAAGTCCTA |
| | 851 900 |
| NC_001463 (pol) | ATAATCTAGG ACCATGTAAA AGATACTATT GGAAAGTGCT GCCACAAGGT |
| AF322109 (pol) | ACAATTTGGG ACCATGTAAA AGGTATTATT GGAAAGTATT ACCCCAGGGA |
| | 901 950 |
| NC_001463 (pol) | TGGAAATTGA GTCCATCTGT ATATCAATTT ACTATGCAGG AGATCTTAGA |
| AF322109 (pol) | TGGAAATTGA GCCCAGCTGT ATATCAATTC ACCATGCAAA GGTTGTTAAA |
| | 951 1000 |
| NC_001463 (pol) | GGATTGGATA CAGCAGCATC CAGAAATTCA ATTTGGCATA TATATGGATG |
| AF322109 (pol) | AGGATGGATA CAACAGCATA AAAACATACA ATTTGGAATA TATATGGATG |
| | 1001 1050 |
| NC_001463 (pol) | ATATTTACAT AGGAAGTGAT TTAGAAATTA AAAAGCATAG AGAAATAGTG |
| AF322109 (pol) | ATATCTATAT TGGAAGTGAT CTAACGATAG CCCAACATAG GAAGATAATA |
| | 1051 1100 |
| NC_001463 (pol) | AAAGATTTAG CCAATTATAT TGCCCAATAT GGATTCACTC TGCCAGAAGA |
| AF322109 (pol) | GAAGAATTAG CCTCATTTAT AGAACAATTT GGGTTTACAT TACCAGAAGA |
| | 1101 1150 |
| NC_001463 (pol) | GAAGAGACAA AAGGGATATC CAGCAAAATG GCTAGGATTT GAACTACACC |
| AF322109 (pol) | TAACAGACAA GAGGGCTATC CAGCAAAATG GCTAGGATTC GAGCTACATC |
| | 1151 1200 |
| NC_001463 (pol) | CGCAGACCTG GAAATTTCAG AAGCATACAT TACCTGAATT AACAAAGGGA |
| AF322109 (pol) | CAGAAAAATG GAAATATCAA AAGCATAAAT TGCCGGAATT ACAAGAGGGG |
| | 1201 1250 |
| NC_001463 (pol) | ACAATAACAT TAAATAAATT ACAGAAATTA GTAGGAGAAT TAGTATGGAG |
| AF322109 (pol) | GTAATAACCC TGAACAAATT ACAGAAGATA GTAGGGGAAT TAGTGTGGAG |
| | 1251 1300 |
| NC_001463 (pol) | ACAATCCATA ATTGGGAAAA GCATTCCTAA CATTCTGAAA TTAATGGAAG |
| AF322109 (pol) | ACAATCCTTG ATAGGAAAGA GCATCCCCAA TATCATAAAA TTAATGGAAG |
| | 1301 1350 |
| NC_001463 (pol) | GAGATAGAGA ATTACAAAGT GAAAGAAAAA TTGAAGAAGT ACATGTGAAA |
| AF322109 (pol) | GAGATCGCGC ATTACAAAGT GAAAGGAAAA TAGAAAGAAT ACATGTACAA |
| | 1351 1400 |
| NC_001463 (pol) | GAATGGGAAG CATGTAGGAA AAAATTAGAA GAAATGGAAG GAAATTATTA |
| AF322109 (pol) | GAATGGGAAG CATGTCAAAA GAAATTAGAT GAAATGGTAG GAAATTATTA |
| | 1401 1450 |
| NC_001463 (pol) | TAATAAAGAC AAAGATGTCT ATGGACAATT GGCTTGGGGA GACAAAGCTA |
| AF322109 (pol) | CAGAGAAGAA GAAGATATCT ATGGACAAAT AACTTGGGGG GATAAGGCAA |
| | 1451 1500 |
| NC_001463 (pol) | TAGAATATAT AGTGTATCAG GAGAAAGGGA AACCATTATG GGTAAATGTG |
| AF322109 (pol) | TAAAATACAT AGTATTCCAA AGGAAAGGGG AACCCCTATG GGTAAATGTA |
| | 1501 1550 |
| NC_001463 (pol) | GTTCACAATA TAAAGAACCT AAGCATCCCG CAACAGGTTA TTAAAGCAGC |
| AF322109 (pol) | GTACATGACA TAAAAAATTT GAGTCTCCCA CAGCAAGTGA TAAAAGCAGC |
| | 1551 1600 |
| NC_001463 (pol) | GCAAAAATTA ACCCAAGAAG TCATCATTAG GACAGGAAAA ATACCATGGA |
| AF322109 (pol) | ACAGAAATTA ACCCAGGAAG TAATCATAAG AACAGGAAAA ATCCCATGGC |
| | 1601 1650 |
| NC_001463 (pol) | TATTGTTGCC AGGGAAAGAA GAAGATTGGA GACTAGAATT GCAATTAGGG |
| AF322109 (pol) | TGCTACTACC AGGAAGAGAA GAAGACTGGA GATTAGAACT GCAGGTAGGG |
| | 1651 1700 |
| NC_001463 (pol) | AACATCACAT GGATGCCAAA ATTTTGGTCC TGTTATCGAG GA.CATACAA |
| AF322109 (pol) | AACATCACGT GGATGCCATC ATTTTGGTCA TGTTATCGAG GAGCACCCAA |
| | 1701 1750 |
| NC_001463 (pol) | GATGGAGAAA AAGAAATATA ATAGAAGAAG TAGTAGAAGG GCCTACATAT |
| AF322109 (pol) | G.TGGAAAAG AAGGAACATA GTGGCAGCAG TGGTAGATGG ACCCACATAT |
| | 1751 1800 |
| NC_001463 (pol) | TATACAGATG GAGGAAAAAA GAATAAAGTA GGAAGTCTAG GGTTCATAGT |
| AF322109 (pol) | TATACAGATG GGGGAAAGAA AAACGCACAG GGAAGCTTTG GCTTCATCTC |
| | 1801 1850 |
| NC_001463 (pol) | ATCAACAGGG GAAAAATTTA GAAAGCATGA AGAGGGCACA AACCAGCAAC |
| AF322109 (pol) | CCCAACAGGA GAAAAGTTCA GAAGGCATGA AGATGGAACT AATCAGGTAT |
| | 1851 1900 |
| NC_001463 (pol) | TAGAATTAAG AGCCATAGAG GAAGCTCTAA AACAAGGGCC TCAAACAATG |
| AF322109 (pol) | TAGAATTAAG GGCAATAGAA GATCCATGTA AACAAGGACC TGAAAGCATG |
| | 1901 1950 |
| NC_001463 (pol) | AATTTAGTAA CAGATAGTAG ATATGCATTT GAATTTTTAT TAAGAAATTG |
| AF322109 (pol) | AACATTGTAA CTGACAGCAG GTATGCTTAT GAATTCATGC TCCGAAACTG |
| | 1951 2000 |
| NC_001463 (pol) | GGATGAAGAA GTAATAAAGA ATCCAATTCA AGCAAGAATT ATGGAAATTG |
| AF322109 (pol) | GGATGAACAG GTCATAAGAA ACCCCATTCA GGCAAGAATC ATGGCAGAAG |
| | 2001 2050 |
| NC_001463 (pol) | CCCACAAGAA AGATAGGATA GGAGTGCATT GGGTGCCAGG ACATAAAGGG |
| AF322109 (pol) | TGCACAAGAA AAAGCAGGTA GGAATACACT GGGTGCCAGG GCATAAAGGA |
| | 2051 2100 |
| NC_001463 (pol) | ATTCCCCAAA ATGAAGAAAT AGACAAATAT ATTTCGGAAA TATTTCTTGC |
| AF322109 (pol) | ATACCTCAGA ATGAAGAGAT AGACCAGTAC ATATCAGAAG TATTCTTAGC |
| | 2101 2150 |
| NC_001463 (pol) | AAAAGAAGGA GAAGGAATTC TCCCAAAAAG AGAAGAGGAT GCAGGGTATG |
| AF322109 (pol) | ACGAGAAGGA ACAGGGATAT GTGAAAAAAG GAAGGAAGAT GCTGGATATG |
| | 2151 2200 |
| NC_001463 (pol) | ATTTAATATG CCCAGAAGAG GTTACCATAG AGCCAGGACA AGTGAAATGC |
| AF322109 (pol) | ATTTATTATG CCCGCATGAG GTAATACTTA AACCCCAAGA AGTAAAACGG |
| | 2201 2250 |
| NC_001463 (pol) | ATCCCCATAG AGCTAAGATT AAATTTAAAG AAATCACAAT GGGCTATGAT |
| AF322109 (pol) | ATCCCAATAG ACCTAAAATT AAAATTGAAA GAAAAGCAAT GGGCCATGAT |
| | 2251 2300 |
| NC_001463 (pol) | TGCTACAAAA AGCAGCATGG CTGCCAAAGG AGTGTTCACA CAAGGAGGAA |
| AF322109 (pol) | AAGTGGGAAA AGTAGCGTTG CAGCAAAAGG AATATTTGTA CAAGGAGGCA |
| | 2301 2350 |
| NC_001463 (pol) | TCATAGACTC AGGATATCAG GGACAAATAC AGGTAATAAT GTATAATAGC |
| AF322109 (pol) | TAATAGATTC AGGGTATCAG GGACAAGTAC AAGTCATCCT ATATAATAGT |
| | 2351 2400 |
| NC_001463 (pol) | AATAAAATAG CAGTAGTCAT ACCCCAAGGG AGAAAATTTG CACAATTAAT |
| AF322109 (pol) | AATAAGATAG AGGTCAAAAT ACCACAAGGC AGGAAATTTG CCCAATTAAT |
| | 2401 2450 |
| NC_001463 (pol) | ATTAATGGAT AAAAAGCATG GAAAATTGGA ACCCTGGGGG GAAAGCAGAA |
| AF322109 (pol) | ATTAATGAAC TTACAACATG AAGAATTAGA AGAATGGGGA AAGGAAAGAA |
| | 2451 2500 |
| NC_001463 (pol) | AAACAGAAAG GGGAGAAAAA GGATTTGGGT CTACAGGAAT GTATTGGATA |
| AF322109 (pol) | AAACAGAAAG AGGAACAAAA GGATTTGGGT CTACAGGAGC ATTTTGGATA |
| | 2501 2550 |
| NC_001463 (pol) | GAAAATATTC CTCTGGCAGA GGAAGACCAC ACAAAATGGC ATCAAGATGC |
| AF322109 (pol) | GAGAATATTC CCCAAGCAGA GGAAGAACAT TACAAATGGC ATCAAGATGC |
| | 2551 2600 |
| NC_001463 (pol) | CCGATCATTG CATCTAGAAT TTGAAATTCC AAGAACAGCA GCAGAAGACA |
| AF322109 (pol) | TAGATCTCTG CAGCTAGAAT TCAAGATACC TAGAGCAGCA GCAGAAGACA |
| | 2601 2650 |
| NC_001463 (pol) | TAGTAAATCA ATGTGAAATA TGCAAAGAAG CGAGGACACC TGCAGTAATT |
| AF322109 (pol) | TTATACAGCA CTGTGAGGTA TGTCAAGAAG GCAAACCCGC AGCGATCACG |
| | 2651 2700 |
| NC_001463 (pol) | AGAGGCGGAA ACAAAAGGGG GGTAAATCAT TGGCAAGTGG ATTATACCCA |
| AF322109 (pol) | AGAGGGGGAA ATAAAAGAGG AATAGATCAT TGGCAGGTAG ACTATACACA |
| | 2701 2750 |
| NC_001463 (pol) | TTATGAAAAT ATCATACTAT TAGTATGGGT AGAAACAAAT TCAGGACTAA |
| AF322109 (pol) | TTACAAAGAA CACATAATAT TAGTATGGGT AGAGACTAAT TCAGGATTAA |
| | 2751 2800 |
| NC_001463 (pol) | TATATGCAGA AAAAGTAAAA GGAGAATCAG GGCAAGAATT CAGAATAAAA |
| AF322109 (pol) | TATTTGCAGA GAAAGTAAAA GGAGAATCAG GACAAGAATT TAGGATGCAG |
| | 2801 2850 |
| NC_001463 (pol) | GTGATGCATT GGTATGCATT ATTTGGTCCA GAGTCATTGC AGTCAGACAA |
| AF322109 (pol) | ACATTGAAAT GGTATGCTTT GTTTCAACCA AAATCAGTGC AATCAGATAA |
| | 2851 2900 |
| NC_001463 (pol) | TGGACCTGCA TTTGCAGCAG AGCCCACACA GCTGTTAATG CAATACCTAG |
| AF322109 (pol) | TGGGACAGCC TTCACAGCTG AGGCTACGCA GCATCTAATG AAGTATTTAG |
| | 2901 2950 |
| NC_001463 (pol) | GAGTAAAACA CACAACAGGC ATACCTTGGA ATCCACAGTC TCAGGCTATA |
| AF322109 (pol) | GGATTCAGCA CACTACGGGT ATTCCGTGGA ACCCCCAGTC ACAAAGTTTA |
| | 2951 3000 |
| NC_001463 (pol) | GTAGAAAGGG CACATCAACT ATTGAAAAGC ACTTTAAAGA AGTTCCAGCC |
| AF322109 (pol) | GTAGAAAGAG CTCATCAAAC ATTAAAACAC ATGTTAGAAA AATTAGAACC |
| | 3001 3050 |
| NC_001463 (pol) | ACAATTTGTC GCTGTAGAAT CAGCCATAGC AGCAGCCCTA GTCGCCATAA |
| AF322109 (pol) | ACAATTTGTG GCCCTACAGT CTGCCATCGC AGCCACTCTA GTTGCGCTCA |
| | 3051 3100 |
| NC_001463 (pol) | ATATAAAAAG AAAGGGTGGG CTGGGGACAA GCCCTATGGA TATTTTTATA |
| AF322109 (pol) | ATATAAAAAG AAAGGGTGGA CTAGGGGCAA GCCCTATGGA TATTTACATA |
| | 3101 3150 |
| NC_001463 (pol) | TATAATAAAG AACAGAAAAG AATAAATAAT AAATATAATA AAAATTCTCA |
| AF322109 (pol) | TATAATAAGG AGCAACAAAG ACAACAACAT AATAGTAATA AATTAATTCA |
| | 3151 3200 |
| NC_001463 (pol) | AAAAATTCAA TTCTGTTATT ACAGAATAAG GAAAAGAGGA CATC.AGGAG |
| AF322109 (pol) | GAAAA...AA TTTTGTTATT ACAGGATCAG AAAAAGAGGC CATCCAGGAG |
| | 3201 3250 |
| NC_001463 (pol) | AGTGGAAAGG ACCAACCCAG GTACTGTGGA AAGGGGAAGG AGCCAATTGT |
| AF322109 (pol) | AGTGGAACGG CCCAACTGAG GTACTGTGGG AAGGGGAAGG AGCCA.TAGT |
| | 3251 3300 |
| NC_001463 (pol) | GGTAAAGGAT ATAGAAAGTG AAAAGTATTT AGTAATACCT TACAAAGATG |
| AF322109 (pol) | AGTTAAAGAC AAAGAAAGTG ATAGATATCT AGTCATCCCA TATAAAGATG |
| 3301 3338 | |
| NC_001463 (pol) | CAAAATTCAT CCCGCCACCA ACAAAAGAAA AGGAATAA |
| AF322109 (pol) | CAAAATTTAT TCCGCCACCG TCGGAACAGA AGGGATAG |

TABLE 5

Pileup
MSF: 408 Type: N Check: 517 . . .
Name: NC_001463 (rev) (SEQ ID NO: 11) Len: 408 Check: 7287 Weight: 0
Name: AF322109 (rev) (SEQ ID NO: 12) Len: 408 Check: 3230 Weight: 0
//

| | |
|---|---|
| | 1 50 |
| NC_001463 (rev) | ATGGATGCTG GGGCCAGATA CATGCGCTTA ACTGGGAAGG AAAACTGGGT |
| AF322109 (rev) | .......... .......... .......... .......... .......... |
| | 51 100 |
| NC_001463 (rev) | TGAAGTAACC ATGGACGGAG AGAAGGAAAG GAAAAGAGAA GGTTTCACTG |
| AF322109 (rev) | .......... ATGG.CGGAG ATAAGAAAAG ..A.AGCAAA GGAGCCACTA |
| | 101 150 |
| NC_001463 (rev) | CGGGACAGCA AGATATACAG AACTCTAAGT ACCCCGACAT ACCAACGGGT |
| AF322109 (rev) | ATCCAGGACC AGGTATCAAC AAGTCAACTT GGTGATGGAG ACC..CGGGT |
| | 151 200 |
| NC_001463(rev) | CACAGTCATC ATGGAAACAA GAGCAGACGT CGCAGGAGAA AATCAGGATT |
| AF322109 (rev) | .......... ..GCAACTAG AAGAAGAAGA AGAAGAAGAC GGAAGGGATG |
| | 201 250 |
| NC_001463 (rev) | TTGGCGATGG CTTAGAGGAA TCAGACAACA GCGAAACAAG CGAAAGAGTG |
| AF322109 (rev) | GTGGAGATGG CTCAGAGAGA TG......CA GCGATCCCGA CAACAAAGGA |
| | 251 300 |
| NC_001463 (rev) | ACAGTACAGA AAGCTTGGAG CCGTGCCTGG GAGCTTTGGC AGAACTCACC |
| AF322109 (rev) | A...TTATGA ACGCCTGGAG GAGAGCTTGG GTGACTTGGA GAAACTCACC |
| | 301 350 |
| NC_001463 (rev) | CTGGAAGGAG CCATGGAAAA GGGGCCTGCT GAGGCTGCTC GTCCTTCCGC |
| AF322109 (rev) | TTGGCAGAAC ACATGGAAGA ATGTGGTGGT GGCGCCGTTG GTGATTCCGC |
| | 351 400 |
| NC_001463 (rev) | TGACGATGGG AATCTGGATA AATGGATGGC TTGGAGAACA CCACAAAAAT |
| AF322109 (rev) | TGACAATCAG AATTTGGCTC CTTGGAGAGA ATGGAGAGAA CCCCTAAAAG |
| | 401 |
| NC_001463 (rev) | AA...... |
| AF322109 (rev) | AAAAATAA |

TABLE 6

Pileup
MSF: 691 Type: N Check: 6528 . . .
Name: NC_001463 (vif) (SEQ ID NO: 13) Len: 691 Check: 5882 Weight: 0
Name: AF322109 (vif) (SEQ ID NO: 14) Len: 691 Check: 646 Weight: 0
//

| | |
|---|---|
| | 1 50 |
| NC_001463 (vif) | ATGCAAAATT CATCCCGCCA CCAACAAAAG AAAAGGAATA AAAAACCTGG |
| AF322109 (vif) | ATGCAAAATT TATTCCGCCA CCGTCGGAAC AGAAGGGATA GAAGAATAGG |
| | 51 100 |
| NC_001463 (vif) | ACCAGAATTA CCCTTAGCAC TATGGATACA TATAGCAGAA AGCATTAATG |
| AF322109 (vif) | TCCAGAATTG CCTTTATCTT TATGGACTTA TACAGCATAC AGCATAAATA |
| | 101 150 |
| NC_001463 (vif) | GGGATAGCTC ATGGTACATA ACAATGAGAC TGCAACAGAT GATGTGGGGA |
| AF322109 (vif) | AAGATCCCGC ATGGTATACA ACCCTAAGAC TGCAGCAAAT GATGTGGCAT |
| | 151 200 |
| NC_001463 (vif) | AAAAGAGGAA ATAAGTTACA ATATAAGAAT GAAGACAGGG AATATGAAAA |
| AF322109 (vif) | AGGAGGGGAA ATAAATTGAC ATATGTCAGG GAAAATGCAC AGTACGAGGA |
| | 201 250 |
| NC_001463 (vif) | TTGGGAAATT ACATCATGGG GATGGAAAAT GCACCTAAGG AGAGTCAAAC |
| AF322109 (vif) | GTGGGAAATG ACCTCGTATG AGTGGAGGAT AAGAATGAGA AGGGACAAAA |
| | 251 300 |
| NC_001463 (vif) | AATGGATACA AGACAACAGG AGAGGAAGC. CCATGGCAGT ACAAAGTAGG |
| AF322109 (vif) | CAAAAAGTCA TC.CAAGAGG GCATACTTCG CCATGGCAAT ATCGGAGACA |
| | 301 350 |
| NC_001463 (vif) | AGGAACATGG AAAAGTATAG GAGTGTGGTT CCTGCAAGCA GGAGATTACA |
| AF322109 (vif) | GGATGGATGG AAGGATGTGG GAACGTGGTT CCTACAGCCA GGCGACTATA |
| | 351 400 |
| NC_001463 (vif) | GAAAGGTAGA CAGGCACTTC TGGTGGGCAT GGAGGATACT GATATGTTCC |
| AF322109 (vif) | GAAAGGCGGA TCAGCAGTTC TGGTTCGCTT GGAGAATAGT GTCGTGTTCA |
| | 401 450 |
| NC_001463 (vif) | TGCAGGAAAG AAAAGTTTGA TATAAGAGAA TTTATGAGAG GAAGACATAG |
| AF322109 (vif) | TGTAAAAAGG AAGGATTTAA CATAAGAGAA TTTATGCTAG GTACCCATAG |
| | 451 500 |
| NC_001463 (vif) | ATGGGATTTG TGCAAATCCT GTGCTCAAGG AGAAGTAGTA AAGCATACTA |
| AF322109 (vif) | ATGGGATTTG TGTAAGTCGT GTTGCCAGGG TGAAGTAGTA AAGAGAACAC |
| | 501 550 |
| NC_001463 (vif) | GAACAAAAAG TCTGGAAAGA CTAGTACTGC TACAGATGGT AGAACAGCAT |
| AF322109 (vif) | AACCCTACAC CTTGCAAAGG CTCACGTGGC TTAAATTAAC AGAAGACCAT |
| | 551 600 |
| NC_001463 (vif) | GTGTTTCAAG TATTGCCATT GTGGAGAGCC AGGAGAAGTA GTACAACAGA |
| AF322109 (vif) | GTATTTCAAG TAATGCCCTT GTGGAGAGCT CGCAAAGGGA TTACCATAGA |
| | 601 650 |
| NC_001463 (vif) | TTTCCCATGG TGCAGGGACA CAACGGGATA CACGCATGCG TGGTCTGTCC |
| AF322109 (vif) | CTTTCCCTGG TGCAGGGACA CAAAAGGATT CCTGGAGCCG TGGACAACGC |
| | 651 691 |
| NC_001463 (vif) | AGGAGTGCTG GTTGATGGAA TATCTCTTAG AGGATGAGTG A |
| AF322109 (vif) | AAGAGTGTTG GCAAATAGAG TATCCCTTGG AGGATGAGTG A |

TABLE 7

Pileup
MSF: 736 Type: N Check: 513 . . .
Name: NC_001463 (gag720bp) (SEQ ID NO: 15) Len: 736 Check: 4701 Weight: 0
Name: >AF015181 (SEQ ID NO: 16) Len: 736 Check: 5812 Weight: 0
//

| | |
|---|---|
| | 1 50 |
| NC_001463 (gag720bp) | .ATGGTGAGT CTAGATAGAG ACATGGCGAG GCAAGTCTCC GGGGGGAAAA |
| >AF015181 | GCTGTAGACT CTGTAATGTT CCAACAA.AT GCAAA....C AGTAGCAATG |
| | 51 100 |
| NC_001463 (gag720bp) | GAGATTATCC TGAGCTCGAA AAATGTATCA AGCATGCATG CAAGATAAAA |
| >AF015181 | CAGCATGGCC TCGTGTCCGA GGATTTTGAA AGACAGTTAG CAT.ATTATG |
| | 101 150 |
| NC_001463 (gag720bp) | GTTCGACTCA CAGGGG..AG CACTTGACAG AAGGAAATTG TTTATGGTGC |
| >AF015181 | CTACTACCTG GACAAGTAAA GACATACTAG AAGTA..TTG GCCATGATGC |
| | 151 200 |
| NC_001463 (gag720bp) | CTTAAAACA. ...TTAGATT ..ACATGTTT GAGGACCAT. .AAAGAGGAA |
| >AF015181 | CTGGGAATAG GGCTCAGAAA GAACTTATTC AAGGGAAATT GAATGAAGAA |
| | 201 250 |
| NC_001463 (gag720bp) | CCTTGGACAA AAGTAAAATT TAGGACAATA TGGCAGAAGG .TGAAGAATC |
| >AF015181 | GCA..GACAG GTGGAGAAG. ..GAACAATC CACCAGGAGG ATTAACAGTG |
| 251 300 | |
| NC_001463 (gag720bp) | TAACTCCTGA GGAGAGTAAC AAAAAAGACT TTATGTCTTT GCAGGCCACA |
| >AF015181 | GATCAAATTA TGGGGGTAGG ACAAACAAAT ..GCA..... GCAGCACAAG |
| | 301 350 |
| NC_001463 (gag720bp) | TTAGCGGGTC TAATGTGTTG CCAAATGGGG ATGAGACCTG AGACATTGCA |
| >AP015181 | CTAACATGGA TCAGGCAA.G ACAAATATGC CT...ACAAT GGGTAATAAA |
| | 351 400 |
| NC_001463 (gag720bp) | AGATGCAATG GCTACAGTAA TCATGAAAGA TGGGTTACTG GAACAAGAGG |
| >AF015181 | CGCCTTAAGA GCAGTAAGGC ATATGGCTCA TAGGCCAGGG AATCCAATGC |
| | 401 450 |
| NC_001463 (gag720bp) | AAAAGAAGGA AGACAAAAGA GAAAAGGAAG AGAGTGTCTT CCCAATAGTA |
| >AF015181 | TAGTAAAGCA A...AAAACA AATGAGCCAT ATGAAGAATT TGCAGCAAGA |
| | 451 500 |
| NC_001463 (gag720bp) | GTGCAAGCAG CA..GGAGGG AGAAGCTGGA AAGCAGTAGA TTCTGTAATG |
| >AF015181 | CTGCTAGAAG CAATAGATGC AGAAGCGGTT ACACAGCCCA TAAAAGAGTA |
| | 501 550 |
| NC_001463 (gag720bp) | T.TCCAGC.A ACTGCAAACA GTAGCAATGC AGCATGGCCT CGTGTCTGAG |
| >AF015181 | TCTAAAGCTA ACATTATCCT ATACAAATGC AGC.....CT CA........ |
| | 551 600 |
| NC_001463 (gag720bp) | GACTTTGAAA GGCAGTTGGC ATATTATGCT ACTACCTGGA CAAGTAAAGA |
| >AF015181 | GATTGTCAAA AGCAAATGG. AGAGAGTGCT AGGACAAAGA ...GTACA.A |
| | 601 650 |
| NC_001463 (gag720bp) | CATACTAGAA GTATTGGCCA TGATGCCTGG AAATAGAGCT CAAAAGGAGT |
| >AF015181 | CAGGCTAGT. GTAGAAAAAA AAATGCAAGC ATGT...... .......... |
| | 651 700 |
| NC_001463 (gag720bp) | TAATTCAAGG GAAATTAAAT GAAGAAGCAG AAAGGTGGAG AAGGAATAAT |
| >AF015181 | .......... .......... .......... .......... .......... |
| | 701 736 |
| NC_001463 (gag720bp) | CCACCACCTC CAGCAGGAGG AGGATTAACA GTGGAT |
| >AF015181 | .......... .......... .......... ...... |

Pileup
MSF: 1347 Type: N Check: 939 . . .
Name: NC_001463 (gag) (SEQ ID NO: 17) Len: 1347 Check: 6959 Weight: 0
Name: >AF015181 (SEQ ID NO: 18) Len: 1347 Check: 3980 Weight: 0
//

| | |
|---|---|
| | 1 50 |
| NC_001463 (gag) | ATGGTGAGTC TAGATAGAGA CATGGCGAGG CAAGTCTCCG GGGGGAAAAG |
| >AF015181 | .......... .......... .......... .......... .......... |
| | 51 100 |
| NC_001463 (gag) | AGATTATCCT GAGCTCGAAA AATGTATCAA GCATGCATGC AAGATAAAAG |
| >AF015181 | .......... .......... .......... .......... .......... |
| | 101 150 |
| NC_001463 (gag) | TTCGACTCAG AGGGGAGCAC TTGACAGAAG GAAATTGTTT ATGGTGCCTT |
| >AF015181 | .......... .......... .......... .......... .......... |
| | 151 200 |
| NC_001463 (gag) | AAAACATTAG ATTACATGTT TGAGGACCAT AAAGAGGAAC CTTGGACAAA |
| >AF015181 | .......... .......... .......... .......... .......... |
| | 201 250 |
| NC_001463 (gag) | AGTAAAATTT AGGACAATAT GGCAGAAGGT GAAGAATCTA ACTCCTGAGG |
| >AF015181 | .......... .......... .......... .......... .......... |
| | 251 300 |
| NC_001463 (gag) | AGAGTAACAA AAAAGACTTT ATGTCTTTGC AGGCCACATT AGCGGGTCTA |
| >AF015181 | .......... .......... .......... .......... .......... |
| | 301 350 |
| NC_001463 (gag) | ATGTGTTGCC AAATGGGGAT GAGACCTGAG ACATTGCAAG ATGCAATGGC |
| >AF015181 | .......... .......... .......... .......... .......... |
| | 351 400 |
| NC_001463 (gag) | TACAGTAATC ATGAAAGATG GGTTACTGGA ACAAGAGGAA AAGAAGGAAG |
| >AF015181 | .......... .......... .......... .......... .......... |
| | 401 450 |
| NC_001463(gag) | ACAAAAGAGA AAAGGAAGAG AGTGTCTTCC CAATAGTAGT GCAAGCAGCA |
| >AF015181 | .......... .......... .......... .......... .......... |
| | 451 500 |
| NC_001463 (gag) | GGAGGGAGAA GCTGGAAAGC AGTAGATTCT GTAATGTTCC AGCAACTGCA |
| >AF015181 | .......... ........GC TGTAGACTCT GTAATGTTCC AACAAATGCA |
| | 501 550 |
| NC_001463 (gag) | AACAGTAGCA ATGCAGCATG GCCTCGTGTC TGAGGACTTT GAAAGGCAGT |
| >AF015181 | AACAGTAGCA ATGCAGCATG GCCTCGTGTC CGAGGATTTT GAAAGACAGT |
| | 551 600 |
| NC_001463 (gag) | TGGCATATTA TGCTACTACC TGGACAAGTA AAGACATACT AGAAGTATTG |
| >AF015181 | TAGCATATTA TGCTACTACC TGGACAAGTA AAGACATACT AGAAGTATTG |
| | 601 650 |
| NC_001463 (gag) | GCCATGATGC CTGGAAATAG AGCTCAAAAG GAGTTAATTC AAGGGAAATT |
| >AF015181 | GCCATGATGC CTGGGAATAG GGCTCAGAAA GAACTTATTC AAGGGAAATT |
| | 651 700 |
| NC_001463 (gag) | AAATGAAGAA GCAGAAAGGT GGAGAAGGAA TAATCCACCA CCTCCAGCAG |
| >AF015181 | GAATGAAGAA GCAGACAGGT GGAGAAGGAA CAATCCACCA .......... |
| | 701 750 |
| NC_001463 (gag) | GAGGAGGATT AACAGTGGAT CAAATTATGG GGGTAGGACA AACAAATCAA |
| >AF015181 | ..GGAGGATT AACAGTGGAT CAAATTATGG GGGTAGGACA AACAAATCAA |
| | 751 800 |
| NC_001463 (gag) | GCAGCAGCAC AAGCTAACAT GGATCAGGCA AGGCAAATAT GCCTGCAATG |
| >AF015181 | GCAGCAGCAC AAGCTAACAT GGATCAGGCA AGACAAATAT GCCTACAATG |
| | 801 850 |
| NC_001463 (gag) | GGTAATAAAT GCATTAAGAG CAGTAAGACA TATGGCGCAC AGGCCAGGGA |
| >AF015181 | GGTAATAAAC GCCTTAAGAG CAGTAAGGCA TATGGCTCAT AGGCCAGGGA |
| | 851 900 |
| NC_001463 (gag) | ATCCAATGCT AGTAAAGCAA AAAACGAATG AGCCATATGA AGATTTTGCA |
| >AF015181 | ATCCAATGCT AGTAAAGCAA AAAACAAATG AGCCATATGA AGAATTTGCA |
| | 901 950 |
| NC_001463 (gag) | GCAAGACTGC TAGAAGCAAT AGATGCAGAG CCAGTTACAC AGCCTATAAA |
| >AF015181 | GCAAGACTGC TAGAAGCAAT AGATGCAGAA GCGGTTACAC AGCCCATAAA |
| | 951 1000 |
| NC_001463 (gag) | AGATTATCTA AAGCTAACAC TATCTTATAC AAATGCATCA GCAGATTGTC |
| >AF015181 | AGAGTATCTA AAGCTAACAT TATCCTATAC AAATGCAGCC TCAGATTGTC |
| | 1001 1050 |
| NC_001463 (gag) | AGAAGCAAAT GGATAGAACA CTAGGACAAA GAGTACAACA AGCTAGTGTA |
| >AF015181 | AAAAGCAAAT GGAGAGAGTG CTAGGACAAA GAGTACAACA GGCTAGTGTA |
| | 1051 1100 |
| NC_001463 (gag) | GAAGAAAAAA TGCAAGCATG TAGAGATGTG GGATCAGAAG GGTTCAAAAT |
| >AF015181 | GAAAAAAAAA TGCAAGCATG T......... .......... .......... |
| | 1101 1150 |
| NC_001463 (gag) | GCAATTGTTA GCACAAGCAT TAAGGCCAGG AAAAGGAAAA GGGAATGGAC |
| >AF015181 | .......... .......... .......... .......... .......... |
| | 1151 1200 |
| NC_001463 (gag) | AGCCACAAAG GTGTTACAAC TGTGGAAAAC CGGGACATCA AGCAAGGCAA |
| >AF015181 | .......... .......... .......... .......... .......... |
| | 1201 1250 |
| NC_001463 (gag) | TGTAGACAAG GAATCATATG TCACAACTGT GGAAAGAGAG GACATATGCA |
| >AF015181 | .......... .......... .......... .......... .......... |
| | 1251 1300 |
| NC_001463 (gag) | AAAAGAATGC AGAGGAAAGA GAGACATAAG GGGAAAACAG CAGGGAAACG |
| >AF015181 | .......... .......... .......... .......... .......... |
| | 1301 1347 |
| NC_001463 (gag) | GGAGGAGGGG GATACGTGTG GTGCCGTCCG CTCCTCCTAT GGAATAA |
| >AF015181 | .......... .......... .......... .......... ....... |

TABLE 8

Pileup
MSF: 727 Type: N Check: 1231 . . .
Name: NC_001463( gag720bp) (SEQ ID NO: 19) Len: 727 Check: 1714 Weight: 0
Name: >AF402664 (SEQ ID NO: 20) Len: 727 Check: 1659 Weight: 0
Name: >AF402665 (SEQ ID NO: 21) Len: 727 Check: 331 Weight: 0
Name: >AF402666 (SEQ ID NO: 22) Len: 727 Check: 7190 Weight: 0
Name: >AF402667 (SEQ ID NO: 23) Len: 727 Check: 9833 Weight: 0
Name: >AF402668 (SEQ ID NO: 24) Len: 727 Check: 504 Weight: 0
//

| | |
|---|---|
| | 1 50 |
| NC_001463 (gag720bp) | ATGGTGAGTC TAGATAGAGA CATGGCGAGG CAAGTCTCCG GGGGGAAAAG |
| >AF402664 | ........TC AAGCAGCAGG .AGGGAGAAG CTGGAAAGCA GTAGACTCAG |
| >AF402665 | ........GC AAGCAGCAGG .AGGGAGAAG CTGGAAAGCA GTAGACTCAG |
| >AF402666 | ........GC AAGCAGCAGG .AGGGAGAAG CTGGAAAGCA GTAGACTCAG |
| >AF402667 | ........GC AAGCAGCAGG .AGGGAGAAG CTGGAAAGCA GTAGACTCAG |
| >AF402668 | ........GC AAGCAGCAGG .AGGGAGAAG CTGGAAAGCA GTAGACTCAG |
| | 51 100 |
| NC_001463 (gag720bp) | AGATTATCCT GAGCTCGAAA AATGTATCAA GCATGCATGC AAGATAAAAG |
| >AF402664 | TGATGTTCCA GCAACTGCAA AATGTAGCAA TGCAGCATGG CCTCGTGTCC |
| >AF402665 | TGATGTTCCA GCAACTGCAA AATGTAGCAA TGCAGCATGG CCTCGTGTCC |
| >AF402666 | TGATGTTCCA GCAACTGCAA AATGTAGCAA TGCAGCATGG CCTCGTGTCC |
| >AF402667 | TGATGTTCCA GCAACTGCAA AATGTAGCAA TGCAGCATGG CCTCGTGTCC |
| >AF402668 | TGATGTTCCA GCAACTGCAA AATGTAGCAA TGCAGCATGG CCTCGTGTCC |
| | 101 150 |
| NC_001463 (gag720bp) | TTCGACTCAG AGGGGAGCAC TTGACAGAAG GAAATTGTTT ATGGTGCCTT |
| >AF402664 | GAGGATTTTG AAAGG..CAG TTAGTATATT ATGCTACTAC CTGGACAAGT |
| >AF402665 | GAGGATTTTG AAAGG..CAG TTAGCATATT ATGCTACTAC CTGGACAAGT |
| >AF402666 | GAGGATTTTG AAAGG..CAG TTGGCATATT ATGCTACTAC CTGGACAAGT |
| >AF402667 | GAGGATTTTG AAAGG..CAG TTAGCATATT ATGCTACTAC CTGGACAAGT |
| >AF402668 | GAGGATTTTG AAAGG..CAG TTAGCATATT ATGCTACTAC CTGGACAAGT |
| | 151 200 |
| NC_001463 (gag720bp) | AAAACATTAG ATTACATGTT TGAGGACCAT AAAGAGGAAC CITGGACAAA |
| >AF402664 | AAAGA..TAT ATTAGAAGTA TTGG..CCAT GATG.....C CTGGAAATAG |
| >AF402665 | AAAGA..TAT ATTAGAAGTA TTGG..CCAT GATG.....C CTGGAAATAG |
| >AF402666 | AAAGA..TAT ATTAGAAGTA TTGG..CCAT GATG.....C CTGGAAACAG |
| >AF402667 | AAAGA..TAT ATTAGAAGTA TTGG..CCAT GATG.....C CTGGAAATAG |
| >AF402668 | AAAGA..TAT ATTAGAAGTA TTGG..CCAT GATG.....C CTGGAAATAG |
| | 201 250 |
| NC_001463 (gag720bp) | AGTAAAATTT AGGACAATAT GGCAGAAGGT GAAGAATCTA ACTCCTGAGG |
| >AF402664 | AGCTCAAAAA GAGTTAATTC AAGGGAAATT GAATGAGGAA GCAGAAAGGT |
| >AF402665 | AGCTCAAAAA GAGTTAATTC AAGGGAAATT GAATGAGGAA GCAGAAAGGT |
| >AF402666 | AGCTCAAAAA GAGTTAATTC AGGGGAAATT GAATAAGGAA GCAGAAAGGT |
| >AF402667 | AGCTCAAAAA GAGTTAATTC AAGGGAAATT GAATGAGGAA GCAGAAAGGT |
| >AF402668 | AGCTCAAAAA GAGTTAATTC AAGGGAAATT GAATGAGGAA GCAGAAAGGT |
| | 251 300 |
| NC_001463 (gag720bp) | AGAGTAACAA AAAAGACTTT ATGTCTTTGC AGGCCACATT AGCGGGTCTA |
| >AF402664 | GGAG.AAGAA ATAATCCACC ACCTCAA.GC AGGCG..... .GAGGATTAA |
| >AF402665 | GGAG.AAGAA ATAATCCACC ACCTCAA.GC AGGCG..... .GAGGATTAA |
| >AF402666 | GGAG.AAGAA ATAATCCACC ACCTCAA.GC AGGCG..... .GAGGATTAA |
| >AF402667 | GGAG.AAGAA ATAATCCACC ACCTCAA.GC AGGCG..... .GAGGATTAA |
| >AF402668 | GGAG.AAGAA ATAATCCACC ACCTCAA.GC AGGCG..... .GAGGATTAA |
| | 301 350 |
| NC_001463(gag720bp) | ATGTGTTGCC AA..ATGGGG ATGAGACCTG AGACATTGCA AGATGCAATG |
| >AF4 02664 | CAGTGGATCA AATTATGGGG GTAGGACAAA CAAATCAAGC AGCGGCACAG |
| >AF4 02665 | CAGTGGATCA AATTATGGGG GTAGGACAAA CAAATCAAGC AGCGGCACAG |
| >AF4 02666 | CAGTGGATCA AATTATGGGG GTAGGACAAA CAAATCAGGC AGCGGCACAG |
| >AF4 02667 | CAGTGGATCA AATTATGGGG GTAGGACAAA CAAATCAAGC AGCGGCACAG |
| >AF4 02668 | CAGTGGATCA AATTATGGGG GTAGGACAAA CAAATCAAGC AGCGGCACAG |
| | 351 400 |
| NC_001463 (gag720bp) | GCTA.CAGTA ATCATGAAAG ATGGGTT..A CTGGAACAAG .AGGAAAAGA |
| >AF402664 | GCTAACATGG ATCAGGCAAG ACAAATATGT CTGCAATGGG TAATAACAGC |
| >AF402665 | GCTAACATGG ATCAGGCAAG ACAAATATGC CTGCAATGGG TAATAACAGC |
| >AF402666 | GCTAACATGG ATCAGGCAAG ACAAATATGC CTGCAATGGG TAATAACAGC |
| >AF402667 | GCTAACATGG ATCAGGCAAG ACAAATATGC CTGCAATGGG TAATAACAGC |
| >AF402668 | GCTAACATGG ATCAGGCAAG ACAAATATGC CTGCAATGGG TAATAACAGC |
| | 401 450 |
| NC_001463 (gag720bp) | AGGAAGACAA AAGAGAAAAG GAAGAGAGTG TCTTCCCAAT AGTAGTGCAA |
| >AF402664 | ACTAAGAGCA GTGAGACATA TGGCTCACAA ACCAGGGAAT CCAA.TGCTA |
| >AF402665 | ACTAAGAGCA GTGAGACATA TGGCTCACAA ACCAGGGAAT CCAA.TGCTA |
| >AF402666 | ACTAAGAGCA GTGAGACATA TGGCTCACAA ACCAGGGAAT CCAA.TGCTA |
| >AF402667 | ACTAAGAGCA GTGAGACATA TGGCTCACAA ACCAGGGAAT CCAA.TGCTA |
| >AF402668 | ACTAAGAGCA GTGAGACATA TGGCTCACAA ACCAGGGAAT CCAA.TGCTA |
| | 451 500 |
| NC_001463 (gag720bp) | GCAGCAGGAG GGAGAAGCTG GAAAGCAGTA GATTCTGTAA TCTTCCAGCA |
| >AF402664 | GTAAAGCAAA AAACAAATGA GTCATATGAA GATTTTGCCG CAAGACTGCT |
| >AF402665 | GTAAAGCAAA AGACAAATGA GTCATATGAA GATTTTGCCG CAAGACTGCT |
| >AF402666 | GTAAAGCAAA AGACAAATGA GTCATATGAA GATTTTGCCG CAAGACTGCT |
| >AF402667 | GTAAAGCAAA AGACAAATGA GTCATATGAA GATTTTGCCG CAAGACTGCT |
| >AF402668 | GTAAAGCAAA AGACAAATGA GTCATATGAA AAATTTTCAG CAAGACTCCT |
| | 501 550 |
| NC_001463 (gag720bp) | ACTGCAAACA GTAGCA.ATG CAGCATGGCC TCGTGTCTGA GGACTTTGAA |
| >AF402664 | AGAAGCAATA GATGCAGAAC CAGTTACACA GCAAATAAAA GAATATTTAA |
| >AF402665 | AGAAGCAATA GATGCAGAAC CAGTTACACA GCAAATAAAA GAATATTTAA |
| >AF402666 | AGAAGCAATA GATGCAGAAC CAGTTACACA GCAAATAAAA GAATATTTAA |
| >AF402667 | AGAAGCAATA GATGCAGAAC CAGTTACACA GCAAATAAA. GAATATTTAA |
| >AF402668 | AGAAGCAATA GATGCAGAAC CAGTTACACA GCCTATAAAA GAATATTTAA |
| | 551 600 |
| NC_001463 (gag720bp) | AGGCAGTTGG CATATTATGC TACTACCTGG ACAAGTAAAG ACATACTAGA |
| >AF402664 | AGTTA..... .ACATTATCT TAC.ACAAAT GCATCCTCAG ACTGTCAGAA |
| >AF402665 | AGTTA..... .ACATTATCT TAC.ACAAAT GCATCCTCAG ACTGTCAAAA |
| >AF402666 | AGTTA..... .ACATTATCT TAC.ACAAAT GCATCCTCAG ACTGTCAGAA |
| >AF402667 | .......... .......... .......... .......... .......... |
| >AF402668 | AGTTA..... .ACATTATCT TAC.ACAAAT GCATCCTCAG ACTGTCAAAA |
| | 601 650 |
| NC_001463 (gag720bp) | AGTATTGGCC ATGATGCCTG GAAATAGAGC TCAAAAGGAG TTAATTCAAG |
| >AF402664 | ACAGATGGAT AGAGTACTAG GACAGAGAGT GCAACAAGCT AGTGTGGAAG |
| >AF402665 | ACAAATGGAT AGAATACTAG GACAGAGAGT GCAACAAGCT AGTGTGGAAG |
| >AF402666 | ACAAATGGAT AGAGTACTAG GACAGAGAGT GCAACAAGCT AGTGTGGAAG |
| >AF402667 | .......... .......... .......... .......... .......... |
| >AF402668 | ACAAATGGAT AGAGTACTAG GACAGAGAGT GCAACAAGCT AGTGTGGAAG |
| | 651 700 |
| NC_001463 (gag720bp) | GGAAATTAAA TGAAGAAGCA GAAAGGTGGA GAAGGAATAA TCCACCACCT |
| >AF402664 | AAAAAATGCA ACCAT..GCA GAGATGTGGG ATCAGAAGGA TTCAGAATGC |
| >AF402665 | AAAAAATGCA ACCAT..GCA GAGATGTGGG ATCAGAAGGG TTCAGAATGC |
| >AF402666 | AAAAAATGCA AGCAT..GCA GAGATGTGGG ATCAGAAGG. .......... |
| >AF402667 | .......... .......... .......... .......... .......... |
| >AF402668 | AAAAAATGCA AGCAT..GCA GAGATGTGGG ATCAGAAGGA TTCAGAATGC |
| | 701 727 |
| NC_001463 (gag720bp) | CCAGCAGGAG GAGGATTAAC AGTGGAT |
| >AF402664 | .......... .......... ....... |
| >AF402665 | .......... .......... ....... |
| >AF402666 | .......... .......... ....... |
| >AF402667 | .......... .......... ....... |
| >AF402668 | .......... .......... ....... |

Pileup
MSF: 1347 Type: N Check: 5320 . . .
Name: NC_001463 (gag) (SEQ ID NO: 25) Len: 1347 Check: 6959 Weight: 0
Name: >AF402664 (SEQ ID NO: 26) Len: 1347 Check: 1590 Weight: 0
Name: >AF402665 (SEQ ID NO: 27) Len: 1347 Check: 9222 Weight: 0
Name: >AF402666 (SEQ ID NO: 28) Len: 1347 Check: 4950 Weight: 0
Name: >AF402667 (SEQ ID NO: 29) Len: 1347 Check: 3156 Weight: 0
Name: >AF402668 (SEQ ID NO: 30) Len: 1347 Check: 9443 Weight: 0
//

| | |
|---|---|
| | 1 50 |
| NC_001463 (gag) | ATGGTGAGTC TAGATAGAGA CATGGCGAGG CAAGTCTCCG GGGGGAAAAG |
| >AF402664 | .......... .......... .......... .......... .......... |
| >AF402665 | .......... .......... .......... .......... .......... |
| >AF402666 | .......... .......... .......... .......... .......... |
| >AF402667 | .......... .......... .......... .......... .......... |
| >AF402668 | .......... .......... .......... .......... .......... |
| | 51 100 |
| NC_001463 (gag) | AGATTATCCT GAGCTCGAAA AATGTATCAA GCATGCATGC AAGATAAAAG |
| >AF402664 | .......... .......... .......... .......... .......... |
| >AF402665 | .......... .......... .......... .......... .......... |
| >AF402666 | .......... .......... .......... .......... .......... |
| >AF402667 | .......... .......... .......... .......... .......... |
| >AF402668 | .......... .......... .......... .......... .......... |
| | 101 150 |
| NC_001463(gag) TTCGACTCAG AGGGGAGCAC TTGACAGAAG GAAATTGTTT ATGGTGCCTT | |
| >AF402664 | .......... .......... .......... .......... .......... |
| >AF402665 | .......... .......... .......... .......... .......... |
| >AF402666 | .......... .......... .......... .......... .......... |
| >AF402667 | .......... .......... .......... .......... .......... |
| >AF402668 | .......... .......... .......... .......... .......... |
| | 151 200 |
| NC_001463(gag) | AAAACATTAG ATTACATGTT TGAGGACCAT AAAGAGGAAC CTTGGACAAA |
| >AF402664 | .......... .......... .......... .......... .......... |
| >AF402665 | .......... .......... .......... .......... .......... |
| >AF402666 | .......... .......... .......... .......... .......... |
| >AF402667 | .......... .......... .......... .......... .......... |
| >AF402668 | .......... .......... .......... .......... .......... |
| | 201 250 |
| NC_001463 (gag) | AGTAAAATTT AGGACAATAT GGCAGAAGGT GAAGAATCTA ACTCCTGAGG |
| >AF402664 | .......... .......... .......... .......... .......... |
| >AF402665 | .......... .......... .......... .......... .......... |
| >AF402666 | .......... .......... .......... .......... .......... |
| >AF402667 | .......... .......... .......... .......... .......... |
| >AF402668 | .......... .......... .......... .......... .......... |
| | 251 300 |
| NC_001463 (gag) | AGAGTAACAA AAAAGACTTT ATGTCTTTGC AGGCCACATT AGCGGGTCTA |
| >AF402664 | .......... .......... .......... .......... .......... |
| >AF402665 | .......... .......... .......... .......... .......... |
| >AF402666 | .......... .......... .......... .......... .......... |
| >AF402667 | .......... .......... .......... .......... .......... |
| >AF402668 | .......... .......... .......... .......... .......... |
| | 301 350 |
| NC_001463 (gag) | ATGTGTTGCC AAATGGGGAT GAGACCTGAG ACATTGCAAG ATGCAATGGC |
| >AF402664 | .......... .......... .......... .......... .......... |
| >AF402665 | .......... .......... .......... .......... .......... |
| >AF402666 | .......... .......... .......... .......... .......... |
| >AF402667 | .......... .......... .......... .......... .......... |
| >AF402668 | .......... .......... .......... .......... .......... |
| | 351 400 |
| NC_001463 (gag) | TACAGTAATC ATGAAAGATG GGTTACTGGA ACAAGAGGAA AAGAAGGAAG |
| >AF402664 | .......... .......... .......... .......... .......... |
| >AF402665 | .......... .......... .......... .......... .......... |
| >AF402666 | .......... .......... .......... .......... .......... |
| >AF402667 | .......... .......... .......... .......... .......... |
| >AF402668 | .......... .......... .......... .......... .......... |
| | 401 450 |
| NC_001463 (gag) | ACAAAAGAGA AAAGGAAGAG AGTGTCTTCC CAATAGTAGT GCAAGCAGCA |
| >AF402664 | .......... .......... .......... .......... TCAAGCAGCA |
| >AF402665 | .......... .......... .......... .......... GCAAGCAGCA |
| >AF402666 | .......... .......... .......... .......... GCAAGCAGCA |
| >AF402667 | .......... .......... .......... .......... GCAAGCAGCA |
| >AF402668 | .......... .......... .......... .......... GCAAGCAGCA |
| | 451 500 |
| NC_001463 (gag) | GGAGGGAGAA GCTGGAAAGC AGTAGATTCT GTAATGTTCC AGCAACTGCA |
| >AF402664 | GGAGGGAGAA GCTGGAAAGC AGTAGACTCA GTGATGTTCC AGCAACTGCA |
| >AF402665 | GGAGGGAGAA GCTGGAAAGC AGTAGACTCA GTGATGTTCC AGCAACTGCA |
| >AF402666 | GGAGGGAGAA GCTGGAAAGC AGTAGACTCA GTGATGTTCC AGCAACTGCA |
| >AF402667 | GGAGGGAGAA GCTGGAAAGC AGTAGACTCA GTGATGTTCC AGCAACTGCA |
| >AF402668 | GGAGGGAGAA GCTGGAAAGC AGTAGACTCA GTGATGTTCC AGCAACTGCA |
| | 501 550 |
| NC_001463(gag) | AACAGTAGCA ATGCAGCATG GCCTCGTGTC TGAGGACTTT GAAAGGCAGT |
| >AF402664 | AAATGTAGCA ATGCAGCATG GCCTCGTGTC CGAGGATTTT GAAAGGCAGT |
| >AF402665 | AAATGTAGCA ATGCAGCATG GCCTCGTGTC CGAGGATTTT GAAAGGCAGT |
| >AF402666 | AAATGTAGCA ATGCAGCATG GCCTCGTGTC CGAGGATTTT GAAAGGCAGT |
| >AF402667 | AAATGTAGCA ATGCAGCATG GCCTCGTGTC CGAGGATTTT GAAAGGCAGT |
| >AF402668 | AAATGTAGCA ATGCAGCATG GCCTCGTGTC CGAGGATTTT GAAAGGCAGT |
| | 551 600 |
| NC_001463 (gag) | TGGCATATTA TGCTACTACC TGGACAAGTA AAGACATACT AGAAGTATTG |
| >AF402664 | TAGTATATTA TGCTACTACC TGGACAAGTA AAGATATATT AGAAGTATTG |
| >AF402665 | TAGCATATTA TGCTACTACC TGGACAAGTA AAGATATATT AGAAGTATTG |
| >AF402666 | TGGCATATTA TGCTACTACC TGGACAAGTA AAGATATATT AGAAGTATTG |
| >AF402667 | TAGCATATTA TGCTACTACC TGGACAAGTA AAGATATATT AGAAGTATTG |
| >AF402668 | TAGCATATTA TGCTACTACC TGGACAAGTA AAGATATATT AGAAGTATTG |
| | 601 650 |
| NC_001463 (gag) | GCCATGATGC CTGGAAATAG AGCTCAAAAG GAGTTAATTC AAGGGAAATT |
| >AF402664 | GCCATGATGC CTGGAAATAG AGCTCAAAAA GAGTTAATTC AAGGGAAATT |
| >AF402665 | GCCATGATGC CTGGAAATAG AGCTCAAAAA GAGTTAATTC AAGGGAAATT |
| >AF402666 | GCCATGATGC CTGGAAACAG AGCTCAAAAA GAGTTAATTC AGGGGAAATT |
| >AF402667 | GCCATGATGC CTGGAAATAG AGCTCAAAAA GAGTTAATTC AAGGGAAATT |
| >AF402668 | GCCATGATGC CTGGAAATAG AGCTCAAAAA GAGTTAATTC AAGGGAAATT |
| | 651 700 |
| NC_001463 (gag) | AAATGAAGAA GCAGAAAGGT GGAGAAGGAA TAATCCACCA CCTCCAGCAG |
| >AF402664 | GAATGAGGAA GCAGAAAGGT GGAGAAGAAA TAATCCACCA CCTCAAGCAG |
| >AF402665 | GAATGAGGAA GCAGAAAGGT GGAGAAGGAA TAATCCACCA CCTCAAGCAG |
| >AF402666 | GAATAAGGAA GCAGAAAGGT GGAGAAGGAA TAATCCACCA CCTCAAGCAC |
| >AF402667 | GAATGAGGAA GCAGAAAGGT GGAGAAGGAA TAATCCACCA CCTCAAGCAG |
| >AF402668 | GAATGAGGAA GCAGAAAGGT GGAGAAGGAA TAATCCACCA CCTCAAGCAG |
| | 701 750 |
| NC_001463 (gag) | GAGGAGGATT AACAGTGGAT CAAATTATGG GGGTAGGACA AACAAATCAA |
| >AF402664 | GCGGAGGATT AACAGTGGAT CAAATTATGG GGGTAGGACA AACAAATCAA |
| >AF402665 | GCGGAGGATT AACAGTGGAT CAAATTATGG GGGTAGGACA AACAAATCAA |
| >AF402666 | AAGGAGGATT AACAGTGGAT CAAATTATGG GGGTAGGACA AACAAATCAG |
| >AF402667 | GCGGAGGATT AACAGTGGAT CAAATTATGG GGGTAGGACA AACAAATCAA |
| >AF402668 | GCGGAGGATT AACAGTGGAT CAAATTATGG GGGTAGGACA AACAAATCAA |
| | 751 800 |
| NC_001463 (gag) | GCAGCAGCAC AAGCTAACAT GGATCAGGCA AGGCAAATAT GCCTGCAATG |
| >AF402664 | GCAGCGGCAC AGGCTAACAT GGATCAGGCA AGACAAATAT GTCTGCAATG |
| >AF402665 | GCAGCGGCAC AGGCTAACAT GGATCAGGCA AGACAAATAT GCCTGCAATG |
| >AF402666 | GCAGCGGCAC AGGCTAACAT GGATCAGGCA AGACAAATAT GCCTGCAATG |
| >AF402667 | GCAGCGGCAC AGGCTAACAT GGATCAGGCA AGACAAATAT GCCTGCAATG |
| >AF402668 | GCAGCGGCAC AGGCTAACAT GGATCAGGCA AGACAAATAT GCCTGCAATG |
| | 801 850 |
| NC_001463 (gag) | GGTAATAAAT GCATTAAGAG CAGTAAGACA TATGGCGCAC AGGCCAGGGA |
| >AF402664 | GGTAATAACA GCACTAAGAG CAGTGAGACA TATGGCTCAC AAACCAGGGA |
| >AF402665 | GGTAATAACA GCACTAAGAG CAGTGAGACA TATGGCTCAC AAACCAGGGA |
| >AF402666 | GGTAATAACA GCACTAAGAG CAGTGAGACA TATGGCTCAC AAACCAGGGA |
| >AF402667 | GGTAATAACA GCACTAAGAG CAGTGAGACA TATGGCTCAC AAACCAGGGA |
| >AF402668 | GGTAATAACA GCACTAAGAG CAGTGAGACA TATGGCTCAC AAACCAGGGA |
| | 852 900 |
| NC_001463 (gag) | ATCCAATGCT AGTAAAGCAA AAAACGAATG AGCCATATGA AGATTTTGCA |
| >AF402664 | ATCCAATGCT AGTAAAGCAA AAAACAAATG AGTCATATGA AGATTTTGCC |
| >AF402665 | ATCCAATGCT AGTAAAGCAA AAGACAAATG AGTCATATGA AGATTTTGCC |
| >AF402666 | ATCCAATGCT AGTAAAGCAA AAGACAAATG AGTCATATGA AGATTTTGCC |
| >AF402667 | ATCCAATGCT AGTAAAGCAA AAGACAAATG AGTCATATGA AGATTTTGCC |
| >AF402668 | ATCCAATGCT AGTAAAGCAA AAGACAAATG AGTCATATGA AAAATTTTCA |
| | 901 950 |
| NC_001463 (gag) | GCAAGACTGC TAGAAGCAAT AGATGCAGAG CCAGTTACAC AGCCTATAAA |
| >AF402664 | GCAAGACTGC TAGAAGCAAT AGATGCAGAA CCAGTTACAC AGCAAATAAA |
| >AF402665 | GCAAGACTGC TAGAAGCAAT AGATGCAGAA CCAGTTACAC AGCAAATAAA |
| >AF402666 | GCAAGACTGC TAGAAGCAAT AGATGCAGAA CCAGTTACAC AGCAAATAAA |
| >AF402667 | GCAAGACTGC TAGAAGCAAT AGATGCAGAA CCAGTTACAC AGCAAATAAA |
| >AF402668 | GCAAGACTCC TAGAAGCAAT AGATGCAGAA CCAGTTACAC AGCCTATAAA |
| | 951 1000 |
| NC_001463 (gag) | AGATTATCTA AAGCTAACAC TATCTTATAC AAATGCATCA GCAGATTGTC |
| >AF402664 | AGAATATTTA AAGTTAACAT TATCTTACAC AAATGCATCC TCAGACTGTC |
| >AF402665 | AGAATATTTA AAGTTAACAT TATCTTACAC AAATGCATCC TCAGACTGTC |
| >AF402666 | AGAATATTTA AAGTTAACAT TATCTTACAC AAATGCATCC TCAGACTGTC |
| >AF402667 | .GAATATTTA A......... .......... .......... .......... |
| >AF402668 | AGAATATTTA AAGTTAACAT TATCTTACAC AAATGCATCC TCAGACTGTC |
| | 1001 1050 |
| NC_001463 (gag) | AGAAGCAAAT GGATAGAACA CTAGGACAAA GAGTACAACA AGCTAGTGTA |
| >AF402664 | AGAAACAGAT GGATAGAGTA CTAGGACAGA GAGTGCAACA AGCTAGTGTG |
| >AF402665 | AAAAACAAAT GGATAGAATA CTAGGACAGA GAGTGCAACA AGCTAGTGTG |
| >AF402666 | AGAAACAAAT GGATAGAGTA CTAGGACAGA GAGTGCAACA AGCTAGTGTG |
| >AF402667 | .......... .......... .......... .......... .......... |
| >AF402668 | AAAAACAAAT GGATAGAGTA CTAGGACAGA GAGTGCAACA AGCTAGTGTG |
| | 1051 1100 |
| NC_001463 (gag) | GAAGAAAAAA TGCAAGCATG TAGAGATGTG GGATCAGAAG GGTTCAAAAT |
| >AF402664 | GAAGAAAAAA TGCAAGCATG CAGAGATGTG GGATCAGAAG GATTCAGAAT |
| >AF402665 | GAAGAAAAAA TGCAAGCATG CAGAGATGTG GGATCAGAAG GGTTCAGAAT |
| >AF402666 | GAAGAAAAAA TGCAAGCATG CAGAGATGTG GGATCAGAAG G......... |
| >AF402667 | .......... .......... .......... .......... .......... |
| >AF402668 | GAAGAAAAAA TGCAAGCATG CAGAGATGTG GGATCAGAAG GATTCAGAAT |
| | 1101 1150 |
| NC_001463 (gag) | GCAATTGTTA GCACAAGCAT TAAGGCCAGG AAAAGGAAAA GGGAATGGAC |
| >AF402664 | GC........ .......... .......... .......... .......... |
| >AF402665 | GC........ .......... .......... .......... .......... |
| >AF402666 | .......... .......... .......... .......... .......... |
| >AF402667 | .......... .......... .......... .......... .......... |
| >AF402668 | GC........ .......... .......... .......... .......... |
| | 1151 1200 |
| NC_001463 (gag) | AGCCACAAAG GTGTTACAAC TGTGGAAAAC CGGGACATCA AGCAAGGCAA |
| >AF402664 | .......... .......... .......... .......... .......... |
| >AF402665 | .......... .......... .......... .......... .......... |
| >AF402666 | .......... .......... .......... .......... .......... |
| >AF402667 | .......... .......... .......... .......... .......... |
| >AF402668 | .......... .......... .......... .......... .......... |
| | 1201 1250 |
| NC_001463 (gag) | TGTAGACAAG GAATCATATG TCACAACTGT GGAAAGAGAG GACATATGCA |
| >AF402664 | .......... .......... .......... .......... .......... |
| >AF402665 | .......... .......... .......... .......... .......... |
| >AF402666 | .......... .......... .......... .......... .......... |
| >AF402667 | .......... .......... .......... .......... .......... |
| >AF402668 | .......... .......... .......... .......... .......... |
| | 1251 1300 |
| NC_001463 (gag) | AAAAGAATGC AGAGGAAAGA GAGACATAAG GGGAAAACAG CAGGGAAACG |
| >AF402664 | .......... .......... .......... .......... .......... |
| >AF402665 | .......... .......... .......... .......... .......... |
| >A~402666 | .......... .......... .......... .......... .......... |
| >AF402667 | .......... .......... .......... .......... .......... |
| >AF402668 | .......... .......... .......... .......... .......... |
| | 1301 1347 |
| NC_001463 (gag) | GGAGGAGGGG GATACGTGTG GTGCCGTCCG CTCCTCCTAT GGAATAA |
| >AF402664 | .......... .......... .......... .......... ....... |
| >AF402665 | .......... .......... .......... .......... ....... |
| >AF402666 | .......... .......... .......... .......... ....... |
| >AF402667 | .......... .......... .......... .......... ....... |
| >AF402668 | .......... .......... .......... .......... ....... |

TABLE 9

Pileup
MSF: 742 Type: N Check: 6523 . . .
Name: NC_001463 (gag720bp) (SEQ ID NO: 31) Len: 742 Check: 3818 Weight: 0
Name: >AJ305040 (SEQ ID NO: 32) Len: 742 Check: 1263 Weight: 0
Name: >AJ305041 (SEQ ID NO: 33) Len: 742 Check: 9126 Weight: 0
Name: >AJ305042 (SEQ ID NO: 34) Len: 742 Check: 2316 Weight: 0
//

| | |
|---|---|
| | 1 50 |
| NC_001463 (gag720bp) | ATGGTGAGTC TAGATAGAGA CATGGCGAGG CAAGTCTC.. CGGGGGGAAA |
| >AJ305040 | ......GCAG TCGATGCTGT AATGTTCCAG CAAATGCAAA CAGTAGCCAT |
| >AJ305041 | ......GCAG TAGACTCAGT AATGTTCCAG CAACTGCAAA CAGTAGCAAT |
| >AJ305042 | ......GCAG TCGATGCTGT AATGTTCCAG CAAATGCAAA CAGTAGCCAT |
| | 51 100 |
| NC_001463 (gag720bp) | AGAGATTATC CTGAGCTCGA AAAATGTATC AAGCATGCAT GCAAGATAAA |
| >AJ305040 | GCAGCATGGT CTTGTGTCTG AGGACTTTGA AAGGCAGTTA GCAT.ATTGT |
| >AJ305041 | GCAGCATGGC CTCGTGTCCG AGGATTTTGA AAGGCAGTTG GCAT.ATTAT |
| >AJ305042 | GCAGCATGGT CTTGTGTCTG AGGACTTTGA AAGGCAGTTA GCAT.ATTAT |
| | 101 150 |
| NC_001463 (gag720bp) | AGTTCGACTC AGAGGGG..A GCACTTGACA GAAGGAAATT GTTTATGGTG |
| >AJ305040 | GCTACTACCT GGACAAGTAA AGATATAITA GAAGTA..TT GGCCATGATG |
| >AJ305041 | GCTACTACCT GGACGAGTAA AGACATACTA GAAGTA..TT GGCCATGATG |
| >AJ305042 | GCTACTACCT GGACAAGTAA AGATATATTA GAAGTA..TT GGCCATGATG |
| | 151 200 |
| NC_001463 (gag720bp) | CCTTAAAACA TTAGATTACA TGTTTGAGGA CCATAAAGAG GAACCTTGGA |
| >AJ305040 | CCTGGAAATA G.AGCTCAAA AA...GAGTT AATTCAAG.G AAAATTAAAC |
| >AJ305041 | CCTGGAAACA G.AGCTCAAA AG...GAGTT AATTCAAG.G GAAATTAAAT |
| >AJ305042 | CCTGGAAATA G.AGCTCAAA AA...GAGTT AATTCAAG.G AAAATTAAAT |
| | 201 250 |
| NC_001463 (gag720bp) | CAAAAGTAAA ATTTAGGACA ATATGGCAGA AGGTGAAGAA TCTAACTCCT |
| >AJ305040 | GAGGAAGCAG AA..AGGTGG AGAAGGAATA A..TCCACCG CCTCCACAAG |
| >AJ305041 | GAAGAGGCAG AA..AGGTGG AGAAGACATA A..TCCACCC CCTCCGGCGG |
| >AJ305042 | GAGGAAGCAG AA..AGGTGG AGAAGGAATA A..TCCACCG CCTCCACAGG |
| | 251 300 |
| NC_001463 (gag720bp) | GAGGAG.AGT AACAAAAAAG .ACTTTATGT CTTTGCAGGC CACATTAGCG |
| >AJ305040 | GAGGGGGATT AACAGTGGAT CAAATTATGG GGAT..AGGA CAAACAAATC |
| >AJ305041 | GAGGAGGATT AACAGTGGAT CAAATTATGG GGGT..AGGA CAAACAAATC |
| >AJ305042 | GAGGGGGATT AACAGTGGAT CAAATTATGG GGAT..AGGA CAAACAAATC |
| | 301 350 |
| NC_001463 (gag720bp) | GGTCTAATGT GTTGCCAAAT GGGGATGAGA CCTGAGACAT .....TGCAA |
| >AJ305040 | AAGCAGCAGC ACAAGCTAAC ATGGATCAGG CAAGACACAT ATGCCTGCAA |
| >AJ305041 | AAGCAGCAGC ACAAGCTAAC ATGGATCAGG CAAGACAAAT ATGCCTGCAA |
| >AJ305042 | AAGCAGCAGC ACAAGCTAAC ATGGATCAGG CAAGACACAT ATGCCTGCAA |
| | 351 400 |
| NC_001463 (gag720bp) | GATGCAATGG CTACAGTAAT ..CA.TGAAA GATGGGTTAC TGGAACAAGA |
| >AJ305040 | TGGGTAATAA CAGCATTAAG AGCAGTAAGA CATATGGCTC ACAGACCAGG |
| >AJ305041 | TGGGTAATAA CAGCATTAAG AGCAGTGAGG TATATGACTC ACAAACCAGG |
| >AJ305042 | TGGGTAATAA CAGCATTAAG AGCAGTAAGA CATATGGCTC ACAGACCAGG |
| | 401 450 |
| NC_001463 (gag720bp) | GGA...AAAG A.AGGAAGAC AAAAGAGAAA AGGAAGAGAG T..GTCTTCC |
| >AJ305040 | GAATCCAATG CTCGTAAAAC AAAAAACAAA TGAGCCATAT GAAGAGTTTG |
| >AJ305041 | GAATCCAATG CTAGTAAAAC AAAAAACAAA TGAAGCATAT GAAGAGTTTA |
| >AJ305042 | GAATCCAATG CTCGTAAAAC AAAAAACAAA TGAGCCATAT GAAGAGTTTG |
| | 451 500 |
| NC_001463 (gag720bp) | CAATAGTAGT GCAAGCAGCA GGAG..GGAG AAGCTGGAAA GCAGTAGATT |
| >AJ305040 | CAGCAAAACT ATTAGAAGCA ATAGATGCAG AACCAGTAAC ACAGCCCATA |
| >AJ305041 | CAGCGAGACT GCTAGAAGCA ATAGATGCAG AGCCAGTAAC ACAGCCCACA |
| >AJ305042 | CAGCAAAACT ATTAGAAGCA ATAGATGCAG AACCAGTAAC ACAGCTCATA |
| | 501 550 |
| NC_001463 (gag720bp) | CTGTAATGTT CCAGCAACTG CAAACAGTAG CAATGCAGCA TGGCCTCGTG |
| >AJ305040 | AAAGACTAT.. CTAAAGTT.. .AACATTAT CT.TATACAA ATGCGTC... |
| >AJ305041 | AAAGAATAT.. CTAAAACT.. .AACATTAT CT.TATACAA ATGCATC... |
| >AJ305042 | AAAGACTAT.. CTAAAGTT.. .AACATTAT CT.TATACAA ATGCGTC... |
| | 551 600 |
| NC_001463 (gag720bp) | TCTGAGGACT TTGAAAGGCA GTTGGCATAT TATGCTACTA CCTGGACAAG |
| >AJ305040 | .CTCAG.ACT GTCAAAAGCA AATGG.ATAG AGTGCTGGGA CAAAG...AG |
| >AJ305041 | .CTCAG.ACT GTCAAAAGCA AATGG.ATAG AGTACTAGGA CAAAG...AG |
| >AJ305042 | .CTCAG.ACT GTCAAAAGCA AATGG.ATAG AGTGCTGGGA CAAAG...AG |
| | 601 650 |
| NC_001463 (gag720bp) | TAAAGACATA CTAGAAGTAT TGGCCATGAT GCCTGGAAAT AGAGCTCAAA |
| >AJ305040 | TGCA.ACAAG CTAGT.GTAG ACGAGAAAAT GCAA...... .......... |
| >AJ305041 | TGCA.ACAAG CTAGT.GTAG AAGAAAAAAT GCAA...... .......... |
| >AJ305042 | TGCA.ACAAG CTAGT.GTAG ACGAGAAGAT GCAA...... .......... |
| | 651 700 |
| NC_001463 (gag720bp) | AGGAGTTAAT TCAAGGGAAA TTAAATGAAG AAGCAGAAAG GTGGAGAAGG |
| >AJ305040 | .......... .......... .......... .......... .......... |
| >AJ305041 | .......... .......... .......... .......... .......... |
| >AJ305042 | .......... .......... .......... .......... .......... |
| | 701 742 |
| NC_001463 (gag72obp) | AATAATCCAC CACCTCCAGC AGGAGGAGGA TTAACAGTGG AT |
| >AJ305040 | .......... .......... .......... .......... .. |
| >AJ305041 | .......... .......... .......... .......... .. |
| >AJ305042 | .......... .......... .......... .......... .. |

PileUp
MSF: 1347 Type: N Check: 9510 . . .
Name: NC_001463 (gag) (SEQ ID NO: 35) Len: 1347 Check: 6959 Weight: 0
Name: >AJ305040 (SEQ ID NO: 36) Len: 1347 Check: 1930 Weight: 0
Name: >AJ305041 (SEQ ID NO: 37) Len: 1347 Check: 7682 Weight: 0
Name: >AJ305042 (SEQ ID NO: 38) Len: 1347 Check: 2939 Weight: 0
//

| | |
|---|---|
| | 1 50 |
| NC_001463 (gag) | ATGGTGAGTC TAGATAGAGA CATGGCGAGG CAAGTCTCCG GGGGGAAAAG |
| >AJ305040 | .......... .......... .......... .......... .......... |
| >AJ305041 | .......... .......... .......... .......... .......... |
| >AJ305042 | .......... .......... .......... .......... .......... |
| | 51 100 |
| NC_001463 (gag) | AGATTATCCT GAGCTCGAAA AATGTATCAA GCATGCATGC AAGATAAAAG |
| >AJ305040 | .......... .......... .......... .......... .......... |
| >AJ305041 | .......... .......... .......... .......... .......... |
| >AJ305042 | .......... .......... .......... .......... .......... |
| | 101 150 |
| NC_001463 (gag) | TTCGACTCAG AGGGGAGCAC TTGACAGAAG GAAATTGTTT ATGGTGCCTT |
| >AJ305040 | .......... .......... .......... .......... .......... |
| >AJ305041 | .......... .......... .......... .......... .......... |
| >AJ305042 | .......... .......... .......... .......... .......... |
| | 151 200 |
| NC_001463 (gag) | AAAACATTAG ATTACATGTT TGAGGACCAT AAAGAGGAAC CTTGGACAAA |
| >AJ305040 | .......... .......... .......... .......... .......... |
| >AJ305041 | .......... .......... .......... .......... .......... |
| >AJ305042 | .......... .......... .......... .......... .......... |
| | 201 250 |
| NC_001463 (gag) | AGTAAAATTT AGGACAATAT GGCAGAAGGT GAAGAATCTA ACTCCTGAGG |
| >AJ305040 | .......... .......... .......... .......... .......... |
| >AJ305041 | .......... .......... .......... .......... .......... |
| >AJ305042 | .......... .......... .......... .......... .......... |
| | 251 300 |
| NC_001463 (gag) | AGAGTAACAA AAAAGACTTT ATGTCTTTGC AGGCCACATT AGCGGGTCTA |
| >AJ305040 | .......... .......... .......... .......... .......... |
| >AJ305041 | .......... .......... .......... .......... .......... |
| >AJ305042 | .......... .......... .......... .......... .......... |
| | 301 350 |
| NC_001463 (gag) | ATGTGTTGCC AAATGGGGAT GAGACCTGAG ACATTGCAAG ATGCAATGGC |
| >AJ305040 | .......... .......... .......... .......... .......... |
| >AJ305041 | .......... .......... .......... .......... .......... |
| >AJ305042 | .......... .......... .......... .......... .......... |
| | 351 400 |
| NC_001463 (gag) | TACAGTAATC ATGAAAGATG GGTTACTGGA ACAAGAGGAA AAGAAGGAAG |
| >AJ305040 | .......... .......... .......... .......... .......... |
| >AJ305041 | .......... .......... .......... .......... .......... |
| >AJ305042 | .......... .......... .......... .......... .......... |
| | 401 450 |
| NC_001463 (gag) | ACAAAAGAGA AAAGGAAGAG AGTGTCTTCC CAATAGTAGT GCAAGCAGCA |
| >AJ305040 | .......... .......... .......... .......... .......... |
| >AJ305041 | .......... .......... .......... .......... .......... |
| >AJ305042 | .......... .......... .......... .......... .......... |
| | 451 500 |
| NC_001463 (gag) | GGAGGGAGAA GCTGGAAAGC AGTAGATTCT GTAATGTTCC AGCAACTGCA |
| >AJ305040 | .......... ........GC AGTCGATGCT GTAATGTTCC AGCAAATGCA |
| >AJ305041 | .......... ........GC AGTAGACTCA GTAATGTTCC AGCAACTGCA |
| >AJ305042 | .......... ........GC AGTCGATGCT GTAATGTTCC AGCAAATGCA |
| | 501 550 |
| NC_001463 (gag) | AACAGTAGCA ATGCAGCATG GCCTCGTGTC TGAGGACTTT GAAAGGCAGT |
| >AJ305040 | AACAGTAGCC ATGCAGCATG GTCTTGTGTC TGAGGACTTT GAAAGGCAGT |
| >AJ305041 | AACAGTAGCA ATGCAGCATG GCCTCGTGTC CGAGGATTTT GAAAGGCAGT |
| >AJ305042 | AACAGTAGCC ATGCAGCATG GTCTTGTGTC TGAGGACTTT GAAAGGCAGT |
| | 551 600 |
| NC_001463 (gag) | TGGCATATTA TGCTACTACC TGGACAAGTA AAGACATACT AGAAGTATTG |
| >AJ305040 | TAGCATATTG TGCTACTACC TGGACAAGTA AAGATATATT AGAAGTATTG |
| >AJ305041 | TGGCATATTA TGCTACTACC TGGACGAGTA AAGACATACT AGAAGTATTG |
| >AJ305042 | TAGCATATTA TGCTACTACC TGGACAAGTA AAGATATATT AGAAGTATTG |
| | 601 650 |
| NC_001463 (gag) | GCCATGATGC CTGGAAATAG AGCTCAAAAG GAGTTAATTC AAGGGAAATT |
| >AJ305040 | GCCATGATGC CTGGAAATAG AGCTCAAAAA GAGTTAATTC AAGGAAAATT |
| >AJ305041 | GCCATGATGC CTGGAAACAG AGCTCAAAAG GAGTTAATTC AAGGGAAATT |
| >AJ305042 | GCCATGATGC CTGGAAATAG AGCTCAAAAA GAGTTAATTC AAGGAAAATT |
| | 651 700 |
| NC_001463 (gag) | AAATGAAGAA GCAGAAAGGT GGAGAAGGAA TAATCCACCA CCTCCAGCAG |
| >AJ305040 | AAACGAGGAA GCAGAAAGGT GGAGAAGGAA TAATCCACCG CCTCCACAAG |
| >AJ305041 | AAATGAAGAG GCAGAAAGGT GGAGAAGACA TAATCCACCC CCTCCGGCGG |
| >AJ305042 | AAATGAGGAA GCAGAAAGGT GGAGAAGGAA TAATCCACCG CCTCCACAGG |
| | 701 750 |
| NC_001463 (gag) | GAGGAGGATT AACAGTGGAT CAAATTATGG GGGTAGGACA AACAAATCAA |
| >AJ305040 | GAGGGGGATT AACAGTGGAT CAAATTATGG GGATAGGACA AACAAATCAA |
| >AJ305041 | GAGGAGGATT AACAGTGGAT CAAATTATGG GGGTAGGACA AACAAATCAA |
| >AJ305042 | GAGGGGGATT AACAGTGGAT CAAATTATGG GGATAGGACA AACAAATCAA |
| | 751 800 |
| NC_001463 (gag) | GCAGCAGCAC AAGCTAACAT GGATCAGGCA AGGCAAATAT GCCTGCAATG |
| >AJ305040 | GCAGCAGCAC AAGCTAACAT GGATCAGGCA AGACACATAT GCCTGCAATG |
| >AJ305041 | GCAGCAGCAC AAGCTAACAT GGATCAGGCA AGACAAATAT GCCTGCAATG |
| >AJ305042 | GCAGCAGCAC AAGCTAACAT GGATCAGGCA AGACACATAT GCCTGCAATG |
| | 801 850 |
| NC_001463(gag) | GGTAATAAAT GCATTAAGAG CAGTAAGACA TATGGCGCAC AGGCCAGGGA |
| >AJ305040 | GGTAATAACA GCATTAAGAG CAGTAAGACA TATGGCTCAC AGACCAGGGA |
| >AJ305041 | GGTAATAACA GCATTAAGAG CAGTGAGGTA TATGACTCAC AAACCAGGGA |
| >AJ305042 | GGTAATAACA GCATTAAGAG CAGTAAGACA TATGGCTCAC AGACCAGGGA |
| | 851 900 |
| NC_001463 (gag) | ATCCAATGCT AGTAAAGCAA AAAACGAATG AGCCATATGA AGATTTTGCA |
| >AJ305040 | ATCCAATGCT CGTAAAACAA AAAACAAATG AGCCATATGA AGAGTTTGCA |
| >AJ305041 | ATCCAATGCT AGTAAAACAA AAAACAAATG AAGCATATGA AGAGTTTACA |
| >AJ305042 | ATCCAATGCT CGTAAAACAA AAAACAAATG AGCCATATGA AGAGTTTGCA |
| | 901 950 |
| NC_001463 (gag) | GCAAGACTGC TAGAAGCAAT AGATGCAGAG CCAGTTACAC AGCCTATAAA |
| >AJ305040 | GCAAAACTAT TAGAAGCAAT AGATGCAGAA CCAGTAACAC AGCCCATAAA |
| >AJ305041 | GCGAGACTGC TAGAAGCAAT AGATGCAGAG CCAGTAACAC AGCCCACAAA |
| >AJ305042 | GCAAAACTAT TAGAAGCAAT AGATGCAGAA CCAGTAACAC AGCTCATAAA |
| | 951 1000 |
| NC_001463 (gag) | AGATTATCTA AAGCTAACAC TATCTTATAC AAATGCATCA GCAGATTGTC |
| >AJ305040 | AGACTATCTA AAGTTAACAT TATCTTATAC AAATGCGTCC TCAGACTGTC |
| >AJ305041 | AGAATATCTA AAACTAACAT TATCTTATAC AAATGCATCC TCAGACTGTC |
| >AJ305042 | AGACTATCTA AAGTTAACAT TATCTTATAC AAATGCGTCC TCAGACTGTC |
| | 1001 1050 |
| NC_001463 (gag) | AGAAGCAAAT GGATAGAACA CTAGGACAAA GAGTACAACA AGCTAGTGTA |
| >AJ305040 | AAAAGCAAAT GGATAGAGTG CTGGGACAAA GAGTGCAACA AGCTAGTGTA |
| >AJ305041 | AAAAGCAAAT GGATAGAGTA CTAGGACAAA GAGTGCAACA AGCTAGTGTA |
| >AJ305042 | AAAAGCAAAT GGATAGAGTG CTGGGACAAA GAGTGCAACA AGCTAGTGTA |
| | 1051 1100 |
| NC_001463 (gag) | GAAGAAAAAA TGCAAGCATG TAGAGATGTG GGATCAGAAG GGTTCAAAAT |
| >AJ305040 | GACGAGAAAA TGCAA..... .......... .......... .......... |
| >AJ305041 | GAAGAAAAAA TGCAA..... .......... .......... .......... |
| >AJ305042 | GACGAGAAGA TGCAA..... .......... .......... .......... |
| | 1101 1150 |
| NC_001463 (gag) | GCAATTGTTA GCACAAGCAT TAAGGCCAGG AAAAGGAAAA GGGAATGGAC |
| >AJ305040 | .......... .......... .......... .......... .......... |
| >AJ305041 | .......... .......... .......... .......... .......... |
| >AJ305042 | .......... .......... .......... .......... .......... |
| | 1151 1200 |
| NC_001463 (gag) | AGCCACAAAG GTGTTACAAC TGTGGAAAAC CGGGACATCA AGCAAGGCAA |
| >AJ305040 | .......... .......... .......... .......... .......... |
| >AJ305041 | .......... .......... .......... .......... .......... |
| >AJ305042 | .......... .......... .......... .......... .......... |
| | 1201 1250 |
| NC_001463 (gag) | TGTAGACAAG GAATCATATG TCACAACTGT GGAAAGAGAG GACATATGCA |
| >AJ305040 | .......... .......... .......... .......... .......... |
| >AJ305041 | .......... .......... .......... .......... .......... |
| >AJ305042 | .......... .......... .......... .......... .......... |
| | 1251 1300 |
| NC_001463 (gag) | AAAAGAATGC AGAGGAAAGA GAGACATAAG GGGAAAACAG CAGGGAAACG |
| >AJ305040 | .......... .......... .......... .......... .......... |
| >AJ305041 | .......... .......... .......... .......... .......... |
| >AJ305042 | .......... .......... .......... .......... .......... |
| | 1301 1347 |
| NC_001463 (gag) | GGAGGAGGGG GATACGTGTG GTGCCGTCCG CTCCTCCTAT GGAATAA |
| >AJ305040 | .......... .......... .......... .......... .......... |
| >AJ305041 | .......... .......... .......... .......... .......... |
| >AJ305042 | .......... .......... .......... .......... .......... |

TABLE 10

Pileup
MSF: 728 Type: N Check: 9403 . . .
Name: NC_001463(gag720bp) (SEQ ID NO: 39) Len: 728 Check: 5765 Weight: 0
Name: >AY047362 (SEQ ID NO: 40) Len: 728 Check: 3638 Weight: 0
//

| | |
|---|---|
| | 1 50 |
| NC_001463 (gag720bp) | ATGGTGAGTC TAGATAGAGA CATGGCGAGG CAAGTCTCCG GGGGGAAAAG |
| >AY047362 | .......... .......... .......... .......... .......... |
| | 51 100 |
| NC_001463 (gag720bp) | AGATTATCCT GAGCTCGAAA AATGTATCAA GCATGCATGC AAGATAAAAG |
| >AY047362 | ......TAAA GATATATTAG AA.GTATTGG CCATG.ATGC CTGGAAATAG |
| | 101 150 |
| NC_001463 (gag720bp) | TTCGACTCAG AGGGGAGCAC TTGACAGAAG GAAATTGTTT ATGGTGCCTT |
| >AY047362 | AGC...TCAA AAAGAGTTAA TTCA...AGG GAAATTGAAT GAAGAAGCAG |
| | 151 200 |
| NC_001463 (gag720bp) | AAAACATTAG ATTACATGTT TGAGGACCAT AAAGAGGAAC CTTGGACAAA |
| >AY047362 | AAAGGTGGAG AAGGAATAAT CCACCACCTC AAGCAGG..C GGAGGATTAA |
| | 201 250 |
| NC_001463 (gag720bp) | AGTAAAATTT AGGACAATAT GGCAGAAGGT GAAG..AATC TAACTCCTGA |
| >AY047362 | C..AG..TGG ATCAAATTAT GGGGGTAGGA CAAACAAATC AAGCAGCGGC |
| | 251 300 |
| NC_001463 (gag720bp) | GGAGAGTAAC AAAAAAGACT TTATGTCTTT GCAGGCCACA TTAGCGGGTC |
| >AY047362 | ACAGGCTAAC ATGGATCAG. .......... GCAAGACAAA TATGC..CTG |
| | 301 350 |
| NC_001463 (gag720bp) | TAATGTGTTG CCAAATGGGG ATGAGACCTG AGACATTGCA AGATGCAATG |
| >AY047362 | CAATGGGTAA TAACAGCACT AAGAGCAGTG AGACAT.... ..A.....TG |
| | 351 400 |
| NC_001463 (gag720bp) | GCTACAGTAA TCATGAAAGA TGGGTTACTG GAACAAGAGG AAAAGAAGGA |
| >AY047362 | GCT.CACAAA CCAGGGA..A TCCGATGCT. .....ACT.. AAAGCAA..A |
| | 401 450 |
| NC_001463 (gag720bp) | AGACAAAAGA GAAA.AGGAA GAGAGTGTCT TCCCAATAGT AGTGCAAGCA |
| >AY047362 | AAACAAATGA GTCATATGAA GATTTTGCCG ...CAAGACT GCTAGAAGCA |
| | 451 500 |
| NC_001463 (gag720bp) | GCAGGAGGGA GAAGCTGGAA AGCAGTAGAT TCTGTAATGT TCCAGCAACT |
| >AY047362 | ATAG.ATGCA GAACCAGTTA CAAAGCAAAT AAAAGAATAT TT.....AAA |
| | 501 550 |
| NC_001463 (gag720bp) | GCAAACAGTA GCAATGCAGC ATGGCCTCGT GTCTGAGGAC TTTGAAAGGC |
| >AY047362 | GTTAACATTA TCT.TACACA AATGCATC.. ..CTCAG.AC TGTAAGAAAC |
| | 551 600 |
| NC_001463 (gag720bp) | AGTTGGCATA TTATGCTACT ACCTGGACAA GTA.AAGACA TACTAGAAGT |
| >AY047362 | AGATGG.ATA GAGTACTAGG ACAGAGAGTG CAACAAGCTA GTGTGGAAGA |
| | 601 650 |
| NC_001463 (gag720bp) | ATTG..GCCA TGATGCCTGG AAATAGAGCT CAAAAGGAGT TA..ATTCAA |
| >AY047362 | AAAAATGCAA GCATGCAGAG ATGT.GGGAT CAGAAGGATT CAGAATGC.. |
| | 651 700 |
| NC_001463 (gag720bp) | GGGAAATTAA ATGAAGAAGC AGAAAGGTGG AGAAGGAATA ATCCACCACC |
| >AY047362 | .......... .......... .......... .......... .......... |
| | 701 728 |
| NC_001463(gag720bp) | TCCAGCAGGA GGAGGATTAA CAGTGGAT |
| >AY047362 | .......... .......... ........ |

Pileup
MSF: 1347 Type: N Check: 3238 . . .
Name: NC_001463 (gag) (SEQ ID NO: 41) Len: 1347 Check: 6959 Weight: 0
Name: >AY047362 (SEQ ID NO: 42) Len: 1347 Check: 6279 Weight: 0
//

| | |
|---|---|
| | 1 50 |
| NC_001463 (gag) | ATGGTGAGTC TAGATAGAGA CATGGCGAGG CAAGTCTCCG GGGGGAAAAG |
| >AY047362 | .......... .......... .......... .......... .......... |
| | 51 100 |
| NC_001463 (gag) | AGATTATCCT GAGCTCGAAA AATGTATCAA GCATGCATGC AAGATAAAAG |
| >AY047362 | .......... .......... .......... .......... .......... |
| | 101 150 |
| NC_001463 (gag) | TTCGACTCAG AGGGGAGCAC TTGACAGAAG GAAATTGTTT ATGGTGCCTT |
| >AY047362 | .......... .......... .......... .......... .......... |
| | 151 200 |
| NC_001463 (gag) | AAAACATTAG ATTACATGTT TGAGGACCAT AAAGAGGAAC CTTGGACAAA |
| >AY047362 | .......... .......... .......... .......... .......... |
| | 201 250 |
| NC_001463 (gag) | AGTAAAATTT AGGACAATAT GGCAGAAGGT GAAGAATCTA ACTCCTGAGG |
| >AY047362 | .......... .......... .......... .......... .......... |
| | 251 300 |
| NC_001463 (gag) | AGAGTAACAA AAAAGACTTT ATGTCTTTGC AGGCCACATT AGCGGGTCTA |
| >AY047362 | .......... .......... .......... .......... .......... |
| | 301 350 |
| NC_001463 (gag) | ATGTGTTGCC AAATGGGGAT GAGACCTGAG ACATTGCAAG ATGCAATGGC |
| >AY047362 | .......... .......... .......... .......... .......... |
| | 351 400 |
| NC_001463 (gag) | TACAGTAATC ATGAAAGATG GGTTACTGGA ACAAGAGGAA AAGAAGGAAG |
| >AY047362 | .......... .......... .......... .......... .......... |
| | 401 450 |
| NC_001463 (gag) | ACAAAAGAGA AAAGGAAGAG AGTGTCTTCC CAATAGTAGT GCAAGCAGCA |
| >AY047362 | .......... .......... .......... .......... .......... |
| | 451 500 |
| NC_001463 (gag) | GGAGGGAGAA GCTGGAAAGC AGTAGATTCT GTAATGTTCC AGCAACTGCA |
| >AY047362 | .......... .......... .......... .......... .......... |
| | 501 550 |
| NC_001463 (gag) | AACAGTAGCA ATGCAGCATG GCCTCGTGTC TGAGGACTTT GAAAGGCAGT |
| >AY047362 | .......... .......... .......... .......... .......... |
| | 551 600 |
| NC_001463 (gag) | TGGCATATTA TGCTACTACC TGGACAAGTA AAGACATACT AGAAGTATTG |
| >AY047362 | .......... .......... ........TA AAGATATATT AGAAGTATTG |
| | 601 650 |
| NC_001463 (gag) | GCCATGATGC CTGGAAATAG AGCTCAAAAG GAGTTAATTC AAGGGAAATT |
| >AY047362 | GCCATGATGC CTGGAAATAG AGCTCAAAAA GAGTTAATTC AAGGGAAATT |
| | 651 700 |
| NC_001463 (gag) | AAATGAAGAA GCAGAAAGGT GGAGAAGGAA TAATCCACCA CCTCCAGCAG |
| >AY047362 | GAATGAAGAA GCAGAAAGGT GGAGAAGGAA TAATCCACCA CCTCAAGCAG |
| | 701 750 |
| NC_001463 (gag) | GAGGAGGATT AACAGTGGAT CAAATTATGG GGGTAGGACA AACAAATCAA |
| >AY047362 | GCGGAGGATT AACAGTGGAT CAAATTATGG GGGTAGGACA AACAAATCAA |
| | 751 800 |
| NC_001463 (gag) | GCAGCAGCAC AAGCTAACAT GGATCAGGCA AGGCAAATAT GCCTGCAATG |
| >AY047362 | GCAGCGGCAC AGGCTAACAT GGATCAGGCA AGACAAATAT GCCTGCAATG |
| | 801 850 |
| NC_001463 (gag) | GGTAATAAAT GCATTAAGAG CAGTAAGACA TATGGCGCAC AGGCCAGGGA |
| >AY047362 | GGTAATAACA GCACTAAGAG CAGTGAGACA TATGGCTCAC AAACCAGGGA |
| | 851 900 |
| NC_001463 (gag) | ATCCAATGCT AGTAAAGCAA AAAACGAATG AGCCATATGA AGATTTTGCA |
| >AY047362 | ATCCGATGCT AGTAAAGCAA AAAACAAATG AGTCATATCA AGATTTTGCC |
| | 901 950 |
| NC_001463 (gag) | GCAAGACTGC TAGAAGCAAT AGATGCAGAG CCAGTTACAC AGCCTATAAA |
| >AY047362 | GCAAGACTGC TAGAAGCAAT AGATGCAGAA CCAGTTACAA AGCAAATAAA |
| | 951 1000 |
| NC_001463 (gag) | AGATTATCTA AAGCTAACAC TATCTTATAC AAATGCATCA GCAGATTGTC |
| >AY047362 | AGAATATTTA AAGTTAACAT TATCTTACAC AAATGCATCC TCAGACTGTA |
| | 1001 1050 |
| NC_001463 (gag) | AGAAGCAAAT GGATAGAACA CTAGGACAAA GAGTACAACA AGCTAGTGTA |
| >AY047362 | AGAAACAGAT GGATAGAGTA CTAGGACAGA GAGTGCAACA AGCTAGTGTG |
| | 1051 1100 |
| NC_001463 (gag) | GAAGAAAAAA TGCAAGCATG TAGAGATGTG GGATCAGAAG GGTTCAAAAT |
| >AY047362 | GAAGAAAAAA TGCAAGCATG CAGAGATGTG GGATCAGAAG GATTCAGAAT |
| | 1101 1150 |
| NC_001463 (gag) | GCAATTGTTA GCACAAGCAT TAAGGCCAGG AAAAGGAAAA GGGAATGGAC |
| >AY047362 | GC........ .......... .......... .......... .......... |
| | 1151 1200 |
| NC_001463 (gag) | AGCCACAAAG GTGTTACAAC TGTGGAAAAC CGGGACATCA AGCAAGGCAA |
| >AY047362 | .......... .......... .......... .......... .......... |
| | 1201 1250 |
| NC_001463 (gag) | TGTAGACAAG GAATCATATG TCACAACTGT GGAAAGAGAG GACATATGCA |
| >AY047362 | .......... .......... .......... .......... .......... |
| | 1251 1300 |
| NC_001463 (gag) | AAAAGAATGC AGAGGAAAGA GAGACATAAG GGGAAAACAG CAGGGAAACG |
| >AY047362 | .......... .......... .......... .......... .......... |
| | 1301 1347 |
| NC_001463 (gag) | GGAGGAGGGG GATACGTGTG CTGCCGTCCG CTCCTCCTAT GGAATAA |
| >AY047362 | .......... .......... .......... .......... .......... |

TABLE 11

Pileup
MSF: 733 Type: N Check: 5855 . . .
Name: NC_001463 (gag720bp) (SEQ ID NO: 43) Len: 733 Check: 9482 Weight: 0
Name: >AY081139 (SEQ ID NO: 44) Len: 733 Check: 6373 Weight: 0
//

| | |
|---|---|
| | 1 50 |
| NC_001463 (gag720bp) | ATGGTGAGTC TAGATAGAGA CATGGCGAGG CAAGTCTCCG GGGGGAAAAG |
| >AY081139 | .......... .......... .......... ......TGCC GTAGACTCTG |
| | 51 100 |
| NC_001463 (gag720bp) AGATTATCCT G.AGCTCGAA AAATGTATCA AGCATGCATG CAAGATAAAA | |
| >AY081139 | TGATGTTCCA CCAGCT.GCA TACAGTAGCA ATGCCGCATG GCCTCGTGTC |
| | 101 150 |
| NC_001463 (gag720bp) | GTTCGACTCA GAGGGGAGCA CTTGACAGAA GGAAATTGTT TATGGTGCCT |
| >AY081139 | TGAGGACTTT GAAAGG..CA GTTGGCATAT TATGCTACTA CCTGGACAAG |
| | 151 200 |
| NC_001463 (gag720bp) | TAAAACATTA GATTACATGT .TTGAGGACC ATAAAGAGGA ACCTTGGACA |
| >AY081139 | TAAAGA..TA TACTGGAAGT ATTGGCCATG ATGCCTGGGA ATAGAGCTCA |
| | 201 250 |
| NC_001463 (gag720bp) | AAAGTAA..A ATTTAGGACA ATATGGCAGA AGGTGAAGAA TCTAACTCCT |
| >AY081139 | AAAAGAATTA ATTCAAGGAA AATTAAATGA AGAAGCAGAA .......... |
| | 251 300 |
| NC_001463 (gag720bp) | GAGGAGAGTA ACAAAAAAGA CTTTATGTCT TTGCAGGCCA CATTAGCGGG |
| >AY081139 | .AGGTGGAGA AGGAATAATC CACCACCTCA A.GCAGGCG. .....GAGGA |
| | 301 350 |
| NC_001463 (gag720bp) | TCTAATGTGT TGCCAA..AT GGGGATGAGA CCTGAGACAT TGCAAGATGC |
| >AY081139 | TTAACAGTGG ATCAAATTAT GGGGGTAGGA CAAACAAATC AAGCAGCTGC |
| | 351 400 |
| NC_001463 (gag720bp) | AATGGCTA.C AGTAATCATG AAAGATGGGT TACTGGAACA AGAGGAAAAG |
| >AY081139 | ACAAGCTAAC ATGGATCAGG CAAGACAAAT A..TGCCTGC AATGGGTAAT |
| | 401 450 |
| NC_001463 (gag720bp) | AAGGAAGACA AAAGAGAAAA GGAAGAGAGT GTCTTCCCAA TAGTAGTGCA |
| >AY081139 | ATC..AGCCT TAAGAGCAGT GAGACATA.T GTCT..CATA AACCAGGG.A |
| | 451 500 |
| NC_001463 (gag720bp) | AGCAGCAGGA GGGAGAAGCT GGAAAGCAGT AGATTCTGTA ATGTTCCAGC |
| >AY081139 | ATCCGCTGCT AGTA.AAGCA AAAAACAAAT GAGTCATATG AAGATTTTGC |
| | 501 550 |
| NC_001463 (gag720bp) | AACTGCAAAC ..AGTAGCAA TGCAGCATGG CCTCGTGTCT GAGGACTTTG |
| >AY081139 | AGCTAGACTG CTAGAAGCAA TAGATCCAGC CCCAGTAGCA CATC.CTATA |
| | 551 600 |
| NC_001463 (gag720bp) | AAAGGCAGTT GGCATATTAT GCTAC....T ACCTGGACAA GTAAAGACAT |
| >AY081139 | AAAGATTATT TAAAGTTAAC ACTATCTTAT ACGAATGCAT CATCAGATTG |
| | 601 650 |
| NC_001463 (gag720bp) | ACTAGAAGTA TTGGCCATGA TGCCTGGAAA TAGAGCTCAA AAGGAGTTAA |
| >AY081139 | TCAAAAGCAA ATGGGTAGAA TGCTAGGATC GAGAGTCCAT CA..AGCCAG |
| | 651 700 |
| NC_001463 (gag720bp) | TTCAAGGGAA ATTAAATGAA GAAGCAGAAA GGTGGAGAAG GAATAATCCA |
| >AY081139 | TGTGGGCCAA AAAA...... .......... .......... .......... |
| | 701 733 |
| NC_001463(gag720bp) | CCACCTCCAG CAGGAGGAGG ATTAACAGTG GAT |
| >AY081139 | .......... .......... .......... ... |

PileUp
MSF: 1347 Type: N Check: 2072 . . .
Name: NC_001463 (gag) (SEQ ID NO: 45) Len: 1347 Check: 6959 Weight: 0
Name: >AY081139 (SEQ ID NO: 46) Len: 1347 Check: 5113 Weight: 0
//

| | |
|---|---|
| | 1 50 |
| NC_001463 (gag) | ATGGTGAGTC TAGATAGAGA CATGGCGAGG CAAGTCTCCG GGGGGAAAAG |
| >AY081139 | .......... .......... .......... .......... .......... |
| | 51 100 |
| NC_001463 (gag) | AGATTATCCT GAGCTCGAAA AATGTATCAA GCATGCATGC AAGATAAAAG |
| >AY081139 | .......... .......... .......... .......... .......... |
| | 101 150 |
| NC_001463 (gag) | TTCGACTCAG AGGGGAGCAC TTGACAGAAG GAAATTGTTT ATGGTGCCTT |
| >AY081139 | .......... .......... .......... .......... .......... |
| | 151 200 |
| NC_001463 (gag) | AAAACATTAG ATTACATGTT TGAGGACCAT AAAGAGGAAC CTTGGACAAA |
| >AY081139 | .......... .......... .......... .......... .......... |
| | 201 250 |
| NC_001463 (gag) | AGTAAAATTT AGGACAATAT GGCAGAAGGT GAAGAATCTA ACTCCTGAGG |
| >AY081139 | .......... .......... .......... .......... .......... |
| | 251 300 |
| NC_001463 (gag) | AGAGTAACAA AAAAGACTTT ATGTCTTTGC AGGCCACATT AGCGGGTCTA |
| >AY081139 | .......... .......... .......... .......... .......... |
| | 301 350 |
| NC_001463 (gag) | ATGTGTTGCC AAATGGGGAT GAGACCTGAG ACATTGCAAG ATGCAATGGC |
| >AY081139 | .......... .......... .......... .......... .......... |
| | 351 400 |
| NC_001463 (gag) | TACAGTAATC ATGAAAGATG GGTTACTGGA ACAAGAGGAA AAGAAGGAAG |
| >AY081139 | .......... .......... .......... .......... .......... |
| | 401 450 |
| NC_001463 (gag) | ACAAAAGAGA AAAGGAAGAG AGTGTCTTCC CAATAGTAGT GCAAGCAGCA |
| >AY081139 | .......... .......... .......... .......... .......... |
| | 451 500 |
| NC_001463 (gag) | GGAGGGAGAA GCTGGAAAGC AGTAGATTCT GTAATGTTCC AGCAACTGCA |
| >AY081139 | .......... .......TGC CGTAGACTCT GTGATGTTCC ACCAGCTGCA |
| | 501 550 |
| NC_001463 (gag) | AACAGTAGCA ATGCAGCATG GCCTCGTGTC TGAGGACTTT GAAAGGCAGT |
| >AY081139 | TACAGTAGCA ATGCCGCATG GCCTCGTGTC TGAGGACTTT GAAAGGCAGT |
| | 551 600 |
| NC_001463 (gag) | TGGCATATTA TGCTACTACC TGGACAAGTA AAGACATACT AGAAGTATTG |
| >AY081139 | TGGCATATTA TGCTACTACC TGGACAAGTA AAGATATACT GGAAGTATTG |
| | 601 650 |
| NC_001463 (gag) | GCCATGATGC CTGGAAATAG AGCTCAAAAG GAGTTAATTC AAGGGAAATT |
| >AY081139 | GCCATGATGC CTGGGAATAG AGCTCAAAAA GAATTAATTC AAGGAAAATT |
| | 651 700 |
| NC_001463 (gag) | AAATGAAGAA GCAGAAAGGT GGAGAAGGAA TAATCCACCA CCTCCAGCAG |
| >AY081139 | AAATGAAGAA GCAGAAAGGT GGAGAAGGAA TAATCCACCA CCTCAAGCAG |
| | 701 750 |
| NC_001463 (gag) | GAGGAGGATT AACAGTGGAT CAAATTATGG GGGTAGGACA AACAAATCAA |
| >AY081139 | GCGGAGGATT AACAGTGGAT CAAATTATGG GGGTAGGACA AACAAATCAA |
| | 751 800 |
| NC_001463 (gag) | GCAGCAGCAC AAGCTAACAT GGATCAGGCA AGGCAAATAT GCCTGCAATG |
| >AY081139 | GCAGCTGCAC AAGCTAACAT GGATCAGGCA AGACAAATAT GCCTGCAATG |
| | 801 850 |
| NC_001463 (gag) | GGTAATAAAT GCATTAAGAG CAGTAAGACA TATGGCGCAC AGGCCAGGGA |
| >AY081139 | GGTAATATCA GCCTTAAGAG CAGTGAGACA TATGTCTCAT AAACCAGGGA |
| | 851 900 |
| NC_001463 (gag) | ATCCAATGCT AGTAAAGCAA AAAACGAATG AGCCATATGA AGATTTTGCA |
| >AY081139 | ATCCGCTGCT AGTAAAGCAA AAAACAAATG AGTCATATGA AGATTTTGCA |
| | 901 950 |
| NC_001463 (gag) | GCAAGACTGC TAGAAGCAAT AGATGCAGAG CCAGTTACAC AGCCTATAAA |
| >AY081139 | GCTAGACTGC TAGAAGCAAT AGATCCAGCC CCAGTAGCAC ATCCTATAAA |
| | 951 1000 |
| NC_001463 (gag) | AGATTATCTA AAGCTAACAC TATCTTATAC AAATGCATCA GCAGATTGTC |
| >AY081139 | AGATTATTTA AAGTTAACAC TATCTTATAC GAATGCATCA TCAGATTGTC |
| | 1001 1050 |
| NC_001463 (gag) | AGAAGCAAAT GGATAGAACA CTAGGACAAA GAGTACAACA AGCTAGTGTA |
| >AY081139 | AAAAGCAAAT GGGTAGAATG CTAGGATCGA GAGTCCATCA AGCCAGTGTG |
| | 1051 1100 |
| NC_001463 (gag) | GAAGAAAAAA TGCAAGCATG TAGAGATGTG GGATCAGAAG GGTTCAPAAT |
| >AY081139 | GGCCAAAAAA .......... .......... .......... .......... |
| | 1101 1150 |
| NC_001463 (gag) | GCAATTGTTA GCACAAGCAT TAAGGCCAGG AAAAGGAAAA GGGAATGGAC |
| >AY081139 | .......... .......... .......... .......... .......... |
| | 1151 1200 |
| NC_001463 (gag) | AGCCACAAAG GTGTTACAAC TGTGGAAAAC CGGGACATCA AGCAAGGCAA |
| >AY081139 | .......... .......... .......... .......... .......... |
| | 1201 1250 |
| NC_001463 (gag) | TGTAGACAAG GAATCATATG TCACAACTGT GGAAAGAGAG GACATATGCA |
| >AY081139 | .......... .......... .......... .......... .......... |
| | 1251 1300 |
| NC_001463 (gag) | AAAAGAATGC AGAGGAAAGA GAGACATAAG GGGAAAACAG CAGGGAAACG |
| >AY081139 | .......... .......... .......... .......... .......... |
| | 1301 1347 |
| NC_001463 (gag) | GGAGGAGGGG GATACGTGTG GTGCCGTCCG CTCCTCCTAT GGAATAA |
| >AY081139 | .......... .......... .......... .......... ....... |

TABLE 12

Pileup
MSF: 731 Type: N Check: 9546 . . .
Name: NC_001463 (gag720bp) (SEQ ID NO: 47) Len: 731 Check: 7595 Weight: 0
Name: >AY101347 (SEQ ID NO: 48) Len: 731 Check: 7962 Weight: 0
Name: >AY101348 (SEQ ID NO: 49) Len: 731 Check: 3989 Weight: 0
//

| | |
|---|---|
| | 1 50 |
| NC_001463 (gag720bp) | ATGGTGAGTC TAGATAGAGA CATGGCGAGG CAAGTCTCCG GGGGGAAAAG |
| >AY101347 | .......... .......... .......... .......... .......... |
| >AY101348 | .......... .......... .......... .......... .......... |
| | 51 100 |
| NC_001463 (gag720bp) | AGATTATCCT GAGCTCGAAA AATGTATCAA GCATGCATGC AAGATAAAAG |
| >AY101347 | .......... .......... .....AGCAG TAGATTCTGT AATGTTCCAG |
| >AY101348 | .......... .......... .....AGCCG TAGATTCTGT AATGTTCCAG |
| | 101 150 |
| NC_001463 (gag720bp) | TTCGACT.CA GAGGGGAGCA CTTGACAGAA GGAAATTGTT TATGGTGCCT |
| >AY101347 | ..CAACTGCA AACAGTAGCA AT..GCAGCA TGGACTCGTG TATGAAGACT |
| >AY101348 | ..CAGCTGCA AACAGTAGCA AT..GCAGCA TGGCCTCGTG TCAGAGGACT |
| | 151 200 |
| NC_001463 (gag720bp) | TAAAACATTA GATTACATGT TTGAGGACCA TAAAGAGGAA CCTTGGACAA |
| >AY101347 | TTGAAAGGCT GTCGGCATAT TATGCTACTA CCTGGAC.AA GTAAAGATAT |
| >AY101348 | TTGAAAGGCT TCCAGCATAT CATGCTACTA CCTGGGC.AA GTAAAGATAT |
| | 201 250 |
| NC_001463 (gag720bp) | AAGTAAAATT TAGGACAATA TGGCAGAAGG TGAAGAATCT AACTCCTGAG |
| >AY101347 | ACTGGAAGTA TTGGCCATGA TGCCTG..G. ....GAATAG AGCTCAAAAA |
| >AY101348 | CTTAGAAGTA CTGGCCATGA TGCCTG..G. ....AAATAG AGCTCAAAAA |
| | 251 300 |
| NC_001463 (gag720bp) | GAGAGTAA.. CAAAAAAGAC TTTATGTCTT TGCAGGCCAC ATTAGCGGGT |
| >AY101347 | GA.ATTAATT CAAGGAAAAT TAAATGAAGA AGCAGAAAGG TGGAGAAGGA |
| >AY101348 | GA.GTTAATT CAAGGGAAAT TAAATGAAGA AGCAGAGAGG TGGAGAAGGA |
| | 301 350 |
| NC_001463 (gag720bp) | CTAATGTGTT GCCAAATGGG GATGAGACCT GAGACATTGC AAGATGCAAT |
| >AY101347 | ATAATCCACC ACCTCAAGCA GGCG.GAGGA TTAACAGTGG ATCAAATTAT |
| >AY101348 | ATAATCCACC ACCTCCAGCA GGAG.GAGGG TTAACAGTGG ATCAAATTAT |
| | 351 400 |
| NC_001463 (gag720bp) | GGCTACAGTA ATCATGAAAG ATGG.GTTAC TGGAACAAGA GGAAAAGAAG |
| >AY101347 | GGGGGTAGGA CAAACAAATC AAGCAGCTGC ACAAGCTAAC ATGGATCAGG |
| >AY101348 | GGGAGTAGGA CAAACAAATC AGGCAGCGGC ACAAGCAAAC ATGGATCAGG |
| | 401 450 |
| NC_001463 (gag720bp) | GAAGACAAAA GAGAAAAGGA AGAG.AGTGT CTTCCC.AAT AGTAGTGCAA |
| >AY101347 | CAAGACAAAT ATGCCTGCAA TGGGTAATAT CAGCCTTAAG AGCAGTGAGA |
| >AY101348 | CAAGACAAAT ATGCCTACAA TGGGTGATAT CAGCACTAAG AGCAGTAAGG |
| | 451 500 |
| NC_001463 (gag720bp) | GCAGCAGGAG GGAGAAGCTG GAAAGCAGTA GATTCTGTAA TGTTCCAGCA |
| >AY101347 | .CATATGTCT CATAAACCAG GGAATCCGCT GCTAGTA.AA GCAAAAAACA |
| >AY101348 | .CATATGGCT CACAAGCCAG GGAATCCAAT GTTAGTA.AA GCAAAAAGCA |
| | 501 550 |
| NC_001463 (gag720bp) | ACTG...CAA ACAGTAGCAA TGCAGCATGG CCTCGTGTCT GAGGACTTTG |
| >AY101347 | AATGAGTCAT ATGAAGATTT TGCAGCAAGA CTGCTAGAAG CAATAGATGC |
| >AY101348 | AATGAGCCAT ATGAAGAATT TGCAGCAAGG CTGCTGGAAG CAATAGATGC |
| | 551 600 |
| NC_001463 (gag720bp) | AAAGGCAGTT GG.CATATTA TGCTACTACC TGGACAAGTA AAGAC.ATAC |
| >AY101347 AGAGCCAGTA GCACATCCTA TAAAAGAATA CTTA.AAGTT AACACTATCT | |
| >AY101348 CGAGCCAGTT AATCAGCCCA TAAAAGAATA TCTA.AAACT AACGTTGTCT | |
| | 601 650 |
| NC_001463 (gag720bp) | TAGAAGTATT GGCCATGATG CCTGGAAATA GAGCTCAAAA GGAGTTAATT |
| >AY101347 | TATACGAATG CATCATCA.G ATTGTCAAAA C....CAAAT GGATAGAATG |
| >AY101348 | TATACGAATG CATCCTCA.C ATTGTCAGAA G....CAAAT GGATAGAACA |
| | 651 700 |
| NC_001463 (gag720bp) | CAAGGGAAAT TAAATCAAGA AGCAGAAAGG TGGAGAAGGA ATAATCCACC |
| >AY101347 | CTGG...AAT CAAGAGTACA ACAAGCTAG. TGTAGAACAA AAAA...... |
| >AY101348 | CTAG...GAC AAAGAGTCAA ACAAGCTAG. TGTAGAACAA AAAA...... |
| | 701 731 |
| NC_001463 (gag720bp) | ACCTCCAGCA GGACGAGGAT TAACAGTGCA T |
| >AY101347 | .......... .......... .......... . |
| >AY101348 | .......... .......... .......... . |

PileUp
MSF: 1347 Type: N Check: 2815 . . .
Name: NC_001463 (gag) (SEQ ID NO: 50) Len: 1347 Check: 6959 Weight: 0
Name: >AY101347 (SEQ ID NO: 51) Len: 1347 Check: 969 Weight: 0
Name: >AY101348 (SEQ ID NO: 52) Len: 1347 Check: 4887 Weight: 0
//

| | |
|---|---|
| | 1 50 |
| NC_001463 (gag) | ATGGTGAGTC TAGATAGAGA CATGGCGAGG CAAGTCTCCG GGGGGAAAAG |
| >AY101347 | .......... .......... .......... .......... .......... |
| >AY101348 | .......... .......... .......... .......... .......... |
| | 51 100 |
| NC_001463 (gag) | AGATTATCCT GAGCTCGAAA AATGTATCAA GCATGCATGC AAGATAAAAG |
| >AY101347 | .......... .......... .......... .......... .......... |
| >AY101348 | .......... .......... .......... .......... .......... |
| | 101 150 |
| NC_001463 (gag) | TTCGACTCAG AGGGGAGCAC TTGACAGAAG GAAATTGTTT ATGGTGCCTT |
| >AY101347 | .......... .......... .......... .......... .......... |
| >AY101348 | .......... .......... .......... .......... .......... |
| | 151 200 |
| NC_001463 (gag) | AAAACATTAG ATTACATGTT TGAGGACCAT AAAGAGGAAC CTTGGACAAA |
| >AY101347 | .......... .......... .......... .......... .......... |
| >AY101348 | .......... .......... .......... .......... .......... |
| | 201 250 |
| NC_001463 (gag) | AGTAAAATTT AGGACAATAT GGCAGAAGGT GAAGAATCTA ACTCCTGAGG |
| >AY101347 | .......... .......... .......... .......... .......... |
| >AY101348 | .......... .......... .......... .......... .......... |
| | 251 300 |
| NC_001463 (gag) | AGAGTAACAA AAAAGACTTT ATGTCTTTGC AGGCCACATT ACCGGGTCTA |
| >AY101347 | .......... .......... .......... .......... .......... |
| >AY101348 | .......... .......... .......... .......... .......... |
| | 301 350 |
| NC_001463 (gag) | ATGTGTTGCC AAATGGGGAT GAGACCTGAG ACATTGCAAG ATGCAATGGC |
| >AY101347 | .......... .......... .......... .......... .......... |
| >AY101348 | .......... .......... .......... .......... .......... |
| | 351 400 |
| NC_001463 (gag) | TACAGTAATC ATGAAAGATG GGTTACTGGA ACAAGAGGAA AAGAAGGAAG |
| >AY101347 | .......... .......... .......... .......... .......... |
| >AY101348 | .......... .......... .......... .......... .......... |
| | 401 450 |
| NC_001463 (gag) | ACAAAAGAGA AAAGGAAGAG AGTGTCTTCC CAATAGTAGT GCAAGCAGCA |
| >AY101347 | .......... .......... .......... .......... .......... |
| >AY101348 | .......... .......... .......... .......... .......... |
| | 451 500 |
| NC_001463 (gag) | GGAGGGAGAA GCTGGAAAGC AGTAGATTCT GTAATGTTCC AGCAACTGCA |
| >AY101347 | .......... .......AGC AGTAGATTCT GTAATGTTCC AGCAACTGCA |
| >AY101348 | .......... .......AGC CGTAGATTCT GTAATGTTCC AGCAGCTGCA |
| | 501 550 |
| NC_001463 (gag) | AACAGTAGCA ATGCAGCATG GCCTCGTGTC TGAGGACTTT GAAAGGCAGT |
| >AY101347 | AACAGTAGCA ATGCAGCATG GACTCGTGTA TGAAGACTTT GAAAGGCTGT |
| >AY101348 | AACAGTAGCA ATGCAGCATG GCCTCGTGTC AGAGGACTTT GAAAGGCTTC |
| | 551 600 |
| NC_001463 (gag) | TGGCATATTA TGCTACTACC TGGACAAGTA AAGACATACT AGAAGTATTG |
| >AY101347 | CGGCATATTA TGCTACTACC TGGACAAGTA AAGATATACT GGAAGTATTG |
| >AY101348 | CAGCATATCA TGCTACTACC TGGGCAAGTA AAGATATCTT AGAAGTACTG |
| | 601 650 |
| NC_001463 (gag) | GCCATGATGC CTGGAAATAG AGCTCAAAAG GAGTTAATTC AAGGGAAATT |
| >AY101347 | GCCATGATGC CTGGGAATAG AGCTCAAAAA GAATTAATTC AAGGAAAATT |
| >AY101348 | GCCATGATGC CTGGAAATAG AGCTCAAAAA GAGTTAATTC AAGGGAAATT |
| | 651 700 |
| NC_001463 (gag) | AAATGAAGAA GCAGAAAGGT GGAGAAGGAA TAATCCACCA CCTCCAGCAG |
| >AY101347 | AAATGAAGAA GCAGAAAGGT GGAGAAGGAA TAATCCACCA CCTCAAGCAG |
| >AY101348 | AAATGAAGAA GCAGAGAGGT GGAGAAGGAA TAATCCACCA CCTCCAGCAG |
| | 701 750 |
| NC_001463 (gag) | GAGGAGGATT AACAGTGGAT CAAATTATGG GGGTAGGACA AACAAATCAA |
| >AY101347 | GCGGAGGATT AACAGTGGAT CAAATTATGG GGGTAGGACA AACAAATCAA |
| >AY101348 | GAGGAGGGTT AACAGTGGAT CAAATTATGG GAGTAGGACA AACAAATCAG |
| | 751 800 |
| NC_001463 (gag) | GCAGCAGCAC AAGCTAACAT GGATCAGGCA AGGCAAATAT GCCTGCAATG |
| >AY101347 | GCAGCTGCAC AAGCTAACAT GGATCAGGCA AGACAAATAT GCCTGCAATG |
| >AY101348 | GCAGCGGCAC AAGCAAACAT GGATCAGGCA AGACAAATAT GCCTACAATG |
| | 801 850 |
| NC_001463 (gag) | GGTAATAAAT GCATTAAGAG CAGTAAGACA TATGGCGCAC AGGCCAGGGA |
| >AY101347 | GGTAATATCA GCCTTAAGAG CAGTGAGACA TATGTCTCAT AAACCAGGGA |
| >AY101348 | GGTGATATCA GCACTAAGAG CAGTAAGGCA TATGGCTCAC AAGCCAGGGA |
| | 851 900 |
| NC_001463 (gag) | ATCCAATGCT AGTAAAGCAA AAAACGAATG AGCCATATGA AGATTTTGCA |
| >AY101347 | ATCCGCTGCT AGTAAAGCAA AAAACAAATG AGTCATATGA AGATTTTGCA |
| >AY101348 | ATCCAATGTT AGTAAAGCAA AAAGCAAATG AGCCATATGA AGAATTTGCA |
| | 901 950 |
| NC_001463 (gag) | GCAAGACTGC TAGAAGCAAT AGATGCAGAG CCAGTTACAC AGCCTATAAA |
| >AY101347 | GCAAGACTGC TAGAAGCAAT AGATGCAGAG CCAGTAGCAC ATCCTATAAA |
| >AY101348 | GCAAGGCTGC TGGAAGCAAT AGATGCCGAG CCAGTTAATC AGCCCATAAA |
| | 951 1000 |
| NC_001463 (gag) | AGATTATCTA AAGCTAACAC TATCTTATAC AAATGCATCA GCAGATTGTC |
| >AY101347 | AGAATACTTA AAGTTAACAC TATCTTATAC GAATGCATCA TCAGATTGTC |
| >AY101348 | AGAATATCTA AAACTAACGT TGTCTTATAC GAATGCATCC TCAGATTGTC |
| | 1001 1050 |
| NC_001463 (gag) | AGAAGCAAAT GGATAGAACA CTAGGACAAA GAGTACAACA AGCTAGTGTA |
| >AY101347 | AAAAGCAAAT GGATAGAATG CTGGAATCAA GAGTACAACA AGCTAGTGTA |
| >AY101348 | AGAAGCAAAT GGATAGAACA CTAGGACAAA GAGTCAAACA AGCTAGTGTA |
| | 1051 1100 |
| NC_001463 (gag) | GAAGAAAAAA TGCAAGCATG TAGAGATGTG GGATCAGAAG GGTTCAAAAT |
| >AY101347 | GAACAAAAAA .......... .......... .......... .......... |
| >AY101348 | GAACAAAAAA .......... .......... .......... .......... |
| | 1101 1150 |
| NC_001463 (gag) | GCAATTGTTA GCACAAGCAT TAAGGCCAGG AAAAGGAAAA GGGAATGGAC |
| >AY101347 | .......... .......... .......... .......... .......... |
| >AY101348 | .......... .......... .......... .......... .......... |
| | 1151 1200 |
| NC_001463 (gag) | AGCCACAAAG GTGTTACAAC TGTGGAAAAC CGGGACATCA AGCAAGGCAA |
| >AY101347 | .......... .......... .......... .......... .......... |
| >AY101348 | .......... .......... .......... .......... .......... |
| | 1201 1250 |
| NC_001463 (gag) | TGTAGACAAG GAATCATATG TCACAACTGT GGAAAGAGAG GACATATGCA |
| >AY101347 | .......... .......... .......... .......... .......... |
| >AY101348 | .......... .......... .......... .......... .......... |
| | 1251 1300 |
| NC_001463 (gag) | AAAAGAATGC AGAGGAAAGA GAGACATAAG GGGAAAACAG CAGGGAAACG |
| >AY101347 | .......... .......... .......... .......... .......... |
| >AY101348 | .......... .......... .......... .......... .......... |
| | 1301 1347 |
| NC_001463 (gag) | GGAGGAGGGG GATACGTGTG GTGCCGTCCG CTCCTCCTAT GGAATAA |
| >AY101347 | .......... .......... .......... .......... ....... |
| >AY101348 | .......... .......... .......... .......... ....... |

TABLE 13

Pileup
MSF: 720 Type: N Check: 3690 . . .
Name: NC_001463 (gag720bp) (SEQ ID NO: 53) Len: 720 Check: 5792 Weight: 0
Name: >L78446 (SEQ ID NO: 54) Len: 720 Check: 272 Weight: 0
Name: >L78447 (SEQ ID NO: 55) Len: 720 Check: 1999 Weight: 0
Name: >L78450 (SEQ ID NO: 56) Len: 720 Check: 9633 Weight: 0
Name: >L78451 (SEQ ID NO: 57) Len: 720 Check: 5177 Weight: 0
Name: >L78453 (SEQ ID NO: 58) Len: 720 Check: 817 Weight: 0
//

| | |
|---|---|
| | 1 50 |
| NC_001463 (gag720bp) | ATGGTGAGTC TAGATAGAGA CATGGCGAGG CAAGTCTCCG GGGGGAAAAG |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 51 100 |
| NC_001463 (gag720bp) | AGATTATCCT GAGCTCGAAA AATGTATCAA GCATGCATGC AAGATAAAAG |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 101 150 |
| NC_001463 (gag720bp) | TTCGACTCAG AGGGGAGCAC TTGACAGAAG GAAATTGTTT ATGGTGCCTT |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 151 200 |
| NC_001463 (gag720bp) | AAAACATTAG ATTACATGTT TGAGGACCAT AAAGAGGAAC CTTGGACAAA |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 201 250 |
| NC_001463 (gag720bp) | AGTAAAATTT AGGACAATAT GGCAGAAGGT GAAGAATCTA ACTCCTGAGG |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 251 300 |
| NC_001463 (gag720bp) | AGAGTAACAA AAAAGACTFF ATGTCTTTGC AGGCCACA~~ AGCGGGTCTA |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 301 350 |
| NC_001463 (gag720bp) | ATGTGITGCC AAATGGGGAT GAGACCTGAG ACATTGCAAG ATGCAATGGC |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 351 400 |
| NC_001463 (gag720bp) | TACAGTAATC ATGAAAGATG GGTTACTGGA ACAAGAGGAA AAGAAGGAAG |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 401 450 |
| NC_001463 (gag720bp) | ACAAAAGAGA AAAGOAAGAG AGTGTCTTCC CAATAGTAGT GCAAGCAGCA |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 451 500 |
| NC_001463 (gag720bp) | GGAGOGAGAA GCTGGAAAGC AGTAGATTCT GTAATGTTCC AGCAACTGCA |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 501 550 |
| NC_001463 (gag720bp) | AACAGTAGCA ATGCAGCATG GCCTCGTGTC TGAGGACTTT GAAAGGCAGT |
| >L78446 | .......... ...CAGCATG GCCTCGTGTC CGAGGACTTT GAAAGGCAGT |
| >L78447 | .......... ...CAGCATG GAATAGTATC AGAAGAGTTA GAGAGGCAAC |
| >L78450 | .......... ...CAACATG GGATAGTATC AGAGGAATTT GAGAGACAAA |
| >L78451 | .......... ...CAGCATG GACTAGTATC AGAAGAATTT GAAAGGCAGC |
| >L78453 | .......... ...CAGCATG GACTTGTGTC CGAAGATTTT GAGAGGCAAT |
| | 551 600 |
| NC_001463 (gag720bp) | TGGCATATTA TGCTACTACC TGGACAAGTA AAGACATACT AGAAGTATTG |
| >L78446 | TGGCATATTA TGCTACTACC TGGACAAGTA AGGACATATT AGAAGTATTG |
| >L78447 | TGTCTTATTA TGCTACCACT TGGACAAGCA AGGATATCTT AGAGGTACTA |
| >L78450 | TGTCTTATTA TGCTACCACA TGOACAAGTA AGGATATTTT AGAAGTACTA |
| >L78451 | TAGCATACTA TGCCACAACG TGGACAAGCA AAGACATACT AGAGGTGTTA |
| >L78453 | TGGCATATTA TGCTACAACC TGGACTAGTG AAGATATATT AGAAGTATTG |
| | 601 650 |
| NC_001463 (gag720bp) | GCCATGATGC CTGGAAATAG AGCTCAAAAG GAGTTAATTC AAGGGAAATT |
| >L78446 | GCCATOATGC CAGGAAATAG AGCTCAAAAG GAGCTAATTC AA........ |
| >L78447 | GCCATGATGC CTGGCAATAG AGCATTAAAA GAGCTAATAC AA........ |
| >L78450 | GCAATGATGC CCGGGAACAG AGCATTAAAG GAGCTGATAC AA........ |
| >L78451 | GCCATGATGC CAGGGAATAG AGCACAAAAA GAACTAATAC AA........ |
| >L78453 | GCTATGATGC CTGGGAATAG AGCACAGAAA GAATTAATAC AA........ |
| | 651 700 |
| NC_001463 (gag720bp) | AAATGAAGAA GCAGAAAGGT GGAGAAGGAA TAATCCACCA CCTCCAGCAG |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 701 720 |
| NC_001463 (gag720bp) | GAGGAGGATT AACAGTGGAT |
| >L78446 | .......... .......... |
| >L78447 | .......... .......... |
| >L78450 | .......... .......... |
| >L78451 | .......... .......... |
| >L78453 | .......... .......... |

PileUp
MSF: 1347 Type: N Check: 6947 . . .
Name: NC_001463 (gag) (SEQ ID NO: 59) Len: 1347 Check: 6959 Weight: 0
Name: >L78446 (SEQ ID NO: 60) Len: 1347 Check: 6690 Weight: 0
Name: >L78447 (SEQ ID NO: 61) Len:1347 Check: 8417 Weight: 0
Name: >L78450 (SEQ ID NO: 62) Len: 1347 Check: 6051 Weight: 0
Name: >L78451 (SEQ ID NO: 63) Len: 1347 Check: 1595 Weight: 0
Name: >L78453 (SEQ ID NO: 64) Len: 1347 Check: 7235 Weight: 0
//

| | |
|---|---|
| | 1 50 |
| NC_001463 (gag) | ATGGTGAGTC TAGATAGAGA CATGGCGAGG CAAGTCTCCG GGGGGAAAAG |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 51 100 |
| NC_001463 (gag) | AGATTATCCT GAGCTCGAAA AATGTATCAA GCATGCATGC AAGATAAAAG |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 101 150 |
| NC_001463 (gag) | TTCGACTCAG AGGGGACCAC TTGACAGAAG GAAATTGTTT ATGGTGCCTT |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 151 200 |
| NC_001463 (gag) | AAAACATTAG ATTACATGTT TGAGGACCAT AAAGAGGAAC CTTGGACAAA |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 201 250 |
| NC_001463 (gag) | AGTAAAATTT AGGACAATAT GGCAGAAGGT GAAGAATCTA ACTCCTGAGG |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 251 300 |
| NC_001463 (gag) | AGAGTAACAA AAAAGACTTT ATGTCTTTGC AGGCCACATT AGCGGGTCTA |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 301 350 |
| NC_001463 (gag) | ATGTGTTGCC AAATGGGGAT GAGACCTGAG ACATTGCAAG ATGCAATGGC |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 351 400 |
| NC_001463 (gag) | TACAGTAATC ATGAAAGATG GGTTACTGGA ACAAGAGGAA AAGAAGGAAG |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 401 450 |
| NC_001463 (gag) | ACAAAAGAGA AAAGGAAGAG AGTGTCTTCC CAATAGTAGT GCAAGCAGCA |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 451 500 |
| NC_001463 (gag) | GGAGGGAGAA GCTGGAAAGC AGTAGAITCT GTAATGTTCC AGCAACTGCA |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 501 550 |
| NC_001463 (gag) | AACAGTAGCA ATGCAGCATG GCCTCGTGTC TGAGGACTTT GAAAGGCAGT |
| >L78446 | .......... ...CAGCATG GCCTCGTGTC CGAGGACTTT GAAAGGCAGT |
| >L78447 | .......... ...CAGCATG GAATAGTATC AGAAGAGTTT GAGAGGCAAC |
| >L78450 | .......... ...CAACATG GGATAGTATC AGAGGAATTT GAGAGACAAA |
| >L78451 | .......... ...CAGCATG GACTAGTATC AGAAGAATTT GAAAGGCAGC |
| >L78453 | .......... ...CAGCATG GACTTGTGTC CGAAGATTTT GAGAGGCAAT |
| | 551 600 |
| NC_001463 (gag) | TGGCATATTA TGCTACTACC TGGACAAGTA AAGACATACT AGAAGTATTG |
| >L78446 | TGGCATATTA TGCTACTACC TGGACAAGTA AGGACATATT AGAAGTATTG |
| >L78447 | TGTCTTATTA TGCTACCACT TCGACAAGCA AGGATATCTT AGAGGTACTA |
| >L78450 | TGTCTTATTA TGCTACCACA TGGACAAGTA AGGATATTTT AGAAGTACTA |
| >L78451 | TAGCATACTA TGCCACAACG TGGACAAGCA AAGACATACT AGAGGTGTTA |
| >L78453 | TGGCATATTA TGCTACAACC TGGACTAGTG AAGATATATT AGAAGTATTG |
| | 601 650 |
| NC_001463 (gag) | GCCATGATGC CTGGAAATAG AGCTCAAAAG GAGTTAATTC AAGGGAAATT |
| >L78446 | GCCATGATGC CAGGAAATAG AGCTCAAAAG GAGCTAATTC AA........ |
| >L78447 | GCCATGATGC CTGGCAATAG AGCATTAAAA GAGCTAATAC AA........ |
| >L78450 | GCAATCATGC CCGGGAACAG AGCATTAAAG GAGCTGATAC AA........ |
| >L78451 | GCCATGATGC CAGGGAATAG AGCACAAAAA GAACTAATAC AA........ |
| >L78453 | GCTATGATGC CTGGGAATAG AGCACAGAAA GAATTAATAC AA........ |
| | 651 700 |
| NC_001463 (gag) | AAATGAAGAA GCAGAAAGGT GGAGAAGGAA TAATCCACCA CCTCCAGCAG |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 701 750 |
| NC_001463 (gag) | GAGGAGGATT AACAGTGGAT CAAATTATGG GGCTAGGACA AACAAATCAA |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 751 800 |
| NC_001463 (gag) | GCAGCAGCAC AAGCTAACAT GGATCAGGCA AGGCAAATAT GCCTGCAATG |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 801 850 |
| NC_001463 (gag) | GGTAATAAAT GCATTAAGAG CAGTAAGACA TATGGCGCAC AGGCCAGGGA |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 851 900 |
| NC_001463 (gag) | ATCCAATGCT AGTAAAGCAA AAAACGAATG AGCCATATGA AGATTTTGCA |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 901 950 |
| NC_001463 (gag) | CCAAGACTGC TAGAAGCAAT AGATOCAGAG CCAGTTACAC AGCCTATAAA |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 951 1000 |
| NC_001463 (gag) | AGATTATCTA AAGCTAACAC TATCTTATAC AAATGCATCA GCAGATTGTC |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 1001 1050 |
| NC_001463 (gag) | AGAAGCAAAT GGATAGAACA CTAGGACAAA GAGTACAACA AGCTAGTGTA |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 1051 1100 |
| NC_001463 (gag) | GAAGAAAAAA TGCAAGCATG TAGAGATGTG GGATCAGAAG GGTTCAAAAT |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 1101 1150 |
| NC_001463 (gag) | GCAATTGTTA GCACAAGCAT TAAGGCCAGG AAAAGGAAAA GGGAATGGAC |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 1151 1200 |
| NC_001463 (gag) | AGCCACAAAG GTGTTACAAC TGTGGAAAAC CGGGACATCA AGCAAGGCAA |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 1201 1250 |
| NC_001463 (gag) | TGTAGACAAG GAATCATATG TCACAACTGT GGAAAGAGAG GACATATGCA |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 1251 1300 |
| NC_001463 (gag) | AAAAGAATGC AGAGGAAAGA GAGACATAAG GGGAAAACAG CAGGGAAACG |
| >L78446 | .......... .......... .......... .......... .......... |
| >L78447 | .......... .......... .......... .......... .......... |
| >L78450 | .......... .......... .......... .......... .......... |
| >L78451 | .......... .......... .......... .......... .......... |
| >L78453 | .......... .......... .......... .......... .......... |
| | 1301 1347 |
| NC_001463 (gag) | GGAGGAGGGG GATACGTGTG GTGCCGTCCG CTCCTCCTAT GCAATAA |
| >L78446 | .......... .......... .......... .......... ....... |
| >L78447 | .......... .......... .......... .......... ....... |
| >L78450 | .......... .......... .......... .......... ....... |
| >L78451 | .......... .......... .......... .......... ....... |
| >L78453 | .......... .......... .......... .......... ....... |

TABLE 14
Tables for alignment of gag sequences
NC_001463(gag720bp) vs. AF015181 Positives: 41.0% Identity: 41.0%

| | NC_001463 | |
|---|---|---|
| | (gag720bp) | >AF015181 |
| NC_001463(gag720bp) | 100 | 41 |
| >AF015181 | | 100 |

NC_001463(gag) vs. AF015181 Positives: 40.6% Identity: 40.6%

| | NC_001463 | |
|---|---|---|
| | (gag) | >AF015181 |
| NC_001463(gag) | 100 | 41 |
| >AF015181 | | 100 |

NC_001463(gag 720bp) vs. AF402664˜8 Positives: 91.1% Identity: 32.2%

| | NC_001463 | | | | | |
|---|---|---|---|---|---|---|
| | (gag720bp) | >AF402664 | >AF402665 | >AF402666 | >AF402667 | >AF402668 |
| NC_001463(gag720bp) | 100 | 43 | 44 | 43 | 33 | 43 |
| >AF402664 | | 100 | 99 | 96 | 80 | 98 |
| >AF402665 | | | 100 | 97 | 80 | 99 |
| >AF402666 | | | | 100 | 81 | 96 |
| >AF402667 | | | | | 100 | 80 |
| >AF402668 | | | | | | 100 |

NC_001463(gag) vs. AF402664˜8 Positives: 49.1% Identity: 35.0%

| | NC_001463 | | | | | |
|---|---|---|---|---|---|---|
| | (gag) | >AF402664 | >AF402665 | >AF402666 | >AF402667 | >AF402668 |
| NC_001463(gag) | 100 | 45 | 46 | 44 | 36 | 45 |
| >AF402664 | | 100 | 99 | 98 | 89 | 99 |
| >AF402665 | | | 100 | 98 | 89 | 99 |
| >AF402666 | | | | 100 | 90 | 98 |
| >AF402667 | | | | | 100 | 89 |
| >AF402668 | | | | | | 100 |

NC_001463(gag720bp) vs. AJ305040˜2 Positives: 80.5% Identity: 38.1%

| | NC_001463 | | | |
|---|---|---|---|---|
| | (gag720bp) | >AJ305040 | >AJ305041 | >AJ305042 |
| NC_001463(gag720bp) | 100 | 39 | 42 | 39 |
| >AJ305040 | | 100 | 93 | 99 |
| >AJ305041 | | | 100 | 93 |
| >AJ305042 | | | | 100 |

NC_001463(gag) vs. AJ305040˜2 Positives: 44.3% Identity: 38.8%

| | NC_001463 | | | |
|---|---|---|---|---|
| | (gag) | >AJ305040 | >AJ305041 | >AJ305042 |
| NC_001463(gag) | 100 | 41 | 41 | 40 |
| >AJ305040 | | 100 | 96 | 100 |
| >AJ305041 | | | 100 | 96 |
| >AJ305042 | | | | 100 |

NC_001463(gag720bp) vs. AY047362 Positives: 40.2% Identity: 40.2%

| | NC_001463 | |
|---|---|---|
| | (gag720bp) | >AY047362 |
| NC_001463(gag720bp) | 100 | 40 |
| >AY047362 | | 100 |

NC_001463(gag) vs. AY047362 Positives: 35.7% Identity: 35.7%

| | NC_001463 | |
|---|---|---|
| | (gag) | >AY047362 |
| NC_001463(gag) | 100 | 36 |
| >AY047362 | | 100 |

NC_001463(gag720bp) vs. AY081139 Positives: 40.0% Identity: 40.0%

| | NC_001463 | |
|---|---|---|
| | (gag720bp) | >AY081139 |
| NC_001463(gag720bp) | 100 | 40 |
| >AY081139 | | 100 |

NC_001463(gag) vs. AY081139 Positives: 39.8% Identity: 39.8%

| | NC_001463 | |
|---|---|---|
| | (gag) | >AY081139 |
| NC_001463(gag) | 100 | 40 |
| >AY081139 | | 100 |

NC_001463(gag720bp) vs. AY101347˜8 Positives: 78.1% Identity: 35.0%

| | NC_001463 | | |
|---|---|---|---|
| | (gag720bp) | >AY101347 | >AY101348 |
| NC_001463(gag720bp) | 100 | 40 | 36 |
| >AY101347 | | 100 | 91 |
| >AY101348 | | | 100 |

NC_001463(gag) vs. AY101347˜8 Positives: 43.9% Identity: 37.9%

| | NC_001463 | | |
|---|---|---|---|
| | (gag) | >AY101347 | >AY101348 |
| NC_001463(gag) | 100 | 41 | 40 |
| >AY101347 | | 100 | 95 |
| >AY101348 | | | 100 |

NC_001463(gag720bp) vs. L78446, 7, 50, 51, 53 Positives: 17.6% Identity: 11.9%

| | NC_001463 | | | | | |
|---|---|---|---|---|---|---|
| | (gag720bp) | >L78446 | >L78447 | >L78450 | >L78451 | >L78453 |
| NC_001463(gag720bp) | 100 | 17 | 14 | 14 | 15 | 15 |
| >L78446 | | 100 | 96 | 96 | 97 | 97 |
| >L78447 | | | 100 | 98 | 97 | 96 |
| >L78450 | | | | 100 | 96 | 96 |
| >L78451 | | | | | 100 | 97 |
| >L78453 | | | | | | 100 |

NC_001463(gag) vs. L78446, 47, 50, 51, 53 Positives: 9.4% Identity: 6.4%

| | NC_001463 | | | | | |
|---|---|---|---|---|---|---|
| | (gag) | >L78446 | >L78447 | >L78450 | >L78451 | >L78453 |
| NC_001463(gag) | 100 | 9 | 7 | 7 | 8 | 8 |
| >L78446 | | 100 | 98 | 98 | 98 | 98 |
| >L78447 | | | 100 | 99 | 98 | 98 |
| >L78450 | | | | 100 | 98 | 98 |
| >L78451 | | | | | 100 | 98 |
| >L78453 | | | | | | 100 |

TABLE 15
NC_001463(full genome) vs.AF322109(full genome)
Positives: 68.2% Identity: 68.2%

| | NC_001463 | AF322109 |
|---|---|---|
| NC_001463 | 100 | 68 |
| AF322109 | | 100 |

NC_001463(gag) vs.AF322109(gag) Positives: 73.1% Identity: 73.1%

| | NC_001463 | AF322109 |
|---|---|---|
| | (gag) | (gag) |
| NC_001463(gag) | 100 | 73 |
| AF322109(gag) | | 100 |

NC_001463(5′LTR region) vs.AF322109(5′LTR region)
Positives: 59.8% Identity: 59.8%

| | NC_001463 | AF322109 |
|---|---|---|
| | (5′) | (5′) |
| NC_001463(5′) | 100 | 60 |
| AF322109(5′) | | 100 |

NC_001463(pol) vs.AF322109(pol) Positives: 74.9% Identity: 74.9%

| | NC_001463 | AF322109 |
|---|---|---|
| | (pol) | (pol) |
| NC_001463(pol) | 100 | 75 |
| AF322109(pol) | | 100 |

NC_001463(rev) vs.AF322109(rev) Positives: 48.3% Identity: 48.3%

| | NC_001463 | AF322109 |
|---|---|---|
| | (rev) | (rev) |
| NC_001463(rev) | 100 | 48 |
| AF322109(rev) | | 100 |

NC_001463(vif) vs.AF322109(vif) Positives: 66.0% Identity: 66.0%

| | NC_001463 | AF322109 |
|---|---|---|
| | (vif) | (vif) |
| NC_001463(vif) | 100 | 66 |
| AF322109(vif) | | 100 |

What is claimed is:

1. A recombinant caprine arthritis encephalitis virus (CAEV)-based transfer vector, comprising:
(a) A (CAEV) packaging sequence consisting essentially of (i) 5′ untranslated region consisting of nucleotides 1 to 511 of the CAEV genome and (ii) nucleotides about 1 to X of the CAEV gag-encoding sequence linked to the 3′ end of the 5′ untranslated region, wherein X is less than about 613; and
(b) cis-acting elements operably linked to the CAEV packaging sequence.

2-5. (canceled)

6. The transfer vector of claim 1, wherein X is about 327.

7. The transfer vector of claim 1, wherein the start codon of the gag-encoding sequence is mutated to prevent translation of gag protein.

8. The transfer vector of claim 7, wherein said start codon is mutated to TAG.

9. The transfer vector of claim 7, wherein the ATG codon of the gag-encoding sequence located 21 base pairs downstream of the start codon ATG is mutated to prevent translation of gag protein.

10. (cancelled)

11. The transfer vector of claim 1, wherein the cis-acting elements comprise one or more sequences selected from the group consisting of RRE (rev-responsive element) region, CAEV 3′ LTR of which U3 region is deleted, and a heterologous promoter.

12. (cancelled)

13. (cancelled)

14. The transfer vector of claim 11, wherein the heterologous promoter is the human cytomegalovirus major immediate early promoter (HCMV MIEP).

15. The transfer vector of claim 1, wherein said vector has the structure of pCAH/SINd1 shown in FIG. 3C.

16. The transfer vector of claim 1, which further comprises a transcription cassette comprising a heterologous gene operably linked to a heterologous promoter.

17. (cancelled)

18. A recombinant CAEV-based vector system comprising the transfer vector of claim 1, and a packaging vector system, wherein said packaging vector system comprises: a first polynucleotide comprising a CAEV gag-pol-encoding sequence and an RRE, and a second polynucleotide comprising a viral envelope-encoding sequence.

19. The vector system of claim 18, wherein said transfer vector further comprises a transcription cassette comprising a heterologous gene operably linked to a heterologous promoter.

20. (cancelled)

21. The vector system of claim 18, wherein said viral envelope-encoding sequence is a non-CAEV envelope-encoding sequence.

22. The vector system of claim 21, wherein said non-CAEV envelope-encoding sequence is VSV-G (vesicular somatitis virus G) glycoprotein-encoding sequence or GaLV (gibbon ape leukemia virus) envelope protein-encoding sequence.

23. (cancelled)

24. (cancelled)

25. The vector system of claim 18, wherein said vector system further comprises a third polynucleotide sequence comprising a rev-encoding sequence.

26. The vector system of claim 18, wherein said vector system further comprises a fourth polynucleotide sequence comprising a vif-encoding sequence.

27.-32. (canceled)

33. The vector system of claim 18, wherein said packaging vector system is devoid of a competent CAEV packaging sequence.

34. (cancelled)

35. The vector system of claim 18, which comprises a first vector comprising said first polynucleotide and a second vector comprising said second polynucleotide.

36.-51. (canceled)

52. A method for delivering a polypeptide into a mammalian cell comprising contacting said mammalian cell with replication-defective vector particles prepared by transfecting a cell with the recombinant CAEV-based vector system of claim 19.

53.-56. (canceled)

57. A method for delivering a polypeptide into a vertebrate comprising administering to the vertebrate replication-defective vector particles prepared by transfecting a cell with the recombinant CAEV-based vector system of claim 19.

58.-72. (canceled)

73. The transfer vector of claim 15, wherein said vector is at least 70% identical to SEQ ID NO: 68.

74.-78. (canceled)
